(19) United States
(12) Patent Application Publication
(10) Pub. No.: US 2012/0115819 A1
(43) Pub. Date: May 10, 2012

(54) Pentacycline Compounds

(76) Inventors: Roger B. Clark, Lexington, MA (US); Diana Katharine Hunt, Cambridge, MA (US); Louis Plamondon, Belmont, MA (US); Cuixiang Sun, Watertown, MA (US); Xiao-Yi Xiao, San Diego, CA (US); Magnus P. Rönn, Melrose, MA (US)

(21) Appl. No.: 13/319,513

(22) PCT Filed: May 13, 2010
(86) PCT No.: PCT/US10/34718

§ 371 Date: Jan. 19, 2012

Related U.S. Application Data
(60) Provisional application No. 61/216,087, filed on May 13, 2009.
(60) Provisional application No. 61/337,710, filed on Feb. 9, 2010.

(51) Int. Cl. A61K 31/65; C07C 275/24; C07D 211/14; C07D 205/04; C07D 295/155; C07C 311/05; C07D 403/12; A61P 31/04; C07D 207/14; C07D 207/12; C07D 207/09; C07D 207/10; C07C 237/26; C07D 213/56

(52) U.S. Cl. 514/152; 552/284; 546/195; 548/950; 548/528; 546/285; 548/518; 544/154; 548/952; 548/953

(57) ABSTRACT

The present invention is directed to a compound represented by Structural Formula (I): or a pharmaceutically acceptable salt thereof. The variables for Structural Formula (I) are defined herein. Also described is a pharmaceutical composition comprising the compound of Structural Formula (I) and its therapeutic use.

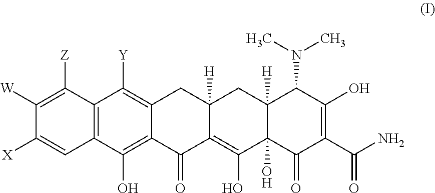

### RELATED APPLICATIONS

This application claims the benefit of U.S. Provisional Application No. 61/216,087, filed on May 13, 2009 and U.S. Provisional Application No. 61/337,710, filed on Feb. 9, 2010. The entire teachings of the above applications are incorporated herein by reference.

### BACKGROUND OF THE INVENTION

The tetracyclines are broad spectrum anti-microbial agents that are widely used in human and veterinary medicine. The total production of tetracyclines by fermentation or semi-synthesis is measured in the thousands of metric tons per year.

The widespread use of tetracyclines for therapeutic purposes has led to the emergence of resistance to these antibiotics, even among highly susceptible bacterial species. Therefore, there is need for new tetracycline analogs with improved antibacterial activities and efficacies against other tetracycline responsive diseases or disorders.

### SUMMARY OF THE INVENTION

Compounds of Formula I are new tetracycline analogs with improved antibacterial activities and efficacies against other tetracycline responsive diseases or

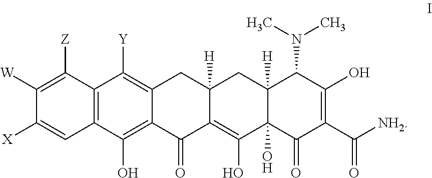

Pharmaceutically acceptable salts of the compound of Formula I are also included. Values for the variables in Formula I are provided below:

X is selected from hydrogen, fluoro, —CH₂OH, —CH₂—N(R¹)(R²), phenyl and pyridyl, wherein:

R¹ is selected from hydrogen, (C₁-C₈)alkyl, (C₀-C₆)alkylene-(C₁-C₆)alkoxy, (C₀-C₆) alkylene-(C₃-C₇)cycloalkyl, —C(O)—(C₁-C₆)alkyl, —C(O)—(C₁-C₆)alkoxy, phenyl, benzyl, pyridyl, —C(O)—N(R²)(R²), and —S(O)ₘ—(C₁-C₆)alkyl, wherein m is 1 or 2;

each R² is independently selected from hydrogen, and (C₁-C₄)alkyl; or

R¹ and R² are taken together with the nitrogen atom to which they are bound to form a (4-7 membered) heterocyclic ring optionally containing one additional heteroatom selected from S, O or N, each (C₁-C₈)alkyl, (C₀-C₆)alkylene, (C₁-C₆)alkoxy and (C₃-C₇)cycloalkyl in the group represented by R¹, R² and the ring formed by R¹ and R² are optionally and independently substituted with up to three substituents independently selected from halo, methyl, —CF₃, —OCH₃, —N(CH₃)₂, —NH—C(O)CH₃, —C(O)NH₂, —C(O)OCH₃, —CN or —OH;

Y is selected from hydrogen, fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy, —N(R³)(R⁴) and —O—C(O)—N(R³)(R⁴), wherein

each of R³ and R⁴ is independently selected from hydrogen, (C₁-C₆)alkyl, (C₃-C₇)cycloalkyl; or

R³ and R⁴ are taken together with the nitrogen atom to which they are bound to form a (4-7 membered) heterocyclic ring optionally containing one additional heteroatom selected from S, O or N,

each (C₁-C₆)alkyl or (C₃-C₇)cycloalkyl in the group represented by R³ and R⁴ and the (4-7 membered) heterocyclic ring formed by R³ and R⁴ are each optionally and independently substituted with halo or —OH;

W is selected from hydrogen, (C₁-C₆)alkoxy, and —N(R³)(R⁴); and

Z is selected from hydrogen, (C₁-C₆)alkoxy, and —N(R³)(R⁴),

wherein each phenyl, benzyl and pyridyl in the group represented by X and R¹ is optionally substituted with halo, unsubstituted (C₁-C₆)alkyl, halo(C₁-C₆)alkyl, unsubstituted (C₁-C₆)alkoxy, halo(C₁-C₆)alkoxy, cyano, amino, —NH—C(O)—(C₁-C₆)alkyl or nitro.

When W, Y and Z are hydrogen, X is not hydrogen, —CH₂NHC(CH₃)₃, —CH₂NH-cyclopropyl, CH₂-morpholin-4-yl, CH₂-4-methylpiperazin-1-yl, CH₂-4-acetylpiperazin-1-yl, CH₂N(CH₃)C(O)CH₃, CH₂-1H-imidazol-1-yl; and

when W and Z are hydrogen and Y is —N(CH₃)₂, X is not —CH₂NHC(CH₃)₃, —CH₂—NH-(1-methylcyclopropyl), —CH₂—NH-(1-methylcyclopentyl), —CH₂—NH-cyclopropyl, —CH₂—NH-cyclopentyl, —CH₂—NH-cyclohexyl, —CH₂NHCH₂C(CH₃)₃, —CH₂-azetidin-1-yl; and/or one of W or Z is not hydrogen. The provisos in these last two paragraphs are meant to apply to all of the structural formulas disclosed herein, including Structural Formula (II), where the positions corresponding to W and Z are occupied by hydrogen, Structural Formula (III), where the positions corresponding to W and Y are occupied by hydrogen and Structural Formula (IV), where the positions corresponding to Y and Z are occupied by hydrogen.

Another embodiment of the present invention is directed to a pharmaceutical composition comprising a pharmaceutically acceptable carrier or diluent and a compound disclosed herein or a pharmaceutically acceptable salt thereof. The pharmaceutical composition is used in therapy, such as treating an infection in a subject.

Another embodiment of the present invention is a method of treating an infection in a subject comprising administering to the subject an effective amount of a compound disclosed herein or a pharmaceutically acceptable salt thereof.

Another embodiment of the present invention is the use of a compound disclosed herein or a pharmaceutically acceptable salt thereof for the manufacture of a medicament for treating an infection in a subject.

Another embodiment of the present invention is the use of a compound disclosed herein or a pharmaceutically acceptable salt thereof for therapy, such as treating an infection in a subject.

### DETAILED DESCRIPTION OF THE INVENTION

The present invention is directed to a compound represented by Formula I or a pharmaceutically acceptable salt thereof. Values and alternative values for the variables in Formula I are defined as the following:

X is hydrogen, fluoro, —CH₂OH, —CH₂—N(R¹)(R²), phenyl or pyridyl. In another embodiment, X is selected from hydrogen, fluoro, —CH₂OH, phenyl and pyridyl. In preferred embodiment, X is —CH₂N(R¹)(R²). In another preferred embodiment, X is phenyl or pyridyl. Alternatively, X is fluoro. In yet another alternative, X is hydrogen.

Y is hydrogen, fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy, —N(R³)(R⁴) or —O—C(O)—N(R³)(R⁴). In another embodiment, Y is hydrogen, fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy or —O—C(O)—N(R³)(R⁴).

In another embodiment, Y is
fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy or —O—C(O)—N(R³)(R⁴). Alternatively, Y is —N(R³)(R⁴) or hydrogen. In another alternative, Y is hydrogen or —N(CH₃)₂. In yet another embodiment, Y is (C₁-C₆)alkoxy, —O—(C₃-C₇)cycloalkyl, —O—(C₁-C₄)alkylene-N(R³)(R⁴), —O—(C₁-C₆)alkylene-(C₁-C₆)alkoxy or —O—C(O)—N(R³)(R⁴). In a preferred embodiment, Y is fluoro, chloro or —NO₂.
Alternatively, Y is hydrogen.

Z is hydrogen, (C₁-C₆)alkoxy or —N(R³)(R⁴). In another embodiment, Z is hydrogen.

W is hydrogen, (C₁-C₆)alkoxy, and —N(R³)(R⁴). In another embodiment, W is hydrogen.

R¹ is hydrogen, (C₁-C₈)alkyl, (C₀-C₆)alkylene-(C₁-C₆)alkoxy, (C₀-C₆) alkylene-(C₃-C₇)cycloalkyl, —C(O)—(C₁-C₆)alkyl, —C(O)—(C₁-C₆)alkoxy, phenyl, benzyl, pyridyl, —C(O)—N(R²)(R²), or —S(O)ₘ—(C₁-C₆)alkyl, and m is 1 or 2. In another embodiment, R¹ is (C₀-C₆)alkylene-(C₁-C₆)alkoxy, —(C₁-C₆)alkylene-(C₃-C₇)cycloalkyl, —C(O)—(C₁-C₆)alkoxy, phenyl, benzyl, pyridyl, —C(O)—N(R²)(R²), and —S(O)ₘ—(C₁-C₆)alkyl, and m is 1 or 2.

R² is hydrogen or (C₁-C₄)alkyl.

Alternatively, R¹ and R² are taken together with the nitrogen atom to which they are bound to form a (4-7 membered) heterocyclic ring optionally containing one additional heteroatom selected from S, O or N.

Each (C₁-C₅)alkyl, (C₀-C₆)alkylene, (C₁-C₆)alkoxy and (C₃-C₇)cycloalkyl in the group represented by R¹, R² and the ring formed by R¹ and R² are optionally and independently substituted with up to three substituents independently selected from halo, methyl, —CF₃, —OCH₃, —N(CH₃)₂, —NH—C(O)CH₃, —C(O)NH₂, —C(O)OCH₃, —CN or —OH.

Each of R³ and R⁴ is hydrogen, (C₁-C₆)alkyl or (C₃-C₇)cycloalkyl.

Alternatively, R³ and R⁴ are taken together with the nitrogen atom to which they are bound to form a (4-7 membered) heterocyclic ring optionally containing one additional heteroatom selected from S, O or N.

Each (C₁-C₆)alkyl or (C₃-C₇)cycloalkyl in the group represented by R³ and R⁴ and the (4-7 membered) heterocyclic ring formed by R³ and R⁴ are each optionally and independently substituted with halo or —OH.

Each phenyl and pyridyl in the group represented by X and R¹ is optionally substituted with halo, unsubstituted C₁-C₆alkyl, halo(C₁-C₆)alkyl, unsubstituted C₁-C₆alkoxy, halo(C₁-C₆)alkoxy, cyano or nitro. Preferably, each phenyl in the group represented by X and R¹ is optionally substituted with halo, unsubstituted C₁-C₆ alkyl, halo(C₁-C₆)alkyl, unsubstituted C₁-C₆ alkoxy or halo(C₁-C₆)alkoxy. More preferably, each phenyl in the group represented by X and R¹ is optionally substituted with fluoro, —CF₃, —OCH₃ or —OCF₃.

When W, Y and Z are hydrogen, X is not hydrogen, —CH₂NHC(CH₃)₃, —CH₂NH-cyclopropyl, CH₂-morpholin-4-yl, CH₂-4-methylpiperazin-1-yl, CH₂-4-acetylpiperazin-1-yl, CH₂N(CH₃)C(O)CH₃, CH₂-1H-imidazol-1-yl; and

when W and Z are hydrogen and Y is —N(CH₃)₂, X is not —CH₂NHC(CH₃)₃, —CH₂—NH-(1-methylcyclopropyl), —CH₂—NH-(1-methylcyclopentyl), —CH₂—NH-cyclopropyl, —CH₂—NH-cyclopentyl, —CH₂—NH-cyclohexyl, —CH₂NHCH₂C(CH₃)₃, —CH₂-azetidin-1-yl; and/or one of W or Z is not hydrogen.

A second embodiment is a compound of Structural Formula II, or a pharmaceutically acceptable salt thereof:

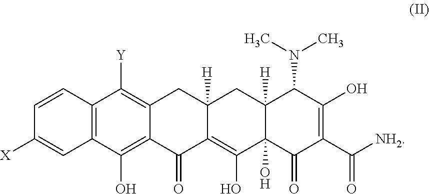

Pharmaceutically acceptable salts of the compound represented by Structural Formula II are also included in the invention. Values and alternative values for the remainder of the variables are as described above for Structural Formula (I).

A third embodiment of the invention is a compound represented by Structural Formulas (I) or (II), or a pharmaceutically acceptable salt thereof: X is —CH₂—N(R¹)(R²); Y is —N(R³)(R⁴); alternatively, Y is hydrogen or —N(CH₃)₂; in another alternative, Y is selected from (C₁-C₆)alkoxy, —O—(C₃-C₇)cycloalkyl, —O—(C₁-C₄)alkylene-N(R³)(R⁴), —O—(C₁-C₆)alkylene-(C₁-C₆)alkoxy and —O—C(O)—N(R³)(R⁴); and in yet another alternative, Y is selected from fluoro, chloro and —NO₂; and values and alternative values for the remainder of the variables are as described above for Structural Formula (I).

A fourth embodiment of the invention is a compound represented by Structural Formulas (I) or (II), or a pharmaceutically acceptable salt thereof: X is selected from hydrogen, fluoro, —CH₂OH, phenyl and pyridyl; each phenyl and pyridyl in the group represented by X is optionally substituted with halo, unsubstituted C₁-C₆-alkyl, halo(C₁-C₆)alkyl, unsubstituted C₁-C₆alkoxy, halo(C₁-C₆)alkoxy, cyano, amino, —NH—C(O)—(C₁-C₆)alkyl or nitro; Y is selected from hydrogen, fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy, —N(R³)(R⁴) and —O—C(O)—N(R³)(R⁴); and values and alternative values for the remainder of the variables are as described above for Structural Formula (I).

A fifth embodiment of the invention is a compound represented by Structural Formulas (I) or (II), or a pharmaceutically acceptable salt thereof: X is selected from phenyl and pyridyl; phenyl and pyridyl in the group represented by X are optionally substituted with up to two substituents independently selected from halo, methyl, —CF₃, —OCH₃, —N(CH₃)₂, or —NH—C(O)CH₃; Y is hydrogen; and values and alternative values for the remainder of the variables are as described above for Structural Formula (I).

A sixth embodiment of the invention is a compound represented by Structural Formulas (I) or (II), or a pharmaceutically acceptable salt thereof: X is —CH₂—N(R¹)(R²); R¹ is selected from (C₀-C₆)alkylene-(C₁-C₆)alkoxy, —(C₁-C₆)alkylene-(C₃-C₇)cycloalkyl, —C(O)—(C₁-C₆)alkoxy, phenyl, benzyl, pyridyl, —C(O)—N(R²)(R²), and —S(O)ₘ—(C₁-C₆)alkyl; m is 1 or 2; each phenyl, benzyl and pyridyl in the group represented R¹ is optionally substituted with halo, C₁-C₆ alkyl, halo(C₁-C₆)alkyl, (C₁-C₆)alkoxy, halo(C₁-C₆)alkoxy, cyano, amino, —NH—C(O)—(C₁-C₆)alkyl or nitro; and each (C₁-C₈)alkyl, (C₀-C₆)alkylene, (C₁-C₆)alkoxy and (C₃-C₇)cycloalkyl in the group represented by R¹ is optionally substituted halo, methyl, —CF₃, —OCH₃, —N(CH₃)₂, —NH—C(O)CH₃, —C(O)NH₂, —C(O)OCH₃, —CN or —OH; values and alternative values for the remainder of the variables are as described for Structural Formula (I).

A seventh embodiment of the invention is a compound represented by Structural Formula (III) or (IV):

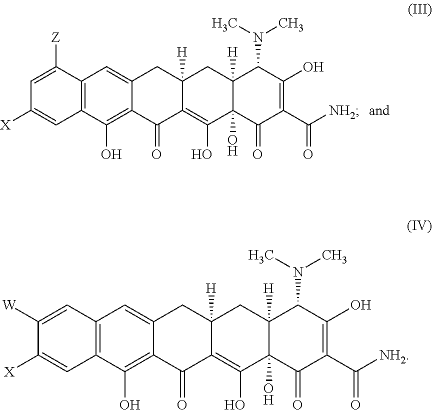

Pharmaceutically acceptable salts of the compound represented by Structural Formula (III) and (IV) are also included in the invention. Values and alternative values for the remainder of the variables are as described above for Structural Formula (I).

An eighth embodiment of the invention is a compound represented by the Structural Formula I, II, III or IV, wherein Z, Y and W are hydrogen and X is:

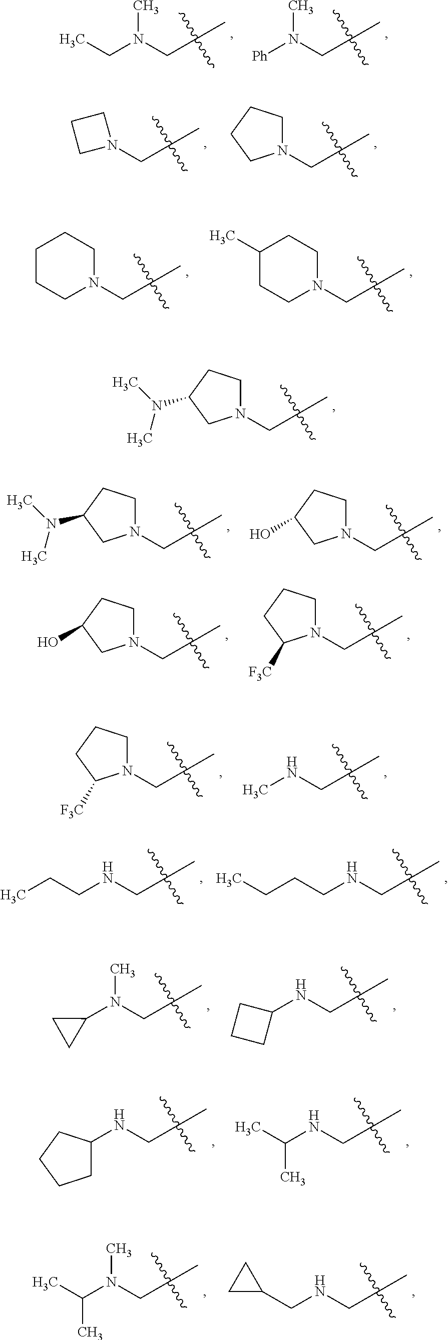

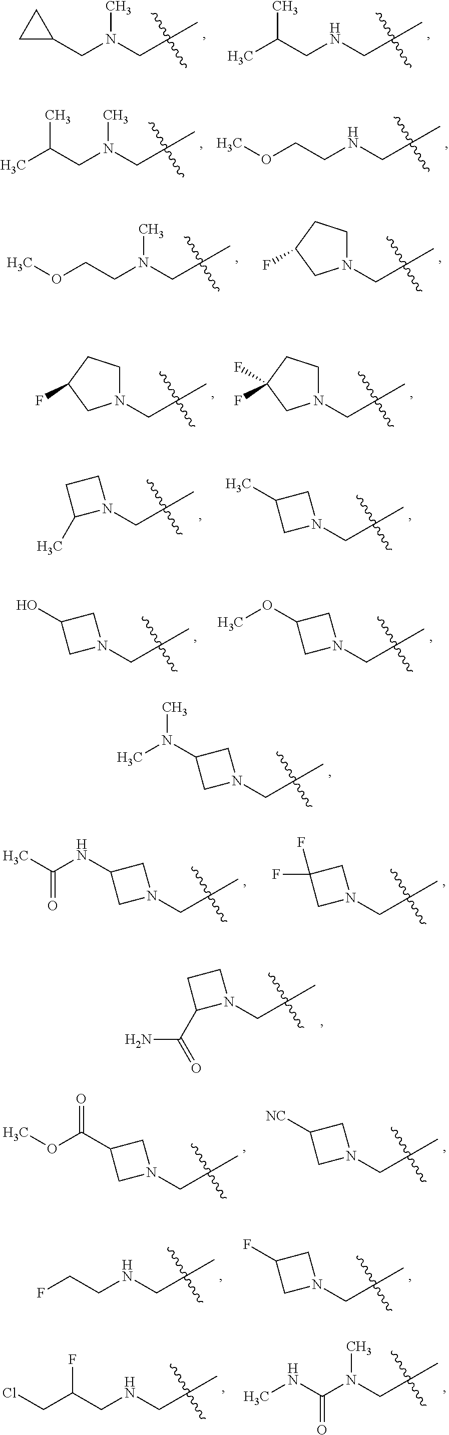

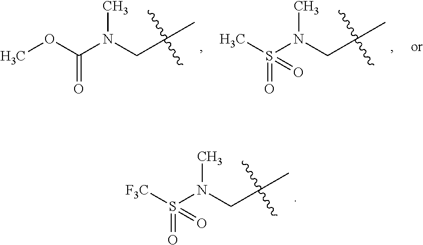

A ninth embodiment of the invention is a compound represented by the Structural Formula I, II, III or IV, wherein Z, Y and W are hydrogen and X is:

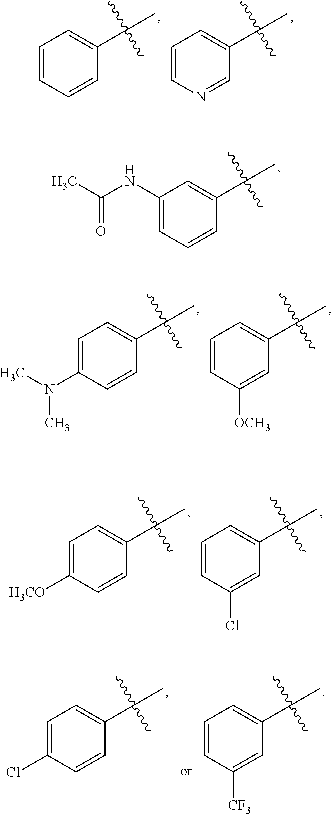

A tenth embodiment of the invention is a compound represented by the Structural Formula I or III, wherein X, Y and W are hydrogen and Z is:

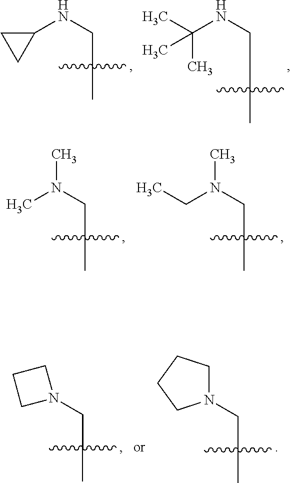

An eleventh embodiment of the invention is a compound represented by the Structural Formula I, II, III or IV, wherein Z and W are hydrogen, Y is —N(CH₃)₂ and X is:

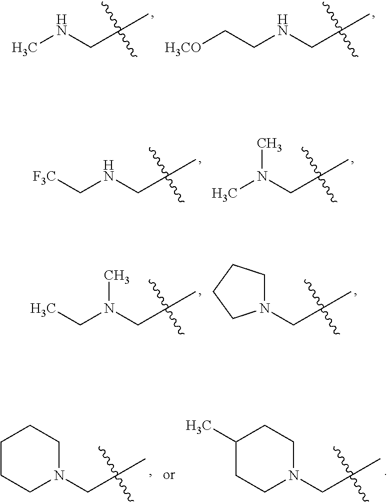

A twelfth embodiment of the invention is a compound represented by the Structural Formula I or II, wherein Z and W are hydrogen, X is

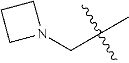

### and Y is:

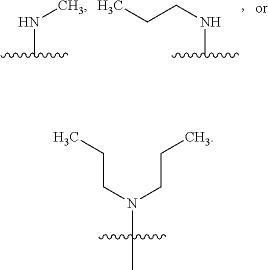

A thirteenth embodiment of the invention is a compound represented by the Structural Formula I or II, wherein Z and W are hydrogen, Y is —OCH₃ and X is:

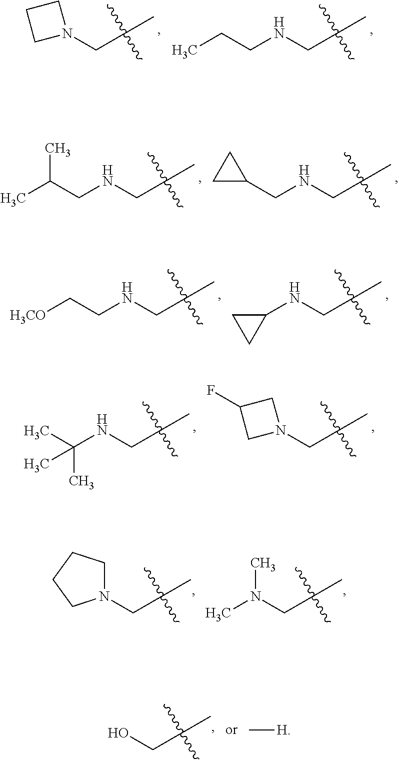

A fourteenth embodiment of the invention is a compound represented by the Structural Formula I or II, wherein Z and W are hydrogen, X is H or

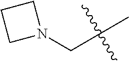

### and Y is:

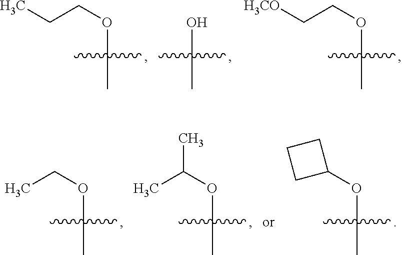

A fifteenth embodiment of the invention is a compound represented by the Structural Formula I or II, wherein Z and W are hydrogen, X is

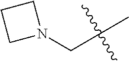

### and Y is:

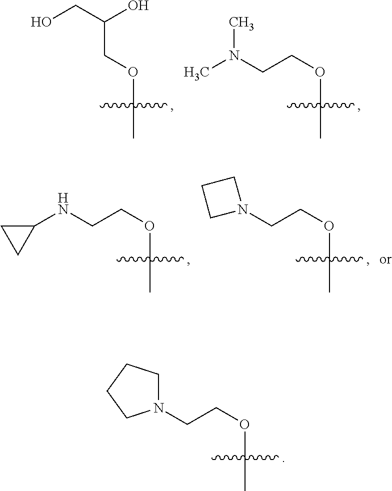

A sixteenth embodiment of the invention is a compound represented by the Structural Formula I or II, wherein X, Z and W are hydrogen and Y is:

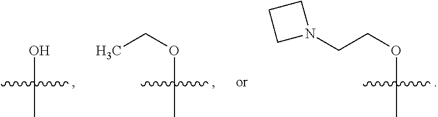

A seventeenth embodiment of the invention is a compound represented by the Structural Formula I or II, wherein Z and W are hydrogen and Y is F and X is:

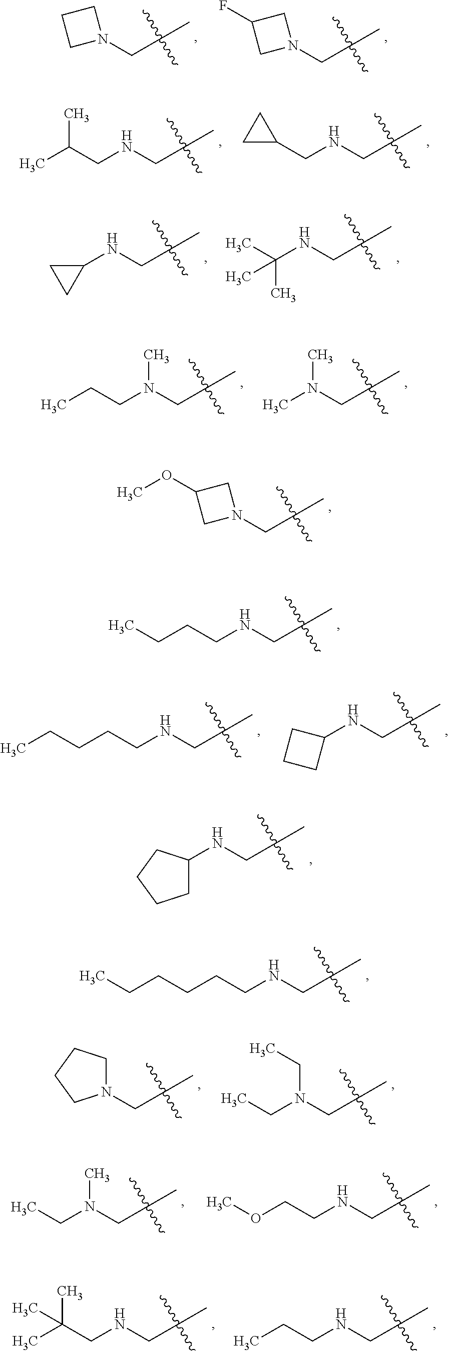

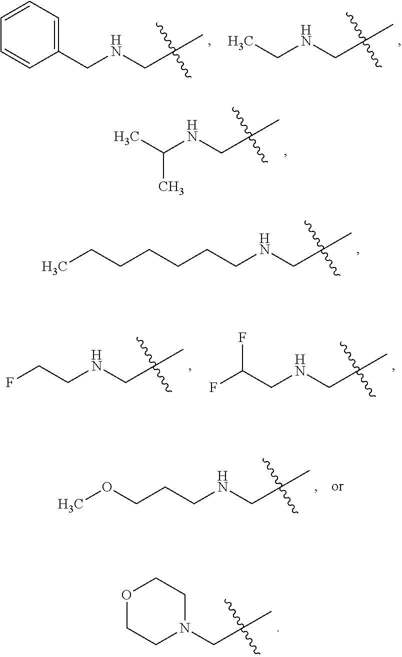

An eighteenth embodiment of the invention is a compound represented by the Structural Formula I or II, wherein Z and W are hydrogen and X is F and Y is:

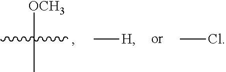

A nineteenth embodiment of the invention is a compound represented by the Structural Formula I or III, wherein X, Y and W are hydrogen and Z is:

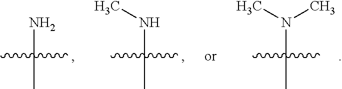

A twentieth embodiment of the invention is a compound represented by the Structural Formula I or IV, wherein X, Y and Z are hydrogen and W is:

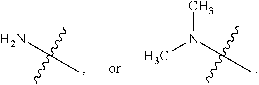

Exemplary compounds of the invention are presented in Tables 1-6.

TABLE 1
Exemplary Compounds of the Invention where Z,
Y and W are hydrogen; and X is —CH₂—N(R¹)(R²)

| Compound | |
|---|---|
| No. | —N(R¹)(R²) |
| 100 | 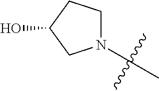 |
| 101 | 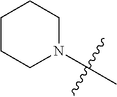 |
| 102 | 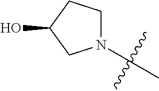 |
| 103 | 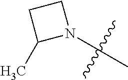 |
| 104 | 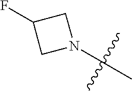 |
| 105 | 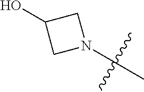 |
| 106 | 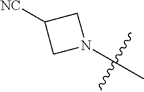 |
| 107 | 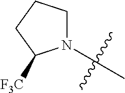 |
| 108 | 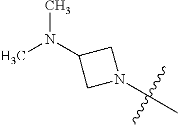 |
| 109 | 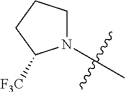 |
| 110 | 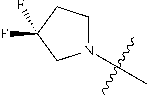 |
| 111 | 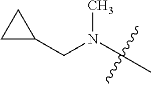 |
| 112 | 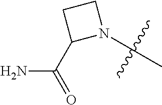 |
| 113 | 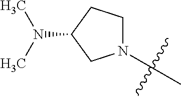 |
| 114 | 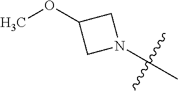 |
| 115 | 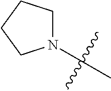 |
| 116 | 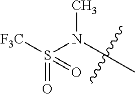 |
| 117 | 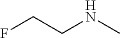 |
| 118 | 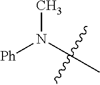 |
| 119 | 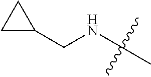 |
| 120 | 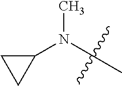 |
| 121 | 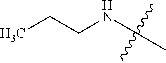 |
| 122 | 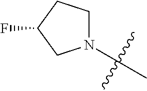 |
| 123 | 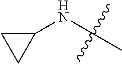 |
| 124 | 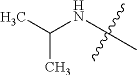 |
| 125 | 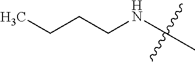 |
| 126 | 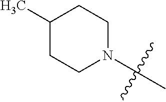 |
| 127 | 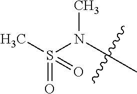 |
| 128 | 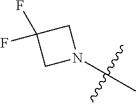 |
| 129 | 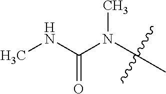 |
| 130 | 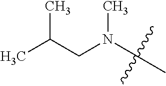 |
| 131 | 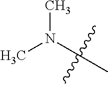 |
| 132 | 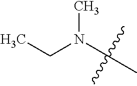 |
| 133 | 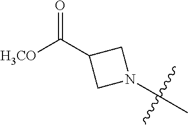 |
| 134 | 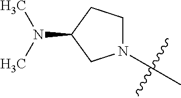 |
| 135 | 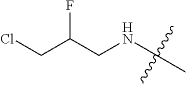 |
| 136 | 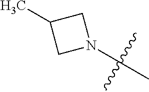 |
| 137 | 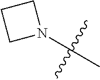 |
| 138 | 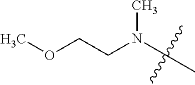 |
| 139 | 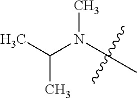 |
| 140 | 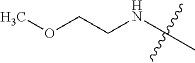 |
| 141 | 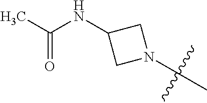 |
| 142 | 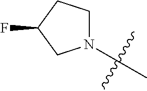 |
| 143 | 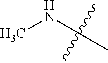 |
| 144 | 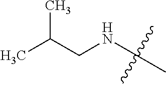 |
| 145 | 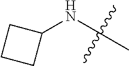 |
| 146 | 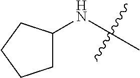 |
| 147 | 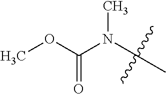 |

TABLE 2
Exemplary Compounds of the Invention where X, Y and Z are hydrogen.

| Compound | |
|---|---|
| No. | W |
| 200 | 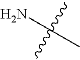 |
| 201 | 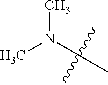 |

TABLE 3
Exemplary Compounds of the Invention where Z and W are hydrogen; Y is
—N(CH₃)₂; and X is —CH₂—N(R¹)(R²)

| Compound | |
|---|---|
| No. | —N(R¹)(R²) |
| 300 | 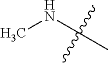 |
| 301 | 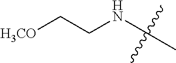 |
| 302 | 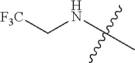 |
| 303 | 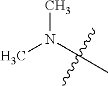 |
| 304 | 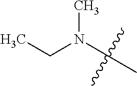 |
| 305 | 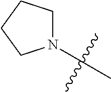 |
| 306 | 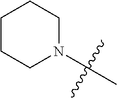 |
| 307 | 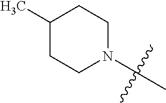 |

TABLE 4
Exemplary Compounds of the Invention where W and Z are hydrogen.

| Cmpd | | |
|---|---|---|
| No | X | Y |
| 400 | 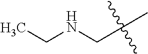 | 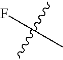 |
| 401 | 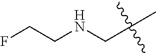 | 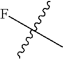 |
| 402 | 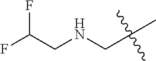 | 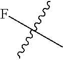 |
| 403 | 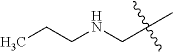 | 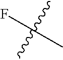 |
| 404 | 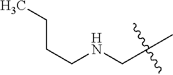 | 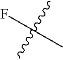 |
| 405 | 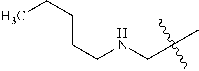 | 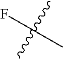 |
| 406 | 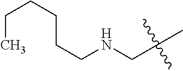 | 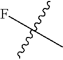 |
| 407 | 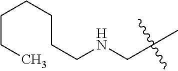 | 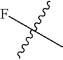 |
| 408 | 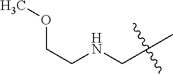 | 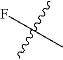 |
| 409 | 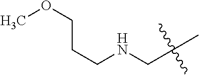 | 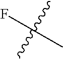 |
| 410 | 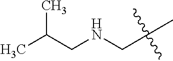 | 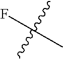 |
| 411 | 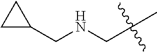 | 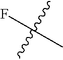 |
| 412 | 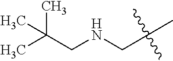 | 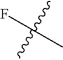 |
| 413 | 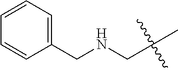 | 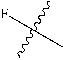 |
| 414 | 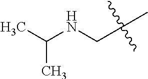 | 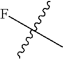 |
| 415 | 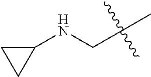 | 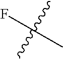 |
| 416 | 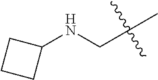 | 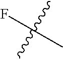 |
| 417 | 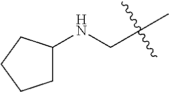 | 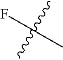 |
| 418 | 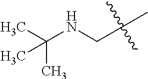 | 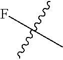 |
| 419 | 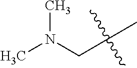 | 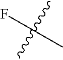 |
| 420 | 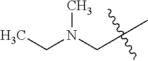 | 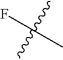 |
| 421 | 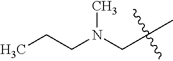 | 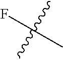 |
| 422 | 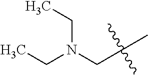 | 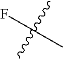 |
| 423 | 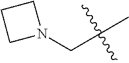 | 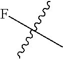 |
| 424 | 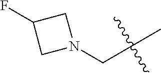 | 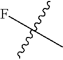 |
| 425 | 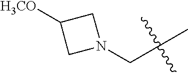 | 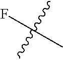 |
| 426 | 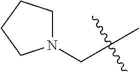 | 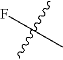 |
| 427 | 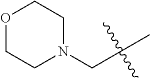 | 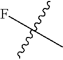 |
| 428 | 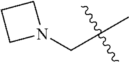 | 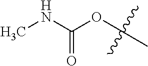 |
| 429 | 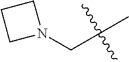 | 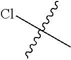 |
| 430 | 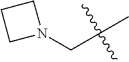 | 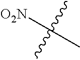 |
| 431 | 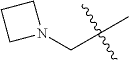 | 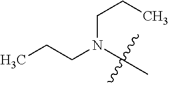 |
| 432 | 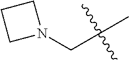 | 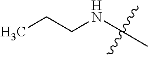 |
| 433 | 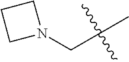 | 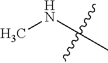 |
| 434 | 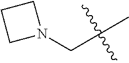 | 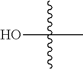 |
| 435 | 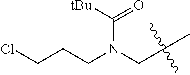 | 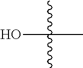 |
| 436 | 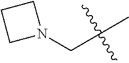 | 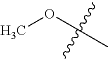 |
| 437 | 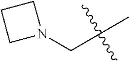 | 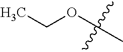 |
| 438 | 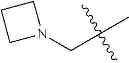 | 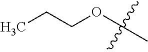 |
| 439 | 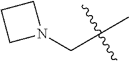 | 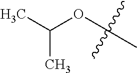 |
| 440 | 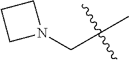 | 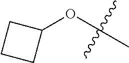 |
| 441 | 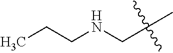 | 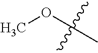 |
| 442 | 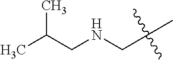 | 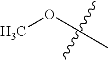 |
| 443 | 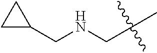 | 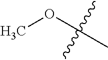 |
| 444 | 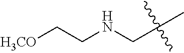 | 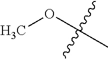 |
| 445 | 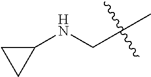 | 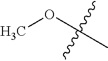 |
| 446 | 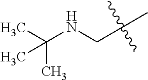 | 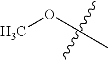 |
| 447 | 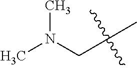 | 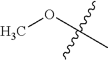 |
| 448 | 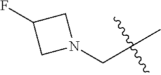 | 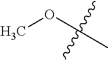 |
| 449 | 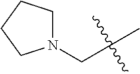 | 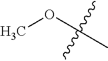 |
| 450 | 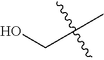 | 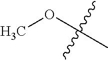 |
| 451 | 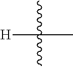 | 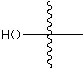 |
| 452 | 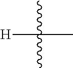 | 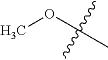 |
| 453 | 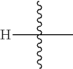 | 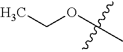 |
| 454 | 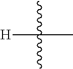 | 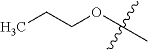 |
| 455 | 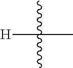 | 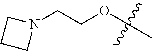 |
| 456 | 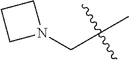 | 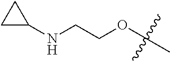 |
| 457 | 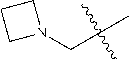 | 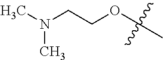 |
| 458 | 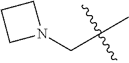 | 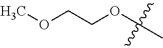 |
| 459 | 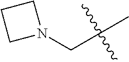 | 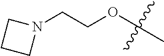 |
| 460 | 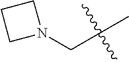 | 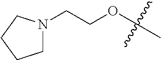 |
| 461 | 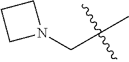 | 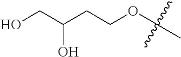 |
| 462 | 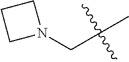 | 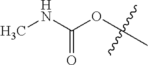 |
| 463 | 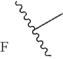 | 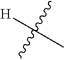 |
| 464 | 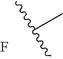 | 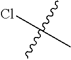 |
| 465 | 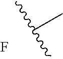 | 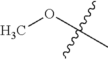 |
| 466 | 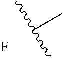 | 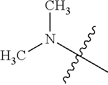 |

TABLE 5
Exemplary Compounds of the Invention where W, Y and X are hydrogen.

| Compound | |
|---|---|
| No | Z |
| 500 | 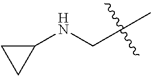 |
| 501 | 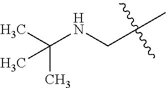 |
| 502 | 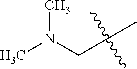 |
| 503 | 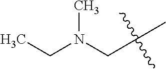 |
| 504 | 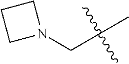 |
| 505 | 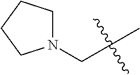 |
| 506 | 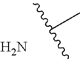 |
| 507 | 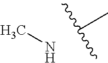 |
| 508 | 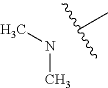 |
| 509 | 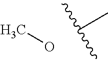 |

TABLE 6
Exemplary Compounds of the Invention where W, Y and Z are hydrogen.

| Compound | |
|---|---|
| No | X |
| 600 | 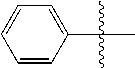 |
| 601 | 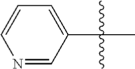 |
| 602 | 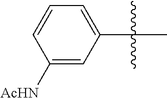 |
| 603 | 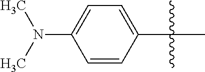 |
| 604 | 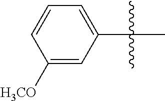 |
| 605 | 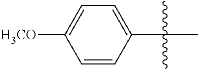 |
| 606 | 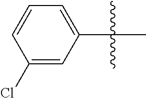 |
| 607 | 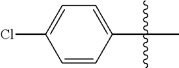 |
| 608 | 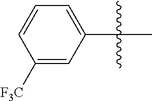 |

Pharmaceutically acceptable salts of the compounds in Tables 1-6 are also included in the invention.

Preferred Examples of the compounds of the invention are selected from any one of Compounds 117, 119, 121, 125, 138, 140, 144, 146, 401, 403, 408, 409, 410, 411, 414, 417, and 418 or a pharmaceutically acceptable salt thereof.

In another embodiment, the preferred Examples of the compounds of the invention are selected from any one of Compounds 117, 119, 121, 125, 138, 140, 144, 401, 403, 408, 409, 410, 411, 414, 417, and 418 or a pharmaceutically acceptable salt thereof.

### DEFINITIONS

“Alkyl” means a saturated aliphatic branched or straight-chain monovalent hydrocarbon radical having the specified number of carbon atoms. Thus, “(C₁-C₆) alkyl” means a radical having from 1-6 carbon atoms in a linear or branched arrangement. “(C₁-C₆)alkyl” includes methyl, ethyl, propyl, butyl, pentyl and hexyl.

“Alkylene” means a saturated aliphatic straight-chain divalent hydrocarbon radical having the specified number of carbon atoms. Thus, “(C₁-C₆)alkylene” means a divalent saturated aliphatic radical having from 1-6 carbon atoms in a linear arrangement. “(C₁-C₆)alkylene” includes methylene, ethylene, propylene, butylene, pentylene and hexylene.

“Heterocycle” means a saturated or partially unsaturated (4-7 membered) monocyclic heterocyclic ring containing one nitrogen atom and optionally 1 additional heteroatom independently selected from N, O or S. When one heteroatom is S, it can be optionally mono- or di-oxygenated (i.e. —S(O)— or —S(O)₂—). Examples of monocyclic heterocycle include, but not limited to, azetidine, pyrrolidine, piperidine, piperazine, hexahydropyrimidine, tetrahydrofuran, tetrahydropyran, morpholine, thiomorpholine, thiomorpholine 1,1-dioxide, tetrahydro-2H-1,2-thiazine, tetrahydro-2H-1,2-thiazine 1,1-dioxide, isothiazolidine, or isothiazolidine 1,1-dioxide.

“Cycloalkyl” means saturated aliphatic cyclic hydrocarbon ring. Thus, “C₃-C₇ cycloalkyl” means (3-7 membered) saturated aliphatic cyclic hydrocarbon ring. C₃-C₇ cycloalkyl includes, but is not limited to cyclopropyl, cyclobutyl, cyclopentyl, cyclohexyl and cycloheptyl.

“Alkoxy” means an alkyl radical attached through an oxygen linking atom. “(C₁-C₆)alkoxy” includes methoxy, ethoxy, propoxy, butoxy, pentoxy and hexoxy.

“Cycloalkoxy” means an cycloalkyl-O— group wherein the cycloalkyl is as defined above. Exemplary (C₃-C₇)cycloalkyloxy groups include cyclopropoxy, cyclobutoxy, cyclopentoxy, cyclohexoxy and cycloheptoxy.

Haloalkyl and halocycloalkyl include mono, poly, and perhaloalkyl groups where each halogen is independently selected from fluorine, chlorine, and bromine.

“Hetero” refers to the replacement of at least one carbon atom member in a ring system with at least one heteroatom selected from N, S, and O. A hetero ring system may have 1 or 2 carbon atom members replaced by a heteroatom.

“Halogen” and “halo” are interchangeably used herein and each refers to fluorine, chlorine, bromine, or iodine.

“Cyano” means —C≡N.

“Nitro” means —NO₂.

Another embodiment of the present invention is a pharmaceutical composition comprising one or more pharmaceutically acceptable carrier and/or diluent and a compound disclosed herein or a pharmaceutically acceptable salt thereof.

“Pharmaceutically acceptable carrier” and “pharmaceutically acceptable diluent” means non-therapeutic components that are of sufficient purity and quality for use in the formulation of a composition of the invention that, when appropriately administered to an animal or human, typically do not produce an adverse reaction, and that are used as a vehicle for a drug substance (i.e. a compound of the present invention).

Pharmaceutically acceptable salts of the compounds of the present invention are also included. For example, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

Salts of the compounds of the present invention containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

The invention also includes various isomers and mixtures thereof. Certain of the compounds of the present invention may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. When a chiral center is not defined as R or S, either a pure enantiomer or a mixture of both configurations is present.

“Racemate” or “racemic mixture” means a compound of equimolar quantities of two enantiomers, wherein such mixtures exhibit no optical activity; i.e., they do not rotate the plane of polarized light.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer that is present divided by the combined weight of the enantiomer that is present and the weight of its optical isomer.

The present invention also provides a method of treating a subject with a tetracycline-responsive disease or disorder comprising administering to the subject an effective amount of a compound of the present invention or a pharmaceutically acceptable salt thereof.

“Tetracycline-responsive disease or disorder” refers to a disease or disorder that can be treated, prevented, or otherwise ameliorated by the administration of a tetracycline compound of the present invention. Tetracycline-responsive disease or disorder includes infections, cancer, inflammatory disorders, autoimmune disease, arteriosclerosis, corneal ulceration, emphysema, arthritis, osteoporosis, osteoarthritis, multiple sclerosis, osteosarcoma, osteomyelitis, bronchiectasis, chronic pulmonary obstructive disease, skin and eye diseases, periodontitis, osteoporosis, rheumatoid arthritis, ulcerative colitis, inflammatory disorders, tumor growth and invasion, metastasis, acute lung injury, stroke, ischemia, diabetes, aortic or vascular aneurysms, skin tissue wounds, dry eye, bone, cartilage degradation, malaria, senescence, diabetes, vascular stroke, neurodegenerative disorders, cardiac disease, juvenile diabetes, acute and chronic bronchitis, sinusitis, and respiratory infections, including the common cold; acute and chronic gastroenteritis and colitis; acute and chronic cystitis and urethritis; acute and chronic dermatitis; acute and chronic conjunctivitis; acute and chronic serositis; uremic pericarditis; acute and chronic cholecystis; cystic fibrosis, acute and chronic vaginitis; acute and chronic uveitis; drug reactions; insect bites; burns and sunburn, bone mass disorder, acute lung injury, chronic lung disorders, ischemia, stroke or ischemic stroke, skin wound, aortic or vascular aneurysm, diabetic retinopathy, hemorrhagic stroke, angiogenesis, and other states for which tetracycline compounds have been found to be active (see, for example, U.S. Pat. Nos. 5,789,395; 5,834,450; 6,277,061 and 5,532,227, each of which is expressly incorporated herein by reference). Compounds of the invention can be used to prevent or control important mammalian and veterinary diseases such as diarrhea, urinary tract infections, infections of skin and skin structure, ear, nose and throat infections, wound infection, mastitis and the like. In addition, methods for treating neoplasms using tetracycline compounds of the invention are also included (van der Bozert et al., Cancer Res., 48: 6686-6690 (1988)).

Infections that can be treated using compounds of the invention or a pharmaceutically acceptable salt thereof include, but are not limited to, skin infections, GI infections, urinary tract infections, genito-urinary infections, respiratory tract infections, sinuses infections, middle ear infections, systemic infections, cholera, influenza, bronchitis, acne, malaria, sexually transmitted disease including syphilis and gonorrhea, Legionnaires' disease, Lyme disease, Rocky Mountain spotted fever, Q fever, typhus, bubonic plague, gas gangrene, hospital acquired infections, leptospirosis, whooping cough, anthrax and infections caused by the agents responsible for lymphogranuloma venereum, inclusion conjunctivitis, or psittacosis. Infections can be bacterial, fungal, parasitic and viral infections (including those which are resistant to other tetracycline compounds).

In one embodiment, the infection can be caused bacteria. In another embodiment, the infection is caused by a Gram-positive bacteria. In a specific aspect of this embodiment, the infection is caused by a Gram-positive bacteria selected from S. aureus, S. pneumoniae, P. granulosum and P. acnes. In another aspect, the infection is caused by a Gram-positive bacterium selected from class Bacilli, including, but not limited to, Staphylococcus spp., Streptococcus spp., Enterococcus spp., Bacillus spp., Listeria spp.; phylum Actinobacteria, including, but not limited to, Propionibacterium spp., Corynebacterium spp., Nocardia spp., Actinobacteria spp., and class Clostridia, including, but not limited to, Clostridium spp.

In another embodiment, the infection is caused by a Gram-negative bacteria. In a specific aspect of this embodiment, the infection is caused by a Gram-negative bacteria selected from E. coli or B. thetaiotaomicron. In one aspect of this embodiment, the infection is caused by a phylum Proteobacteria (e.g., Betaproteobacteria and Gammaproteobacteria), including Escherichia coli, Salmonella, Shigella, other Enterobacteriaceae, Pseudomonas, Moraxella, Helicobacter, Stenotrophomonas, Bdellovibrio, acetic acid bacteria, Legionella or alpha-proteobacteria such as Wolbachia. In another aspect, the infection is caused by a Gram-negative bacterium selected from cyanobacteria, spirochaetes, green sulfur or green non-sulfur bacteria. In a specific aspect of this embodiment, the infection is caused by a Gram-negative bacteria selected from Enterobactericeae (e.g., E. coli, Klebsiella pneumoniae including those containing extended-spectrum β-lactamases and/or carbapenemases), Bacteroidetes (e.g., Bacteroides fragilis), Vibrionaceae (Vibrio cholerae), Pasteurellaceae (e.g., Haemophilus influenzae), Pseudomonadaceae (e.g., Pseudomonas aeruginosa), Neisseriaceae (e.g. Neisseria meningitidis), Rickettsiae, Moraxellaceae (e.g., Moraxella catarrhalis), any species of Proteeae, Acinetobacter spp., Helicobacter spp., and Campylobacter spp. In a particular embodiment, the infection is caused by Gram-negative bacterium selected from the group consisting of Enterobactericeae (e.g., E. coli, Klebsiella pneumoniae), Pseudomonas, and Acinetobacter spp. In another embodiment, the infection is caused by an organism selected from the group consisting of K. pneumoniae, Salmonella, E. hirae, A. baumanii, M catarrhalis, H. influenzae, P. aeruginosa, E. faecium, E. coli, S. aureus, and E. faecalis.

In one embodiment, the infection is caused by an organism that grows intracellularly as part of its infection process.

In another embodiment, the infection is caused by an organism selected from the group consisting of K. pneumoniae, Salmonella, E. hirae, A. baumanii, B. catarrhalis, H. influenzae, P. aeruginosa, E. faecium, E. coli, S. aureus, and E. faecalis. In another embodiment, the infection is caused by an organism selected from the group consisting of rickettsiae, chlamydiae, and Mycoplasma pneumoniae. Alternatively, the infection is caused by an organism selected from the group consisting of order Rickettsiales; phylum Chlamydiae; order Chlamydiales; Legionella spp.; class Mollicutes, including, but not limited to, Mycoplasma spp. (e.g. Mycoplasma pneumoniae); Mycobacterium spp. (e.g. Mycobacterium tuberculosis); and phylum Spriochaetales (e.g. Borrelia spp. and Treponema spp.).

In another embodiment, the infection is caused by an organism resistant to tetracycline. In another embodiment, the infection is caused by an organism resistant to methicillin. In another embodiment, the infection is caused by an organism resistant to vancomycin. In another embodiment the infection is a Bacillus anthracis infection. “Bacillus anthracis infection” includes any state, diseases, or disorders caused or which result from exposure or alleged exposure to Bacillus anthracis or another member of the Bacillus cereus group of bacteria.

Additional infections that can be treated using compounds of the invention or a pharmaceutically acceptable salt thereof include, but are not limited to, anthrax, botulism, bubonic plague, and tularemia.

In another embodiment, the infection is caused by a Category B Biodefense organism as described at http://www.bt.cdc.gov/agent/agentlist-category.asp, the entire teachings of which are incorporated herein by reference. Examples of Category B organisms include, but are not limited to, Brucella spp, Clostridium perfringens, Salmonella spp., Escherichia coli O157:H7, Shigella spp., Burkholderia mallei, Burkholderia pseudomallei, Chlamydia psittaci, Coxiella burnetii, Staphylococcal enterotoxin B, Rickettsia prowazekii, Vibrio cholerae, and Cryptosporidium parvum.

Additional infections that can be treated using compounds of the invention or a pharmaceutically acceptable salt thereof include, but are not limited to, Brucellosis, Clostridium perfringens, food-borne illnesses, Glanders, Melioidosis, Psittacosis, Q fever, and water-borne illnesses.

In yet another embodiment, the infection can be caused by one or more than one organism described above. Examples of such infections include, but are not limited to, intra-abdominal infections (often a mixture of a gram-negative species like E. coli and an anaerobe like B. fragilis), diabetic foot (various combinations of Streptococcus, Serratia, Staphylococcus and Enterococcus spp., anaerobes (S. E. Dowd, et al., PloS one 2008; 3:e3326, the entire teachings of which are incorporated herein by reference) and respiratory disease (especially in patients that have chronic infections like cystic fibrosis—e.g., S. aureus plus P. aeruginosa or H. influenzae, atypical pathogens), wounds and abscesses (various gram-negative and gram-positive bacteria, notably MSSA/MRSA, coagulase-negative staphylococci, enterococci, Acinetobacter, P. aeruginosa, E. coli, B. fragilis), and bloodstream infections (13% were polymicrobial (H. Wisplinghoff, et al., Clin. Infect. Dis. 2004; 39:311-317, the entire teachings of which are incorporated herein by reference)).

In one embodiment, the infection is caused by an organism resistant to one or more antibiotics.

In another embodiment, the infection is caused by an organism resistant to tetracycline or any member of first and second generation of tetracycline antibiotics (e.g., doxycycline or minocycline).

In another embodiment, the infection is caused by an organism resistant to a quinolone or fluoroquinolone.

In another embodiment, the infection is caused by an organism resistant to tigecycline or any other tetracycline derivative. In a particular embodiment, the infection is caused by an organism resistant to tigecycline.

In another embodiment, the infection is caused by an organism resistant to a β-lactam or cephalosporin antibiotic or an organism resistant to penems or carbapenems.

In another embodiment, the infection is caused by an organism resistant to an antimicrobial peptide or a biosimilar therapeutic treatment. Antimicrobial peptides (also called host defense peptides) are an evolutionarily conserved component of the innate immune response and are found among all classes of life. In this case, antimicrobial peptide refers to any naturally occurring molecule or any semi/synthetic molecule that are analogs of anionic peptides, linear cationic α-helical peptides, cationic peptides enriched for specific amino acids (i.e, rich in proline, arginine, phenylalanine, glycine, tryptophan), and anionic and cationic peptides that contain cystein and form disulfide bonds.

In another embodiment, the infection is caused by an organism resistant to macrolides, lincosamides, streptogramin antibiotics, oxazolidinones, and pleuromutilins.

In another embodiment, the infection is caused by an organism resistant to PTK0796 (7-dimethylamino, 9-(2,2-dimethyl-propyl)-aminomethylcycline).

In another embodiment, the infection is caused by a multidrug-resistant pathogen (having intermediate or full resistance to any two or more antibiotics).

In a further embodiment, the tetracycline responsive disease or disorder is not a bacterial infection. In another embodiment, the tetracycline compounds of the invention are essentially non-antibacterial. For example, non-antibacterial compounds of the invention may have MIC values of greater than 4 g/ml (as measured by assays known in the art and/or the assay given in Example 1.

Tetracycline responsive disease or disorder also includes diseases or disorders associated with inflammatory process associated states (IPAS). The term “inflammatory process associated state” includes states in which inflammation or inflammatory factors (e.g., matrix metalloproteinases (MMPs), nitric oxide (NO), TNF, interleukins, plasma proteins, cellular defense systems, cytokines, lipid metabolites, proteases, toxic radicals, adhesion molecules, etc.) are involved or are present in an area in aberrant amounts, e.g., in amounts which may be advantageous to alter, e.g., to benefit the subject. The inflammatory process is the response of living tissue to damage. The cause of inflammation may be due to physical damage, chemical substances, micro-organisms, tissue necrosis, cancer or other agents. Acute inflammation is short-lasting, lasting only a few days. If it is longer lasting however, then it may be referred to as chronic inflammation.

IPASs include inflammatory disorders. Inflammatory disorders are generally characterized by heat, redness, swelling, pain and loss of function. Examples of causes of inflammatory disorders include, but are not limited to, microbial infections (e.g., bacterial and fungal infections), physical agents (e.g., burns, radiation, and trauma), chemical agents (e.g., toxins and caustic substances), tissue necrosis and various types of immunologic reactions.

Examples of inflammatory disorders can be treated using the compounds of the invention or a pharmaceutically acceptable salt thereof include, but are not limited to, osteoarthritis, rheumatoid arthritis, acute and chronic infections (bacterial and fungal, including diphtheria and pertussis); acute and chronic bronchitis, sinusitis, and upper respiratory infections, including the common cold; acute and chronic gastroenteritis and colitis; inflammatory bowel disorder; acute and chronic cystitis and urethritis; vasculitis; sepsis; nephritis; pancreatitis; hepatitis; lupus; inflammatory skin disorders including, for example, eczema, dermatitis, psoriasis, pyoderma gangrenosum, acne rosacea, and acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; insect bites; burns (thermal, chemical, and electrical); and sunburn.

IPASs also include matrix metalloproteinase associated states (MMPAS). MMPAS include states characterized by aberrant amounts of MMPs or MMP activity. Examples of matrix metalloproteinase associated states (“MMPAS's”) can be treated using compounds of the invention or a pharmaceutically acceptable salt thereof,

include, but are not limited to, arteriosclerosis, corneal ulceration, emphysema, osteoarthritis, multiple sclerosis (Liedtke et al., Ann. Neurol. 1998, 44: 35-46; Chandler et al., J. Neuroimmunol. 1997, 72: 155-71), osteosarcoma, osteomyelitis, bronchiectasis, chronic pulmonary obstructive disease, skin and eye diseases, periodontitis, osteoporosis, rheumatoid arthritis, ulcerative colitis, inflammatory disorders, tumor growth and invasion (Stetler-Stevenson et al., Annu. Rev. Cell Biol. 1993, 9: 541-73; Tryggvason et al., Biochim. Biophys. Acta 1987, 907: 191-217; Li et al., Mol. Carcillog, 1998, 22: 84-89)), metastasis, acute lung injury, stroke, ischemia, diabetes, aortic or vascular aneurysms, skin tissue wounds, dry eye, bone and cartilage degradation (Greenwald et al., Bone 1998, 22: 33-38; Ryan et al., Curr. Op. Rheumatol. 1996, 8: 238-247). Other MMPAS include those described in U.S. Pat. Nos. 5,459,135; 5,321,017; 5,308,839; 5,258,371; 4,935,412; 4,704,383, 4,666,897, and RE 34,656, incorporated herein by reference in their entirety.

In a further embodiment, the IPAS includes disorders described in U.S. Pat. Nos. 5,929,055; and 5,532,227, incorporated herein by reference in their entirety.

Tetracycline responsive disease or disorder also includes diseases or disorders associated with NO associated states. The term “NO associated states” includes states which involve or are associated with nitric oxide (NO) or inducible nitric oxide synthase (iNOS). NO associated state includes states which are characterized by aberrant amounts of NO and/or iNOS. Preferably, the NO associated state can be treated by administering tetracycline compounds of the invention. The disorders, diseases and states described in U.S. Pat. Nos. 6,231,894; 6,015,804; 5,919,774; and 5,789,395 are also included as NO associated states. The entire contents of each of these patents are hereby incorporated herein by reference.

Examples of diseases or disorders associated with NO associated states can be treated using the compounds of the present invention or a pharmaceutically acceptable salt thereof include, but are not limited to, malaria, senescence, diabetes, vascular stroke, neurodegenerative disorders (Alzheimer's disease and Huntington's disease), cardiac disease (reperfusion-associated injury following infarction), juvenile diabetes, inflammatory disorders, osteoarthritis, rheumatoid arthritis, acute, recurrent and chronic infections (bacterial, viral and fungal); acute and chronic bronchitis, sinusitis, and respiratory infections, including the common cold; acute and chronic gastroenteritis and colitis; acute and chronic cystitis and urethritis; acute and chronic dermatitis; acute and chronic conjunctivitis; acute and chronic serositis (pericarditis, peritonitis, synovitis, pleuritis and tendonitis); uremic pericarditis; acute and chronic cholecystis; cystic fibrosis, acute and chronic vaginitis; acute and chronic uveitis; drug reactions; insect bites; burns (thermal, chemical, and electrical); and sunburn.

In another embodiment, the tetracycline responsive disease or disorder is cancer. Examples of cancers that can be treated using the compounds of the invention or a pharmaceutically acceptable salt thereof include all solid tumors, i.e., carcinomas e.g., adenocarcinomas, and sarcomas. Adenocarcinomas are carcinomas derived from glandular tissue or in which the tumor cells form recognizable glandular structures. Sarcomas broadly include tumors whose cells are embedded in a fibrillar or homogeneous substance like embryonic connective tissue. Examples of carcinomas which may be treated using the methods of the invention include, but are not limited to, carcinomas of the prostate, breast, ovary, testis, lung, colon, and breast. The methods of the invention are not limited to the treatment of these tumor types, but extend to any solid tumor derived from any organ system. Examples of treatable cancers include, but are not limited to, colon cancer, bladder cancer, breast cancer, melanoma, ovarian carcinoma, prostate carcinoma, lung cancer, and a variety of other cancers as well. The methods of the invention also cause the inhibition of cancer growth in adenocarcinomas, such as, for example, those of the prostate, breast, kidney, ovary, testes, and colon. In one embodiment, the cancers treated by methods of the invention include those described in U.S. Pat. Nos. 6,100,248; 5,843,925; 5,837,696; or 5,668,122, incorporated herein by reference in their entirety.

Alternatively, the tetracycline compounds may be useful for preventing or reducing the likelihood of cancer recurrence, for example, to treat residual cancer following surgical resection or radiation therapy. The tetracycline compounds useful according to the invention are especially advantageous as they are substantially non-toxic compared to other cancer treatments.

In a further embodiment, the compounds of the invention are administered in combination with standard cancer therapy, such as, but not limited to, chemotherapy.

Examples of tetracycline responsive states can be treated using the compounds of the invention or a pharmaceutically acceptable salt thereof also include neurological disorders which include both neuropsychiatric and neurodegenerative disorders, but are not limited to, such as Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis (ALS), progressive supranuclear palsy, epilepsy, and Creutzfeldt-Jakob disease; autonomic function disorders such as hypertension and sleep disorders, and neuropsychiatric disorders, such as depression, schizophrenia, schizoaffective disorder, Korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), bipolar affective neurological disorders, e.g., migraine and obesity.

Further neurological disorders include, for example, those listed in the American Psychiatric Association's Diagnostic and Statistical manual of Mental Disorders (DSM), the most current version of which is incorporated herein by reference in its entirety.

In another embodiment, the tetracycline responsive disease or disorder is diabetes. Diabetes that can be treated using the compounds of the invention or a pharmaceutically acceptable salt thereof include, but are not limited to, juvenile diabetes, diabetes mellitus, diabetes type I, or diabetes type II. In a further embodiment, protein glycosylation is not affected by the administration of the tetracycline compounds of the invention. In another embodiment, the tetracycline compound of the invention is administered in combination with standard diabetic therapies, such as, but not limited to insulin therapy.

In another embodiment, the tetracycline responsive disease or disorder is a bone mass disorder. Bone mass disorders that can be treated using the compounds of the invention or a pharmaceutically acceptable salt thereof include disorders where a subjects bones are disorders and states where the formation, repair or remodeling of bone is advantageous. For examples bone mass disorders include osteoporosis (e.g., a decrease in bone strength and density), bone fractures, bone formation associated with surgical procedures (e.g., facial reconstruction), osteogenesis imperfecta (brittle bone disease), hypophosphatasia, Paget's disease, fibrous dysplasia, osteopetrosis, myeloma bone disease, and the depletion of calcium in bone, such as that which is related to primary hyperparathyroidism. Bone mass disorders include all states in which the formation, repair or remodeling of bone is advantageous to the subject as well as all other disorders associated with the bones or skeletal system of a subject which can be treated with the tetracycline compounds of the invention. In a further embodiment, the bone mass disorders include those described in U.S. Pat. Nos. 5,459,135; 5,231,017; 5,998,390; 5,770,588; RE 34,656; 5,308,839; 4,925,833; 3,304,227; and 4,666,897, each of which is hereby incorporated herein by reference in its entirety.

In another embodiment, the tetracycline responsive disease or disorder is acute lung injury. Acute lung injuries that can be treated using the compounds of the invention or a pharmaceutically acceptable salt thereof include adult respiratory distress syndrome (ARDS), post-pump syndrome (PPS), and trauma. Trauma includes any injury to living tissue caused by an extrinsic agent or event. Examples of trauma include, but are not limited to, crush injuries, contact with a hard surface, or cutting or other damage to the lungs.

The tetracycline responsive disease or disorders of the invention also include chronic lung disorders. Examples of chronic lung disorders that can be treated using the compounds of the invention or a pharmaceutically acceptable salt thereof include, but are not limited, to asthma, cystic fibrosis, chronic obstructive pulmonary disease (COPD), and emphysema. In a further embodiment, the acute and/or chronic lung disorders that can be treated using the compounds of the invention or a pharmaceutically acceptable salt thereof include those described in U.S. Pat. Nos. 5,977,091; 6,043,231; 5,523,297; and 5,773,430, each of which is hereby incorporated herein by reference in its entirety.

In yet another embodiment, the tetracycline responsive disease or disorder is ischemia, stroke, or ischemic stroke.

In a further embodiment, the tetracycline compounds of the invention or a pharmaceutically acceptable salt thereof can be used to treat such disorders as described above and in U.S. Pat. Nos. 6,231,894; 5,773,430; 5,919,775 and 5,789,395, incorporated herein by reference.

In still a further embodiment, the tetracycline compounds of the invention or a pharmaceutically acceptable salt thereof can be used to treat pain, for example, inflammatory, nociceptive or neuropathic pain. The pain can be either acute or chronic.

In another embodiment, the tetracycline responsive disease or disorder is a skin wound. The invention also provides a method for improving the healing response of the epithelialized tissue (e.g., skin, mucosae) to acute traumatic injury (e.g., cut, burn, scrape, etc.). The method includes using a tetracycline compound of the invention or a pharmaceutically acceptable salt thereof to improve the capacity of the epithelialized tissue to heal acute wounds. The method may increase the rate of collagen accumulation of the healing tissue. The method may also decrease the proteolytic activity in the epithelialized tissue by decreasing the collagenolytic and/or gellatinolytic activity of MMPs. In a further embodiment, the tetracycline compound of the invention or a pharmaceutically acceptable salt thereof is administered to the surface of the skin (e.g., topically). In a further embodiment, the tetracycline compound of the invention or a pharmaceutically acceptable salt thereof is used to treat a skin wound, and other such disorders as described in, for example, U.S. Pat. Nos. 5,827,840; 4,704,383; 4,935,412; 5,258,371; 5,308,839, 5,459,135; 5,532,227; and 6,015,804; each of which is incorporated herein by reference in its entirety.

In yet another embodiment, the tetracycline responsive disease or disorder is an aortic or vascular aneurysm in vascular tissue of a subject (e.g., a subject having or at risk of having an aortic or vascular aneurysm, etc.). The tetracycline compound or a pharmaceutically acceptable salt thereof may be effective to reduce the size of the vascular aneurysm or it may be administered to the subject prior to the onset of the vascular aneurysm such that the aneurysm is prevented. In one embodiment, the vascular tissue is an artery, e.g., the aorta, e.g., the abdominal aorta. In a further embodiment, the tetracycline compounds of the invention are used to treat disorders described in U.S. Pat. Nos. 6,043,225 and 5,834,449, incorporated herein by reference in their entirety.

The compounds of the invention or a pharmaceutically acceptable salt thereof can be used alone or in combination with one or more therapeutic agent in the methods of the invention disclosed herein.

The language “in combination with” another therapeutic agent or treatment includes co-administration of the tetracycline compound and with the other therapeutic agent or treatment as either a single combination dosage form or as multiple, separate dosage forms, administration of the tetracycline compound first, followed by the other therapeutic agent or treatment and administration of the other therapeutic agent or treatment first, followed by the tetracycline compound.

The other therapeutic agent may be any agent that is known in the art to treat, prevent, or reduce the symptoms of a tetracycline-responsive disease or disorder. The choice of additional therapeutic agent(s) is based upon the particular tetracycline-responsive disease or disorder being treated. Such choice is within the knowledge of a treating physician. Furthermore, the other therapeutic agent may be any agent of benefit to the patient when administered in combination with the administration of a tetracycline compound.

As used herein, the term “subject” means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of the specified treatment.

As used herein, the term “treating” or “treatment” refers to obtaining desired pharmacological and/or physiological effect. The effect can be prophylactic or therapeutic, which includes achieving, partially or substantially, one or more of the following results: partially or totally reducing the extent of the disease, disorder or syndrome; ameliorating or improving a clinical symptom or indicator associated with the disorder; delaying, inhibiting or decreasing the likelihood of the progression of the disease, disorder or syndrome; or partially or totally delaying, inhibiting or reducing the likelihood of the onset or development of disease, disorder or syndrome.

“Effective amount” means that amount of active compound agent that elicits the desired biological response in a subject. Such response includes alleviation of the symptoms of the disease or disorder being treated. In one embodiment, the effective amount of a compound of the invention is from about 0.01 mg/kg/day to about 1000 mg/kg/day, from about 0.1 mg/kg/day to about 100 mg/kg/day, or from about 0.5 mg/kg/day to about 50 mg/kg/day.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains an effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound disclosed herein may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

In certain embodiments, the composition of this invention includes one or more additional agents. The other therapeutic agent may be any agent that is capable of treating, preventing or reducing the symptoms of a tetracycline-responsive disease or disorder. Alternatively, the other therapeutic agent may be any agent of benefit to a patient when administered in combination with the tetracycline compound in this invention.

### EXEMPLIFICATION

The following abbreviations and the terms have the indicated meanings:

| Abbreviation/Term | Meaning |
|---|---|
| Ac | acetyl |
| AIBN | 2,2′-azobis(2-methylpropionitrile) |
| aq | aqoues |
| Bn | benzyl |
| brine | saturated aqueous sodium chloride |
| Boc | tert-butoxy carbonyl or t-butoxy carbonyl |
| BPO | 2-(4-biphenyl)5-phenyloxazole |
| (Boc)₂O | di-tert-butyl dicarbonate |
| BBr₃ | boron tribromide |
| CH₂Cl₂ | methylene chloride |
| CH₃CN or MeCN | acetonitrile |
| dba | dibenzylideneacetone |
| DCE | dichloroethane |
| DIAD | diisopropyl azodicarboxylate |
| dppf | diphenylphosphino ferrocene |
| DMAP | 4-(dimethylamino)pyridine |
| DMF | N,N-dimethylformamide |
| equiv. | equivalent |
| ESI | electrospray ionization |
| Et | ethyl |
| Et₂O | ethyl ether |
| EtOAc | ethyl acetate |
| h, hr | hour |
| HCl | hydrochloric acid |
| HPLC | high performance liquid chromatography |
| KHPO₄ | potassium hydrogenphosphate |
| HPLC | high performance liquid chromatography |
| LDA | lithium diisopropylamide |
| LHMDS | lithium bis(trimethylsilyl)amide |
| Me | methyl |
| MeOH | methanol |
| MeI | methyl iodide |
| min | minute |
| MS | mass spectrum |
| MW | molecular weight |
| NaHCO₃ | sodium bicarbonate |
| NaOH | sodium hydroxide |
| Na₂SO₄ | sodium sulfate |
| NBS | N-bromosuccinimide |
| NCS | N-chlorosuccinimide |
| NDMBA | N,N-dimethylbarbituric acid |
| NFSI | N-fluorobenzenesulfonimide |
| NIS | N-iodosuccinimide |
| NMO | N-methylmorpholine-N-oxide |
| NMR | nuclear magnetic resonance spectrometry |
| Ph | phenyl |
| Piv | pivaloyl |
| RP | reverse phase |
| TBS | tert-butyldimethylsilyl |
| TFA | trifluoroacetic acid |
| THF | tetrahydrofuran |
| TLC | thin layer chromatography |
| TMEDA | N,N,N′N′-tetramethylethylenediamine |

The compounds of the invention were prepared according to the synthetic schemes shown in Schemes 1-17.

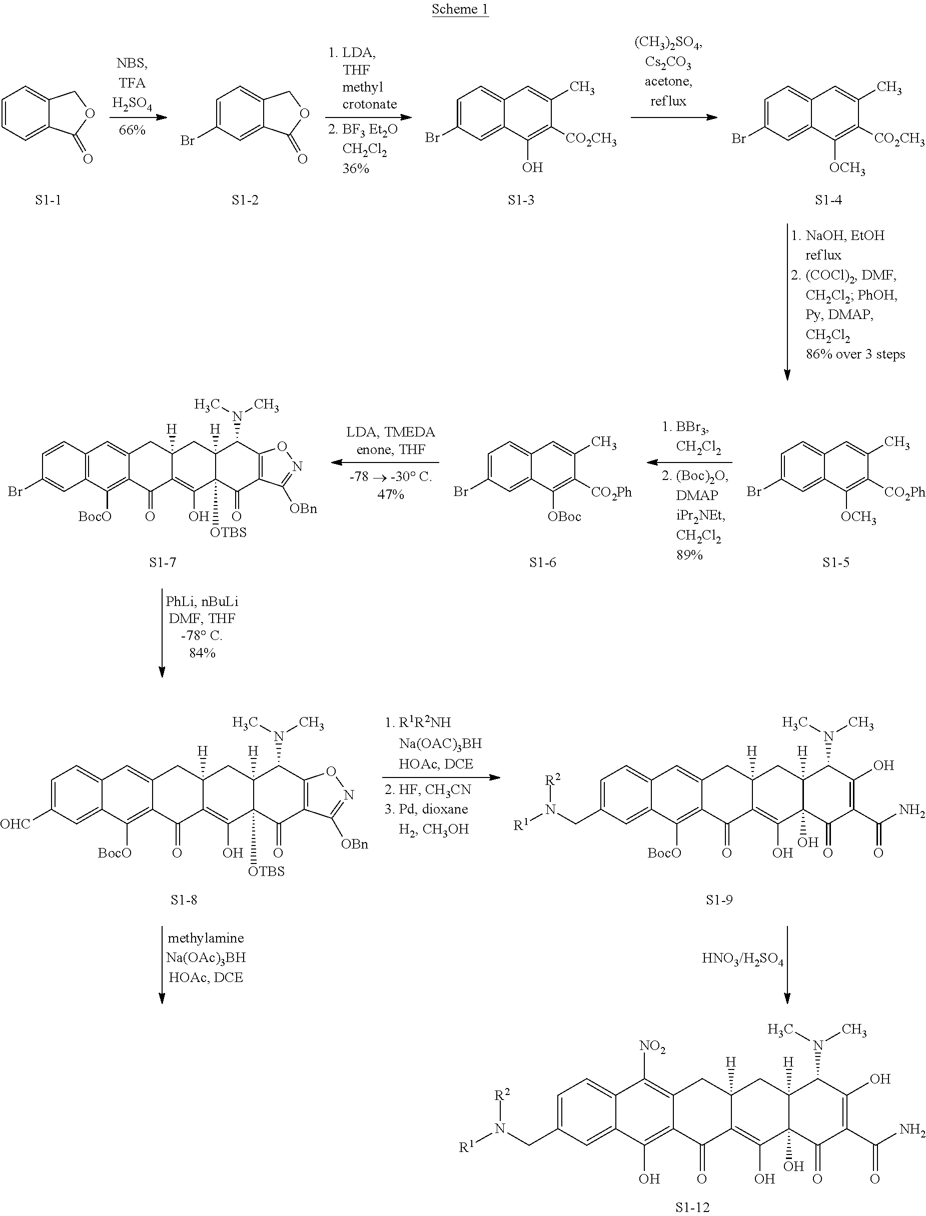

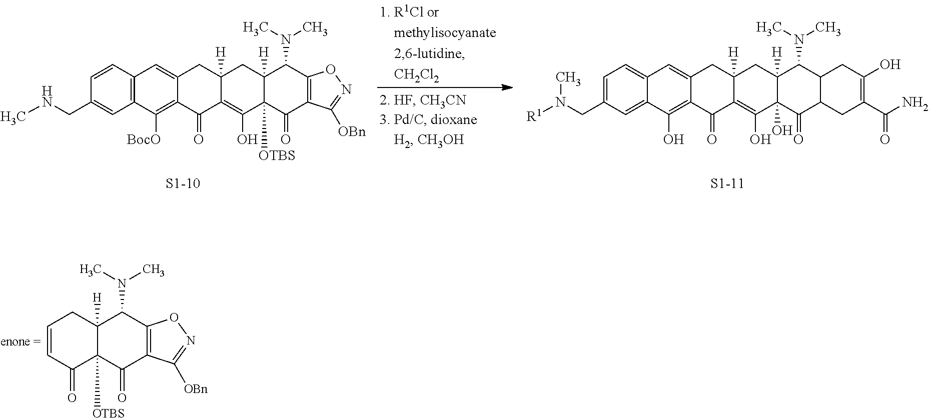

R¹ and R² are as defined for compounds of structural Formula I, or in particular, as in compounds of Formulas S1-9 and S1-12, R¹ is (C₁-C₈)alkyl, (C₀-C₆)alkylene-(C₁-C₆)alkoxy, (C₀-C₆) alkylene-(C₃-C₇)cycloalkyl, —C(O)—(C₁-C₆)alkyl, phenyl, benzyl or pyridyl and R² is hydrogen or (C₁-C₄)alkyl, or R¹ and R² are taken together with the nitrogen atom to which they are bound to form a (4-7 membered) heterocyclic ring optionally containing one additional heteroatom selected from S, O or N. Additionally as in compound of Formula S1-11, R¹ is —C(O)—(C₁-C₆)alkoxy, —C(O)—N(R²)(R²) or —S(O)ₘ—(C₁-C₆)alkyl, and R² is CH₃.

The following compounds were prepared according to Scheme 1.

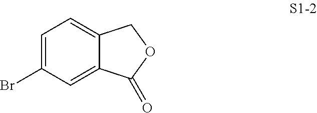

NBS (53.4 g, 300 mmol, 1.5 eq) was added portion wise to a solution of phthalide (S1-1) (26.83 g, 200 mmol, 1.0 eq) in a mixture of TFA (100 mL) and sulfuric acid (45 mL) at rt over 9 h. The reaction mixture (an orange solution) was stirred at rt for about 60 h. (Crude NMR showed the reaction is complete.) Then the reaction mixture was poured onto ice, extracted with methylene chloride (3×300 mL). The combined organic phase was dried over MgSO₄, filtered and concentrated to afford a yellow solid. The residue was purified by flash-column chromatography (5-10% ethyl acetate-hexanes) to afford the desired product S1-2 (white solid, 28.17 g, 66%) and the other regioisomer S3-1 (white solid, 12.7 g, 30%). ¹H NMR (400 MHz, CDCl₃) δ 8.04 (d, J=1.8 Hz, 1H), 7.78 (dd, J=1.8, 7.9 Hz, 1H), 7.37 (d, J=7.9 Hz, 1H), 5.26 (s, 2H).

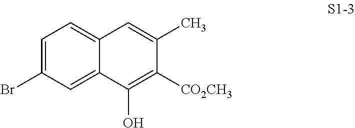

A solution of n-butyllithium in hexanes (2.50 M, 48.4 mL, 121.1 mmol, 1.2 eq) was added to a solution of diisopropylamine (16.97 mL, 121.1 mmol, 1.2 eq) in tetrahydrofuran (400 mL) at −78° C. The resulting mixture was stirred vigorously at −78° C. for 30 min, warmed up to 0° C. for 5 min, and cooled back to −78° C. A solution of the bromophthalide S1-2 (21.5 g, 100.9 mmol, 1.0 eq) in tetrahydrofuran (200 mL) was added slowly via a cannula. The resulting dark solution was allowed to warm slowly to −50° C. over 3 h. Methyl crotonate (11.77 mL, 111.02 mmol, 1.1 eq) was added slowly and the resulting mixture was allowed to warm up to rt without removing the cooling bath. The reaction mixture was poured into 1 N HCl solution (600 mL) and extracted with ethyl acetate (3×200 mL). The organic extracts were combined and dried over anhydrous MgSO₄. The dried solution was filtered and the filtrate was concentrated, dried under high vacuum, providing an orange foamy solid (27.5 g), which was used for the next reaction without purification.

The above crude product was dissolved in methylene chloride (250 mL). BF₃-Et₂O (2.53 mL, 20.18 mmol, 0.2 eq) was added drop-wise at rt. The resulting brownish mixture was stirred at rt for 1 h (Monitored by TLC. Desired product is less polar) and poured into 0.5 N HCl solution (600 mL). The organic layer was separated and the aqueous layer was extracted with methylene chloride (2×200 mL). The organic extracts were combined and dried over anhydrous MgSO₄. The dried solution was filtered and the filtrate was concentrated, providing a thick brownish oil. The product S1-3 was purified by flash-column chromatography (5-10% ethyl acetate-hexanes) to afford compound S1-3 as an off-white solid (6.32 g, 22%, two steps), ¹H NMR (500 MHz, CDCl₃) δ 12.68 (s, 1H), 8.52 (s, 1H), 7.63-7.61 (d, J=8.5 Hz, 1H), 7.52-7.50 (d, J=8.5 Hz, 1H), 7.07 (s, 1H), 4.01 (s, 3H), 2.63 (s, 3H).

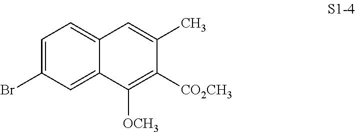

Cs₂CO₃ powder (9.06 g, 27.79 mmol, 1.3 eq) and dimethyl sulfate (2.43 mL, 25.66 mmol, 1.2 eq) were added to a solution of S1-3 (6.31 g, 21 mmol, 1.0 eq) in acetone (60 mL). The mixture was heated at reflux for 1 h (Monitored by TLC. Desired product is slightly more polar than starting material), cooled to rt, and filtered through a short pad of Celite. The Celite cake was washed thoroughly with acetone. The filtrate was concentrated to afford S1-4 as a white solid, which was used for the next reaction directly without further purification.

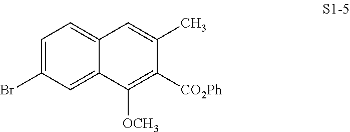

A mixture of EtOH (10 mL) and 4 N NaOH solution (10 mL) was added to compound S1-4. The mixture was stirred at 85° C. overnight (initially a suspension, became homogeneous upon heating). The reaction mixture was cooled to rt, acidified with about 11 mL of 4 N HCl solution to pH=3, and extracted with methylene chloride (4×30 mL). The combined organic extracts were dried over MgSO₄, filtered, and concentrated to afford the carboxylic acid as a pale yellow solid.

Oxalic chloride (2.24 mL, 25.68 mmol, 1.2 eq) was added to a solution of the above carboxylic acid in anhydrous methylene chloride (100 mL) at rt, followed by a couple of drops of DMF (gas evolution). The mixture was stirred at rt for 1 h and the solvent was evaporated. The residue was dried under high vacuum. The crude acid chloride was re-dissolved in methylene chloride (100 mL). Pyridine (3.46 mL, 42.8 mmol, 2.0 eq), phenol (2.11 g, 22.47 mmol, 1.05 eq) and DMAP (cat.) were added. The reaction mixture was stirred for several hours at rt, added with 1 N HCl solution (100 mL), and extracted with methylene chloride (3×50 mL). The combined organic extracts were washed with brine (40 mL) and dried over anhydrous MgSO₄. The dried solution was filtered and the filtrate was concentrated. The residue was purified by flash-column chromatography (5-10% ethyl acetate-hexanes) to afford the desired product S1-5 as a white solid (6.86 g, 86%, three steps). ¹H NMR (400 MHz, CDCl₃) δ 8.22 (d, J=1.8 Hz, 1H), 7.60-7.52 (m, 2H), 7.44-7.39 (m, 3H), 7.27-7.24 (m, 3H), 4.05 (s, 3H), 2.53 (s, 3H).

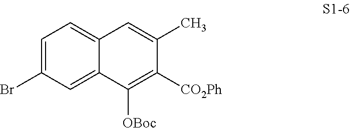

A solution of BBr₃ in CH₂Cl₂ (1.0 M, 5.83 mL, 5.83 mmol, 2.0 eq) was added slowly over 4 min to a solution of compound S1-5 (1.08 g, 2.91 mmol, 1.0 eq) in methylene chloride (60 mL) at −70° C. The resulting orange solution was allowed to warm to 45° C. in 5 h (monitored by LCMS and TLC (product is slightly less polar)), poured into saturated NaHCO₃ solution (100 mL). The mixture was stirred at rt for 10 min and extracted with methylene chloride (3×60 mL). The organic extracts were combined and dried over anhydrous MgSO₄. The dried solution was filtered, and the filtrate was concentrated, providing the desired product as a white solid, which was used in the next reaction without further purification.

Di-tert-butyl dicarbonate (670 mg, 3.20 mmol, 1.1 eq), diisopropylethyl amine (1.01 mL, 5.82 mmol, 2.0 eq), and N,N-dimethylaminopyridine (20 mg, 0.16 mmol, 0.05 eq) were added to a solution of the above compound in methylene chloride (60 mL). The resulting mixture was stirred at rt overnight (monitored by LCMS and TLC (product is slightly more polar). Reaction might had completed within 1 h) and concentrated under reduced pressure. The residue was purified by flash-column chromatography (5% ethyl acetate-hexanes) to afford the Boc protection product S1-6 as a white solid (1.19 g, 89%, two steps). ¹H NMR (400 MHz, CDCl₃) δ 8.03 (d, J=1.8 Hz, 1H), 7.63-7.55 (m, 3H), 7.43-7.39 (m, 2H), 7.28-7.24 (m, 3H), 2.60 (s, 3H), 1.44 (s, 9H); ¹³C NMR (100 MHz, CDCl₃) δ. 164.7, 151.2, 150.5, 144.9, 134.0, 133.4, 131.6, 129.5, 128.9, 126.9, 126.7, 126.3, 124.5, 124.0, 121.7, 120.9, 84.6, 27.5, 20.7.

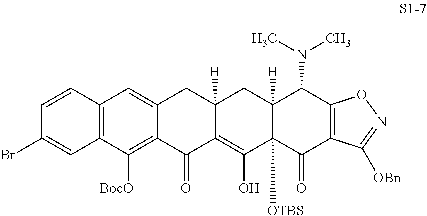

Michael-Dieckmann Cyclization. General Procedure A. A solution of lithium diisopropylamide (1.8 M, 4.30 mL, 7.74 mmol, 3.0 eq) in heptane/ethylbenzene/THF was added drop wise via syringe to a solution of ester S1-6 (2.36 g, 5.16 mol, 2.0 eq), enone (1.24 g, 2.58 mol, 1.0 eq) and TMEDA (2.32 mL, 15.48 mmol, 6.0 eq) in tetrahydrofuran (100 mL) at −78° C. The resulting red orange reaction mixture was allowed to warm to −10° C. over 2 h, then was diluted with aqueous potassium phosphate buffer solution (pH 7.0, 0.2 M, 50 mL) and saturated ammonium chloride. The resulting mixture was extracted with ethyl acetate (2×200 mL). The organic extracts were combined and the combined solution was dried over anhydrous sodium sulfate. The dried solution was filtered and the filtrate was concentrated, affording a red oil. The product was purified by flash-column chromatography (5-10% ethyl acetate-hexanes) to afford the Michael-Dieckmann cyclization product S1-7 as an orange solid (1.02 g, 47%). ¹H NMR (500 MHz, CDCl₃) δ 15.98 (br s, 1H), 8.24 (br s, 1H), 7.64 (s, 2H), 7.51-7.49 (m, 3H), 7.39-7.33 (m, 3H), 5.37 (d, J=12.0 Hz, 1H), 5.34 (d, J=12.0 Hz, 1H), 3.96 (d, 10.5 Hz, 1H), 3.11-3.06 (m, 2H), 2.93 (t, J=15.5 Hz, 1H), 2.56 (dd, J=11.0, 5.0 Hz, 1H), 2.49 (s, 6H), 2.49-2.46 (m, 1H), 2.14 (d, J=14.0 Hz, 1H), 1.59 (s, 9H), 0.82 (s, 9H), 0.27 (s, 3H), 0.12 (s, 3H); MS (ESI) m/z 845.70, 847.69 (M+H).

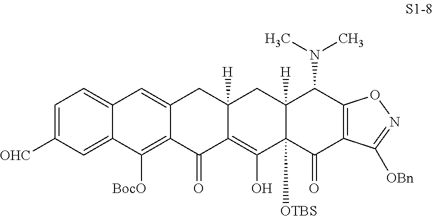

Formylation. General Procedure B. A solution of phenyllithium in di-n-butyl ether (1.8 M, 200 μL, 0.362 mmol, 2.0 eq) was added drop wise to a solution of compound S1-7 (153 mg, 0.181 mmol, 1.0 eq) in tetrahydrofuran (9 mL) at −78° C., forming a red solution. After 5 min, a solution of n-butyllithium in hexanes (2.5 M, 94 μL, 0.235 mmol, 1.3 eq) was added drop wise at −78° C. followed 5 min later by N,N-dimethylformamide (69 μL, 0.904 mmol, 5.0 eq). The deep red reaction mixture was stirred at −78° C. for 1 h. Saturated aqueous ammonium chloride solution (10 mL) was added drop wise at −78° C., followed by aqueous potassium phosphate buffer solution (pH 7.0, 0.2 M, 10 mL). The reaction mixture was allowed to warm up to 23° C., then was extracted with methylene chloride (3×30 mL). The organic extracts were combined and the combined solution was dried over anhydrous sodium sulfate. The dried solution was filtered and the filtrate was concentrated. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; gradient: 90→95% B over 10 min, then 100% B for 5 min; mass-directed fraction collection]. Fractions with the desired MW were collected and concentrated on a RotaVap at rt to afford the desired product S1-8 (120.8 mg, 84%) as a yellow solid: R_{f}=0.15 (25% ethyl acetate-hexanes); ¹H NMR (400 MHz, CDCl₃) δ 15.98 (br s, 1H), 10.17 (s, 1H), 8.60 (br s, 1H), 8.06 (dd, J=8.4, 1.6 Hz, 1H), 7.88 (d, J=8.4 Hz, 1H), 7.62 (s, 1H), 7.52-7.50 (m, 2H), 7.41-7.34 (m, 3H), 5.38 (d, J=12.4 Hz, 1H), 5.35 (d, J=12.4 Hz, 1H), 3.98 (d, J=10.4 Hz, 1H), 3.18-3.13 (m, 2H), 3.03-2.96 (m, 1H), 2.61-2.56 (m, 1H), 2.51 (s, 6H), 2.50-2.48 (m, 1H), 2.18 (d, J=14.4 Hz, 1H), 1.62 (s, 9H), 0.84 (s, 9H), 0.29 (s, 3H), 0.15 (s, 3H); ¹³C NMR (100 MHz, CDCl₃) δ191.5, 187.2, 181.7, 167.5, 140.5, 138.7, 135.0, 134.7, 129.5, 128.5, 128.48, 128.45, 128.42, 126.8, 126.0, 123.9, 120.7, 108.5, 108.4, 84.9, 81.8, 72.5, 61.2, 46.4, 41.8, 39.9, 28.4, 27.7, 26.0, 22.7, 19.0, −2.6, −3.8; MS (ESI) m/z 795.7 (M+H).

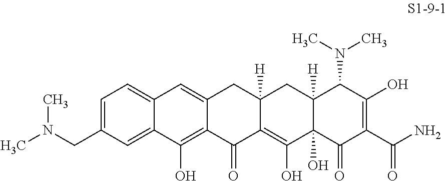

One-Pot Reductive Amination. General Procedure C.

A solution of Me₂NH in THF (2.0 M, 44 μL, 0.088 mmol, 7.0 eq), acetic acid (5 μL, 0.088 mmol, 7.0 eq) and sodium triacetoxyborohydride (18.7 mg, 0.088 mmol, 7.0 eq) were added in sequence to a solution of aldehyde S1-8 (10 mg, 0.012 mmol, 1.0 eq) in 1,2-dichloroethane (1 mL) at 23° C. After stirring for 2 h, the reaction mixture was poured into an aqueous potassium phosphate buffer solutions (pH=7.0, 10 mL). The product was extracted into methylene chloride (3×15 mL). The combined organic extracts were dried over anhydrous sodium sulfate. The dried solution was filtered and the filtrate was concentrated, which was used directly for the next reactions without purification.

HF reaction. General Procedure D. Concentrated aqueous hydrofluoric acid solution (48 wt %, 0.3 mL) was added to a solution of the above reductive amination product in acetonitrile (0.6 mL) in a polypropylene reaction vessel at 23° C. The resulting mixture was stirred vigorously at 23° C. overnight, then was poured into water (25 mL) containing dipotassium hydrogenphosphate (3.6 g). The product was extracted into ethyl acetate (3×20 mL). The combined organic extracts were dried over anhydrous sodium sulfate. The dried solution was filtered and the filtrate was concentrated, affording a yellow solid.

Hydrogenation. General Procedure E. Methanol (3.0 mL) was added to the above residue, forming a yellow solution. Pd black (cat) was added in one portion at 23° C. An atmosphere of hydrogen was introduced by briefly evacuating the flask, then flushing with pure hydrogen (1 atm). The reaction mixture was stirred at 23° C. for 4 h, then was filtered through a pad of Celite. The filtrate was concentrated, affording a yellow solid. The residue was purified by a preparative reverse phase HPLC on a Waters Autopurification system using Phenomenex Polymerx 10μ RP-1 100A column [10 μm, 150×21.20 mm; flow rate, 20 mL/min; Solvent A: 0.05 N HCl/water; Solvent B: CH₃CN; injection volume: 3.0 mL (0.05 N HCl/water containing 10 mg oxalic acid); gradient: 10→35% B over 10 min; mass-directed fraction collection]. Fractions with the desired MW were collected and freeze-dried to yield 8,9-(3a-dimethylaminomethyl)benzo-6-deoxy-6-demethyltetracycline hydrochloride (Compound 131) as a yellow solid (3.63 mg, 51%, three steps). ¹H NMR (400 MHz, CD₃OD, hydrochloride) δ 8.48 (br s, 1H), 7.83 (d, J=7.9 Hz, 1H), 7.74 (d, J=6.9 Hz, 1H), 7.16 (br s, 1H), 4.49 (br s, 2H), 4.10 (br s, 1H), 3.03-2.95 (m, 9H), 2.87 (s, 6H), 2.64-2.57 (m, 1H), 2.25-2.22 (m, 1H), 1.60-1.58 (m, 1H); MS (ESI) m/z 522.50 (M+H).

The following compounds were prepared similarly to Compound 131 using general procedures C, D, and E from aldehyde S1-8.

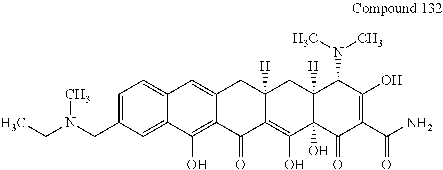

Compound 132: ¹H NMR (400 MHz, CD₃OD, hydrochloride) δ 8.49 (br s, 1H), 7.83 (d, J=7.3 Hz, 1H), 7.75 (d, J=7.8 Hz, 1H), 7.16 (br s, 1H), 4.59 (m, 1H), 4.41 (m, 1H), 4.10 (br s, 1H), 3.30-2.95 (m, 11H), 2.78 (s, 3H), 2.64-2.58 (m, 1H), 2.28-2.18 (m, 1H), 1.60-1.58 (m, 1H), 1.38 (m, 3H); MS (ESI) m/z 536.59 (M+H).

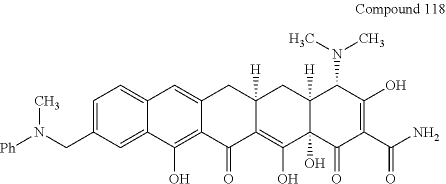

Compound 118: ¹H NMR (400 MHz, CD₃OD, hydrochloride) δ 8.28 (s, 1H), 7.80 (d, J=8.2 Hz, 1H), 7.51-7.43 (m, 6H), 7.13 (s, 1H), 4.96 (s, 2H), 4.08 (s, 1H), 3.41 (s, 3H), 3.03-2.96 (m, 9H), 2.64 (t, J=14.6 Hz, 1H), 2.20-2.19 (m, 1H), 1.67-1.57 (m, 1H); MS (ESI) m/z 584.54 (M+H).

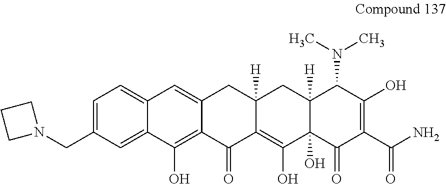

Compound 137: ¹H NMR (400 MHz, CD₃OD, hydrochloride) δ 8.44 (s, 1H), 7.70 (d, J=8.7 Hz, 1H), 7.68 (dd, J=1.8, 8.7 Hz, 1H), 7.13 (s, 1H), 4.55 (s, 2H), 4.29-4.22 (m, 2H), 4.12-4.08 (m, 3H), 3.10-2.96 (m, 9H), 2.63-2.55 (m, 2H), 2.50-2.46 (m, 1H), 2.26-2.21 (m, 1H), 1.65-1.56 (m, 1H); MS (ESI) m/z 534.51 (M+H).

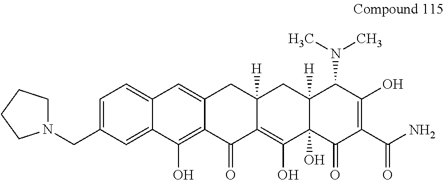

Compound 115: ¹H NMR (400 MHz, CD₃OD, hydrochloride) δ 8.49 (s, 1H), 7.81-7.78 (m, 2H), 7.15 (s, 1H), 4.54 (s, 2H), 4.10 (s, 1H), 3.49 (m, 2H), 3.22 (m, 2H), 3.03-2.95 (m, 9H), 2.63-2.58 (m, 1H), 2.25-2.17 (m, 3H), 2.02-1.98 (m, 2H), 1.64-1.54 (m, 1H); MS (ESI) m/z 548.58 (M+H).

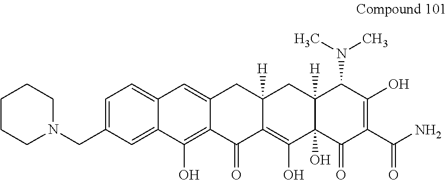

Compound 101: ¹H NMR (400 MHz, CD₃OD, hydrochloride) δ 8.50 (s, 1H), 7.84-7.76 (m, 2H), 7.17 (s, 1H), 4.45 (s, 2H), 4.10 (s, 1H), 3.46-3.44 (m, 2H), 3.10-2.96 (m, 11H), 2.65-2.59 (m, 1H), 2.25-2.17 (m, 1H), 1.92-1.79 (m, 5H), 1.60-1.52 (m, 2H); MS (ESI) m/z 562.54 (M+H).

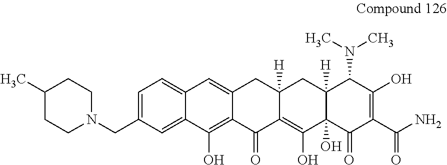

Compound 126: ¹H NMR (400 MHz, CD₃OD, hydrochloride) δ 8.48 (s, 1H), 7.83-7.75 (m, 2H), 7.16 (s, 1H), 4.44 (s, 2H), 4.09 (s, 1H), 3.46-3.44 (m, 2H), 3.10-2.95 (m, 11H), 2.64-2.59 (m, 1H), 2.26-2.19 (m, 1H), 1.88-1.85 (m, 2H), 1.69-1.60 (m, 2H), 1.45-1.43 (m, 2H), 0.96 (d, J=5.5 Hz, 3H); MS (ESI) m/z 576.57 (M+H).

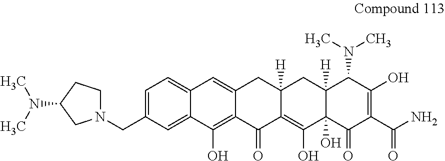

Compound 113: ¹H NMR (400 MHz, CD₃OD, hydrochloride) δ 8.59 (s, 1H), 7.86-7.84 (m, 2H), 7.21 (s, 1H), 4.71 (s, 2H), 4.68 (m, 1H), 4.10 (s, 1H), 3.95-3.90 (m, 2H), 3.71-3.69 (m, 2H), 3.14-2.96 (m, 15H), 2.70-2.50 (m, 3H), 2.25-2.22 (m, 1H), 1.68-1.59 (m, 1H); MS (ESI) m/z 591.72 (M+H).

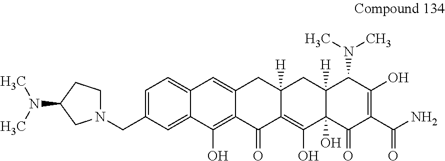

Compound 134: ¹H NMR (400 MHz, CD₃OD, hydrochloride) δ 8.59 (s, 1H), 7.88 (d, J=8.2 Hz, 1H), 7.85 (d, J=9.2 Hz, 1H), 7.22 (s, 1H), 4.70 (s, 2H), 4.27 (m, 1H), 4.10 (s, 1H), 3.95-3.90 (m, 2H), 3.73-3.69 (m, 2H), 3.14-2.92 (m, 15H), 2.67 (t, J=14.2 Hz, 1H), 2.64-2.45 (m, 2H), 2.25-2.21 (m, 1H), 1.68-1.59 (m, 1H); MS (ESI) m/z 591.60 (M+H).

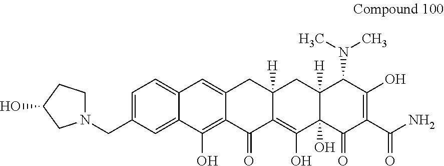

Compound 100: ¹H NMR (400 MHz, CD₃OD, hydrochloride) δ 8.55 (s, 1H), 7.86 (d, J=8.7 Hz, 1H), 7.78-7.75 (m, 1H), 7.21 (s, 1H), 4.70-4.51 (m, 3H), 4.11 (s, 1H), 3.72-3.47 (m, 2H), 3.41-3.24 (m, 2H), 3.12-2.97 (m, 9H), 2.67 (t, J=14.2 Hz, 1H), 2.44-2.42 (m, 0.5H), 2.26-2.15 (m, 2H), 2.04-1.99 (m, 0.5H), 1.68-1.59 (m, 1H); MS (ESI) m/z 564.51 (M+H).

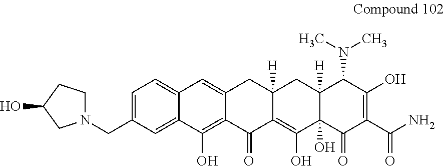

Compound 102: ¹H NMR (400 MHz, CD₃OD, hydrochloride) δ 8.54 (s, 1H), 7.86 (d, J=8.2 Hz, 1H), 7.78-7.75 (m, 1H), 7.20 (s, 1H), 4.67-4.51 (m, 3H), 4.11 (s, 1H), 3.74-3.47 (m, 2H), 3.41-3.23 (m, 2H), 3.12-2.97 (m, 9H), 2.66 (t, J=14.6 Hz, 1H), 2.48-2.39 (m, 0.5H), 2.25-2.22 (m, 1H), 2.16-2.14 (m, 1H), 2.06-1.99 (m, 0.5H), 1.68-1.58 (m, 1H); MS (ESI) m/z 564.51 (M+H).

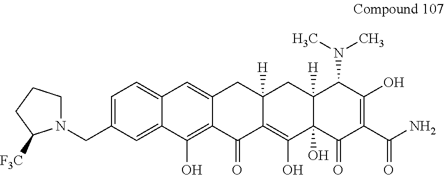

Compound 107: ¹H NMR (400 MHz, CD₃OD, hydrochloride) δ 8.57 (s, 1H), 7.87 (d, J=7.8 Hz, 1H), 7.77 (d, J=7.3 Hz, 1H), 7.22 (s, 1H), 4.82-4.56 (m, 3H), 4.10 (s, 1H), 3.51 (m, 2H), 3.15-2.97 (m, 9H), 2.68 (t, J=13.7 Hz, 1H), 2.59-2.53 (m, 1H), 2.26-2.07 (m, 4H), 1.68-1.58 (m, 1H); MS (ESI) m/z 616.52 (M+H).

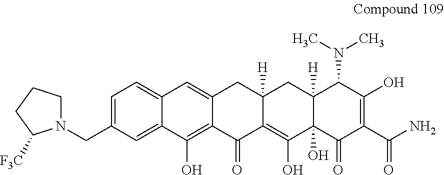

Compound 109: ¹H NMR (400 MHz, CD₃OD, hydrochloride) δ 8.51 (s, 1H), 7.85 (d, J=8.7 Hz, 1H), 7.74 (d, J=8.7 Hz, 1H), 7.20 (s, 1H), 4.69 (d, J=13.3 Hz, 1H), 4.48 (d, J=12.8 Hz, 1H), 4.38 (m, 1H), 4.09 (s, 1H), 3.42-3.30 (m, 2H), 3.12-2.96 (m, 9H), 2.68 (t, J=13.7 Hz, 1H), 2.51-2.44 (m, 1H), 2.23-2.03 (m, 4H), 1.68-1.58 (m, 1H); MS (ESI) m/z 616.51 (M+H).

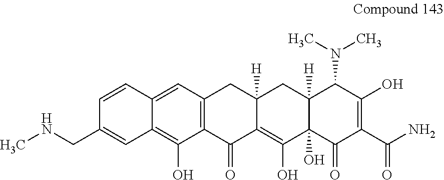

Compound 143: ¹H NMR (400 MHz, CD₃OD:hydrochloride salt) δ 8.51 (s, 1H), 7.86 (d, J=8.5 Hz, 1H), 7.71 (dd, J=1.8, 8.5 Hz, 1H), 7.21 (s, 1H), 4.37 (s, 2H), 4.10 (s, 1H), 3.17-2.96 (m, 9H), 2.77 (s, 3H), 2.67 (t, J=13.4 Hz, 1H), 2.28-2.21 (m, 1H), 1.69-1.57 (m, 1H); MS (ESI) m/z 508.45 (M+H).

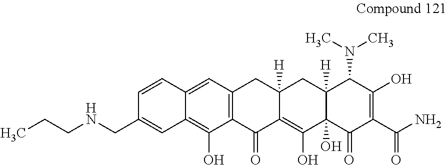

Compound 121: ¹H NMR (400 MHz, CD₃OD:hydrochloride salt) δ 8.52 (s, 1H), 7.86 (d, J=8.5 Hz, 1H), 7.74 (dd, J=1.8, 8.5 Hz, 1H), 7.21 (s, 1H), 4.38 (s, 2H), 4.11 (s, 1H), 3.17-2.96 (m, 11H), 2.67 (t, J=13.4 Hz, 1H), 2.28-2.20 (m, 1H), 1.82-1.71 (m, 2H), 1.69-1.57 (m, 1H), 1.04 (t, J=7.9 Hz, 3H); MS (ESI) m/z 536.50 (M+H).

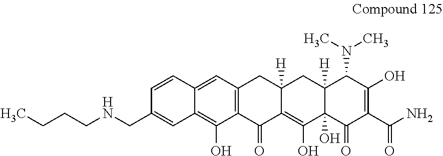

Compound 125: ¹H NMR (400 MHz, CD₃OD:hydrochloride salt) δ 8.52 (s, 1H), 7.86 (d, J=8.5 Hz, 1H), 7.74 (dd, J=1.8, 8.5 Hz, 1H), 7.21 (s, 1H), 4.38 (s, 2H), 4.11 (s, 1H), 3.17-2.96 (m, 11H), 2.67 (t, J=13.4 Hz, 1H), 2.28-2.20 (m, 1H), 1.78-1.58 (m, 3H), 1.50-1.39 (m, 2H), 0.99 (t, J=7.3 Hz, 3H); MS (ESI) m/z 550.52 (M+H).

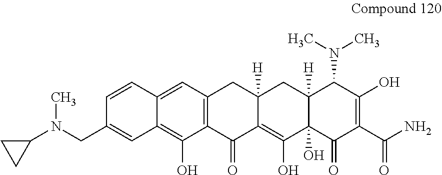

Compound 120: ¹H NMR (400 MHz, CD₃OD:hydrochloride salt) δ 8.54 (s, 1H), 7.84 (d, J=8.5 Hz, 1H), 7.78 (d, J=8.5 Hz, 1H), 7.19 (s, 1H), 4.76-4.57 (m, 2H), 4.12 (s, 1H), 3.18-2.87 (m, 13H), 2.64 (t, J=14.0 Hz, 1H), 2.28-2.19 (m, 1H), 1.69-1.55 (m, 1H), 1.06-0.83 (m, 3H), 0.83-0.71 (m, 1H); MS (ESI) m/z 548.58 (M+H).

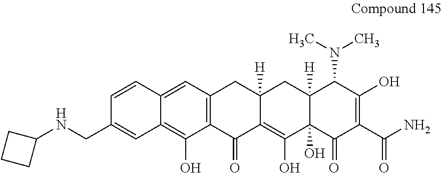

Compound 145: ¹H NMR (400 MHz, CD₃OD:hydrochloride salt) δ 8.48 (s, 1H), 7.83 (d, J=8.5 Hz, 1H), 7.70 (dd, J=1.2, 8.5 Hz, 1H), 4.27 (s, 2H), 4.11 (s, 1H), 3.92-3.78 (m, 1H), 3.17-2.88 (m, 10H), 2.70-2.59 (m, 1H), 2.42-2.18 (m, 5H), 2.01-1.86 (m, 1H), 1.69-1.55 (m, 1H); MS (ESI) m/z 548.42 (M+H).

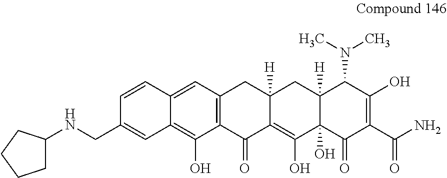

Compound 146: ¹H NMR (400 MHz, CD₃OD:hydrochloride salt) δ 8.53 (s, 1H), 7.86 (d, J=8.5 Hz, 1H), 7.74 (dd, J=1.8, 8.5 Hz, 1H), 7.20 (s, 1H), 4.39 (s, 2H), 4.11 (s, 1H), 3.71-3.59 (m, 1H), 3.18-2.92 (m, 9H), 2.73-2.62 (m, 1H), 2.28-2.14 (m, 3H), 1.91-1.56 (m, 7H); MS (ESI) m/z 562.49 (M+H).

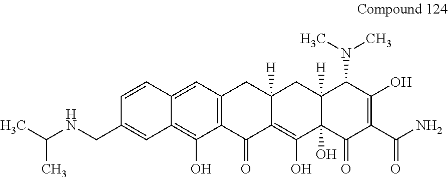

Compound 124: ¹H NMR (400 MHz, CD₃OD:hydrochloride salt) δ 8.53 (s, 1H), 7.85 (d, J=8.5 Hz, 1H), 7.74 (dd, J=1.8, 8.5 Hz, 1H), 7.20 (s, 1H), 4.39 (s, 2H), 4.11 (s, 1H), 3.55-3.45 (m, 1H), 3.16-2.91 (m, 9H), 2.67 (t, J=14.0 Hz, 1H), 2.28-2.19 (m, 1H), 1.69-1.56 (m, 1H), 1.43 (d, J=6.7 Hz, 6H); MS (ESI) m/z 536.53 (M+H).

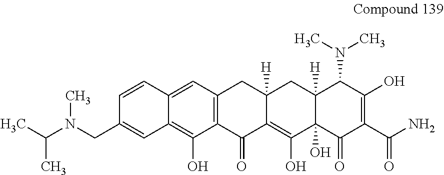

Compound 139: ¹H NMR (400 MHz, CD₃OD:hydrochloride salt) δ 8.55 (s, 1H), 7.88 (d, J=8.5 Hz, 1H), 7.74 (dd, J=1.8, 8.5 Hz, 1H), 7.22 (s, 1H), 4.64 (d, J=12.8 Hz, 1H), 4.36 (d, J=13.4 Hz, 1H), 4.10 (s, 1H), 3.74-3.64 (m, 1H), 3.17-2.93 (m, 9H), 2.78-2.62 (m, 4H), 2.28-2.18 (m, 1H), 1.70-1.56 (m, 1H), 1.47 (d, J=6.7 Hz, 3H), 1.43 (d, J=6.7 Hz, 3H); MS (ESI) m/z 550.55 (M+H).

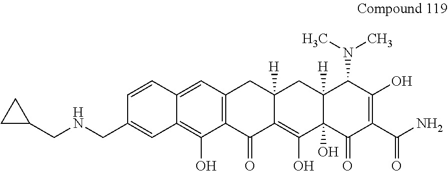

Compound 119: ¹H NMR (400 MHz, CD₃OD:hydrochloride salt) δ 8.52 (s, 1H), 7.85 (d, J=8.5 Hz, 1H), 7.72 (dd, J=1.8, 8.5 Hz, 1H), 7.20 (s, 1H), 4.39 (s, 2H), 4.09 (s, 1H), 3.17-2.91 (m, 12H), 2.67 (t, J=14.0 Hz, 1H), 2.27-2.18 (m, 1H), 1.70-1.56 (m, 1H), 1.20-1.07 (m, 1H), 0.75-0.69 (m, 2H), 0.45-0.39 (m, 2H); MS (ESI) m/z 548.53 (M+H).

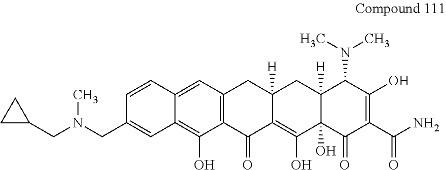

Compound 111: ¹H NMR (400 MHz, CD₃OD:hydrochloride salt) δ 8.55 (s, 1H), 7.88 (d, J=8.5 Hz, 1H), 7.75 (dd, J=1.8, 8.5 Hz, 1H), 7.23 (s, 1H), 4.70 (d, J=12.8 Hz, 1H), 4.43 (d, J=13.4 Hz, 1H), 4.11 (s, 1H), 3.24-2.95 (m, 12H), 2.90 (s, 3H), 2.68 (t, J=14.0 Hz, 1H), 2.29-2.20 (m, 1H), 1.71-1.57 (m, 1H), 1.27-1.13 (m, 1H), 0.88-0.73 (m, 2H), 0.53-0.36 (m, 2H); MS (ESI) m/z 562.55 (M+H).

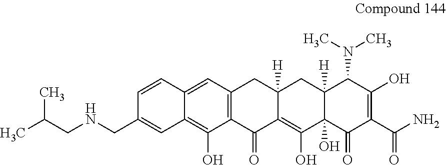

Compound 144: ¹H NMR (400 MHz, CD₃OD:hydrochloride salt) δ 8.53 (s, 1H), 7.84 (d, J=8.5 Hz, 1H), 7.74 (dd, J=1.8, 8.5 Hz, 1H), 7.19 (s, 1H), 4.39 (s, 2H), 4.10 (s, 1H), 3.17-2.90 (m, 9H), 2.66 (t, J=14.0 Hz, 1H), 2.28-2.18 (m, 1H), 2.12-2.00 (m, 1H), 1.70-1.57 (m, 1H), 1.03 (d, J=6.7 Hz, 6H); MS (ESI) m/z 550.49 (M+H).

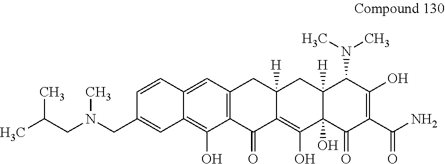

Compound 130: ¹H NMR (400 MHz, CD₃OD:hydrochloride salt) δ 8.55 (s, 1H), 7.89 (d, J=8.5 Hz, 1H), 7.74 (dd, J=1.8, 8.5 Hz, 1H), 7.23 (s, 1H), 4.62 (d, J=13.4 Hz, 1H), 4.45 (d, J=12.8 Hz, 1H), 4.10 (s, 1H), 3.19-2.93 (m, 11H), 2.88 (2, 3 H), 2.69 (t, J=14.0 Hz, 1H), 2.28-2.15 (m, 2H), 1.70-1.57 (m, 1H), 1.05 (d, J=6.7 Hz, 3H), 1.00 (d, J=6.7 Hz, 3H); MS (ESI) m/z 564.51 (M+H).

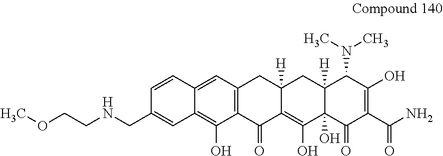

Compound 140: ¹H NMR (400 MHz, CD₃OD:hydrochloride salt) δ 8.51 (s, 1H), 7.84 (d, J=8.5 Hz, 1H), 7.72 (dd, J=1.8, 8.5 Hz, 1H), 7.18 (s, 1H), 4.41 (s, 2H), 4.11 (s, 1H), 3.67 (t, J=4.9 Hz, 2H), 3.41 (s, 3H), 3.28-3.24 (m, 2H), 3.15-2.92 (m, 9H), 2.65 (t, J=14.0 Hz, 1H), 2.27-2.18 (m, 1H), 1.69-1.54 (m, 1H); MS (ESI) m/z 552.47 (M+H).

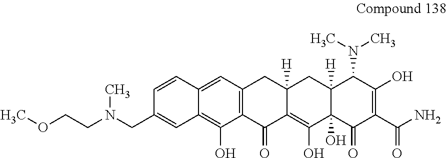

Compound 138: ¹H NMR (400 MHz, CD₃OD:hydrochloride salt) δ 8.54 (s, 1H), 7.88 (d, J=8.5 Hz, 1H), 7.73 (d, J=8.5 Hz, 1H), 7.22 (s, 1H), 4.64 (d, J=12.8 Hz, 1H), 4.47 (d, J=12.8 Hz, 1H), 4.11 (s, 1H), 3.80-3.70 (m, 2H), 3.51-3.31 (m, 5H), 3.17-2.93 (m, 9H), 2.90 (s, 3H), 2.68 (t, J=14.0 Hz, 1H), 2.29-2.19 (m, 1H), 1.70-1.57 (m, 1H); MS (ESI) m/z 566.49 (M+H).

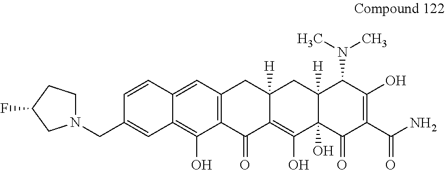

Compound 122. To a suspension of aldehyde S1-8 (23.7 mg, 0.0298 mmol, 1.0 eq) in methylene chloride (1 mL) was added to (R)-(−)-3-fluoropyrrolidine hydrochloride (19.3 mg, 0.143 mmol, 5 eq) was added triethylamine (41 μL, 0.298 mmol, 10 eq) was added. After 65 min, sodium triacetoxyborohydride (33.8 mg, 0.159 mmol, 5 eq) was added. After 1 h, the solution was poured into an aqueous potassium phosphate buffer solution (pH 7.0, 0.2 M, 10 mL) and extracted with ethyl acetate (3×20 mL). The combined organic extracts were dried over anhydrous sodium sulfate. The dried solution was filtered and the filtrate concentrated. The residue was used directly without purification. The remaining two deprotection steps were performed as in General Procedure A to provide S1-9-28 as a yellow solid on purification (5.8 mg, 34%, three steps). ¹H NMR (400 MHz, CD₃OD:hydrochloride salt) δ 8.54 (s, 1H), 7.89 (d, J=8.5 Hz, 1H), 7.78 (t, J=8.5 Hz, 1H), 7.22-7.16 (m, 1H), 5.57-5.33 (m, 1H), 4.73-4.57 (m, 2H), 4.12 (s, 1H), 3.94-3.42 (m, 4H), 3.17-2.90 (m, 9H), 2.72-2.15 (m, 4H), 1.67-1.56 (m, 1H); MS (ESI) m/z 566.36 (M+H).

The following compounds were prepared similarly to Compound 122.

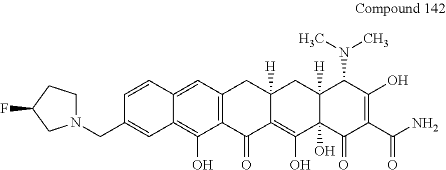

Compound 142: ¹H NMR (400 MHz, CD₃OD:hydrochloride salt) δ 8.55 (s, 1H), 7.89 (d, J=8.5 Hz, 1H), 7.78 (t, J=8.5 Hz, 1H), 7.23-7.18 (m, 1H), 5.57-5.35 (m, 1H), 4.73-4.55 (m, 2H), 4.11 (s, 1H), 3.94-3.41 (m, 4H), 3.17-2.92 (m, 9H), 2.72-2.17 (m, 4H), 1.69-1.55 (m, 1H); MS (ESI) m/z 566.34 (M+H).

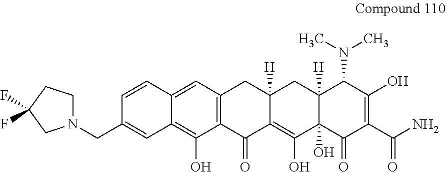

Compound 110: ¹H NMR (400 MHz, CD₃OD:hydrochloride salt) δ 8.58 (s, 1H), 7.89 (d, J=8.5 Hz, 1H), 7.77 (t, J=8.5 Hz, 1H), 7.23 (s, 1H), 4.69 (s, 2H), 4.09 (s, 1H), 4.00-3.90 (m, 2H), 3.80-3.68 (m, 2H), 3.15-2.92 (m, 9H), 2.72-2.61 (m, 3H), 2.27-2.18 (m, 1H), 1.71-1.55 (m, 1H); MS (ESI) m/z 584.33 (M+H).

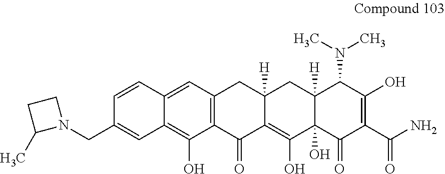

Compound 103: ¹H NMR (400 MHz, CD₃OD:hydrochloride salt) δ 8.51 (s, 1H), 7.86 (d, J=8.5 Hz, 1H), 7.70 (d, J=8.5 Hz, 1H), 7.21 (s, 1H), 4.71-4.61 (s, 1H), 4.53 (q, J=13.2, 2 H), 4.16-4.04 (m, 2H), 4.00-3.87 (m, 1H), 3.17-2.91 (m, 9H), 2.74-2.53 (m, 2H), 2.31-2.18 (m, 2H), 1.69-1.57 (m, 1H), 1.30 (d, J=6.7 Hz, 3H); MS (ESI) m/z 548.56 (M+H).

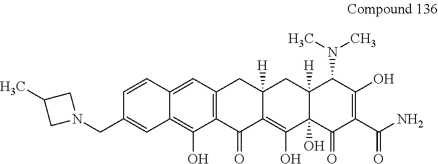

Compound 136: ¹H NMR of compound suggests a mixture of two compounds in a ratio of 1.5:1. LCMS data suggests a single product. ¹H NMR (400 MHz, CD₃OD:hydrochloride salt) δ 8.51-8.46 (m, 1H), 7.88-7.81 (m, 1H), 7.72-7.66 (m, 1H), 7.20 (s, 1H), 4.57 (s, 0.8H), 4.51 (s, 1.2H), 4.30 (m, 0.8H), 4.17-4.08 (m, 2.2H), 3.94-3.86 (m, 1.2H), 3.83-3.76 (m, 0.8H), 3.16-2.92 (m, 10H), 2.72-2.61 m, 1H), 2.27-2.18 (m, 1H), 1.70-1.55 (m, 1H), 1.33 (d, J=7.3 Hz, 1.2H), 1.27 (d, J=6.7 Hz, 1.8 Hz); MS (ESI) m/z 548.55 (M+H).

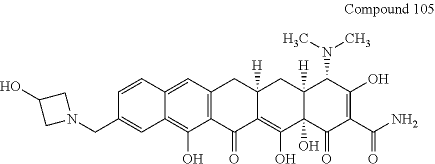

Compound 105: ¹H NMR (400 MHz, CD₃OD:hydrochloride salt) δ 8.49 (s, 1H), 7.85 (d, J=8.5 Hz, 1H), 7.72-7.66 (m, 1H), 7.19 (s, 1H), 4.74-4.51 (m, 3H), 4.42-4.28 (m, 2H), 4.10 (s, 1H), 4.08-3.94 (m, 2H), 3.17-2.91 (m, 9H), 2.66 (t, J=14.7 Hz, 1H), 2.28-2.18 (m, 1H), 1.67-1.57 (m, 1H); MS (ESI) m/z 550.54 (M+H).

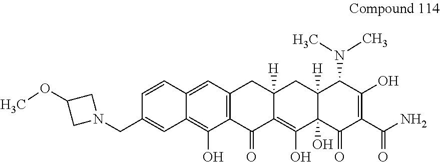

Compound 114: ¹H NMR (400 MHz, CD₃OD:hydrochloride salt) δ 8.48 (s, 1H), 7.84 (d, J=8.5 Hz, 1H), 7.76-7.70 (m, 1H), 7.20-7.15 (m, 1H), 4.61 (s, 3H), 4.47-4.28 (m, 3H), 4.16 (s, 1H), 4.12-4.01 (m, 2H), 3.17-2.92 (m, 9H), 2.69-2.56 (m, 1H), 2.33-2.23 (m, 1H), 1.68-1.53 (m, 1H); MS (ESI) m/z 564.57 (M+H).

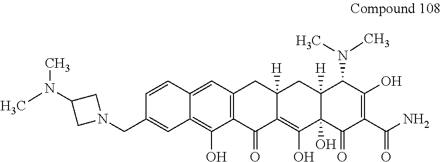

Compound 108: ¹H NMR (400 MHz, CD₃OD:hydrochloride salt) δ 8.57 (s, 1H), 7.89-7.76 (m, 2H), 7.19 (s, 1H), 4.91-4.74 (m, 4H), 4.59-4.41 (m, 3H), 4.15 (s, 1H), 3.15-2.95 (m, 9H), 2.91 (s, 6H), 2.68-2.56 (m, 1H), 2.32-2.23 (m, 1H), 1.67-1.54 (m, 1H); MS (ESI) m/z 577.60 (M+H).

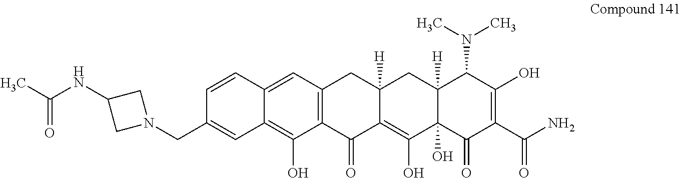

Compound 141: ¹H NMR (400 MHz, CD₃OD:hydrochloride salt) δ 8.52-8.48 (m, 1H), 7.89-7.82 (m, 1H), 7.74-7.66 (m, 1H), 7.23-7.16 (m, 1H), 4.67-4.53 (m, 3H), 4.47-4.21 (m, 4H), 4.10 (s, 1H), 3.16-2.92 (m, 9H), 2.72-2.60 (m, 1H), 2.27-2.18 (m, 1H), 2.00 (s, 3H), 1.69-1.56 (m, 1H); MS (ESI) m/z 591.58 (M+H).

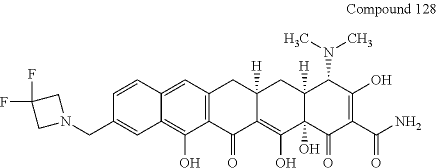

Compound 128: ¹H NMR (400 MHz, CD₃OD:hydrochloride salt) δ 8.55 (s, 1H), 7.86 (d, J=8.5 Hz, 1H), 7.82-7.74 (m, 1H), 7.20 (s, 1H), 4.90-4.52 (m, 5H), 4.15 (s, 1H), 4.11-3.70 (m, 1H), 3.14-2.93 (m, 9H), 2.68-2.56 (m, 1H), 2.32-2.21 (m, 1H), 1.67-1.52 (m, 1H); MS (ESI) m/z 570.54 (M+H).

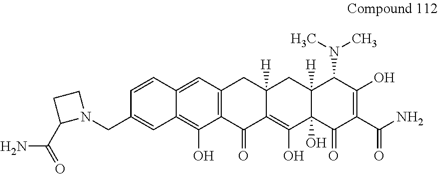

Compound 112: ¹H NMR (400 MHz, CD₃OD:hydrochloride salt) δ 8.51 (s, 1H), 7.83 (d, J=8.5 Hz, 1H), 7.72-7.66 (m, 1H), 7.20 (s, 1H), 5.14 (t, J=9.2 Hz, 1H), 4.59, 4.54 (ABq, J=12.8 Hz, 2H), 4.28, 4.23 (q, J=9.8 Hz, 1H), 4.10 (s, 1H), 4.03-3.92 (m, 1H), 3.17-2.93 (m, 9H), 2.84-2.72 (m, 1H), 2.73-2.62 (m, 1H), 2.60-2.42 (m, 1H), 2.26-2.18 (m, 1H), 1.70-1.58 (m, 1H); MS (ESI) m/z 577.42 (M+H).

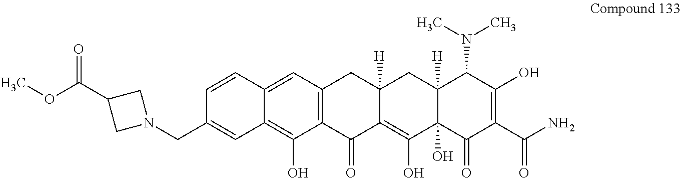

Compound 133: ¹H NMR (400 MHz, CD₃OD:hydrochloride salt) δ 8.51 (s, 1H), 7.87 (d, J=8.5 Hz, 1H), 7.70 (d, J=8.5 Hz, 1H), 7.22 (s, 1H), 4.64-4.57 (m, 2H), 4.50-4.29 (m, 4H), 4.10 (s, 1H), 3.82-3.72 (m, 4H), 3.14-2.92 (m, 9H), 2.75-2.62 (m, 1H), 2.28-2.18 (m, 1H), 1.70-1.59 (m, 1H); MS (ESI) m/z 592.43 (M+H).

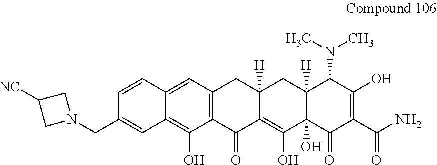

Compound 106: ¹H NMR (400 MHz, CD₃OD:hydrochloride salt) δ 8.51 (s, 1H), 7.88 (d, J=8.5 Hz, 1H), 7.69 (dd, J=1.8, 8.5 Hz, 1H), 7.22 (s, 1H), 4.64 (s, 2H), 4.59-4.37 (m, 3H), 4.10 (s, 1H), 4.08-3.95 (m, 1H), 3.12-2.93 (m, 10H), 2.73-2.62 (m, 1H), 2.28-2.18 (m, 1H), 1.70-1.56 (m, 1H); MS (ESI) m/z 559.47 (M+H).

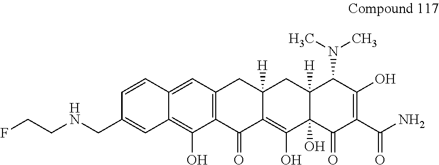

Compound 117: ¹H NMR (400 MHz, CD₃OD:hydrochloride salt) δ 8.55 (s, 1H), 7.86 (d, J=8.5 Hz, 1H), 7.75 (dd, J=1.8, 8.5 Hz, 1H), 7.21 (s, 1H), 4.84 (t, J=5.5 Hz, 1H), 4.73 (t, J=5.0 Hz, 1H), 4.46 (s, 2H), 4.11 (s, 1H), 3.52 (t, J=4.3 Hz, 1H), 3.45 (t, J=4.3 Hz, 1H), 3.18-2.83 (m, 10H), 2.69 (t, J=13.4 Hz, 1H), 2.27-2.17 (m, 1H), 1.70-1.56 (m, 1H); MS (ESI) m/z 540.33 (M+H).

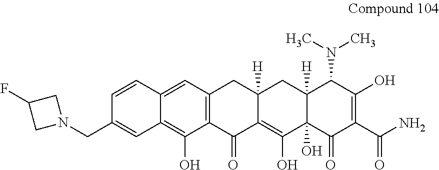

A solution of aldehyde S1-8 (1.08 g, 1.36 mmol, 1.0 eq) in methylene chloride (6.8 mL) was added to 3-fluoroazetidine hydrochloride (214 mg, 1.92 mmol, 1.4 eq), and the mixture was placed under nitrogen atmosphere. Triethylamine (568 μL, 4.08 mmol, 3 eq) was added. After 75 min, sodium triacetoxyborohydride (740 mg, 3.49 mmol, 2.5 eq) was added. After 16 h, the solution was poured into an aqueous potassium phosphate buffer solution (pH 7.0, 0.2 M, 15 mL) and extracted with ethyl acetate (3×50 mL). The combined organic extracts were dried over anhydrous sodium sulfate. The dried solution was filtered and the filtrate concentrated. The residue was used directly without purification.

Concentrated aqueous hydrofluoric acid solution (48 wt %, 3.3 mL) was added to the above reductive amination product in acetonitrile (10 mL) in a polypropylene reaction vessel at 23° C. After 40 h, the solution was poured into an aqueous solution of dipotassium hydrogenphosphate (52 g in 210 mL water) and extracted with ethyl acetate (2×100 mL). The aqueous layer was further diluted with an aqueous saturated sodium bicarbonate solution (40 mL) and extracted with ethyl acetate. The combined organic extracts were dried over anhydrous sodium sulfate. The dried solution was filtered and the filtrate concentrated. The yellow residue was used directly without purification.

The above residue was dissolved in methanol (50 mL) and dioxane (10 mL), and Pd—C (10 wt %, 510 mg) was added in one portion. An atmosphere of hydrogen was introduced by briefly evacuating the flask, then flushing with pure hydrogen (1 atm) from a balloon three times. The reaction was stirred under a hydrogen balloon for 2 h, then filtered through a pad of Celite with a methanol wash. The filtrate was concentrated, affording a yellow oil. The residue was purified by preparative reverse phase HPLC on a Waters Autopurification system using a Phenomenex Polymerx 10μ RP-1 100A column [10 μm, 150×21.20; flow rate, 20 mL/min; Solvent A: 0.5% TFA in water; Solvent B: CH₃CN; injection volume: 8×4 mL (0.5% TFA in water); gradient: 10-38% B over 20 min; mass-directed fraction collection]. Fractions with the desired molecular weight were collected and freeze-dried to yield the TFA salt of Compound 104 (474 mg, 45%, three steps).

To a solution of the TFA salt of Compound 104 (269 mg, 0.345 mmol, 1 eq) in methanol (5 mL) was added methanesulfonic acid (44.8 μL, 0.691 mmol, 2 eq). The solvent was removed under reduced pressure, the residue was dissolved in water (3.75 mL) and CH₃CN (1.25 mL) and then freeze-dried to provide the desired product as the dimesylate salt of Compound 104 (256 mg, 100%). ¹H NMR (400 MHz, CD₃OD:dimesylate salt) δ 8.55-8.47 (m, 1H), 7.87 (d, J=8.5 Hz, 1H), 7.73-7.65 (m, 1H), 7.21 (s, 1H), 5.58-5.28 (m, 1H), 4.72-4.27 (m, 6H), 4.09 (s, 1H), 3.18-2.92 (m, 10H), 2.74-2.62 (3, 7 H), 2.27-2.18 (m, 1H), 1.71-1.57 (m, 1H); MS (ESI) m/z 552.18 552.35 (M+H).

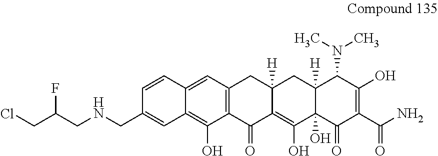

Treatment of Compound 104 with aqueous HCl resulted in a minor impurity of Compound 135. Purification of this compound was accomplished by preparative reverse phase HPLC on a Waters Autopurification system using a Phenomenex Polymerx 10μ RP-1 100A column [10 μm, 150×21.20; flow rate, 20 mL/min; Solvent A: 0.05N HCl in water; Solvent B: CH₃CN; injection volume: 8×4 mL (0.05N HCl in water); gradient: 0-35% B over 15 min; mass-directed fraction collection]. Fractions with the molecular weight (minor product) were collected and freeze-dried to yield Compound 135 as a yellow solid. ¹H NMR (400 MHz, CD₃OD:hydrochloride salt) δ 8.54 (s, 1H), 7.85 (d, J=8.5 Hz, 1H), 7.75 (dd, J=1.8, 8.5 Hz, 1H), 7.19 (s, 1H), 5.26-5.06 (m, 1H), 4.48 (s, 2H), 4.11 (s, 1H), 3.98-3.76 (m, 2H), 3.61-3.38 (m, 2H), 3.16-2.92 (m, 9H), 2.65 (d, J=14.0 Hz, 1H), 2.28-2.18 (m, 1H), 1.69-1.55 (m, 1H); MS (ESI) m/z 588.32 (M+H).

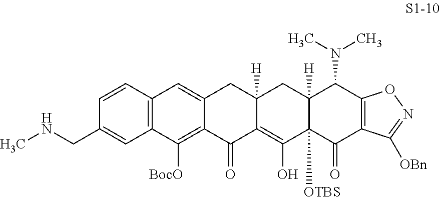

A solution of MeNH₂ in EtOH (68 μL, 0.543 mmol, 7.0 eq), acetic acid (31 μL, 0.543 mmol, 7.0 eq) and sodium triacetoxyborohydride (115 mg, 0.543 mmol, 7.0 eq) were added in sequence to a solution of aldehyde S1-8 (61.7 mg, 0.078 mmol, 1.0 eq) in 1,2-dichloroethane (4 mL) at 23° C. After stirring for 5 h, the reaction mixture was quenched by the addition of saturated aqueous sodium bicarbonate (10 mL) and aqueous potassium phosphate buffer solutions (pH 7.0, 0.2 M, 10 mL). The product was extracted into methylene chloride (3×15 mL). The combined organic extracts were dried over anhydrous sodium sulfate. The dried solution was filtered and the filtrate was concentrated to afford product S1-10, which was used directly for the next reactions without purification.

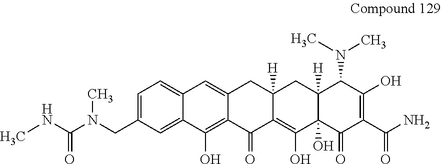

2,6-Lutidine (5.7 μL, 0.049 mmol, 3.0 eq) and methylisocyanate (2.9 μL, 0.049 mmol, 3.0 eq) were added to a solution of compound S1-10 (13.2 mg, 0.016 mmol, 1.0 eq) in anhydrous methylene chloride (1 mL). The resulting reaction mixture was stirred at rt overnight and concentrated. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent CH₃CN with 0.1% HCO₂H; injection volume: 2.0 mL (CH₃CN); gradient: 90→100% B over 10 min, then 100% B for 5 min; mass-directed fraction collection]. Fractions with the desired product and product-Boc MW, were collected and concentrated on a RotaVap. The residue was used directly for the next reaction.

Concentrated aqueous hydrofluoric acid solution (48 wt %, 0.2 mL) was added to a solution of the above products in acetonitrile (0.5 mL) in a polypropylene reaction vessel at 23° C. The resulting mixture was stirred vigorously at 23° C. overnight, then was poured into water (20 mL) containing dipotassium hydrogenphosphate (2.4 g). The product was extracted into ethyl acetate (2×25 mL). The combined organic extracts were dried over anhydrous sodium sulfate. The dried solution was filtered and the filtrate was concentrated, affording a yellow solid.

Methanol (1.0 mL) and dioxane (0.2 mL) were added to the above residue, forming a yellow solution. Pd—C (10 wt %, 5.0 mg) was added in one portion at 23° C. An atmosphere of hydrogen was introduced by briefly evacuating the flask, then flushing with pure hydrogen (1 atm). The resulting mixture was stirred at 23° C. for 1 h 15 min, then was filtered through a pad of Celite. The filtrate was concentrated, affording a yellow solid. The residue was purified by preparative reverse phase HPLC on a Waters Autopurification system using a Phenomenex Polymerx 10μ RP-1 100A column [10 μm, 150×21.20 mm; flow rate, 20 mL/min; Solvent A: 0.05 N HCl/water; Solvent B: CH₃CN; injection volume: 3.0 mL (0.05 N HCl/water); gradient: 15→70% B over 10 min; mass-directed fraction collection]. Fractions with the desired MW were collected and freeze-dried to yield product Compound 129 (0.79 mg, 8%, 3 steps). ¹H NMR (400 MHz, CD₃OD, hydrochloride) δ 8.16 (s, 1H), 7.70 (d, J=8.2 Hz, 1H), 7.51 (d, J=8.2 Hz, 1H), 7.11 (s, 1H), 4.66 (s, 2H), 4.07 (s, 1H), 107-2.94 (m, 9H), 2.86 (s, 3H), 2.78 (s, 3H), 2.63 (t, J=14.6 Hz, 1H), 2.21-2.18 (m, 1H), 1.66-1.56 (m, 1H); MS (ESI) m/z 565.46 (M+H).

The following compounds were prepared similarly to Compound 129.

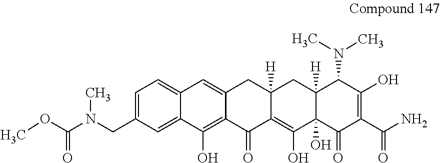

Compound 147: ¹H NMR (400 MHz, CD₃OD, hydrochloride) δ 8.19 (br s, 1H), 7.56 (d, J=8.2 Hz, 1H), 7.53 (br s, 1H), 7.12 (s, 1H), 4.64 (s, 2H), 4.08 (s, 1H), 3.76 (s, 3H), 3.09-2.91 (m, 12H), 2.65 (t, J=14.6 Hz, 1H), 2.22-2.19 (m, 1H), 1.67-1.57 (m, 1H); MS (ESI) m/z 566.45 (M+H).

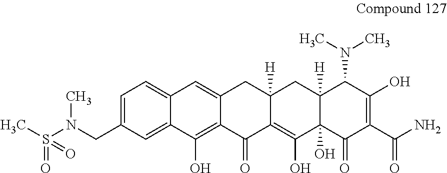

2,6-Lutidine (4.3 μL, 0.037 mmol, 3.0 eq) and methanesulfonylchloride (2.9 μL, 0.037 mmol, 3.0 eq) were added to a solution of compound S1-10 (10 mg, 0.012 mmol, 1.0 eq) in anhydrous methylene chloride (0.6 mL). The resulting reaction mixture was stirred at rt for 6 h and concentrated. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; injection volume: 2.0 mL (CH₃CN); gradient: 90→100% B over 10 min, then 100% B for 5 min; mass-directed fraction collection]. Fractions with the desired product and product-Boc MW, were collected and concentrated on a RotaVap. The residue was used directly for the next reaction (HF and hydrogenation reactions).

Compound 127 (2.4 mg, 34%, yellow solid): ¹H NMR (400 MHz, CD₃OD, hydrochloride) δ 8.31 (s, 1H), 7.75 (d, J=8.2 Hz, 1H), 7.66 (d, J=8.2 Hz, 1H), 7.13 (s, 1H), 4.46 (s, 2H), 4.08 (s, 1H), 3.08-2.95 (m, 12H), 2.77 (s, 3H), 2.64 (t, J=14.6 Hz, 1H), 2.24-2.19 (m, 1H), 1.67-1.57 (m, 1H); MS (ESI) m/z 586.44 (M+H).

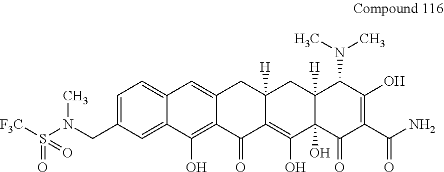

Compound 116: ¹H NMR (400 MHz, CD₃OD, hydrochloride) δ 8.34 (s, 1H), 7.80 (d, J=8.7 Hz, 1H), 7.64 (d, J=8.2 Hz, 1H), 7.16 (s, 1H), 4.89 (s, 2H), 4.07 (s, 1H), 3.03-2.95 (m, 12H), 2.77 (s, 3H), 2.66 (t, J=13.7 Hz, 1H), 2.22-2.19 (m, 1H), 1.68-1.58 (m, 1H); MS (ESI) m/z 640.43 (M+H).

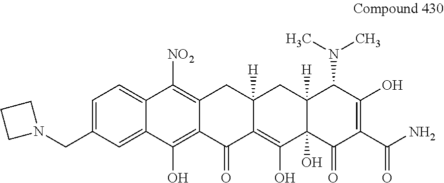

Compound 430. A mixture of conc. HNO₃ (68-70%)/H₂SO₄ (v/v 4:5, 6.5 μL, 0.043 mmol, 5.0 equiv) was added to a solution of Compound 137 (4.5 mg, 0.008 mmol, 1.0 equiv) in conc. H₂SO₄ (200 μL) at 0° C. The resulting red reaction mixture was stirred for 2 min, diluted with water, and purified by preparative reverse phase HPLC on a Waters Autopurification system using a Phenomenex Polymerx 10μ RP-1 100A column [10 μm, 150×21.20 mm; flow rate, 20 mL/min; Solvent A: 0.05 N HCl/water; Solvent B: CH₃CN; injection volume: 3.0 mL (0.05 N HCl/water); gradient: 5→40% B over 10 min; mass-directed fraction collection]. Fractions with the desired MW were collected and freeze-dried to yield Compound 430 (1.90 mg, 39%): ¹H NMR (400 MHz, CD₃OD, hydrochloride) δ 8.65 (d, J=1.4 Hz, 1H), 7.89 (dd, J=1.4, 8.7 Hz, 1H), 7.57 (d, J=8.7 Hz, 1H), 4.61 (s, 2H), 4.28 (q, J=9.6 Hz, 2H), 4.17-4.11 (m, 3H), 3.34-3.32 (m, 1H), 3.04-2.95 (m, 8H), 2.70-2.56 (m, 2H), 2.53-2.47 (m, 1H), 2.24-2.21 (m, 1H), 1.71-1.61 (m, 1H); MS (ESI) m/z 579.50 (M+H).

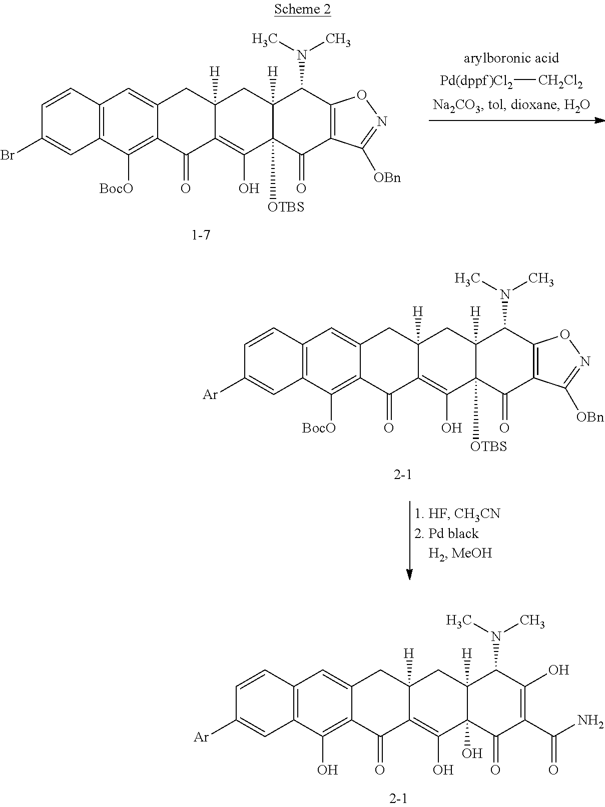

The following compounds were prepared according lo Scheme 2.

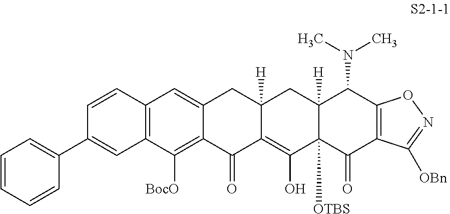

Bromide S1-7 (20.0 mg, 0.0236 mmol, 1.0 eq), phenyl boronic acid (14.4 mg, 0.118 mmol, 5.0 eq), Na₂CO₃ (12.5 mg, 0.118 mmol, 5.0 eq) and Pd(dppf)Cl₂.CH₂Cl₂ (1.0 mg, 0.0012 mmol, 0.05 eq) were dissolved in a mixture of toluene (1 mL)/dioxane (1 mL)/water (0.2 mL). The resulting reaction mixture was then stirred at 80° C. for 1 h. The resulting orange solution was then cooled to rt, diluted with methylene chloride (30 mL), washed with aqueous potassium phosphate buffer solutions (pH 7.0, 0.2 M, 10 mL). The organic phase was dried over anhydrous sodium sulfate. The dried solution was filtered and the filtrate was concentrated. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; injection volume: 2.0 mL (CH₃CN); gradient: 80→100% B over 7 min, then 100% B for 8 min; mass-directed fraction collection]. Fractions with the desired MW, eluting at 7.8-8.7 min, were collected and concentrated on a RotaVap at rt to afford the desired product S2-1-1 (15.6 mg, 78%) as a yellow solid: ¹H NMR (400 MHz, CDCl₃) δ 16.09 (s, 1H), 8.23 (s, 1H), 7.83 (s, 2H), 7.68-7.66 (m, 2H), 7.56 (s, 1H), 7.51-7.47 (m, 4H), 7.41-7.32 (m, 4H), 5.36 (s, 2H), 4.01 (d, J=10.4 Hz, 1H), 3.15-3.08 (m, 2H), 2.97 (t, J=15.3 Hz, 1H), 2.59-2.47 (m, 8H), 2.15 (d, J=14.0 Hz, 1H), 1.58 (s, 9H), 0.82 (s, 9H), 0.27 (s, 3H), 0.14 (s, 3H); MS (ESI) m/z 843.78 (M+H).

The following compounds were prepared similarly to S2-1-1.

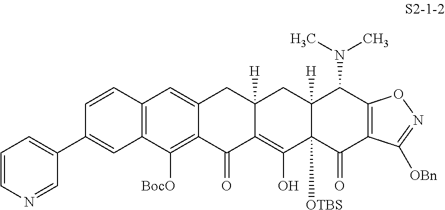

S2-1-2: (86%) ¹H NMR (400 MHz, CDCl₃) δ 16.03 (s, 1H), 8.93 (d, J=1.8 Hz, 1H), 8.64 (dd, J=1.2, 4.9 Hz, 1H), 8.24 (s, 1H), 8.02 (dt, J=7.9, 1.8 Hz, 1H), 7.89 (d, J=8.5 Hz, 1H), 7.79 (dd, J=8.5, 1.2 Hz, 1H), 7.59 (s, 1H), 7.50-7.46 (m, 3H), 7.39-7.32 (m, 3H), 5.37, 5.34 (ABq, J=12.2 Hz, 2H), 3.98 (d, J=11.0 Hz, 1H), 3.15-3.10 (m, 2H), 2.98 (t, J=15.3 Hz, 1H), 2.58-2.48 (m, 8H), 2.15 (d, J=14.0 Hz, 1H), 1.58 (s, 9H), 0.82 (s, 9H), 0.27 (s, 3H), 0.13 (s, 3H); MS (ESI) m/z 844.74 (M+H).

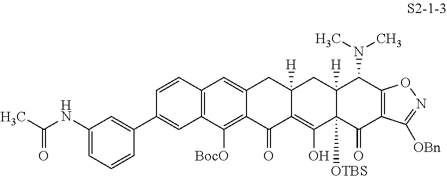

S2-1-3: (74%) ¹H NMR (400 MHz, CDCl₃) δ 16.11 (s, 1H), 8.16 (d, J=1.8 Hz, 1H), 7.79-7.74 (m, 2H), 7.63-7.60 (m, 2H), 7.51-7.48 (m, 4H), 7.7.42-7.33 (m, 5H), 5.38, 5.34 (ABq, J=12.2 Hz, 2H), 4.22 (d, J=9.2 Hz, 1H), 3.11-3.08 (m, 2H), 2.92 (t, J=15.3 Hz, 1H), 2.67 (s, 6H), 2.57-2.50 (m, 1H), 2.16 (s, 3H), 2.14 (d, J=14.0 Hz, 1H), 1.58 (s, 9H), 0.81 (s, 9H), 0.21 (s, 3H), 0.13 (s, 3H); MS (ESI) m/z 900.86 (M+H).

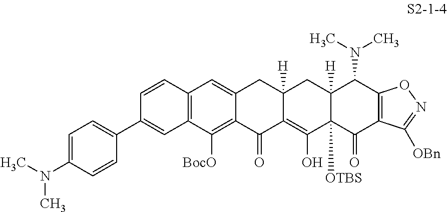

S2-1-4: (77%) ¹H NMR (400 MHz, CDCl₃) δ 16.11 (s, 1H), 8.16 (s, 1H), 7.83-7.77 (m, 2H), 7.61-7.59 (m, 2H), 7.52-7.49 (m, 3H), 7.39-7.32 (m, 3H), 6.92-6.90 (m, 2H), 5.37, 5.34 (ABq, J=12.2 Hz, 2H), 4.01 (d, J=10.4 Hz, 1H), 3.19-2.93 (m, 9H), 2.58-2.46 (m, 8H), 2.14 (d, J=14.0 Hz, 1H), 1.58 (s, 9H), 0.82 (s, 9H), 0.27 (s, 3H), 0.13 (s, 3H); MS (EST) m/z 886.90 (M+H).

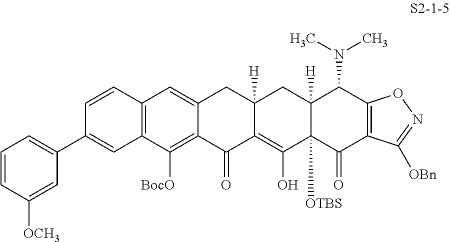

S2-1-5: (84%) ¹H NMR (400 MHz, CDCl₃) δ 16.08 (s, 1H), 8.23 (s, 1H), 7.84-7.79 (m, 2H), 7.75 (s, 1H), 7.51-7.48 (m, 2H), 7.42-7.30 (m, 4H), 7.25-7.24 (m, 1H), 7.19-7.18 (m, 1H), 6.94 (dd, J=1.8, 7.3 Hz, 1H), 5.37, 5.34 (ABq, J=12.2 Hz, 2H), 4.01 (d, J=10.4 Hz, 1H), 3.87 (s, 3H), 3.15-3.08 (m, 2H), 2.97 (t, J=15.3 Hz, 1H), 2.59-2.46 (m, 8H), 2.15 (d, J=14.0 Hz, 1H), 1.58 (s, 9H), 0.82 (s, 9H), 0.27 (s, 3H), 0.13 (s, 3H); MS (ESI) m/z 873.86 (M+H).

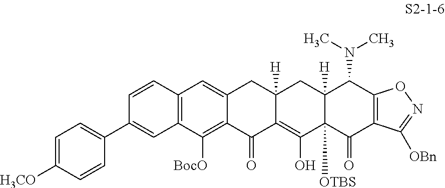

S2-1-6: (83%) ¹H NMR (400 MHz, CDCl₃) δ 16.10 (s, 1H), 8.17 (s, 1H), 7.82-7.78 (m, 2H), 7.61-7.59 (m, 2H), 7.54 (s, 1H), 7.51-7.48 (m, 2H), 7.39-7.30 (m, 3H), 7.03-7.01 (m, 2H), 7.19-7.18 (m, 1H), 6.94 (dd, J=1.8, 7.3 Hz, 1H), 5.37, 5.34 (ABq, J=12.2 Hz, 2H), 4.01 (d, J=10.4 Hz, 1H), 3.86 (s, 3H), 3.15-3.08 (m, 2H), 2.97 (t, J=15.3 Hz, 1H), 2.59-2.46 (m, 8H), 2.14 (d, J=14.0 Hz, 1H), 1.58 (s, 9H), 0.82 (s, 9H), 0.27 (s, 3H), 0.13 (s, 3H); MS (ESI) m/z 873.82 (M+H).

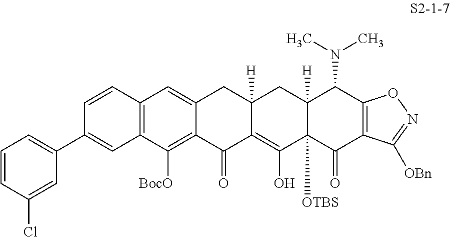

S2-1-7: (69%) ¹H NMR (400 MHz, CDCl₃) δ 16.06 (s, 1H), 8.20 (s, 1H), 7.84 (d, J=8.5 Hz, 1H), 7.77 (dd, J=1.2, 8.5 Hz, 1H), 7.64 (t, J=1.8 Hz, 1H), 7.56 (s, 1H), 7.54-7.48 (m, 3H), 7.43-7.32 (m, 5H), 5.37, 5.34 (ABq, J=12.2 Hz, 2H), 4.01 (d, J=10.4 Hz, 1H), 3.15-3.09 (m, 2H), 2.97 (t, J=15.3 Hz, 1H), 2.60-2.47 (m, 8H), 2.14 (d, J=14.0 Hz, 1H), 1.59 (s, 9H), 0.82 (s, 9H), 0.26 (s, 3H), 0.14 (s, 3H); MS (ESI) m/z 877.73 (M+H).

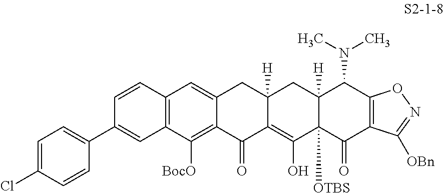

S2-1-8: (64%) ¹H NMR (400 MHz, CDCl₃) δ 16.07 (s, 1H), 8.18 (s, 1H), 7.84 (d, J=8.5 Hz, 1H), 7.77 (dd, J=1.2, 8.5 Hz, 1H), 7.60-7.56 (m, 3H), 7.50-7.44 (m, 4H), 7.39-7.32 (m, 3H), 5.37, 5.34 (ABq, J=12.2 Hz, 2H), 4.01 (d, J=10.4 Hz, 1H), 3.15-3.09 (m, 2H), 2.97 (t, J=15.3 Hz, 1H), 2.60-2.48 (m, 8H), 2.14 (d, J=14.0 Hz, 1H), 1.59 (s, 9H), 0.82 (s, 9H), 0.26 (s, 3H), 0.13 (s, 3H); MS (ESI) m/z 877.76 (M+H).

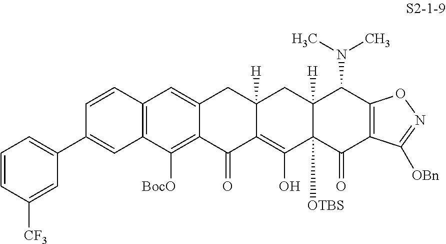

S2-1-9: (76%) ¹H NMR (400 MHz, CDCl₃) δ 16.06 (s, 1H), 8.22 (s, 1H), 7.88-7.79 (m, 4H), 7.65-7.58 (m, 3H), 7.50-7.48 (m, 2H), 7.39-7.32 (m, 3H), 5.37, 5.34 (ABq, J=12.2 Hz, 2H), 4.01 (d, J=10.4 Hz, 1H), 3.15-3.10 (m, 2H), 2.97 (t, J=15.3 Hz, 1H), 2.60-2.49 (m, 8H), 2.14 (d, J=14.0 Hz, 1H), 1.60 (s, 9H), 0.82 (s, 9H), 0.26 (s, 3H), 0.14 (s, 3H); MS (ESI) m/z 911.82 (M+H).

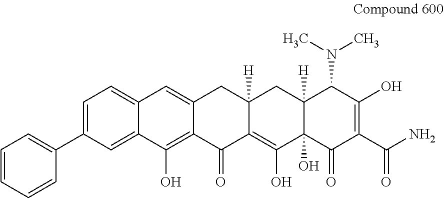

Concentrated aqueous hydrofluoric acid (48 wt %, 0.2 mL) was added to a solution of compound S2-1-1 (15.6 mg, 0.018 mmol, 1.0 eq) in acetonitrile (0.4 mL) in a polypropylene reaction vessel at 23° C. The resulting mixture was stirred vigorously at 23° C. overnight and poured into aqueous dipotassium hydrogenphosphate (2.5 g dissolved in 20 mL water). The mixture was extracted with ethyl acetate (2×25 mL). The combined organic extracts were dried over anhydrous sodium sulfate, filtered, and concentrated. The residue was used directly in the final step without further purification.

Pd—C (10 wt %, 8 mg) was added in one portion into the yellow solution of the above crude product in a mixture of MeOH (1 mL) and dioxane (1 mL) at 23° C. The reaction vessel was sealed and purged with hydrogen by briefly evacuating the flask followed by flushing with hydrogen gas (1 atm). The resulting mixture was stirred at 23° C. for 30 min. LCMS analysis indicated the reaction complete. The reaction mixture was then filtered through a small Celite pad. The filtrate was concentrated. The residue was purified by preparative reverse phase HPLC on a Waters Autopurification system using a Phenomenex Polymerx 10μ RP-1 100A column [10 μm, 150×21.20 mm; flow rate, 20 mL/min; Solvent A: 0.05 N HCl/water; Solvent B: CH₃CN; injection volume: 3.0 mL (0.05 N HCl/water); gradient: 15→100% B over 10 min; mass-directed fraction collection]. Fractions with the desired MW were collected and freeze-dried to yield Compound 600 (7.8 mg, 73% for 2 steps): ¹H NMR (400 MHz, CD₃OD, hydrochloride) δ 8.48 (d, J=1.8 Hz, 1H), 7.88 (dd, J=1.8, 8.7 Hz, 1H), 7.76 (d, J=8.4 Hz, 1H), 7.71 (d, J=7.3 Hz, 2H), 7.48 (t, J=7.3 Hz, 2H), 7.37 (t, J=73 Hz, 1H), 7.09 (s, 1H), 4.10 (s, 1H), 3.09-2.93 (m, 9H), 2.58 (t, J=14.2 Hz, 1H), 2.23-2.20 (m, 1H), 1.64-1.54 (m, 1H); MS (ESI) m/z 541.43 (M+H).

The following compounds were prepared similarly to Compound 600.

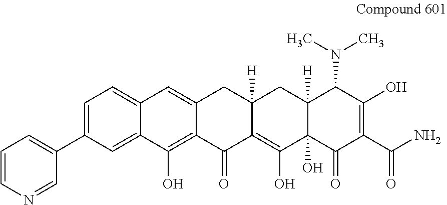

Compound 601: ¹H NMR (400 MHz, CD₃OD, hydrochloride) δ 9.33 (s, 1H), 9.07 (d, J=6.4 Hz, 1H), 8.87 (d, J=6.4 Hz, 1H), 8.76 (s, 1H), 8.22 (t, J=7.8 Hz, 1H), 8.04 (d, J=7.8 Hz, 1H), 7.92 (d, J=7.8 Hz, 1H), 7.19 (s, 1H), 4.12 (s, 1H), 3.12-2.97 (m, 9H), 2.64 (t, J=13.3 Hz, 1H), 2.25-2.23 (m, 1H), 1.67-1.58 (m, 1H); MS (ESI) m/z 542.41 (M+H).

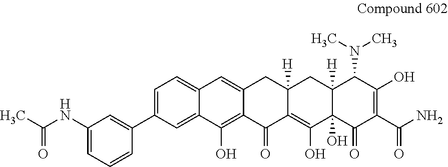

Compound 602: (mixture of rotamers, ˜3:2)¹H NMR (400 MHz, CD₃OD, hydrochloride) δ 8.44 (s, 0.6H), 8.38 (s, 0.4H), 7.95 (s, 0.6H), 7.92 (s, 0.4H), 7.83-7.77 (m, 1 μl), 7.72-7.66 (m, 1H), 7.53 (s, 1H), 7.41-7.38 (m, 2H), 7.04 (s, 0.6H), 6.97 (s, 0.4H), 4.08 (s, 1H), 3.04-2.88 (m, 9H), 2.55-2.43 (m, 1H), 2.17 (m, 4H), 1.60-1.52 (m, 1H); MS (ESI) m/z 598.50 (M+H).

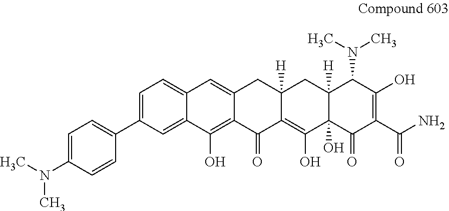

Compound 603: ¹H NMR (400 MHz, CD₃OD, hydrochloride) δ 8.49 (s, 1H), 7.92-7.84 (m, 3H), 7.77-7.75 (m, 3H), 7.08 (s, 1H), 4.06 (s, 1H), 3.30 (s, 6H), 3.08-2.92 (m, 9H), 2.57 (t, J=13.7 Hz, 1H), 2.20-2.17 (m, 1H), 1.60-1.51 (m, 1H); MS (ESI) m/z 584.51 (M+H).

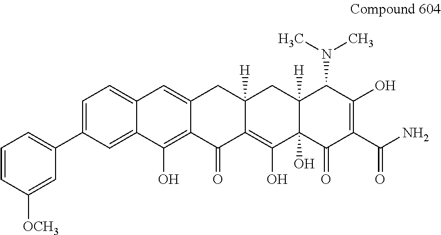

Compound 604: ¹H NMR (400 MHz, CD₃OD, hydrochloride) δ 8.48 (d, J=1.4 Hz, 1H), 7.88 (dd, J=1.4, 8.7 Hz, 1H), 7.39 (t, J=7.8 Hz, 1H), 7.28 (d, J=7.8 Hz, 1H), 7.23 (s, 1H), 7.10 (s, 1H), 6.95 (dd, J=2.3, 8.2 Hz, 1H), 4.09 (s, 1H), 3.87 (s, 3H), 3.04-2.94 (m, 9H), 2.61 (t, J=13.7 Hz, 1H), 2.23-2.20 (m, 1H), 1.65-1.55 (m, 1H): MS (ESI) m/z 571.46 (M+H).

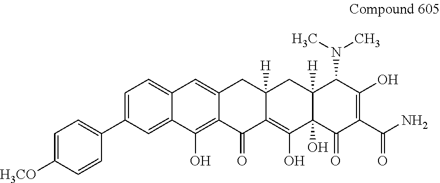

Compound 605: ¹H NMR (400 MHz, CD₃OD, hydrochloride) δ 8.45 (s, 1H), 7.87 (d, J=8.7 Hz, 1H), 7.75 (d, J=8.7 Hz, 1H), 7.66 (d, J=8.7 Hz, 2H), 7.10 (s, 1H), 7.04 (d, J=8.7 Hz, 2H), 4.09 (s, 1H), 3.85 (s, 3H), 3.05-2.94 (m, 9H), 2.62 (t, J=14.2 Hz, 1H), 2.23-2.20 (m, 1H), 1.66-1.56 (m, 1H); MS (ESI) m/z 571.48 (M+H).

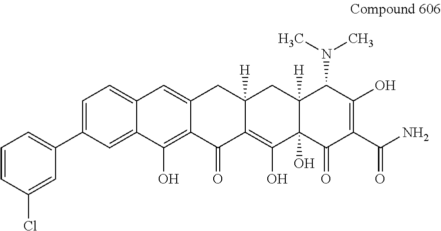

Compound 606: ¹H NMR (400 MHz, CD₃OD, hydrochloride) δ 8.44 (s, 1H), 7.84 (d, J=7.8 Hz, 1H), 7.76 (d, J=7.8 Hz, 1H), 7.69 (s, 1H), 7.63 (d, J=7.3 Hz, 1H), 7.56 (t, J=7.8 Hz, 1H), 7.37 (d, J=7.3 Hz, 1H), 4.10 (s, 1H), 3.87 (s, 3H), 3.06-2.94 (m, 9H), 2.58 (t, J=13.7 Hz, 1H), 2.25-2.22 (m, 1H), 1.65-1.55 (m, 1H); MS (ESI) m/z 575.44 (M+H).

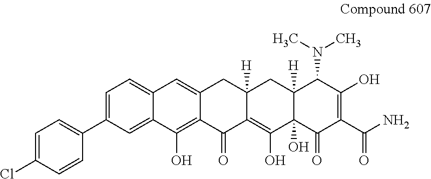

Compound 607: ¹H NMR (400 MHz, CD₃OD, hydrochloride) δ 8.52 (s, 1H), 7.89 (d, J=8.2 Hz, 1H), 7.80 (d, J=8.2 Hz, 1H), 7.72 (d, J=8.2 Hz, 2H), 7.48 (d, J=8.2 Hz, 2H), 7.14 (s, 1H), 4.10 (s, 1H), 3.12-2.98 (m, 9H), 2.63 (t, J=14.2 Hz, 1H), 2.25-2.22 (m, 1H), 1.65-1.55 (m, 1H); MS (ESI) m/z 575.47 (M+H).

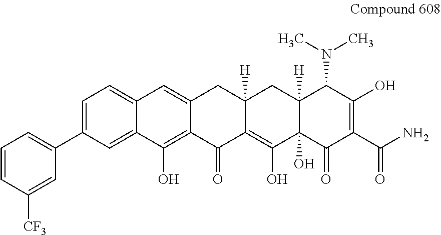

Compound 608: ¹H NMR (400 MHz, CD₃OD, hydrochloride) δ 8.44 (s, 1H), 7.94-7.92 (m, 2H), 7.87 (d, J=7.8 Hz, 1H), 7.78 (d, J=7.8 Hz, 1H), 7.67-7.66 (m, 2H), 7.09 (s, 1H), 4.11 (s, 1H), 3.87 (s, 3H), 3.06-2.94 (m, 9H), 2.57 (t, J=13.7 Hz, 1H), 2.25-2.23 (m, 1H), 1.65-1.55 (m, 1H); MS (ESI) m/z 609.50 (M+H).

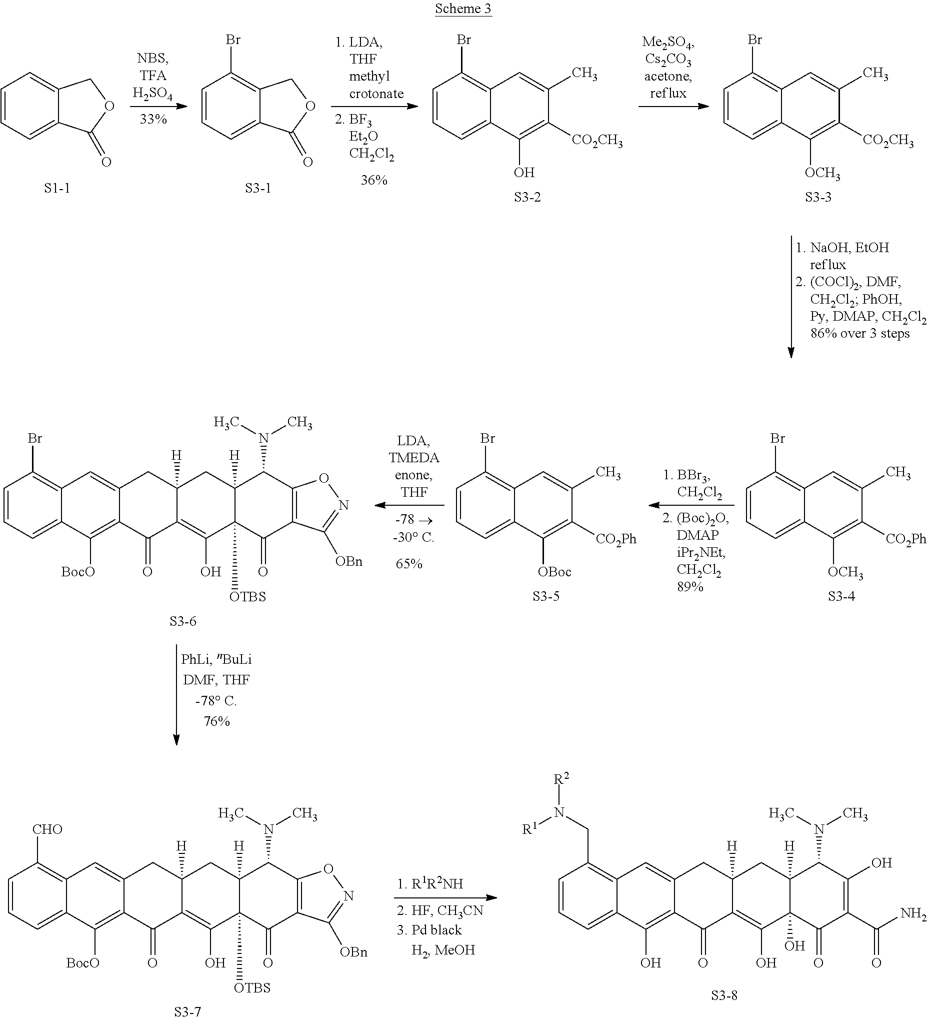

The following compounds were prepared according to Scheme 3.

Following the same procedure as described in Scheme 1,8-aminomethyl substituted pentacyclines of the formula S3-8 were synthesized from phthalide

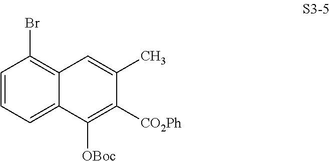

S3-5: ¹H NMR (400 MHz, CDCl₃) δ 8.00 (s, 1H), 7.87 (d, J=8.7 Hz, 1H), 7.80 (dd, J=1.4, 7.8 Hz, 1H), 7.42-7.38 (m, 2H), 7.31 (dd, J=7.3, 8.2 Hz, 1H), 7.26-7.23 (m, 3H), 2.66 (s, 3H), 1.41 (s, 9H).

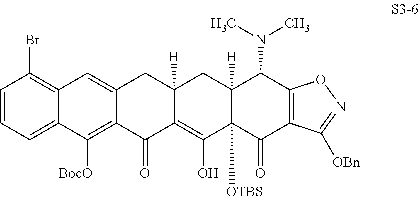

S3-6: ¹H NMR (400 MHz, CDCl₃) δ 15.99 (br s, 1H), 8.09 (d, J=8.5 Hz, 1H), 7.97 (s, 1H), 7.86 (d, J=6.7 Hz, 1H), 7.51-7.48 (m, 2H), 7.39-7.33 (m, 4H), 5.38, 5.34 (ABq, J=12.0 Hz, 2H), 3.97 (d, J=11.0 Hz, 1H), 3.23-3.11 (m, 2H), 3.00 (t, J=13.4 Hz, 1H), 2.60-2.46 (m, 8H), 2.22-2.16 (m, 1H), 1.58 (s, 9H), 0.82 (s, 9H), 0.28 (s, 3H), 0.14 (s, 3H); MS (ESI) m/z 845.61, 847.61 (M+H).

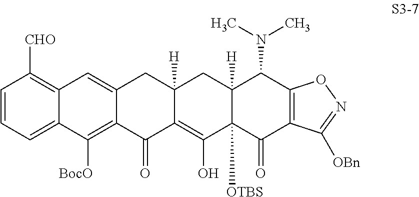

S3-7: ¹H NMR (400 MHz, CDCl₃) δ 15.93 (br s, 1H), 10.32 (s, 1H), 9.04 (s, 1H), 8.40 (d, J=8.5 Hz, 1H), 8.07 (d, J=6.7 Hz, 1H), 7.69 (dd, J=7.3, 7.9 Hz, 1H), 7.50-7.48 (m, 2H), 7.39-7.32 (m, 2H), 5.37, 5.33 (ABq, J=12.2 Hz, 2H), 3.96 (d, J=10.4 Hz, 1H), 3.25-3.11 (m, 2H), 3.00 (t, J=14.0 Hz, 1H), 2.58-2.40 (m, 8H), 2.20-2.15 (m, 1H), 1.57 (s, 9H), 0.81 (s, 9H), 0.26 (s, 3H), 0.12 (s, 3H); MS (ESI) m/z 795.58 (M+H).

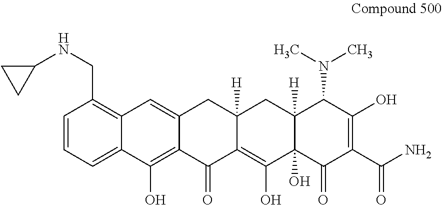

Compound 500: ¹H NMR (400 MHz, CD₃OD, hydrochloride) δ 8.47 (d, J=8.2 Hz, 1H), 7.83 (d, J=8.2 Hz, 1H), 7.54 (t, J=8.2 Hz, 1H), 7.46 (s, 1H), 4.75 (s, 2H), 4.11 (s, 1H), 3.20-2.85 (m, 10H), 2.69 (m, 1H), 2.35-2.25 (m, 1H), 1.67-1.58 (m, 1H); MS (ESI) m/z 534.52 (M+H).

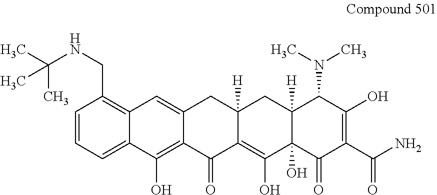

Compound 501: ¹H NMR (400 MHz, CD₃OD, hydrochloride) δ 8.45 (d, J=7.8 Hz, 1H), 7.82 (d, J=7.8 Hz, 1H), 7.54 (t, J=7.8 Hz, 1H), 7.38 (s, 1H), 4.59 (s, 2H), 4.10 (s, 1H), 3.14-2.96 (m, 9H), 2.68 (m, 1H), 2.28 (m, 1H), 1.61-1.47 (m, 10H); MS (ESI) m/z 550.56 (M+H).

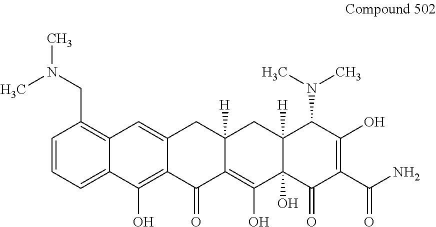

Compound 502: ¹H NMR (400 MHz, CD₃OD, hydrochloride) δ 8.52-8.49 (m, 1H), 7.86 (s, 1H), 7.55-7.51 (m, 2H), 4.77 (s, 2H), 4.10 (s, 1H), 3.19-2.90 (m, 15H), 2.67 (m, 1H), 2.28 (m, 1H), 1.62 (m, 1H); MS (ESI) m/z 522.50 (M+H).

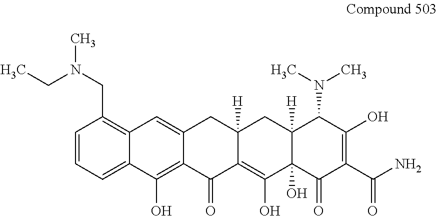

Compound 503: ¹H NMR (400 MHz, CD₃OD, hydrochloride) δ 8.53 (d, J=8.2 Hz, 1H), 7.88 (d, J=6.9 Hz, 1H), 7.58 (t, J=7.8 Hz, 1H), 7.49 (s, 1H), 4.68-4.65 (m, 2H), 4.11 (s, 1H), 3.20-2.97 (m, 11H), 2.79 (s, 3H), 2.69 (m, 1H), 2.28 (m, 1H), 1.65 (m, 1H), 1.41 (m, 3H); MS (ESI) m/z 536.60 (M+H).

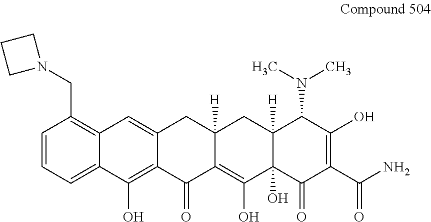

Compound 504: ¹H NMR (400 MHz, CD₃OD, hydrochloride) δ 8.45 (d, J=8.2 Hz, 1H), 7.80 (d, J=5.0 Hz, 1H), 7.52-7.48 (m, 2H), 4.82 (s, 2H), 4.27 (s, 2H), 4.10 (s, 3H), 3.19-2.96 (m, 9H), 2.66-2.45 (m, 3H), 2.26 (m, 1H), 1.62 (m, 1H); MS (ESI) m/z 534.51 (M+H).

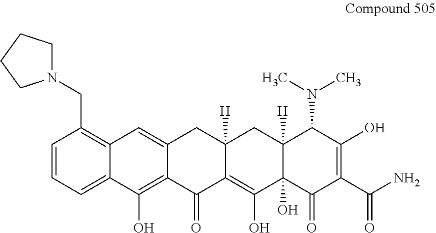

Compound 505: ¹H NMR (400 MHz, CD₃OD, hydrochloride) δ 8.41 (br s, 1H), 7.81 (br s, 1H), 7.48 (br s, 2H), 4.74 (s, 2H), 4.04 (s, 1H), 3.30-2.89 (m, 13H), 2.61 (m, 1H), 2.12-2.90 (m, 5H), 1.58 (m, 1H); MS (ESI) m/z 548.54 (M+H).

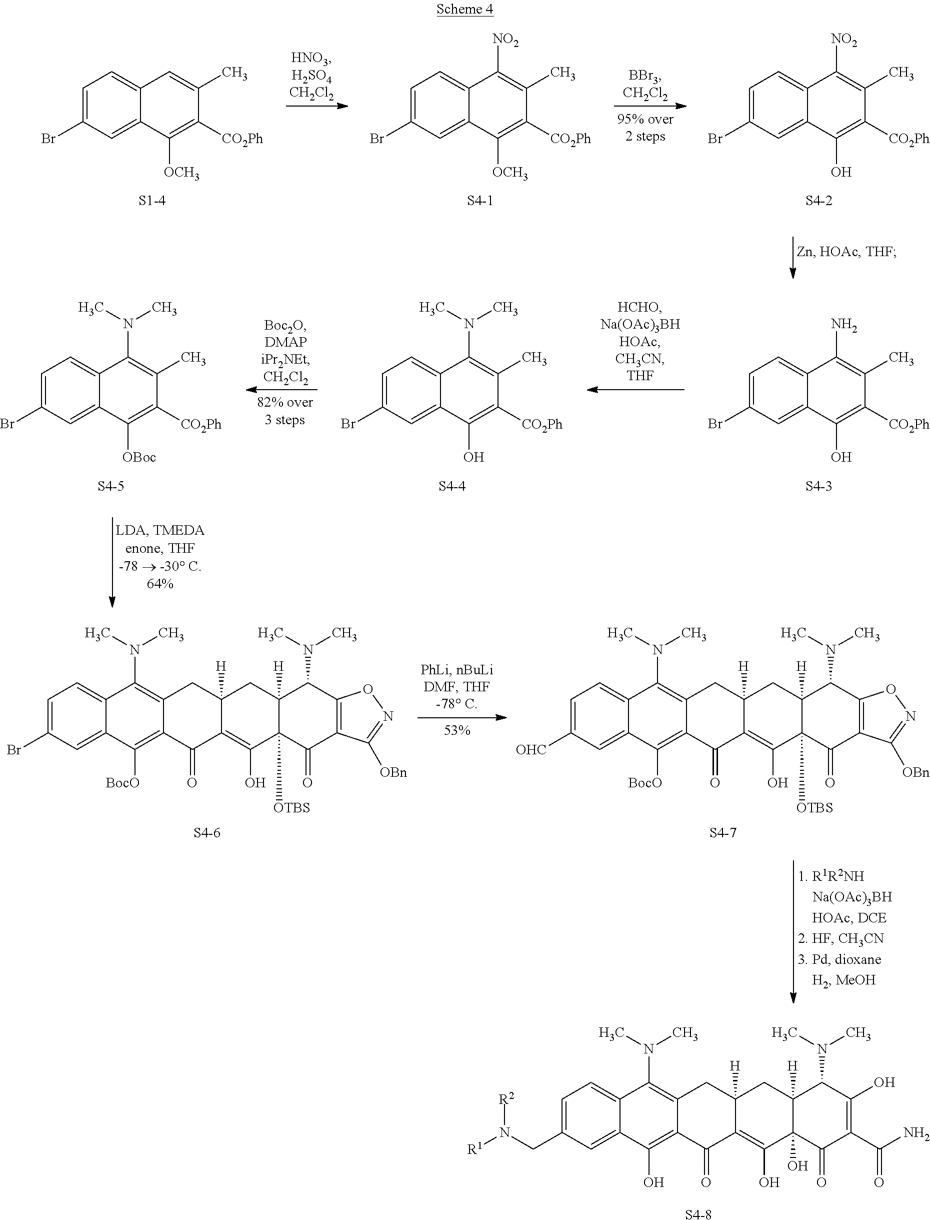

The following compounds were prepared according to Scheme 4.

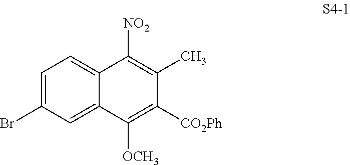

A mixture of freshly mixed HNO₃ (68-70%) and H₂SO₄ (concentrated) (4:5 v/v, 0.6 mL) was added drop-wise to a solution of compound S1-4 (2.74 g, 7.38 mmol, 1.0 eq) in methylene chloride (25 mL) at 0° C. The resulting yellow solution was stirred at 0° C. for 25 min and another portion of HNO₃ (68-70%) and H₂SO₄ (concentrated) (4:5 v/v, 0.66 mL) was added drop-wise. The reaction mixture was stirred at 0° C. for 1.5 h (monitored by LCMS or TLC using Acetonitrile/toluene as solvent), and neutralized with 6 N NaOH solution (5.5 mL) and saturated NaHCO₃ (100 mL). The organic layer was separated and the aqueous layer was extracted with methylene chloride (2×80 mL). The organic extracts were combined and dried over anhydrous MgSO₄. The dried solution was filtered and the filtrate was concentrated, providing compound S4-1 as a light yellow solid, which is pure enough for the next reaction. ¹H NMR (600 MHz, CDCl₃) δ 8.30 (s, 1H), 7.72 (d, J=8.7 Hz, 1H), 7.55 (d, J=8.7 Hz, 1H), 7.44-7.42 (m, 2H), 7.30-7.28 (m, 1H), 7.24-7.22 (m, 2H), 4.09 (s, 3H), 2.48 (s, 3H).

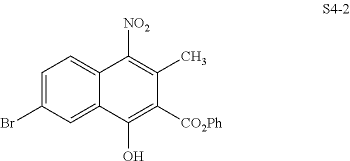

A solution of BBr₃ in CH₂Cl₂ (1.0 M, 14.76 mL, 14.76 mmol, 2.0 eq) was added slowly over 7 min to a solution of compound S4-1 in methylene chloride (74 mL) at −70° C. The resulting red solution was allowed to warm to 15° C. in 2 h (monitored by LCMS or TLC (product is slightly less polar)), and was poured into saturated NaHCO₃ solution (200 mL). The mixture was stirred at rt for 30 min and extracted with methylene chloride (2×300 mL) (emulsion). The aqueous layer was further extracted with ethyl acetate (2×150 mL). The organic extracts were combined and dried over anhydrous MgSO₄. The dried solution was filtered through a short plug of silica gel, and the filtrate was concentrated, providing the crude product S4-2 as a yellow solid (2.62 g, the yellow solid partially turned to brownish over time, probably due to limited stability). The crude product was used directly for the next reaction.

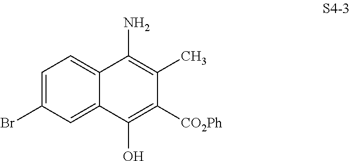

Zinc dust (1.93 g, 29.52 mmol, 4.0 eq) was added slowly to a solution of the above product S4-2 in a mixture of THF (30 mL) and HOAc (6 mL) in one portion at 0° C. The resulting mixture was stirred vigorously for 60 h at rt (monitored by LCMS and TLC (product is a lot more polar). Reaction might have completed within 36 h). The reaction mixture was filtered though a pad of Celite, and the cake was washed thoroughly with ethyl acetate. The filtrated was washed with saturated NaHCO₃ (350 mL) and brine (100 mL). The organic phase was dried over anhydrous MgSO₄. The dried solution was filtered, and the filtrate was concentrated, providing compound S4-3 as a yellow solid (˜2.73 g, almost quantitative over three steps. LCMS indicated that the product is >95% pure).

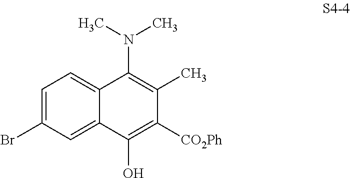

HCHO (37 wt % in water, 782 μL, 10.5 mmol, 5.4 eq), HOAc (602 μL, 10.5 mmol, 5.4 eq) and Na(OAc)₃BH (2.23 g, 10.5 mmol, 5.4 eq) were added in sequence to a solution of the crude compound S4-3 (730 mg, 1.96 mmol, 1.0 eq) in a mixture of Acetonitrile (35 mL) and THF (20 mL). The reaction mixture was stirred at rt for 3.5 h (monitored by LCMS and TLC (product is much less polar)) and quenched by slowly adding saturated NaHCO₃ (80 mL). The resulting clear mixture was extracted with ethyl acetate (3×50 mL). The organic extracts were combined and dried over anhydrous MgSO₄. The dried solution was filtered, and the filtrate was concentrated, providing compound S4-4 as a yellow solid, which was used in the next reaction without further purification.

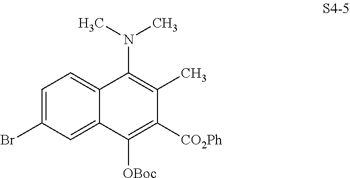

Di-tert-butyl dicarbonate (470 mg, 2.16 mmol, 1.1 eq), diisopropylethyl amine (609 mL, 3.50 mmol, 1.8 eq), and N,N-dimethylaminopyridine (20 mg, 0.16 mmol, 0.08 eq) were added to a solution of the above compound S4-4 in methylene chloride (35 mL). The resulting mixture was stirred for 1 h at rt (monitored by LCMS and TLC (product is slightly more polar)), diluted with methylene chloride (100 mL), and washed with a mixture of brine and sat NaHCO₃ (1:1, 100 mL). The organic phase was separated and dried over magnesium sulfate. The dried solution was filtered, and the filtrate was concentrated. The residue was purified by flash-column chromatography (1-5% ethyl acetate-hexanes) to afford the Boc protection product S4-5 as a yellow foamy solid (976 mg, 100%, two steps). ¹H NMR (600 MHz, CDCl₃) δ 8.14-8.10 (m, 2H), 7.68-7.66 (m, 1H), 7.52-7.55 (m, 2H), 7.39-7.37 (m, 3H), 3.05 (s, 6H), 2.58 (s, 3H), 1.57 (s, 9H).

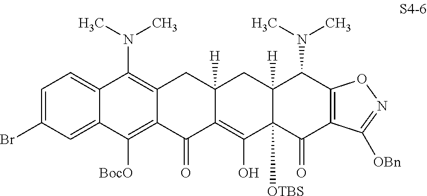

A solution of lithium diisopropylamide (1.8 M, 1.27 mL, 2.28 mmol, 3.0 eq) in heptane/ethylbenzene/THF was added drop wise via syringe to a solution of ester S4-5 (760 mg, 1.52 mol, 2.0 eq), enone (366 mg, 0.759 mol, 1.0 eq) and TMEDA (682 μL, 4.55 mmol, 6.0 eq) in tetrahydrofuran (38 mL) at −78° C. The resulting orange mixture was allowed to warm slowly to −50° C. over 3.5 h, then was poured into a mixture of saturated ammonium chloride (30 mL) and brine (30 mL). The resulting mixture was extracted with EtOAc (3×40 mL). The organic extracts were combined and the combined solution was dried over anhydrous sodium sulfate. The dried solution was filtered and the filtrate was concentrated. The residue was purified by preparative reverse phase HPLC on a Waters Autopurification system using a Sunfire Prep C18 OBD column [5 μm, 19×50 mm; flow rate, 20 mL/min; Solvent A: H₂O; Solvent B: MeOH; injection volume: 3.0 mL (CH₃CN); gradient: 90% B for 2 min, 90→100% B over 6 min, then 100% B for 7 min; mass-directed fraction collection]. Fractions with the desired MW, were collected and concentrated on a RotaVap at rt to afford the desired product S4-6 (430 mg, 64%) as a yellow solid: R_{f}=0.50 (30% ethyl acetate-hexanes). ¹H NMR (600 MHz, CDCl₃) δ 15.91 (br s, 1H), 8.28 (br s, 1H), 8.04 (d, J=9.0 Hz, 1H), 7.69 (dd, J=9.0, 2.4 Hz, 1H), 7.54 (d, J=7.2 Hz, 2H), 7.44-7.37 (m, 3H), 5.42 (d, J=12.0 Hz, 1H), 5.39 (d, J=12.0 Hz, 1H), 4.07 (d, J=10.8 Hz, 1H), 3.33 (dd, J=15.6, 3.6 Hz, 1H), 3.05 (br s, 7H), 2.69 (t, J=15.6 Hz, 1H), 2.63-2.54 (m, 8H), 2.24 (d, J=13.8 Hz, 1H), 1.63 (s, 9H), 0.89 (s, 9H), 0.33 (s, 3H), 0.19 (s, 3H). HRMS-ESI (m/z) [M+H]⁺ calcd for C₄₅H₅₅BrN₃O₉Si, 888.2891. found, 888.2856.

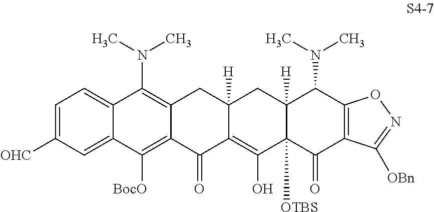

A solution of phenyllithium in di-n-butyl ether (1.8 M, 537 μL, 0.967 mmol, 2.0 eq) was added drop wise to a solution of bromide S4-6 (430 mg, 0.484 mmol, 1.0 eq) in tetrahydrofuran (24 mL) at −78° C., forming a dark red solution. After 5 min, a solution of n-butyllithium in hexanes (2.5 M, 252 μL, 0.629 mmol, 1.3 eq) was added drop wise at −78° C. followed 5 min later by N,N-dimethylformamide (185 μL, 2.42 mmol, 5.0 eq). The reaction mixture was stirred at −78° C. for 90 min. Saturated aqueous ammonium chloride solution (10 mL) was added drop wise at −78° C., followed by aqueous potassium phosphate buffer solution (pH 7.0, 0.2 M, 10 mL). The reaction mixture was allowed to warm up to 23° C., then was extracted with EtOAc (3×40 mL). The organic extracts were combined and the combined solution was dried over anhydrous sodium sulfate. The dried solution was filtered and the filtrate was concentrated, affording a yellow oil. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; injection volume: 2.0 mL (CH₃CN); gradient: 90→100% B over 8 min, then 100% B for 7 min; mass-directed fraction collection]. Fractions with the desired MW, were collected and concentrated on a RotaVap at rt to afford the desired product S4-7 (214 mg, 53%) as a yellow solid: R_{f}=0.45 (35% ethyl acetate-hexanes). ¹H NMR (400 MHz, CDCl₃) δ 15.85 (br s, 1H), 10.17 (s, 1H), 8.60 (br s, 1H), 8.24 (d, J=8.8 Hz, 1H), 8.06 (dd, J=8.8, 1.2 Hz, 1H), 7.51 (dd, J=8.0, 1.2 Hz, 2H), 7.40-7.33 (m, 3H), 5.39 (d, J=12.0 Hz, 1H), 5.35 (d, J=12.0 Hz, 1H), 4.04 (d, J=10.4 Hz, 1H), 3.36 (dd, J=15.6, 4.0 Hz, 1H), 3.03 (br s, 6H), 3.08-2.97 (m, 1H), 2.71 (t, J=15.2 Hz, 1H), 2.63-2.54 (m, 8H), 2.23 (d, J=10.0 Hz, 1H), 1.61 (s, 9H), 0.86 (s, 9H), 0.30 (s, 3H), 0.17 (s, 3H). ¹³C NMR (100 MHz, CDCl₃) δ 191.7, 187.2, 182.5, 181.7, 167.5, 151.3, 145.3, 143.6, 138.4, 134.9, 134.4, 129.8, 128.5, 128.4, 127.3, 125.8, 125.4, 121.3, 108.3, 84.7, 81.7, 72.5, 61.0, 46.4, 43.2, 41.8, 35.7, 28.2, 27.6, 26.0, 22.7, 18.9, −2.6, −3.8. HRMS-ESI (m/z) [M+H]⁺ calcd for C₄₆H₅₆N₃O₁₀Si, 838.3735. found, 888.3721.

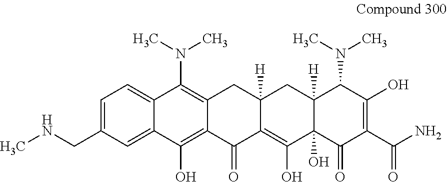

A solution of MeNH₂ in EtOH (33 wt %, 14 μL, 0.112 mmol, 7.0 eq), acetic acid (6.4 μL, 0.112 mmol, 7.0 eq) and sodium triacetoxyborohydride (25 mg, 0.112 mmol, 7.0 eq) were added to a solution of the aldehyde S4-7 (13.7 mg, 0.016 mmol, 1.0 eq) in methylene chloride (2 mL) at 23° C. The resulting orange mixture was stirred overnight, and quenched by the addition of saturated aqueous sodium bicarbonate (10 mL) and aqueous potassium phosphate buffer solutions (pH 7.0, 0.2 M, 10 mL). The product was extracted into methylene chloride (3×15 mL). The combined organic extracts were dried over anhydrous sodium sulfate. The dried solution was filtered and the filtrate was concentrated. The residue was used directly without purification.

Concentrated aqueous hydrofluoric acid solution (48 wt %, 0.3 mL) was added to a solution of the above reductive amination product in acetonitrile (0.6 mL) in a polypropylene reaction vessel at 23° C. The resulting mixture was stirred vigorously at 23° C. overnight, then was poured into water (15 mL) containing dipotassium hydrogenphosphate (3.6 g). The product was extracted into ethyl acetate (3×15 mL). The combined organic extracts were dried over anhydrous sodium sulfate. The dried solution was filtered and the filtrate was concentrated, affording a yellow solid.

Methanol (4.0 mL) was added to the above residue, forming a yellow solution. Pd black (5 mg) was added in two portions at 23° C. [In some cases, Pd—C was used.] An atmosphere of hydrogen was introduced by briefly evacuating the flask, then flushing with pure hydrogen (1 atm). The resulting mixture was stirred at 23° C. for 4 h, then was filtered through a plug of cotton. The filtrate was concentrated, affording a yellow solid. The residue was purified by a preparative reverse phase HPLC on a Waters Autopurification system using a Phenomenex Polymerx 10μ RP-1 100A column [10 μm, 150×21.20 mm; flow rate, 20 mL/min; Solvent A: 0.05 N HCl/water; Solvent B: CH₃CN; injection volume: 3.0 mL (0.05 N HCl/water); gradient: 10→35% B over 10 min; mass-directed fraction collection]. Fractions with the desired MW, eluting at 5.1-5.7 min, were collected and freeze-dried to yield Compound 300 (4.35 mg, 41% for 3 steps): ¹H NMR (400 MHz, CD₃OD, hydrochloride) δ 8.72 (br s, 1H), 8.47 (br s, 1H), 8.02 (br s, 1H), 4.42 (br s, 2H), 4.17 (br s, 1H), 3.66-3.54 (m, 8H), 3.26-3.20 (m, 1H), 3.06-2.90 (m, 7H), 2.77 (s, 3H), 2.69 (m, 1H), 2.42 (br s, 1H), 1.70 (br s, 1H); MS (ESI) m/z 551.45 (M+H).

The following compounds were prepared similarly to Compound 300.

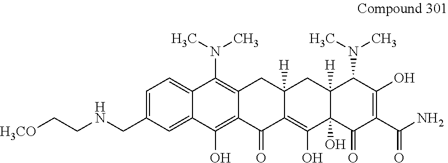

Compound 301: ¹H NMR (400 MHz, CD₃OD, hydrochloride) δ 8.65 (br s, 1H), 8.37 (br s, 1H), 7.94 (br s, 1H), 4.37 (br s, 2H), 4.08 (br s, 1H), 3.59-3.45 (m, 10H), 3.30 (s, 4H), 2.96-2.86 (m, 8H), 2.61 (m, 1H), 2.34 (m, 1H), 1.62 (m, 1H); MS (ESI) m/z 595.48 (M+H).

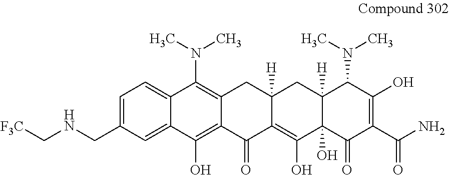

Compound 302: ¹H NMR (400 MHz, CD₃OD, hydrochloride) δ 8.77 (s, 1H), 8.46 (br s, 1H), 8.04 (br s, 1H), 4.97 (s, 2H), 4.12-4.10 (m, 3H), 3.62-3.50 (m, 8H), 3.04-2.95 (m, 7H), 2.69-2.65 (m, 1H), 2.41 (m, 1H), 1.70 (m, 1H); MS (ESI) m/z 619.45 (M+H).

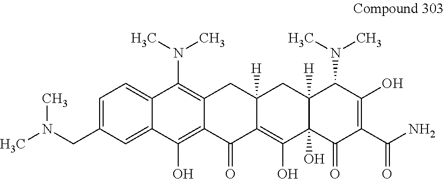

Compound 303: ¹H NMR (400 MHz, CD₃OD, hydrochloride) δ 8.76 (s, 1H), 8.50 (br s, 1H), 8.07 (br s, 1H), 4.57 (s, 2H), 4.18 (s, 1H), 3.68-3.56 (m, 7H), 3.06-2.90 (m, 14H), 2.71-2.70 (m, 1H), 2.43 (m, 1H), 1.70 (m, 1H); MS (ESI) m/z 565.54 (M+H).

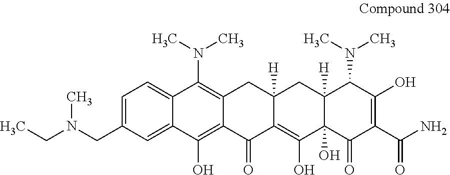

Compound 304: ¹H NMR (400 MHz, CD₃OD, hydrochloride) δ 8.77 (s, 1H), 8.50 (br s, 1H), 8.09 (br s, 1H), 4.69 (d, J=11.9 Hz, 1H), 4.49 (d, J=11.9 Hz, 1H), 4.18 (s, 1H), 3.68-3.55 (m, 9H), 3.30-2.96 (m, 8H), 2.82 (s, 3H), 2.72 (m, 1H), 2.44 (m, 1H), 1.71 (m, 1H); MS (ESI) m/z 579.47 (M+H).

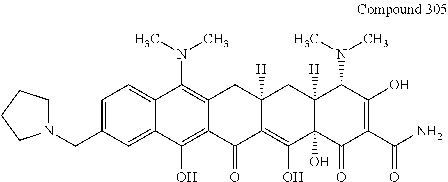

Compound 305: ¹H NMR (400 MHz, CD₃OD, hydrochloride) δ 8.76 (s, 1H), 8.45 (br s, 1H), 8.08 (d, J=6.4 Hz, 1H), 4.62 (s, 2H), 4.17 (s, 1H), 3.63-3.51 (m, 10H), 3.30-2.95 (m, 9H), 2.69 (m, 1H), 2.40 (m, 1H), 2.18-2.01 (m, 4H), 1.69 (m, 1H); MS (ESI) m/z 591.56 (M+H).

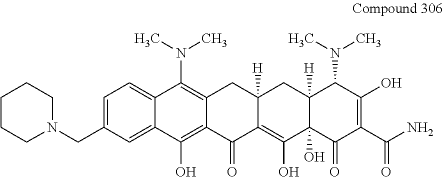

Compound 306: ¹H NMR (400 MHz, CD₃OD, hydrochloride) δ 8.76 (s, 1H), 8.47 (br s, 1H), 8.10 (br s, 1H), 4.54 (s, 2H), 4.18 (s, 1H), 3.66-3.47 (m, 10H), 3.06-2.96 (m, 9H), 2.71-2.66 (m, 1H), 2.41-2.40 (m, 1H), 1.94-1.71 (m, 6H), 1.53-1.50 (m, 1H); MS (ESI) m/z 605.60 (M+H).

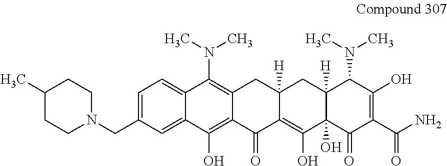

Compound 307: ¹H NMR (400 MHz, CD₃OD, hydrochloride) δ 8.74 (s, 1H), 8.45 (br s, 1H), 8.07 (br s, 1H), 4.52 (s, 2H), 4.16 (s, 1H), 3.64-3.51 (m, 10

H), 3.05-2.95 (m, 9H), 2.68-2.66 (m, 1H), 2.41-2.40 (m, 1H), 1.90-1.86 (m, 2H), 1.70 (m, 2H), 1.48 (m, 1H), 0.97 (d, J=3.7 Hz, 3H); MS (ESI) m/z 619.64 (M+H).

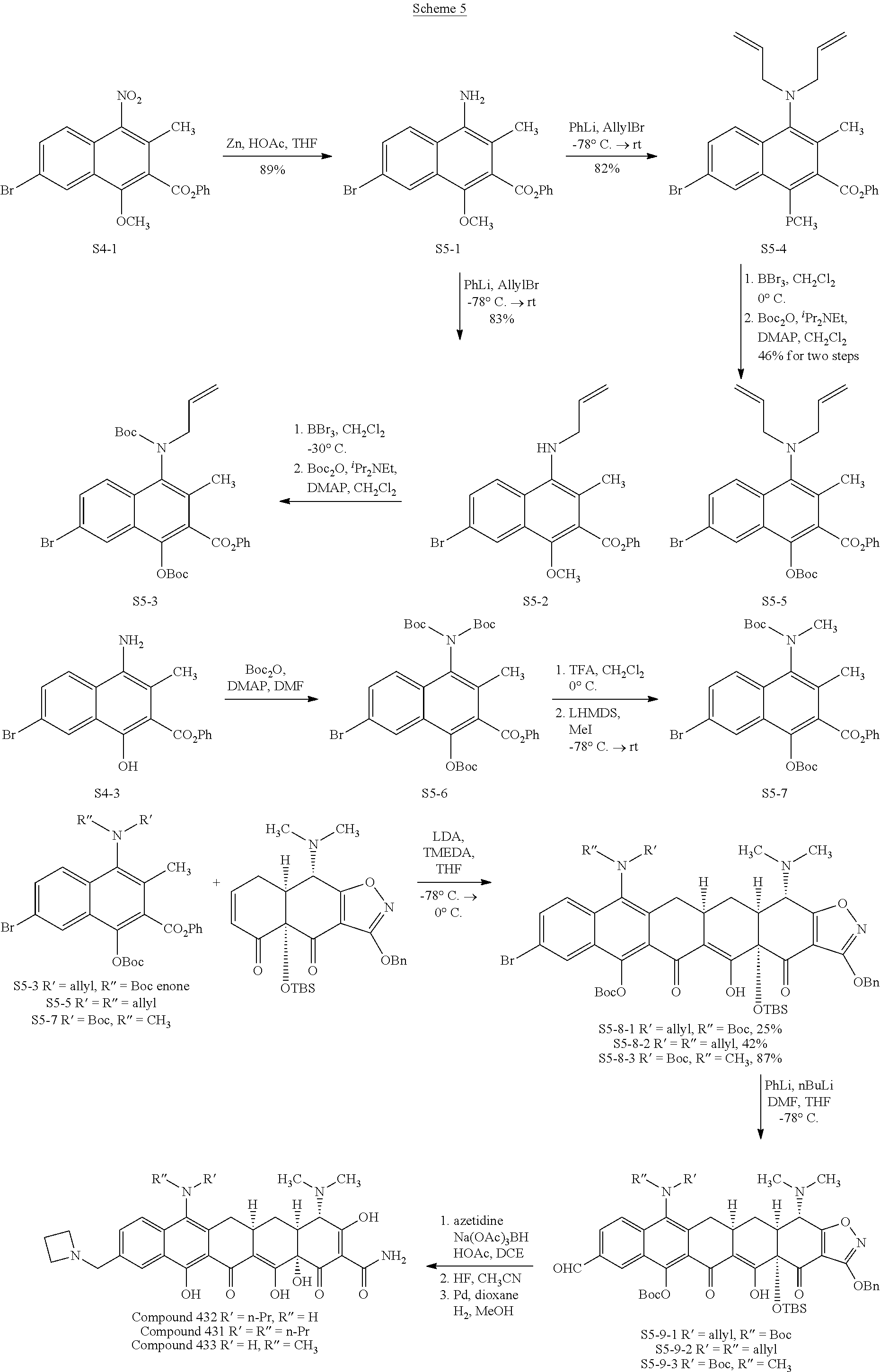

The following compounds were prepared according to Scheme 5.

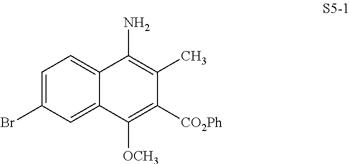

HOAc (2 mL) and Zn dust (622 mg, 9.52 mmol, 4 eq) were added to a solution of compound S4-1 (990 mg, 2.38 mmol, 1.0 eq) in THF (10 mL) at rt. The resulting reaction mixture was stirred at rt for about 15 h. And more Zn dust (311 mg, 4.76 mmol, 2 eq) was added. The reaction mixture was stirred for another 5 h and filtered through Celite, washed with ethyl acetate. The filtrate was washed with saturated NaHCO₃ and brine. The organic phase was dried over sodium sulfate. The dried solution was filtered, and the filtrate was concentrated. The residue was purified by flash-column chromatography (20% ethyl acetate-hexanes containing 10% methylene chloride) to afford the aniline product S5-1 (791 mg, 86%) as a yellow solid.

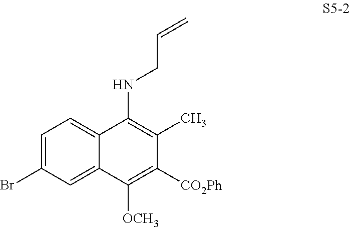

A solution of phenyllithium in di-n-butyl ether (1.8 M, 611 μL, 1.10 mmol, 1.2 eq) was added drop wise to a solution of aniline S5-1 (354 mg, 0.916 mmol, 1.0 eq) in tetrahydrofuran (18 mL) at −78° C. After 5 min, allylbromide (111 μL, 1.28 mmol, 1.4 eq) was added drop wise at −78° C. The reaction mixture was allowed to warm up to rt over 1 h 40 min, and saturated aqueous ammonium chloride solution was added. The resulting mixture was stirred for 5 min, then was extracted with EtOAc (3×40 mL). The organic extracts were combined and the combined solution was dried over anhydrous sodium sulfate. The dried solution was filtered and the filtrate was concentrated. The residue was purified by flash-column chromatography (0→5% ethyl acetate/hexanes) to afford the allylation product S5-2 (325 mg, 83%) as a colorless solid. ¹H NMR (400 MHz, CDCl₃) δ 8.26 (d, J=1.8 Hz, 1H), 7.99 (d, J=9.2 Hz, 1H), 7.60 (dd, J=1.8, 9.2 Hz, 1H), 7.49-7.44 (m, 2H), 7.32-7.28 (m, 3H), 6.11-6.01 (m, 1H), 5.39-5.34 (m, 1H), 5.21-5.18 (m, 1H), 4.06 (s, 3H), 3.69 (dt, J=6.0, 1.4 Hz, 2H), 2.49 (s, 3H); ¹³C NMR (100 MHz, CDCl₃) δ 166.6, 150.6, 148.5, 139.7, 136.0, 130.4, 129.7, 129.3, 128.2, 126.3, 126.2, 125.6, 125.1, 123.4, 121.5, 120.3, 116.4, 63.8, 53.0, 15.1.

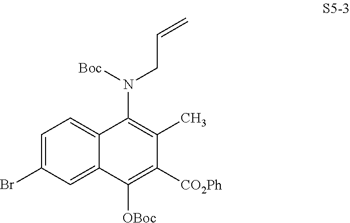

A solution of BBr₃ in CH₂Cl₂ (1.0 M, 610 μL, 0.610 mmol, 2.0 eq) was added drop wise to a solution of compound S5-2 (130 mg, 0.305 mmol, 1.0 eq) in methylene chloride (3 mL) at −30° C. The resulting yellow solution was stirred at −30° C. to −25° C. for 1.5 h and was poured into saturated NaHCO₃ solution. The mixture was warmed up to rt and extracted with methylene chloride (3×25 mL). The organic extracts were combined and dried over anhydrous Na₂SO₄. The dried solution was concentrated. The crude product was used directly for the next reaction. Crude ¹H NMR (400 MHz, CDCl₃) δ 11.77 (s, 1H), 8.58 (d, J=1.8 Hz, 1H), 7.94 (d, J=9.2 Hz, 1H), 7.71 (dd, J=1.8, 9.2 Hz, 1H), 7.50-7.46 (m, 2H), 7.36-7.32 (m, 1H), 7.26-7.23 (m, 2H), 6.13-6.03 (m, 1H), 5.39-5.34 (m, 1H), 5.21-5.18 (m, 1H), 3.60 (dt, J=5.5, 1.4 Hz, 2H), 2.73 (s, 3H).

Di-tert-butyl dicarbonate (96 mg, 0.442 mmol, 2.0 eq), and N,N-dimethylaminopyridine (3 mg, 0.02 mmol, 0.1 eq) were added to a solution of the above product in DMF (1 mL). The resulting mixture was stirred for at rt overnight, diluted with EtOAc (50 mL), washed with water (4×30 mL). The organic phase was dried over anhydrous Na₂SO₄. The dried solution was concentrated. The residue was purified by flash-column chromatography (5→7% ethyl acetate-hexanes) to afford the Boc protection product S5-3 (colorless oil, 88.3 mg, 47%, two steps, 3:1 rotamers). ¹H NMR (400 MHz, CDCl₃) δ 8.09 (m, 1H), 7.68-7.67 (m, 2H), 7.49-7.45 (m, 2H), 7.34-7.28 (m, 3H), 6.02-5.90 (m, 1H), 5.09-5.04 (m, 2H), 4.21 (d, J=6.9 Hz, 1.5H), 4.12 (d, J=6.9 Hz, 1H), 2.49 (s, 0.75H), 2.48 (s, 2.25H), 1.57 (s, 2.25H), 1.48 (s, 9H), 1.25 (s, 6.75H).

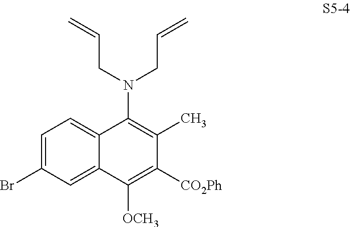

A solution of phenyllithium in di-n-butyl ether (1.8 M, 354 μL, 0.636 mmol, 1.2 eq) was added drop wise to a solution of aniline S5-1 (206 mg, 0.530 mmol, 1.0 eq) in tetrahydrofuran (10 mL) at −78° C. After 3 min, allylbromide (160 μL, 1.86 mmol, 1.5 eq) was added drop wise at −78° C. The reaction mixture was allowed to warm up to rt over 30 min, and re-cooled to −78° C. A solution of phenyllithium in di-n-butyl ether (1.8 M, 354 μL, 0.636 mmol, 1.2 eq) was added drop wise to give a red solution followed by, 3 min later, allylbromide (160 μL, 1.86 mmol, 1.5 eq). The reaction mixture was allowed to warm up to rt over 40 min and saturated aqueous ammonium chloride solution was added. The resulting mixture was stirred for 5 min, then was extracted with EtOAc (3×40 mL). The organic extracts were combined and the combined solution was dried over anhydrous sodium sulfate. The dried solution was filtered and the filtrate was concentrated. The residue was purified by flash-column chromatography (0→2% ethyl acetate/hexanes) to afford the diallylation product S5-4 (205 mg, 82%) as a pale yellow oil. ¹H NMR (400 MHz, CDCl₃) δ 8.25 (d, J=1.8 Hz, 1H), 8.06 (d, J=9.2 Hz, 1H), 7.60 (dd, J=1.8, 9.2 Hz, 1H), 7.49-7.44 (m, 2H), 7.32-7.28 (m, 3H), 5.82-5.91 (m, 2H), 5.14-5.04 (m, 4H), 4.06 (s, 3H), 3.84-3.75 (m, 4H), 2.51 (s, 3H).

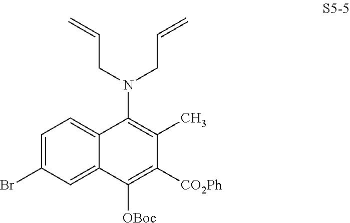

A solution of BBr₃ in CH₂Cl₂ (1.0 M, 2.22 mL, 2.22 mmol, 2.0 eq) was added drop wise to a solution of compound S5-4 (517 mg, 1.11 mmol, 1.0 eq) in methylene chloride (11 mL) at −40° C. The resulting light orange suspension was stirred at −40° C. to −30° C. for 1 h and was poured into saturated NaHCO₃ solution. The mixture was warmed up to rt and stirred for 30 min. The resulting mixture was extracted with methylene chloride (3×60 mL). The organic extracts were combined and dried over anhydrous MgSO₄. The dried solution was concentrated. The crude product was used directly for the next reaction.

Di-tert-butyl dicarbonate (291 mg, 1.33 mmol, 1.2 eq), diisopropylethylamine (232 μL, 1.33 mmol, 1.2 eq) and N,N-dimethylaminopyridine (16 mg, 0.13 mmol, 0.1 eq) were added to a solution of the above product in methylene chloride (20 mL). The resulting mixture was stirred for at rt for 20 min, and concentrated. The residue was purified by flash-column chromatography (0→5% ethyl acetate-hexanes) to afford the Boc protection product S5-5 (pale yellow solid, 280 mg, 46%, two steps). ¹H NMR (400 MHz, CDCl₃) δ 8.04 (d, J=9.2 Hz, 1H), 8.02 (d, J=1.8 Hz, 1H), 7.62 (dd, J=1.8, 9.2 Hz, 1H), 7.47-7.43 (m, 2H), 7.31-7.27 (m, 3H), 5.90-5.80 (m, 2H), 5.15-5.04 (m, 4H), 2.54 (s, 3H), 1.48 (s, 9H).

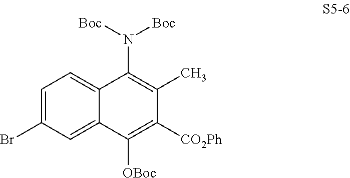

The crude material S4-3 (˜2.0 g, 5.37 mmol, 1.0 eq) was dissolved in DMF (30 mL). Di-tert-butyl dicarbonate (2.34 g, 10.75 mmol, 2.0 eq), and N,N-dimethylaminopyridine (30 mg, 0.24 mmol, 0.04 eq) were added at rt. The brownish reaction mixture was stirred at rt for 3.5 h. And a work-up procedure was carried out. The reaction mixture was diluted with ethyl acetate (300 mL), washed with brine (3×150 mL). The organic phase was dried over magnesium sulfate. The dried solution was filtered, and the filtrate was concentrated. (TLC showed many spots) The residue was re-dissolved in DMF (30 mL). Di-tert-butyl dicarbonate (2.34 g, 10.75 mmol, 2.0 eq), and N,N-dimethylaminopyridine (20 mg, 0.16 mmol, 0.03 eq) were added at rt. The resulting mixture was stirred at rt for 42 h. The reaction mixture was diluted with ethyl acetate (300 mL), washed with water (2×200 mL) then with brine (200 mL). The organic phase was dried over magnesium sulfate. The dried solution was filtered, and the filtrate was concentrated. The residue was purified by flash-column chromatography (15-25% ethyl acetate-hexanes) to afford the tri-Boc protection product S5-6 (white solid, 1.24 g, 34%). [Note: The Boc protection step was carried out in methylene chloride before. The reaction was too slow and most of the starting material 9 decomposed. It seemed to me that the starting material S4-3 was not very stable. The yield of this step could be improved by longer reaction time with more than 3 eq of Boc₂O.] ¹H NMR (400 MHz, CDCl₃) δ 8.10 (t, J=1.4 Hz, 1H), 7.69 (d, J=1.4 Hz, 2H), 7.47-7.43 (m, 2H), 7.32-7.25 (m, 3H), 2.47 (s, 3H), 1.46 (s, 9H), 1.32 (s, 18H).

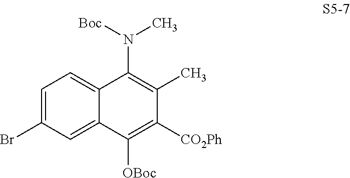

TFA (710, 9.22 mmol, 5.0 eq) was added to a solution of tri-Boc compound S5-6 (1.24 g, 1.84 mmol, 1.0 eq) in methylene chloride (60 mL) at 0° C. The resulting mixture was stored in fridge (4° C.) overnight. Sat. NaHCO₃ solution was added slowly at 0° C., and the mixture was warmed to rt. The mixture was then extracted with methylene chloride (3×50 mL). The organic extracts were combined and dried over anhydrous MgSO₄. The dried solution was filtered, and the filtrate was concentrated, providing a white foamy solid, which was used for the next reaction.

The above crude compound was dissolved in THF (30 mL). A solution of LHMDS in THF (1.0 M, 2.76 mL, 2.76 mmol, 1.5 eq) was added drop wise to the reaction at −78° C. The resulting orange mixture was stirred at −78° C. for 20 min, and then MeI (229 μL, 3.68 mmol, 2.0 eq) was added slowly. The reaction mixture was slowly warmed to rt and stirred overnight. Sat. NH₄Cl solution was added, and the reaction mixture was extracted with ethyl acetate (100 mL, then 2×50 mL). The organic extracts were combined and dried over anhydrous MgSO₄. The dried solution was filtered, and the filtrate was concentrated. The residue was purified by flash-column chromatography (10-20% ethyl acetate-hexanes) to afford the methylation product S5-7 (6.5:1 rotamers, yellow foamy solid, 724 mg, 67%, two steps), ¹H NMR (400 MHz, CDCl₃) δ 8.06-8.05 (m, 1H), 7.66-7.59 (m, 2H), 7.42-7.36 (m, 2H), 7.26-7.21 (m, 3H), 3.15 (s, 2.6H), 3.147 (s, 0.4H), 2.43 (s, 2.6H), 2.427 (s, 0.4H), 1.42 (s, 9H), 1.38 (s, 1.2H), 1.21 (s, 7.8H).

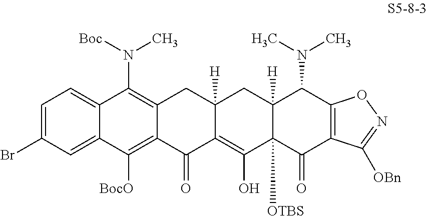

A solution of lithium diisopropylamide (1.8 M, 1.10 mL, 1.97 mmol, 3.0 eq) in heptane/ethylbenzene/THF was added drop wise via syringe to a solution of ester S5-7 (772 mg, 1.32 mol, 2.0 eq), enone (318 mg, 0.658 mol, 1.0 eq) and TMEDA (592 μL, 3.95 mmol, 6.0 eq) in tetrahydrofuran (30 mL) at −78° C. The resulting orange mixture was allowed to warm slowly to −25° C. over 5.5 h, then was poured into a mixture of saturated ammonium chloride (30 mL) and pH=7 potassium phosphate buffer solution (30 mL). The resulting mixture was extracted with EtOAc (3×30 mL). The organic extracts were combined and the combined solution was dried over anhydrous sodium sulfate. The dried solution was filtered and the filtrate was concentrated. The residue was purified by preparative reverse phase HPLC on a Waters Autopurification system using a Sunfire Prep C18 OBD column [5 μm, 19×50 mm; flow rate, 20 mL/min; Solvent A: H₂O; Solvent B: MeOH; injection volume: 3.0 mL (CH₃CN); gradient: 85→100% B over 8 min, then 100% B for 7 min; mass-directed fraction collection]. Fractions with the desired MW, were collected and concentrated on a RotaVap at rt to afford the desired product S5-8-3 (560 mg, 87%, a mixture of rotamers) as a yellow solid. ¹H NMR of the major rotamer (400 MHz, CDCl₃) δ 15.82 (br s, 1H), 8.29 (br s, 1H), 7.74 (dd, J=1.8, 8.5 Hz, 1H), 7.64 (dd, J=8.5 Hz, 1H), 7.55-7.51 (m, 2H), 7.42-7.35 (m, 3H), 5.36, 5.39 (ABq, J=12.2 Hz, 2H), 3.93 (d, J=10.7 Hz, 1H), 3.15 (s, 3H), 3.12-3.09 (m, 1H), 3.04-2.98 (m, 1H), 2.62-2.46 (m, 9H), 2.19-2.15 (m, 1H), 1.61 (s, 9H), 1.27 (s, 9H), 0.82 (s, 9H), 0.28 (s, 3H), 0.15 (s, 3H); MS (ESI) m/z 974.62, 976.62 (M+H).

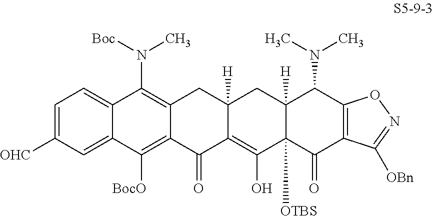

A solution of phenyllithium in di-n-butyl ether (1.8 M, 106 μL, 0.191 mmol, 2.0 eq) was added drop wise to a solution of bromide S5-8-3 (93 mg, 0.096 mmol, 1.0 eq) in tetrahydrofuran (4.5 mL) at −78° C., forming a dark red solution. After 5 min, a solution of n-butyllithium in hexanes (2.5 M, 57 μL, 0.143 mmol, 1.5 eq) was added drop wise at −78° C. followed 5 min later by N,N-dimethylformamide (36 μL, 0.478 mmol, 5.0 eq). The reaction mixture was stirred at −78° C. for 90 min. Saturated aqueous ammonium chloride solution (10 mL) was added drop wise at −78° C., followed by aqueous potassium phosphate buffer solution (pH 7.0, 0.2 M, 10 mL). The reaction mixture was allowed to warm up to 23° C., then was extracted with methylene chloride (3×15 mL). The organic extracts were combined and the combined solution was dried over anhydrous sodium sulfate. The dried solution was filtered and the filtrate was concentrated, affording a yellow oil. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; injection volume: 2.0 mL (CH₃CN); gradient: 92→98% B over 10 min, then 100% B for 5 min; mass-directed fraction collection]. Fractions with the desired MW, were collected and concentrated on a RotaVap at rt to afford the desired product S5-9-3 (44.9 mg, 51%, a mixture of rotamers) as a yellow solid. ¹H NMR (400 MHz, CDCl₃) δ 15.84, 15.97 (s, 1H), 10.21, 10.19 (s, 1H), 8.69, 8.65 (s, 1H), 8.16-8.14 (m, 1H), 7.90-7.84 (m, 1H), 7.53-7.50 (m, 2H), 7.42-7.35 (m, 3H), 5.38 (s, 2H), 4.02, 3.99 (d, J=10.4 Hz, 1H), 3.28, 3.19 (s, 3H), 3.28-2.99 (m, 2H), 2.68-2.49 (m, 9H), 2.18-2.14 (m, 1H), 1.62, 1.61 (s, 9H), 1.27, 1.24 (s, 9H), 0.85, 0.82 (s, 9H), 0.26, 0.25 (s, 3H), 0.16 (s, 3H); MS (ESI) m/z 924.68 (M+H).

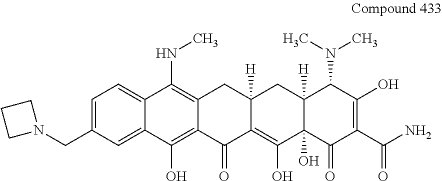

Compound 433 was prepared from compound S5-9-3 using general procedures C, D, and E. Yield: 23% over 3 steps. ¹H NMR (400 MHz, CD₃OD, hydrochloride) δ 8.68 (br s, 1H), 8.17 (br d, J=7.3 Hz, 1H), 8.01 (br s, J=7.3 Hz, 1H), 4.62 (br s, 2H), 4.27-4.25 (m, 2H), 4.17-4.12 (m, 3H), 3.20-2.98 (m, 12H), 2.60-2.38 (m, 4H), 1.62-1.72 (m, 1H); MS (ESI) m/z 563.50 (M+H).

The following compounds were prepared similarly to S5-8-3.

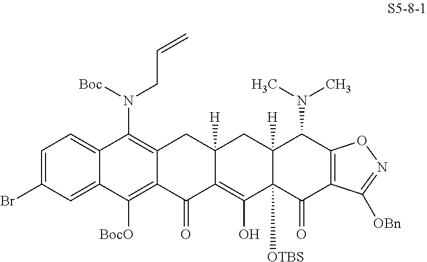

¹H NMR (400 MHz, CDCl₃) δ 15.90, 15.82 (s, 1H), 8.32-8.26 (m, 1H), 7.74-7.69 (m, 1H), 7.65-7.62 (m, 1H), 7.52-7.50 (m, 2H), 7.41-7.33 (m, 3H), 6.04-5.87 (m, 1H), 5.40-5.34 (m, 2H), 5.09-4.95 (m, 2H), 4.56-4.45 (m, 1H), 3.96-3.81 (m, 2H), 3.22-3.09 (m, 1H), 3.01-2.91 (m, 1H), 2.62-2.35 (m, 9H), 2.19-2.10 (m, 1H), 1.61, 1.60 (s, 9H), 1.27, 1.26 (s, 9H), 0.84, 0.81 (s, 9H), 0.29, 0.28 (s, 3H), 0.15, 0.14 (s, 3H),

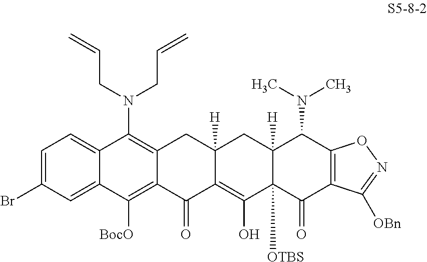

¹H NMR (400 MHz, CDCl₃) δ 15.88 (br s, 1H), 8.25 (br s, 1H), 7.80 (d, J=9.2 Hz, 1H), 7.66 (dd, J=1.8, 9.2 Hz, 1H), 7.52-7.50 (m, 2H), 7.41-7.34 (m, 3H), 5.96-5.89 (m, 1H), 5.79-5.72 (m, 1H), 5.36 (s, 2H), 5.19-4.99 (m, 4H), 4.00 (d, J=10.4 Hz, 1H), 3.86-3.67 (m, 4H), 3.45 (dd, J=4.3, 15.3 Hz, 1H), 3.00-2.92 (m, 1H), 2.62-2.48 (m, 9H), 2.18 (d, J=14.6 Hz, 1H), 1.60 (s, 9H), 1.26 (s, 9H), 0.85 (s, 9H), 0.29 (s, 3H), 0.15 (s, 3H).

The following compounds were prepared similarly to S5-9-3.

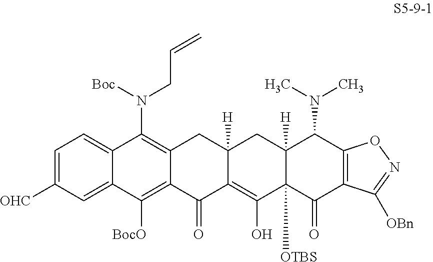

¹H NMR (400 MHz, CDCl₃) δ 15.86, 15.79 (s, 1H), 10.21, 10.19 (s, 1H), 8.66-8.62 (m, 1H), 8.14-8.10 (m, 1H), 7.90-7.81 (m, 1H), 7.52-7.50 (m, 2H), 7.41-7.30 (m, 3H), 6.05-5.87 (m, 1H), 5.41-5.32 (m, 2H), 5.10-4.95 (m, 2H), 4.60-4.48 (m, 1H), 4.06-3.84 (m, 2H), 3.28-3.15 (m, 1H), 3.04-2.94 (m, 1H), 2.76-2.44 (m, 9H), 2.21-2.12 (m, 1H), 1.62, 1.61 (s, 9H), 1.27, 1.26 (s, 9H), 0.84, 0.82 (s, 9H), 0.29, 0.28 (s, 3H), 0.16, 0.14 (s, 3H).

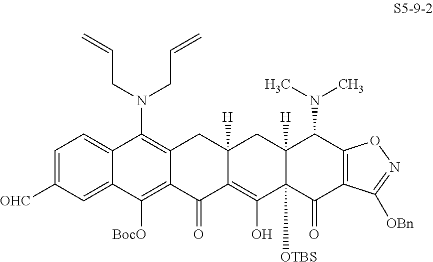

¹H NMR (400 MHz, CDCl₃) δ 15.85 (br s, 1H), 10.18 (s, 1H), 8.61 (br s, 1H), 8.23 (d, J=8.5 Hz, 1H), 8.07 (dd, J=1.2, 8.5 Hz, 1H), 7.52-7.50 (m, 2H), 7.41-7.34 (m, 3H), 5.99-5.89 (m, 1H), 5.83-5.73 (m, 1H), 5.39, 5.35 (ABq, J=12.2 Hz, 2H), 5.20-5.01 (m, 4H), 4.00 (d, J=10.4 Hz, 1H), 3.94-3.71 (m, 4H), 3.52 (dd, J=4.3, 15.9 Hz, 1H), 3.02-2.97 (m, 1H), 2.67-2.46 (m, 9H), 2.20 (d, J=14.0 Hz, 1H), 1.61 (s, 9H), 0.86 (s, 9H), 0.30 (s, 3H), 0.16 (s, 3H).

The following compounds were prepared similarly to Compound 433.

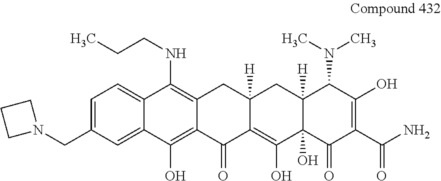

¹H NMR (400 MHz, CD₃OD, hydrochloride) δ 8.69 (d, J=1.4 Hz, 1H), 8.14 (dd, J=1.4, 8.7 Hz, 1H), 8.00 (d, J=8.7 Hz, 1H), 4.63 (br s, 2H), 4.31-4.24 (m, 2H), 4.16-4.12 (m, 3H), 3.63-2.98 (m, 11H), 2.68-2.34 (m, 4H), 1.94-1.86 (m, 2H), 1.72-1.42 (m, 1H), 1.06-1.00 (m, 3H); MS (ESI) m/z 591.47 (M+H).

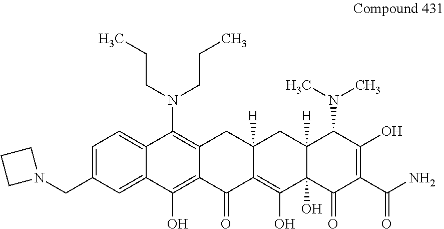

¹H NMR (400 MHz, D₂O, hydrochloride) δ 8.35 (br s, 1H), 8.05 (d, J=8.2 Hz, 1H), 7.61 (d, J=8.2 Hz, 1H), 4.31 (br s, 2H), 3.98-3.86 (m, 5H), 3.58-3.40 (m, 2H), 3.12 (dd, J=3.7, 15.6 Hz, 1H), 2.96-2.85 (m, 2H), 2.76 (s, 3H), 2.72-2.71 (m, 2H), 2.67 (s, 3H), 2.42-2.26 (m, 2H), 2.22-2.14 (m, 1H), 2.06-2.00 (m, 1H), 1.52-1.43 (m, 1H), 1.34-1.18 (m, 2H), 1.06-0.80 (m, 2H), 0.55 (t, J=7.3 Hz, 3H), 0.42 (t, J=7.1 Hz, 3H); MS (ESI) m/z 633.50 (M+H).

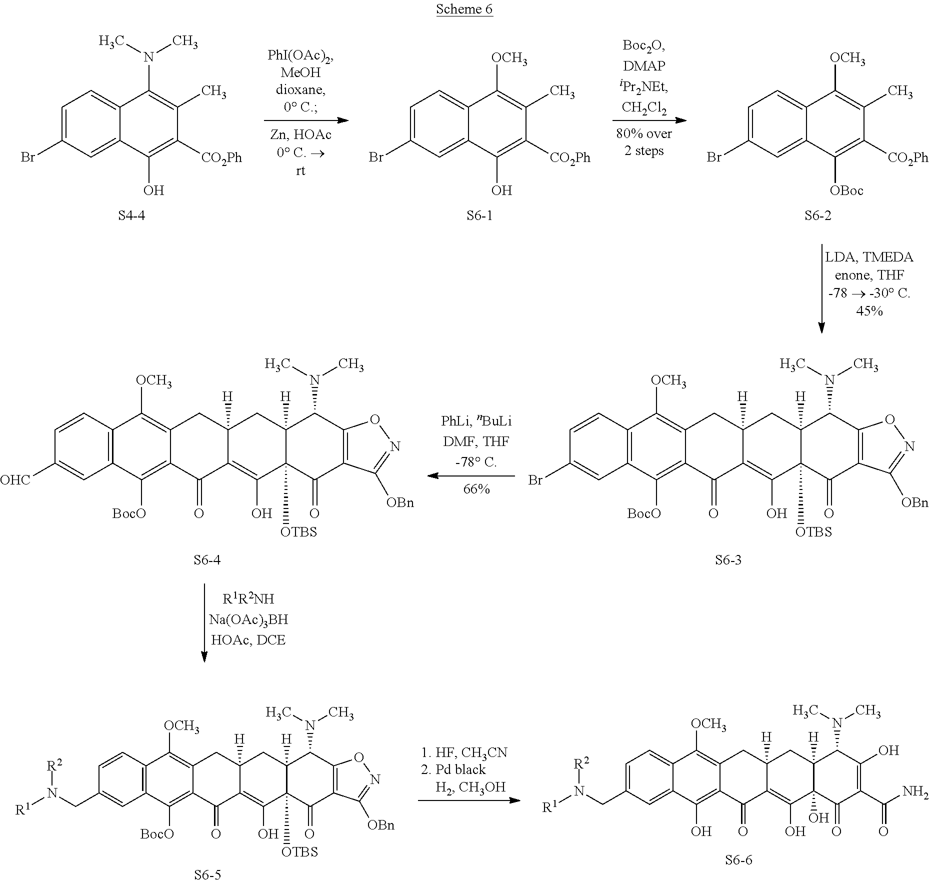

The following compounds were prepared according to Scheme 6.

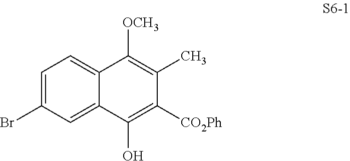

PhI(OAc)₂ (2.20 g, 6.84 mmol, 2.0 eq) was added in one portion to a solution of S4-4 (1.37 g, 3.42 mmol, 1.0 eq) in a mixture of MeOH (20 mL) and dioxane (20 mL) at 0° C. The resulting reaction mixture was stirred at that temperature for 8 min. HOAc (4 mL) and Zn dust (1.34 g, 20.5 mmol, 6.0 eq) were added at 0° C. The resulting reaction mixture was stirred for 10 min and filtered through a pad of Celite. The cake was washed thoroughly with EtOAc. The filtrate was washed with saturated sodium bicarbonate (120 mL), aqueous NaOH solution (6 N, 11 mL), and brine (50 mL). The resulting organic phase was dried over anhydrous MgSO₄. The dried solution was filtered, and the filtrate was concentrated, providing the product S6-1 as a yellow solid. The crude product was used directly for the next reaction. Crude ¹H NMR (400 MHz, CDCl₃) δ 12.10 (s, 1H), 8.06 (d, J=9.2 Hz, 1H), 8.02 (d, J=1.8 Hz, 1H), 7.63 (dd, J=1.8, 9.2 Hz, 1H), 7.42-7.38 (m, 2H), 7.27-7.22 (m, 3H), 2.63 (s, 3H), 1.43 (s, 9H); MS (ESI) m/z 385.21, 387.27 (M−H).

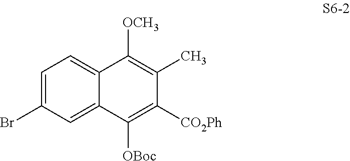

Di-tert-butyl dicarbonate (784 mg, 3.59 mmol, 1.05 eq), and N,N-dimethylaminopyridine (12 mg, 0.10 mmol, 0.03 eq) were added to a solution of the above product S6-1 in methylene chloride (30 mL). The resulting mixture was stirred for 25 min at rt and concentrated. The residue was purified by flash-column chromatography (5-10% ethyl acetate-hexanes) to afford the Boc protection product S6-2 (white solid, 1.33 g, 80%, two steps). ¹H NMR (400 MHz, CDCl₃) δ 8.02 (d, J=1.8 Hz, 1H), 7.94 (d, J=8.7 Hz, 1H), 7.62 (dd, J=1.8, 8.7 Hz, 1H), 7.43-7.39 (m, 2H), 7.27-7.24 (m, 3H), 3.84 (s, 3H), 2.51 (s, 3H), 1.43 (s, 9H); ¹³C NMR (100 MHz, CDCl₃) δ 164.4, 152.4, 151.3, 150.5, 140.5, 131.5, 129.5, 128.1, 127.8, 126.3, 125.3, 124.8, 124.7, 124.2, 121.6, 121.4, 84.6, 61.7, 27.5, 13.4.

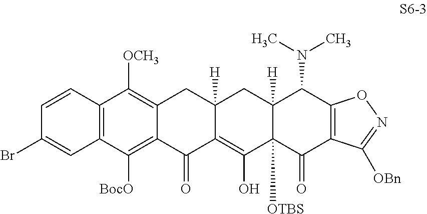

A suspension of LDA in hexanes (10 wt %, 7.96 mL, 5.32 mmol, 2.5 eq) was added slowly over 20 min to a solution of phenyl ester S6-2 (1.56 g, 3.20 mmol, 1.5 eq), enone (1.03 g, 2.13 mmol, 1.0 eq) and TMEDA (1.60 mL, 10.65 mmol, 5.0 eq) in tetrahydrofuran (50 mL) at −78° C. The resulting dark red solution was allowed to warm slowly to −40° C. over 3 h. The reaction mixture was then partitioned between saturated aqueous ammonium chloride solution (100 mL) and ethyl acetate (100 mL). The phases were separated and the aqueous phase was further extracted with ethyl acetate (100 mL). The organic extracts were combined, dried over anhydrous sodium sulfate, filtered, and concentrated. The residue was purified by flash column chromatography (2-20% ethyl acetate-hexanes) to afford the Michael-Dieckmann product S6-3 as a yellow solid (829 mg, 45%). ¹H NMR (400 MHz, CDCl₃) δ 15.94 (br s, 1H), 8.23 (br s, 1H), 7.94 (d, J=8.5 Hz, 1H), 7.67 (dd, J=2.4, 8.5 Hz, 1H), 7.50-7.48 (m, 2H), 7.39-7.32 (m, 3H), 5.37, 5.33 (ABq, J=12.2 Hz, 2H), 3.98 (d, J=11.0 Hz, 1H), 3.85 (s, 3H), 3.48 (dd, J=4.9, 15.3 Hz, 1H), 3.06-2.98 (m, 1H), 2.62-2.44 (m, 9H), 2.20 (d, J=14.0 Hz, 1H), 1.58 (s, 9H), 0.82 (s, 9H), 0.27 (s, 3H), 0.13 (s, 3H); MS (ESI) m/z 875.55, 877.52 (M+H).

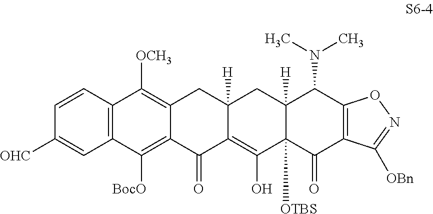

A solution of phenyllithium in di-n-butyl ether (1.8 M, 747 μL, 1.34 mmol, 2.0 eq) was added drop wise to a solution of bromide S6-3 (589 mg, 0.672 mmol, 1.0 eq) in tetrahydrofuran (30 mL) at −78° C., forming a red orange solution. After 5 min, a solution of n-butyllithium in hexanes (2.5 M, 403 μL, 1.01 mmol, 1.5 eq) was added drop wise at −78° C. followed 3 min later by the addition of N,N-dimethylformamide (257 μL, 3.36 mmol, 5.0 eq). The resulting dark red reaction mixture was stirred at −78° C. for 70 min. Saturated aqueous ammonium chloride solution (10 mL) was added drop wise at −78° C. The reaction mixture was allowed to warm up to 23° C., diluted with saturated aqueous ammonium chloride solution (˜60 mL), and extracted with ethyl acetate (100 mL, then 20 mL). The organic extracts were combined and the combined solution was dried over anhydrous sodium sulfate, filtered, and concentrated. The residue was purified by flash column chromatography (10-14% ethyl acetate-hexanes) to yield the formylated product S6-4 as a yellow solid (366 mg, 66%) and the protodebromination byproduct S6-4-1 (51.9 mg, 10%): ¹H NMR (400 MHz, CDCl₃) δ 15.90 (br s, 1H), 10.17 (s, 1H), 8.58 (br s, 1H), 8.18 (d, J=8.5 Hz, 1H), 8.08 (dd, J=1.2, 8.5 Hz, 1H), 7.50-7.48 (m, 2H), 7.39-7.32 (m, 3H), 5.37, 5.33 (ABq, J=12.2 Hz, 2H), 3.97 (d, J=11.0 Hz, 1H), 3.89 (s, 3H), 3.54 (dd, J=4.3, 15.3 Hz, 1H), 3.07-3.02 (m, 1H), 2.67-2.45 (m, 9H), 2.21 (d, J=14.0 Hz, 1H), 1.59 (s, 9H), 0.82 (s, 9H), 0.27 (s, 3H), 0.13 (s, 3H); MS (ESI) m/z 825.80 (M+H).

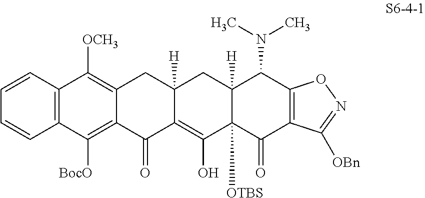

S6-4-1: ¹H NMR (400 MHz, CDCl₃) δ 16.07 (s, 1H), 8.11-8.07 (m, 2H), 7.63 (t, J=6.7 Hz, 1H), 7.55 (t, J=7.3 Hz, 1H), 7.50-7.48 (m, 2H), 7.39-7.33 (m, 3H), 5.38, 5.34 (ABq, J=12.2 Hz, 2H), 4.14 (d, J=9.8 Hz, 1H), 3.88 (s, 3H), 3.52 (dd, J=4.3, 15.3 Hz, 1H), 3.04-2.98 (m, 1H), 2.71-2.51 (m, 9H), 2.18 (d, J=14.6 Hz, 1H), 1.58 (s, 9H), 0.80 (s, 9H), 0.22 (s, 3H), 0.12 (s, 3H); MS (ESI) m/z 797.78 (M+H).

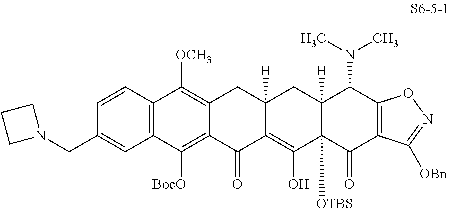

Azetidine (45 μL, 0.66 mmol, 2.0 eq), acetic acid (38 μL, 0.66 mmol, 2.0 eq) and sodium triacetoxyborohydride (91 mg, 0.43 mmol, 1.3 eq) were added sequentially to a solution of aldehyde S6-4 (273 mg, 0.33 mmol, 1.0 eq) in 1,2-dichloroethane (8 mL) at 23° C. After stirring for 1 h, the reaction mixture was quenched by the addition of saturated aqueous sodium bicarbonate (15 mL) and extracted with methylene chloride (3×20 mL). The combined organic extracts were dried over anhydrous sodium sulfate, filtered, and concentrated to yield the crude aminated product S6-5-1 (MS (ESI) m/z 866.76 (M+H)), which was used directly in the next step without further purification.

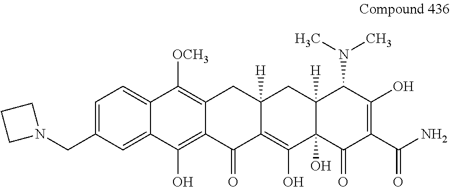

Concentrated aqueous hydrofluoric acid (48 wt %, 0.4 mL) was added to a solution of the above aminated product S6-5-1 in acetonitrile (1.5 mL) in a polypropylene reaction vessel at 23° C. The resulting mixture was stirred vigorously at 23° C. overnight and poured into aqueous dipotassium hydrogenphosphate (5 g dissolved in 40 mL water). The mixture was extracted with ethyl acetate (30 mL), and then methylene chloride (3×30 mL). The combined organic extracts were dried over anhydrous sodium sulfate, filtered, and concentrated, affording the crude TBS and Boc-deprotected product as a yellow solid. The residue was dissolved in MeOH and HCl/MeOH (0.5 N, 66 μL, 2 eq), and concentrated to give an orange solid.

Methanol (10 mL) was added to the above crude product. Pd—C (10 wt %, 25 mg) was added in one portion into the yellow solution at 23° C. The reaction vessel was sealed and purged with hydrogen by briefly evacuating the flask followed by flushing with hydrogen gas (1 atm). The yellow mixture was stirred at 23° C. for 2 h. The reaction mixture then was filtered through a small Celite pad. The filtrate was concentrated, affording the crude product as a yellow solid. The residue was purified by preparative reverse phase HPLC using a Waters Autopurification system (mass-directed fraction collection) on a Phenomenex Polymerx 10μ RP-1 100A column [10 μm, 150×21.20 mm; flow rate: 20 mL/min; Solvent A: 0.05 N aq. HCl; Solvent B: acetonitrile; injection volume 3 mL (0.05 N aq. HCl); gradient: 5→40% B over 10 min]. The peak with the desired MW was collected and freeze-dried, affording the HCl salt of the desired product Compound 436 as a yellow solid (155.5 mg, 74%, three steps). ¹H NMR (400 MHz, CD₃OD, hydrochloride) δ 8.51 (d, J=1.8 Hz, 1H), 8.07 (d, J=8.7 Hz, 1H), 7.80 (dd, J=1.8, 8.7 Hz, 1H), 4.59 (s, 2H), 4.27 (q, J=10.0 Hz, 2H), 4.17 (s, 1H), 4.15-4.10 (m, 2H), 3.79 (s, 3H), 3.39 (dd, J=4.1, 15.6 Hz, 1H), 3.08-2.98 (m, 8H), 2.63-2.56 (m, 1H), 2.53-2.45 (m, 1H), 2.37-2.30 (m, 2H), 1.69-1.60 (m, 1H); MS (ESI) m/z 564.42 (M+H).

The following compounds were prepared similarly to Compound 436 from S6-4.

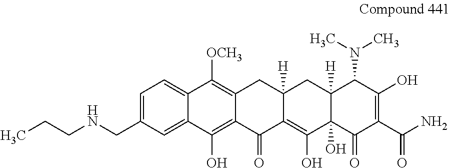

Compound 441: ¹H NMR (400 MHz, CD₃OD, hydrochloride) δ 8.56 (d, J=1.8 Hz, 1H), 8.11 (d, J=8.7 Hz, 1H), 7.81 (dd, J=1.8, 8.7 Hz, 1H), 4.40 (s, 2H), 4.13 (s, 1H), 3.81 (s, 3H), 3.41 (dd, J=4.1, 15.6 Hz, 10H), 3.09-2.97 (m, 10H), 138 (t, J=13.7 Hz, 1H), 2.28 (ddd, J=2.8, 4.6, 13.7 Hz, 1H), 1.82-1.74 (m, 2H), 1.72-1.62 (m, 1H), 1.04 (t, J=7.3 Hz, 3H); MS (ESI) m/z 566.42 (M+H).

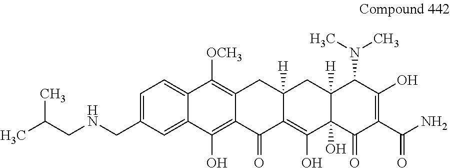

Compound 442: ¹H NMR (400 MHz, CD₃OD, hydrochloride) δ 8.59 (d, J=1.8 Hz, 1H), 8.12 (d, J=8.7 Hz, 1H), 7.81 (dd, J=1.8, 8.7 Hz, 1H), 4.41 (s, 2H), 4.08 (s, 1H), 3.81 (s, 3H), 3.42 (dd, J=4.1, 15.6 Hz, 1H), 3.07-2.94 (m, 10H), 2.41 (t, J=13.7 Hz, 1H), 2.29-2.24 (m, 1H), 2.11-2.01 (m, 1H), 1.73-1.64 (m, 1H), 1.04 (d, J=6.4 Hz, 6H); MS (ESI) m/z 580.48 (M+H).

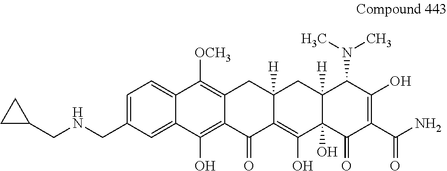

Compound 443: ¹H NMR (400 MHz, CD₃OD, hydrochloride) δ 8.56 (d, J=1.8 Hz, 1H), 8.11 (d, J=8.7 Hz, 1H), 7.82 (dd, J=1.8, 8.7 Hz, 1H), 4.42 (s, 2H), 4.13 (s, 1H), 3.81 (s, 3H), 3.41 (dd, J=4.1, 15.6 Hz, 1H), 3.06-2.98 (m, 10H), 2.38 (t, J=13.7 Hz, 1H), 2.30-2.26 (m, 1H), 1.72-1.62 (m, 1H), 1.19-1.13 (m, 1H), 0.75-0.71 (m, 2H), 0.45-0.42 (m, 2H); MS (ESI) m/z 578.41 (M+H).

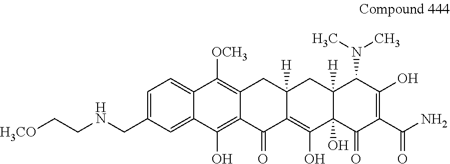

Compound 444: ¹H NMR (400 MHz, CD₃OD, hydrochloride) δ 8.54 (d, J=1.8 Hz, 1H), 8.08 (d, J=8.7 Hz, 1H), 7.81 (dd, J=1.8, 8.7 Hz, 1H), 4.43 (s, 2H), 4.14 (s, 1H), 3.80 (s, 3H), 3.69 (t, J=5.0 Hz, 1H), 3.41 (s, 3H), 3.39 (dd, J=4.1, 15.6 Hz, 1H), 3.29-3.26 (m, 1H), 3.06-2.98 (m, 9H), 2.38-2.27 (m, 2H), 1.70-1.61 (m, 1H); MS (ESI) m/z 582.44 (M+H).

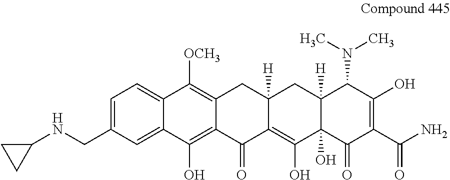

Compound 445: ¹H NMR (400 MHz, CD₃OD, hydrochloride) δ 8.56 (d, J=1.8 Hz, 1H), 8.11 (d, J=8.7 Hz, 1H), 7.82 (dd, J=1.8, 8.7 Hz, 1H), 4.52 (s, 2H), 4.13 (s, 1H), 3.80 (s, 3H), 3.41 (dd, J=4.6, 15.6 Hz, 1H), 3.06-2.97 (m, 8H), 2.86-2.81 (m, 1H), 2.38 (t, J=13.7 Hz, 1H), 2.30-2.25 (m, 1H), 1.72-1.62 (m, 1H), 0.94-0.92 (m, 4H); MS (ESI) m/z 564.53 (M+H).

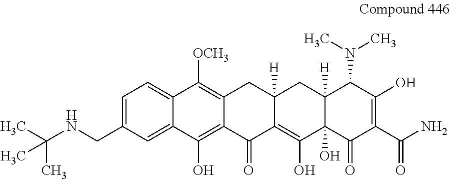

Compound 446: ¹H NMR (400 MHz, CD₃OD, hydrochloride) δ 8.59 (s, 1H), 8.13 (d, J=8.2 Hz, 1H), 7.82 (d, J=8.2 Hz, 1H), 4.39 (s, 2H), 4.12 (s, 1H), 3.82 (s, 3H), 3.42 (dd, J=3.2, 15.6 Hz, 1H), 3.05-2.97 (m, 8H), 2.40 (t, J=14.6 Hz, 1H), 2.29-2.25 (m, 1H), 1.73-1.63 (m, 1H), 1.50 (s, 9H); MS (ESI) m/z 580.64 (M+H).

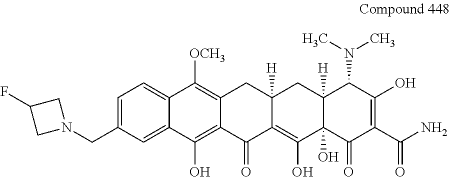

Compound 448: ¹H NMR (400 MHz, CD₃OD, trifluoroacetic acid salt) δ 8.53 (d, J=1.8 Hz, 1H), 8.11 (d, J=8.7 Hz, 1H), 7.75 (dd, J=1.8, 8.7 Hz, 1H), 5.51-5.36 (m, 1H), 4.65 (s, 2H), 4.65-4.53 (m, 1H), 4.42-4.34 (m, 1H), 4.10 (s, 1H), 3.80 (s, 3H), 3.40 (dd, J=4.1, 15.1 Hz, 1H), 3.06-2.94 (m, 8H), 2.38 (t, J=15.1 Hz, 1H), 2.28-2.24 (m, 1H), 1.72-1.62 (m, 1H); MS (ESI) m/z 582.38 (M+H).

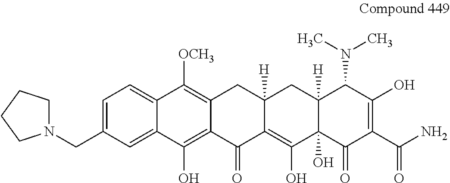

Compound 449: ¹H NMR (400 MHz, CD₃OD, hydrochloride) δ 8.58 (s, 1H), 8.12 (d, J=8.2 Hz, 1H), 7.85 (d, J=8.2 Hz, 1H), 4.59 (s, 2H), 4.12 (s, 1H), 3.81 (s, 3H), 3.56-3.51 (m, 2H), 3.41 (dd, J=4.1, 15.1 Hz, 1H), 3.27-3.21 (m, 2H), 3.06-2.97 (m, 8H), 2.38 (t, J=14.6 Hz, 1H), 2.29-2.21 (m, 3H), 2.05-2.02 (m, 2H), 1.72-1.62 (m, 1H); MS (ESI) m/z 578.59 (M+H).

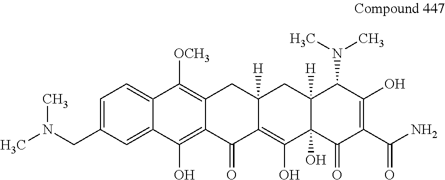

Compound 447: ¹H NMR (400 MHz, CD₃OD, hydrochloride) δ 8.57 (s, 1H), 8.14 (d, J=8.7 Hz, 1H), 7.81 (dd, J=1.8, 8.7 Hz, 1H), 4.52 (s, 2H), 4.12 (s, 1H), 3.82 (s, 3H), 3.42 (dd, J=4.1, 15.6 Hz, 1H), 3.05-2.97 (m, 8H), 2.91 (s, 6H), 2.40 (dd, J=13.7, 15.1 Hz, 1H), 2.30-2.25 (m, 1H), 1.73-1.63 (m, 1H); MS (ESI) m/z 552.55 (M+H).

Compound 450 was isolated as a byproduct when Compound 449 was prepared.

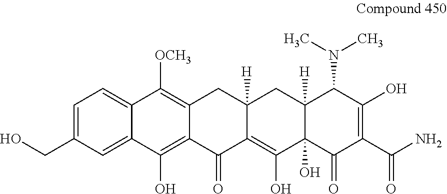

Compound 450: ¹H NMR (400 MHz, CD₃OD, hydrochloride) δ 8.37 (s, 1H), 8.00 (d, J=8.2 Hz, 1H), 7.70 (d, J=8.2 Hz, 1H), 4.78 (s, 2H), 4.10 (s, 1H), 3.80 (s, 3H), 3.41-3.37 (m, 1H), 3.04-2.97 (m, 8H), 2.36 (t, J=13.7 Hz, 1H), 2.26-2.23 (m, 1H), 1.71-1.62 (m, 1H); MS (ESI) m/z 578.59 (M+H).

Compound 452 was prepared similarly to Compound 436 from S6-4-1.

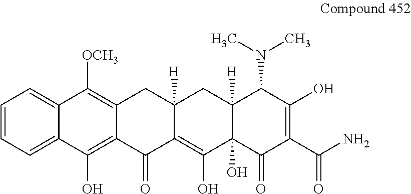

Compound 452: ¹H NMR (400 MHz, CD₃OD, hydrochloride) δ 8.34 (d, J=8.2 Hz, 1H), 7.97 (d, J=8.2 Hz, 1H), 7.66 (t, J=8.2 Hz, 1H), 7.49 (t, J=8.2 Hz, 1H), 4.07 (s, 1H), 3.76 (s, 3H), 3.38-3.34 (m, 1H), 3.01-2.93 (m, 8H), 2.32 (t, J=15.1 Hz, 1H), 2.23-2.20 (m, 1H), 1.67-1.58 (m, 1H); MS (ESI) m/z 495.42 (M+H).

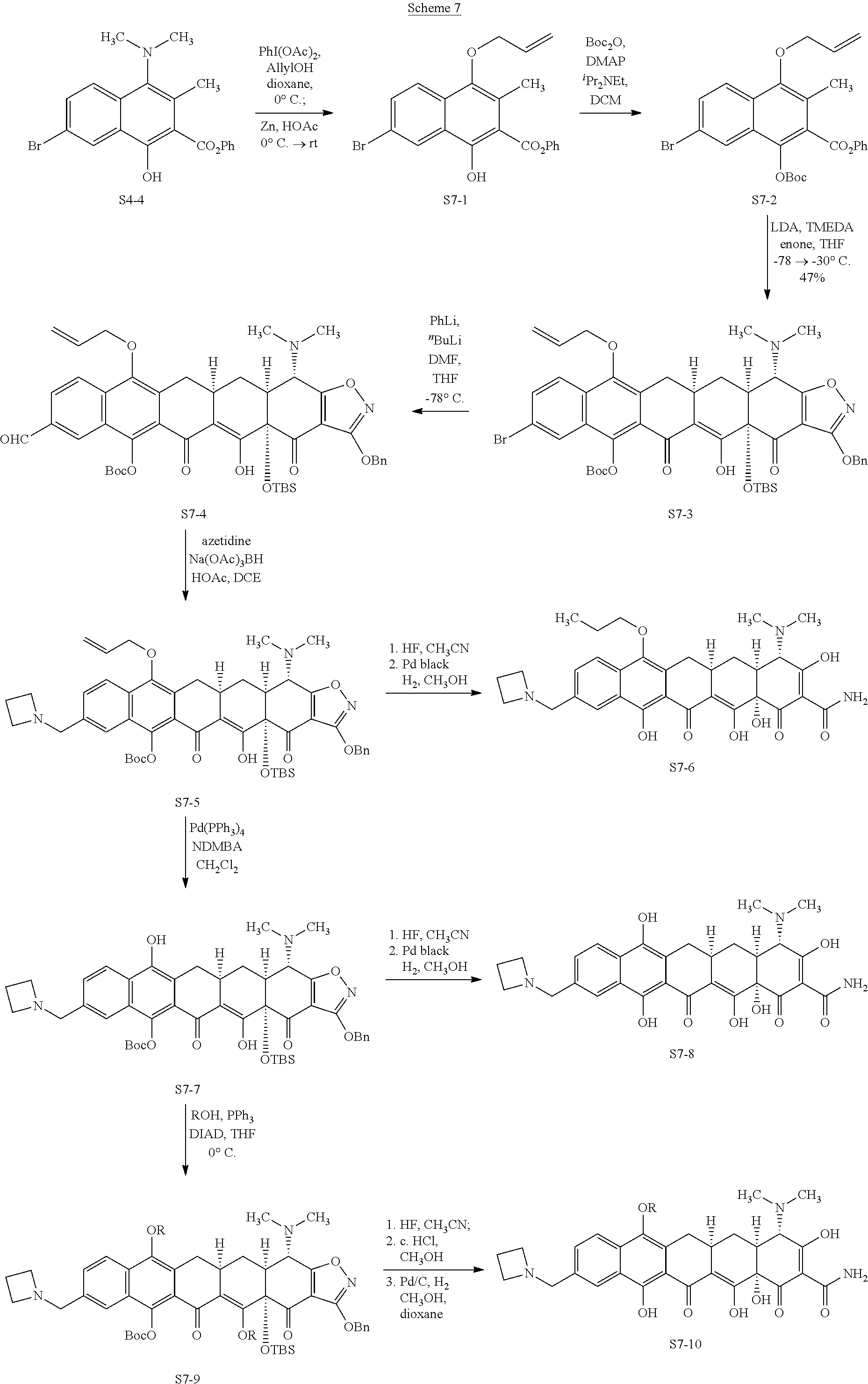

For compounds S7-9 and S7-10 R=e.g., —(C₁-C₆)alkyl, —(C₃-C₇)cycloalkyl, —(C₁-C₆)alkylene-N(R³)(R⁴), —(C₁-C₆)alkylene-(C₁-C₆)alkoxy and —C(O)—N(R³)(R⁴).

The following compounds were prepared according to Scheme 7.

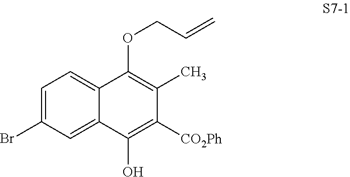

PhI(OAc)₂ (2.58 g, 8.00 mmol, 2.0 eq) was added in one portion to a solution of S4-4 (1.60 g, 4.00 mmol, 1.0 eq) in a mixture of allylalcohol (20 mL) and dioxane (20 mL) at 0° C. The resulting reaction mixture was stirred at that temperature for 30 min. HOAc (4 mL) and Zn dust (1.57 g, 24.0 mmol, 6.0 eq) were added at 0° C. The resulting reaction mixture was stirred for 25 min and filtered through a pad of Celite. The cake was washed thoroughly with EtOAc. The filtrate was washed with saturated sodium bicarbonate (120 mL), aqueous NaOH solution (6 N, 11.5 mL), and brine (50 mL). The resulting organic phase was dried over anhydrous MgSO₄. The dried solution was filtered, and the filtrate was concentrated, providing the product S7-1 as an orange solid. The crude product was used directly for the next reaction.

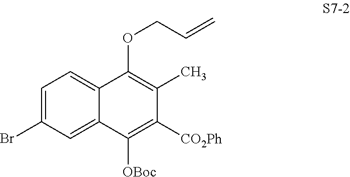

Di-tert-butyl dicarbonate (917 mg, 4.20 mmol, 1.05 eq), and N,N-dimethylaminopyridine (cat.) were added to a solution of the above product S7-1 in methylene chloride (40 mL). The resulting mixture was stirred for 10 min at rt and concentrated. The residue was purified by flash-column chromatography (1-4% ethyl acetate-hexanes) to afford the Boc protection product S7-2 (white solid, 1.64 g, 80%, two steps). ¹H NMR (400 MHz, CDCl₃) δ 8.02 (s, 1H), 7.92 (d, J=8.7 Hz, 1H), 7.60 (dd, J=1.8, 8.7 Hz, 1H), 7.42-7.38 (m, 2H), 7.26-7.18 (m, 3H), 6.16-6.07 (m, 1H), 5.46 (d, J=17.4 Hz, 1H), 5.28 (d, J=10.5 Hz, 1H), 4.41 (d, J=5.5 Hz, 2H), 2.50 (s, 3H), 1.43 (s, 9H); ¹³C NMR (100 MHz, CDCl₃) δ 164.4, 151.3, 151.2, 150.4, 140.5, 133.1, 131.4, 129.5, 128.3, 127.7, 126.3, 125.2, 125.0, 124.6, 124.3, 121.6, 121.4, 118.0, 84.5, 75.0, 27.4, 13.7.

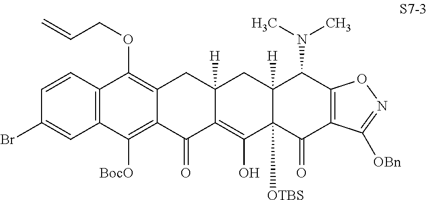

A suspension of LDA in hexanes (1.8 M, 269 μL, 0.485 mmol, 2.5 eq) was added slowly to a solution of phenyl ester S7-2 (149.6 g, 0.291 mmol, 1.5 eq), enone (94 mg, 0.194 mmol, 1.0 eq) and TMEDA (145 μL, 0.97 mmol, 5.0 eq) in tetrahydrofuran (7 mL) at −78° C. The resulting dark red brownish solution was allowed to warm slowly to −10° C. over 1 h. The reaction mixture was diluted with saturated aqueous ammonium chloride solution and pH=7 phosphate buffer solution. The resulting mixture was extracted with methylene chloride (3×15 mL). The organic extracts were combined, dried over anhydrous sodium sulfate, filtered, and concentrated. The residue was purified by flash column chromatography (1-10% ethyl acetate-hexanes) to afford the Michael-Dieckmann product S7-3 as a yellow solid (83 mg, 47%). ¹H NMR (400 MHz, CDCl₃) δ 15.93 (br s, 1H), 8.23 (br s, 1H), 7.92 (d, J=9.2 Hz, 1H), 7.67 (dd, J=1.8, 9.2 Hz, 1H), 7.50-7.48 (m, 2H), 7.39-7.30 (m, 3H), 6.20-6.10 (m, 1H), 5.45 (dd, J=1.2, 15.1 Hz, 1H), 5.38-5.32 (m, 3H), 4.47-4.38 (m, 2H), 3.96 (d, J=11.0 Hz, 1H), 3.49 (dd, J=4.3, 15.3 Hz, 1H), 3.03-2.98 (m, 1H), 2.60-2.44 (m, 9H), 2.17 (d, J=14.6 Hz, 1H), 1.58 (s, 9H), 0.81 (s, 9H), 0.26 (s, 3H), 0.12 (s, 3H); MS (ESI) m/z 901.77, 903.78 (M+H),

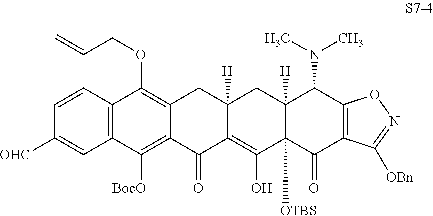

A solution of phenyllithium in di-n-butyl ether (1.8 M, 948 μL, 1.71 mmol, 2.0 eq) was added drop wise to a solution of bromide S7-3 (769 mg, 0.853 mmol, 1.0 eq) in tetrahydrofuran (30 mL) at −78° C., forming a red orange solution. After 5 min, a solution of n-butyllithium in hexanes (2.5 M, 409 μL, 1.02 mmol, 1.2 eq) was added drop wise at −78° C. followed 3 min later by the addition of N,N-dimethylformamide (326 μL, 4.26 mmol, 5.0 eq). The resulting dark red reaction mixture was stirred at −78° C. for 1 h. Saturated aqueous ammonium chloride solution (10 mL) was added drop wise at −78° C. The reaction mixture was allowed to warm up to 23° C., diluted with saturated aqueous ammonium chloride solution (˜60 mL), and extracted with ethyl acetate (100 mL, then 50 mL). The organic extracts were combined and the combined solution was dried over anhydrous sodium sulfate, filtered, and concentrated. The residue was purified by flash column chromatography (5-15% ethyl acetate-hexanes) to yield the formylated product S7-4 as a yellow solid (519 mg, 72%) and the protodebromination by-product S10-1 (83 mg, 12%).

S7-4: ¹H NMR (400 MHz, CDCl₃) δ 15.90 (br s, 1H), 10.16 (s, 1H), 8.58 (br s, 1H), 8.17 (d, J=8.5 Hz, 1H), 8.07 (dd, J=1.2, 8.5 Hz, 1H), 7.50-7.48 (m, 2H), 7.39-7.30 (m, 3H), 6.22-6.12 (m, 1H), 5.48 (dd, J=1.2, 17.1 Hz, 1H), 5.38-5.32 (m, 3H), 4.51-4.45 (m, 2H), 3.98 (d, J=10.4 Hz, 1H), 3.55 (dd, J=4.3, 15.3 Hz, 1H), 3.08-3.00 (m, 1H), 2.65-2.45 (m, 3H), 2.51 (s, 6H), 2.19 (d, J=14.6 Hz, 1H), 1.60 (s, 9H), 0.82 (s, 9H), 0.27 (s, 3H), 0.13 (s, 3H); MS (ESI) m/z 851.67 (M+H).

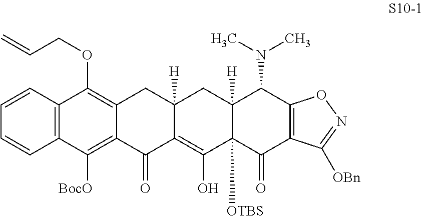

S10-1: ¹H NMR (400 MHz, CDCl₃) δ 16.03 (br s, 1H), 8.10 (br d, J=7.9 Hz, 1H), 8.06 (d, J=7.9 Hz, 1H), 7.62 (t, J=7.9 Hz, 1H), 7.55 (t, J=7.9 Hz, 1H), 7.50-7.48 (m, 2H), 7.39-7.32 (m, 3H), 6.23-6.13 (m, 1H), 5.48 (d, J=17.7 Hz, 1H), 5.38-5.32 (m, 3H), 4.45 (d, J=5.5 Hz, 2H), 4.00 (d, J=10.4 Hz, 1H), 3.53 (dd, J=3.7, 14.6 Hz, 1H), 3.03-2.98 (m, 1H), 2.64-2.46 (m, 3H), 2.51 (s, 6H), 2.18 (d, J=14.0 Hz, 1H), 1.58 (s, 9H), 0.81 (s, 9H), 0.27 (s, 3H), 0.12 (s, 3H); MS (ESI) m/z 823.68 (M+H).

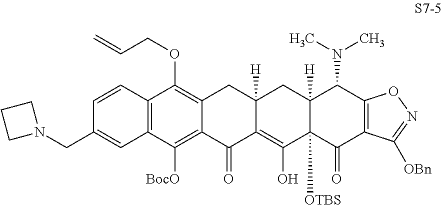

Compound S7-5 was prepared from compound S7-4 using general procedure C.

S7-5: ¹H NMR (400 MHz, CDCl₃) δ 16.02 (br s, 1H), 8.00 (d, J=8.5 Hz, 1H), 7.91 (br s, 1H), 7.57 (d, J=8.5 Hz, 1H), 7.49-7.48 (m, 2H), 7.38-7.30 (m, 3H), 6.21-6.12 (m, 1H), 5.46 (d, J=17.1 Hz, 1H), 5.38-5.27 (m, 3H), 4.43 (d, J=5.5 Hz, 2H), 3.98 (d, J=11.0 Hz, 1H), 3.72 (q, J=12.8 Hz, 2H), 3.50 (dd, J=4.3, 15.3 Hz, 1H), 3.23 (t, J=7.3 Hz, 4H), 3.01-2.96 (m, 1H), 2.62-2.43 (m, 3H), 2.49 (s, 6H), 2.17 (d, J=14.0 Hz, 1H), 2.08 (p, J=7.3 Hz, 2H), 1.58 (s, 9H), 0.81 (s, 9H), 0.26 (s, 3H), 0.12 (s, 3H); MS (ESI) m/z 892.83 (M+H).

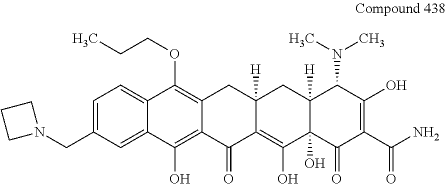

Compound 438 was prepared from compound S7-5 using general procedures D and E.

Compound 438: ¹H NMR (400 MHz, CD₃OD, hydrochloride) δ 8.53 (br s, 1H), 8.11 (d, J=8.7 Hz, 1H), 7.75 (d, J=8.7 Hz, 1H), 4.56 (s, 2H), 4.26 (q, J=9.6 Hz, 2H), 4.12-4.10 (m, 3H), 3.85-3.79 (m, 2H), 3.42 (dd, J=4.1, 15.6 Hz, 1H), 3.12-2.96 (m, 8H), 2.62-2.55 (m, 1H), 2.52-2.47 (m, 1H), 2.40 (t, J=14.6 Hz, 1H), 2.28-2.24 (m, 1H), 1.94-1.85 (m, 2H), 1.73-1.63 (m, 1H), 1.31 (t, J=7.8 Hz, 3H); MS (ESI) m/z 592.42 (M+H).

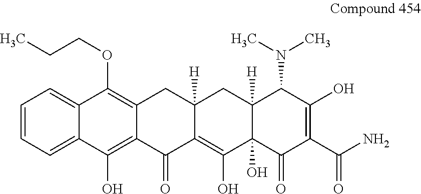

Compound 454 was prepared from compound S10-1 using general procedures D and E. Compound 454: ¹H NMR (400 MHz, CD₃OD, hydrochloride) δ 8.37 (d, J=8.2 Hz, 1H), 8.00 (d, J=8.7 Hz, 1H), 7.69 (t, J=7.8 Hz, 1H), 7.52 (t, J=7.8 Hz, 1H), 4.09 (s, 1H), 3.83-3.78 (m, 2H), 3.39 (dd, J=4.6, 15.1 Hz, 1H), 3.04-2.95 (m, 8H), 2.34 (t, J=14.2 Hz, 1H), 2.26-2.21 (m, 1H), 1.93-1.84 (m, 2H), 1.71-1.61 (m, 1H), 1.12 (t, J=7.3 Hz, 3H); MS (ESI) m/z 523.48 (M+H).

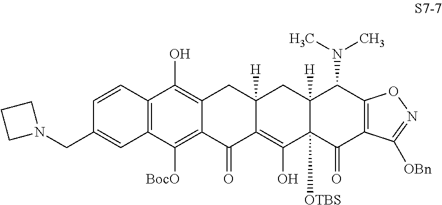

Deallylation. General Procedure F. A mixture of crude product S7-5 (0.328 mmol, 1.0 eq), Pd(PPh₃)₄ (7.6 mg, 0.0066 mmol, 0.02 eq) and N,N-dimethylbarbituric acid (256 mg, 1.64 mmol, 5.0 eq) was dissolved in degassed methylene chloride (8 mL) under nitrogen. The resulting orange reaction solution was stirred at rt for 1 h and diluted with saturated sodium bicarbonate solution. The resulting mixture was then extracted with methylene chloride (3×15 mL). The organic extracts were combined, dried over anhydrous sodium sulfate, filtered, and concentrated. The residue was purified by preparative reverse phase HPLC on a Waters Autopurification system using a Sunfire Prep C18 OBD column [5 μM, 19×50 mm; flow rate, 20 mL/min; Solvent A: H₂O with 0.1% HCO₂H; Solvent B: CH₃CN with 0.1% HCO₂H; gradient: 20→100% B over 10 min, then 100% B for 5 min; mass-directed fraction collection]. Fractions with the desired MW were collected and concentrated on a RotaVap at rt. The residue was neutralized with saturated sodium bicarbonate solution, extracted with methylene chloride (3×15 mL). The organic extracts were combined, dried over anhydrous sodium sulfate, filtered, and concentrated to afford the desired product S7-7 as an orange solid (263 mg, 86% over two steps). ¹H NMR (400 MHz, CDCl₃) δ 16.05 (br s, 1H), 7.81 (br s, 1H), 7.68-7.65 (m, 1H), 7.49-7.47 (m, 2H), 7.38-7.29 (m, 3H), 7.12-7.10 (m, 1H), 5.32, 5.35 (ABq, J=12.2 Hz, 2H), 3.97 (d, J=11.0 Hz, 1H), 3.67 (s, 2H), 3.44-3.41 (m, 1H), 3.32 (t, J=7.3 Hz, 4H), 3.05-3.00 (m, 1H), 2.55-2.42 (m, 3H), 2.46 (s, 6H), 2.16-2.10 (m, 3H), 1.58 (s, 9H), 0.81 (s, 9H), 0.26 (s, 3H), 0.12 (s, 3H); MS (ESI) m/z 852.94 (M+H).

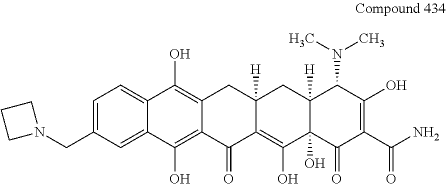

Compound 434 was prepared from compound S7-7 using general procedures D and E.

Compound 434: ¹H NMR (400 MHz, CD₃OD, hydrochloride) δ 8.48 (s, 1H), 8.25 (d, J=8.7 Hz, 1H), 7.71 (d, J=8.7 Hz, 1H), 4.56 (s, 2H), 4.26 (q, J=10.0 Hz, 2H), 4.15-4.09 (m, 3H), 3.43 (dd, J=4.1, 15.1 Hz, 1H), 3.05-2.98 (m, 8H), 2.64-2.55 (m, 1H), 2.51-2.48 (m, 1H), 2.31-2.24 (m, 2H), 1.70-1.62 (m, 1H); MS (ESI) m/z 550.64 (M+H).

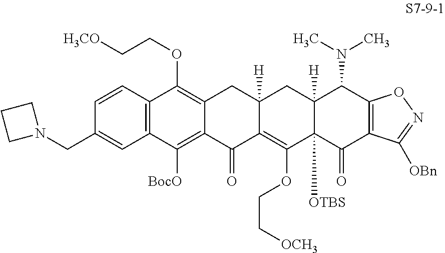

Mitsunobu Reaction. General Procedure G. DIAD (27 μL, 0.14 mmol, 5.0 eq) was added drop wise to a solution of compound S7-7 (23.8 mg, 0.028 mmol, 1.0 eq), PPh₃ (36.7 mg, 0.14 mmol, 5.0 eq) and 2-methoxyethanol (11 μL, 0.14 mmol, 5.0 eq) in THF (1 mL) at 0° C. The resulting dark red solution was then stirred at 0° C. for 1 h. Saturated sodium bicarbonate and brine (1:1, 20 mL) were added. The resulting mixture was then extracted with methylene chloride (3×15 mL). The organic extracts were combined, dried over anhydrous sodium sulfate, filtered, and concentrated. The crude product S7-9-1 was used directly for the next reaction. MS (ESI) m/z 968.81 (M+H).

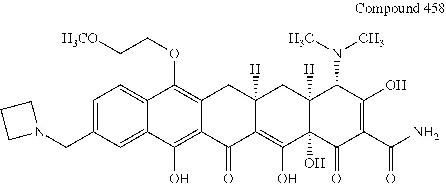

The general procedure D was applied on S7-9-1. The crude product was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; gradient: 0→100% B over 10 min, then 100% B for 5 min; mass-directed fraction collection]. Fractions with the desired MW were collected and concentrated on a RotaVap at rt. The residue was neutralized with saturated sodium bicarbonate solution, extracted with methylene chloride (3×15 mL). The organic extracts were combined, dried over anhydrous sodium sulfate, filtered, and concentrated to afford the desired product.

The above product was dissolved in MeOH (0.5 mL). Concentrated HCl (0.5 mL) was added slowly at rt. The resulting reaction mixture was stirred at rt for about 36 h, and poured into a solution of aqueous dipotassium hydrogenphosphate (3 g dissolved in 30 mL water). The mixture was extracted with methylene chloride (5×15 mL). The combined organic extracts were dried over anhydrous sodium sulfate, filtered, and concentrated. The residue was subjected to hydrogenation reaction (general procedure E) to yield Compound 458 (4.30 mg, 25% over 4 steps): ¹H NMR (400 MHz, CD₃OD, hydrochloride) δ 8.51 (d, J=1.4 Hz, 1H), 8.22 (d, J=8.7 Hz, 1H), 7.76 (dd, J=1.4, 8.7 Hz, 1H), 4.57 (s, 2H), 4.26 (q, J=9.6 Hz, 2H), 4.15-4.10 (m, 3H), 4.09-4.04 (m, 1H), 3.99-3.94 (m, 1H), 3.79-3.70 (m, 2H), 3.47 (dd, J=4.1, 15.6 Hz, 1H), 3.45 (s, 3H), 3.05-2.97 (m, 8H), 2.63-2.55 (m, 1H), 2.53-2.45 (m, 1H), 2.36 (dd, J=13.7, 15.1 Hz, 1H), 2.28-2.25 (m, 1H), 1.71-1.62 (m, 1H); MS (ESI) m/z 608.47 (M+H).

The following compounds were prepared similarly to Compound 458 from S7-7.

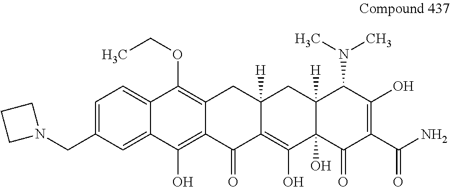

Compound 437: ¹H NMR (400 MHz, CD₃OD, hydrochloride) δ 8.52 (d, J=1.8 Hz, 1H), 8.11 (d, J=8.7 Hz, 1H), 7.74 (dd, J=1.8, 8.7 Hz, 1H), 4.56 (s, 2H), 4.24 (q, J=10.5 Hz, 2H), 4.15-4.09 (m, 3H), 3.98-3.89 (m, 2H), 3.41 (dd, J=4.1, 15.1 Hz, 1H), 3.04-2.96 (m, 8H), 2.62-2.55 (m, 1H), 2.52-2.45 (m, 1H), 2.39 (dd, J=13.8, 15.0 Hz, 1H), 2.28-2.24 (m, 1H), 1.72-1.63 (m, 1H), 1.46 (t, J=7.3 Hz, 3H); MS (ESI) m/z 578.53 (M+H).

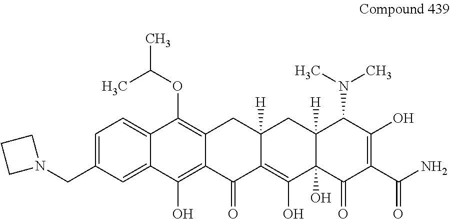

Compound 439: ¹H NMR (400 MHz, CD₃OD, hydrochloride) δ 8.52 (d, J=1.8 Hz, 1H), 8.12 (d, J=8.7 Hz, 1H), 7.73 (dd, J=1.8, 8.7 Hz, 1H), 4.56 (s, 2H), 4.29-4.22 (m, 3H), 4.15-4.10 (m, 3H), 3.42 (dd, J=4.1, 15.1 Hz, 1H), 3.04-2.91 (m, 8H), 2.62-2.55 (m, 1H), 2.53-2.44 (m, 1H), 2.37 (t, J=14.4 Hz, 1H), 2.27-2.22 (m, 1H), 1.72-1.62 (m, 1H), 1.32 (d, J=6.4 Hz, 3H), 1.27 (d, J=6.4 Hz, 3H); MS (ESI) m/z 592.53 (M+H).

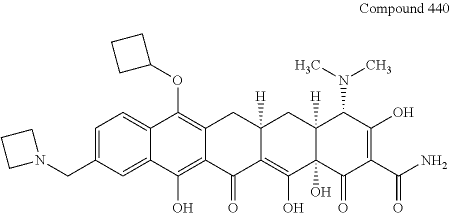

Compound 440: ¹H NMR (400 MHz, CD₃OD, hydrochloride) δ 8.51 (d, J=1.8 Hz, 1H), 8.10 (d, J=8.7 Hz, 1H), 7.73 (dd, J=1.8, 8.7 Hz, 1H), 4.55 (s, 2H), 4.32-4.22 (m, 3H), 4.13-4.09 (m, 3H), 3.41 (dd, J=3.7, 15.1 Hz, 1H), 3.04-2.96 (m, 8H), 2.62-2.48 (m, 2H), 2.48-2.22 (m, 6H), 1.74-1.62 (m, 2H), 1.47-1.40 (m, 1H); MS (ESI) m/z 604.43 (M+H).

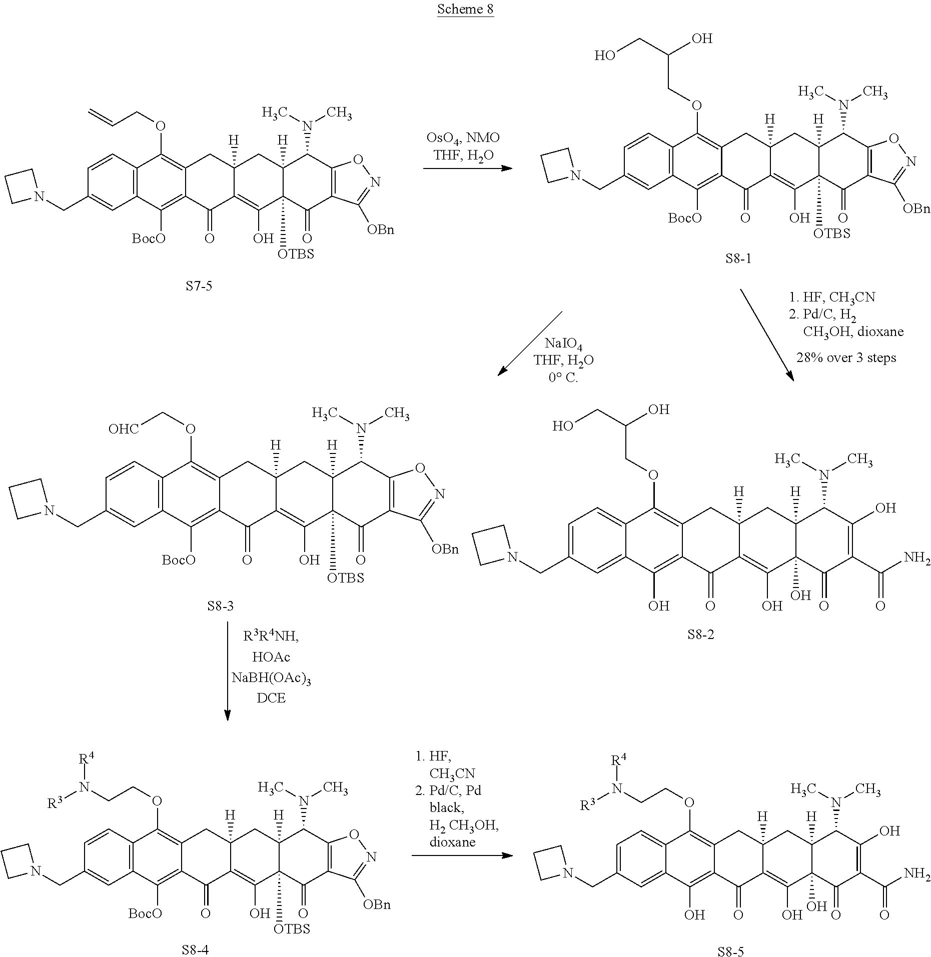

The following compounds were prepared according to Scheme 8.

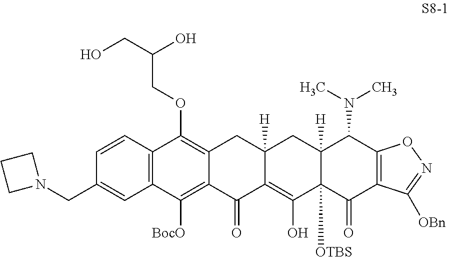

N-Methylmorpholine-N-oxide (60.4 mg, 0.516 mmol, 4.0 eq) was added to a solution of compound S7-5 (115 mg, 0.129 mmol, 1.0 eq) in a mixture of THF (3 mL) and water (0.6 mL). Then a solution of OsO₄ in water (4 wt %, 30 μL, 0.04 eq) was added. The resulting reaction mixture was stirred at rt overnight, and diluted with aqueous Na₂S₂O₃ solution (2M, 10 mL) and brine (10 mL). The resulting mixture was extracted with methylene chloride (2×20 mL). The organic extracts were combined, dried over anhydrous sodium sulfate, filtered, and concentrated. The crude product S8-1 was used directly for the next reactions. MS (ESI) m/z 926.92 (M+H).

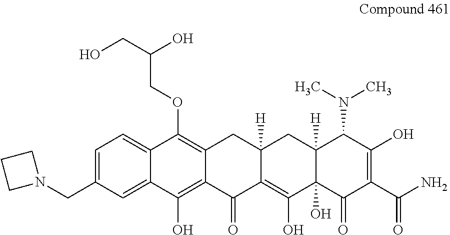

Compound 461 was prepared from ⅛ of compound S8-1 using general procedures D and E.

Compound 461 (28% over 3 steps): ¹H NMR (400 MHz, CD₃OD, hydrochloride) δ 8.52 (s, 1H), 8.27 (dd, J=2.3, 8.2 Hz, 1H), 7.75 (d, J=8.2 Hz, 1H), 4.56 (s, 2H), 4.26 (q, J=10.1 Hz, 2H), 4.15-4.09 (m, 3H), 4.06-4.02 (m, 1H), 3.98 (dd, J=3.2, 9.6 Hz, 0.5H), 3.92-3.91 (m, 1H), 3.84 (dd, J=6.0, 9.6 Hz, 0.5H), 3.73-3.70 (m, 2H), 3.53-3.47 (m, 1H), 3.05-2.97 (m, 8H), 2.62-2.55 (m, 1H), 2.52-2.45 (m, 1H), 2.36 (dd, J=14.2, 14.6 Hz, 1H), 2.28-2.25 (m, 1H), 1.71-1.62 (m, 1H); MS (ESI) m/z 624.61 (M+H).

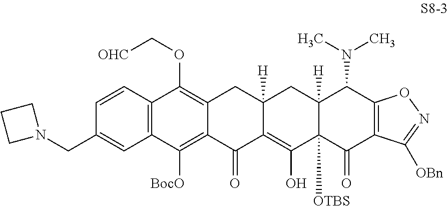

NaIO₄ (72.4 mg, 0.339 mmol, 3.0 eq) was added to a solution of compound S8-1 (⅞ of the above material, 0.113 mmol, 1.0 eq) in a mixture of THF (1.5 mL) and water (1.5 mL) at 0° C. The resulting reaction mixture was stirred at 0→8° C. for about 24 h, and diluted with aqueous Na₂S₂O₃ solution (2M, 15 mL) and brine (15 mL). The resulting mixture was extracted with methylene chloride (3×20 mL). The organic extracts were combined, dried over anhydrous sodium sulfate, filtered, and concentrated. The crude product S8-3 was used directly for the next reactions.

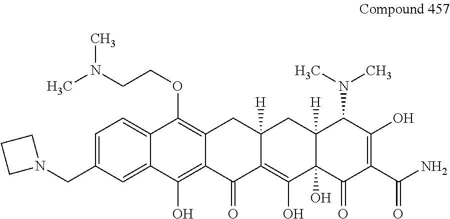

Compound 457 was prepared from ¼ of compound S8-3 using general procedures C, D, and E.

Compound 457: ¹H NMR (400 MHz, CD₃OD, hydrochloride) δ 8.56 (d, J=1.8 Hz, 1H), 8.15 (d, J=8.2 Hz, 1H), 7.83 (dd, J=1.8, 8.2 Hz, 1H), 4.58 (s, 2H), 4.30-4.23 (m, 4H), 4.15-4.09 (m, 3H), 3.77-3.71 (m, 2H), 3.43-3.38 (m, 1H), 3.10-2.98 (m, 14H), 2.63-2.55 (m, 1H), 2.52-2.45 (m, 2H), 2.39-2.36 (m, 1H), 1.73-1.63 (m, 1H); MS (ESI) m/z 621.66 (M+H).

The following compounds were prepared similarly to Compound 457 from S8-3.

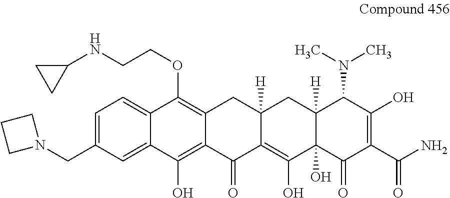

Compound 456: ¹H NMR (400 MHz, CD₃OD, hydrochloride) δ 8.54 (s, 1H), 8.16 (d, J=8.7 Hz, 1H), 7.83 (d, J=8.7 Hz, 1H), 4.58 (s, 2H), 4.30-4.09 (m, 7H), 3.70-3.68 (m, 2H), 3.46 (dd, J=3.7, 15.1 Hz, 1H), 3.07-2.91 (m, 9H), 2.63-2.55 (m, 1H), 2.53-2.37 (m, 3H), 1.71-1.61 (m, 1H), 1.12-1.07 (m, 2H), 1.00-1.96 (m, 2H); MS (ESI) m/z 633.64 (M+H).

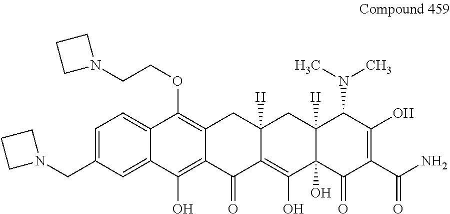

Compound 459: ¹H NMR (400 MHz, CD₃OD, hydrochloride) δ 8.53 (s, 1H), 8.12 (d, J=7.8 Hz, 1H), 7.84 (d, J=7.8 Hz, 1H), 4.58 (s, 2H), 4.48-4.43 (m, 2H), 4.38-4.22 (m, 4H), 4.17-4.08 (m, 5H), 3.79 (br s, 2H), 3.47-3.40 (m, 1H), 3.12-2.98 (m, 8H), 2.73-2.69 (m, 1H), 2.60-2.42 (m, 5H), 1.72-1.62 (m, 1H); MS (ESI) m/z 633.61 (M+H).

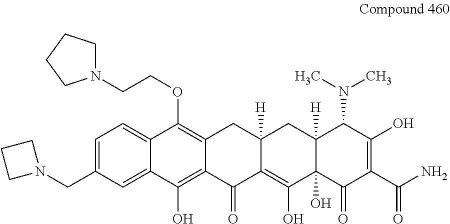

Compound 460: ¹H NMR (400 MHz, CD₃OD, hydrochloride) δ 8.51 (s, 1H), 8.14 (d, J=8.2 Hz, 1H), 7.84 (d, J=8.2 Hz, 1H), 4.58 (s, 2H), 4.30. 4.09 (m, 7H), 3.89-3.80 (m, 4H), 3.47 (dd, J=3.2, 15.1 Hz, 1H), 3.36-3.32 (m, 2H), 3.08-2.98 (m, 8H), 2.63-2.55 (m, 1H), 2.53-2.38 (m, 3H), 2.23-2.12 (m, 4H), 1.72-1.62 (m, 1H); MS (ESI) m/z 647.70 (M+H).

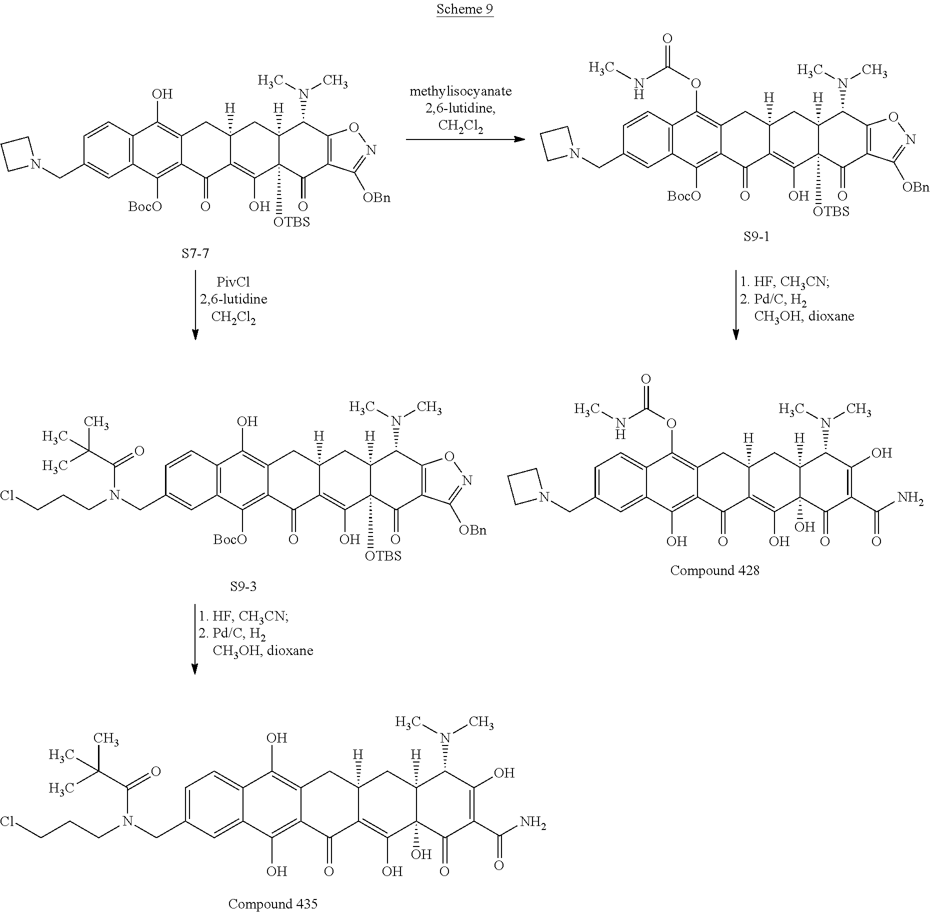

The following compounds were prepared according to Scheme 9.

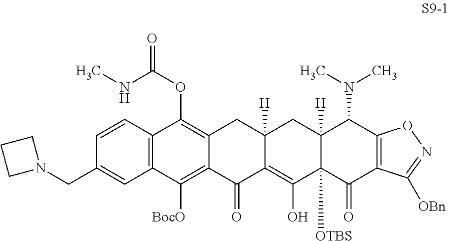

2,6-Lutidine (6.3 μL, 0.055 mmol, 3.0 eq) and methylisocyanate (3.2 μL, 0.055 mmol, 3.0 eq) were added to a solution of compound S7-7 (15.5 mg, 0.018 mmol, 1.0 eq) in a solution of methylene chloride (1 mL) at rt. The resulting reaction mixture was stirred rt for 1 h, and more methylisocyanate (5.0 μL, 0.085 mmol, 4.7 eq) was added. The resulting reaction mixture was stirred at rt for 3 h, and diluted with saturated aqueous NaHCO₃ solution (10 mL) and pH=7 potassium phosphate buffer solution (10 mL). The resulting mixture was extracted with methylene chloride (3×15 mL). The organic extracts were combined, dried over anhydrous sodium sulfate, filtered, and concentrated. The crude product S9-1 was used directly for the next reaction. MS (ESI) m/z 909.86 (M+H).

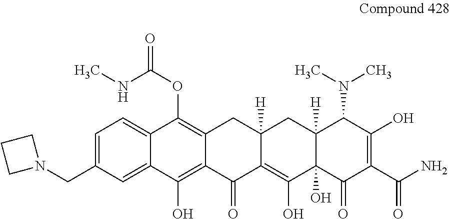

Compound 428 was prepared from compound S9-1 using general procedures D and E: ¹H NMR (400 MHz, CD₃OD, hydrochloride) δ 8.53 (d, J=1.8 Hz, 1H), 7.87 (d, J=8.2 Hz, 1H), 7.77 (dd, J=1.8, 8.2 Hz, 1H), 4.56 (s, 2H), 4.25 (q, J=9.6 Hz, 2H), 4.14-4.08 (m, 3H), 3.14-3.09 (m, 1H), 3.06-2.96 (m, 8H), 2.82 (s, 3H), 2.64-2.54 (m, 1H), 2.52-2.43 (m, 1H), 2.32 (dd, J=13.7, 14.6 Hz, 1H), 2.24-2.21 (m, 1H), 1.68-1.58 (m, 1H); MS (ESI) m/z 607.47 (M+H).

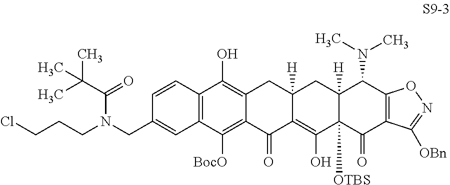

2,6-Lutidine (5.3 μL, 0.046 mmol, 3.0 eq) and pivaloyl chloride (5.6 μL, 0.046 mmol, 3.0 eq) were added to a solution of compound S7-7 (13 mg, 0.015 mmol, 1.0 eq) in a solution of methylene chloride (1 mL) at rt. The resulting reaction mixture was stirred rt overnight, and diluted with saturated aqueous NaHCO₃ solution (7 mL) and brine (15 mL). The resulting mixture was extracted with methylene chloride (2×20 mL). The organic extracts were combined, dried over anhydrous sodium sulfate, filtered, and concentrated. The crude product S9-3 was used directly for the next reaction (MS (ESI) m/z 972.76 (M+H)). No O-acylated products were detected by LC/MS.

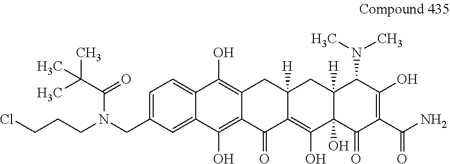

Compound 435 was prepared from compound S9-3 using general procedures D and E (mixture of rotamers 1:0.7); ¹H NMR (400 MHz, CD₃OD, hydrochloride) δ 8.17-8.09 (m, 2.4H), 7.71 (d, J=8.7 Hz, 1H), 7.65 (s, 0.7H), 7.51 (d, J=8.7 Hz, 1H), 4.20 (d, J=4.1 Hz, 0.7H), 4.09 (s, 1H), 3.70-3.47 (m, 10.2H), 3.42-3.38 (m, 1.7H), 3.09-2.96 (m, 13.6H), 2.27-2.20 (m, 2H), 2.18-2.08 (m, 4.8H), 1.69-1.60 (m, 1H), 1.52-1.43 (m, 0.7H), 1.36 (s, 15.3H); MS (ESI) m/z 670.55 (M+H).

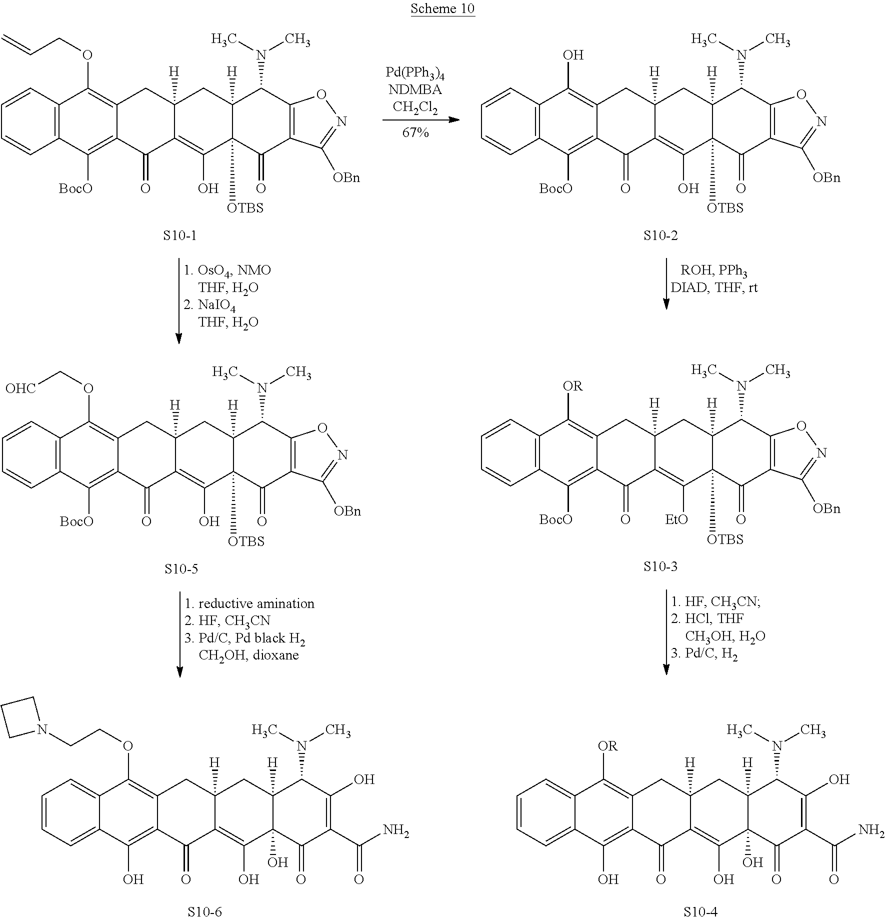

For compounds of Formulas S10-3 and S10-4 R=e.g., —(C₁-C₆)alkyl, —(C₃-C₇)cycloalkyl, —(C₁-C₆)alkylene-N(R³)(R⁴), —(C₁-C₆)alkylene-(C₁-C₆)alkoxy and —C(O)—N(R³)(R⁴).

The following compounds were prepared according to Scheme 10.

The following compounds were prepared similarly to the compounds in Scheme 7 and 8.

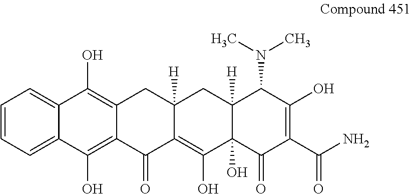

Compound 451: ¹H NMR (400 MHz, CD₃OD, hydrochloride) δ 8.34 (d, J=8.2 Hz, 1H), 8.12 (d, J=8.7 Hz, 1H), 7.63 (t, J=8.2 Hz, 1H), 7.51 (t, J=7.3 Hz, 1H), 4.09 (s, 1H), 3.42 (dd, J=4.1, 15.1 Hz, 1H), 3.05-2.96 (m, 8H), 2.28-2.20 (m, 2H), 1.69-1.59 (m, 1H); MS (ESI) m/z 481.42 (M+H).

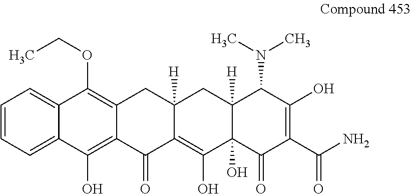

Compound 453: ¹H NMR (400 MHz, CD₃OD, hydrochloride) δ 8.37 (d, J=8.2 Hz, 1H), 7.99 (d, J=8.2 Hz, 1H), 7.69 (t, J=8.2 Hz, 1H), 7.52 (t, J=8.2 Hz, 1H), 4.09 (s, 1H), 3.95-3.88 (m, 2H), 3.38 (dd, J=3.7, 14.6 Hz, 1H), 3.08-2.94 (m, 8H), 2.34 (t, J=14.2 Hz, 1H), 2.25-2.22 (m, 1H), 1.70-1.61 (m, 1H), 1.44 (t, 6.9 Hz, 3H); MS (ESI) m/z 509.48 (M+H).

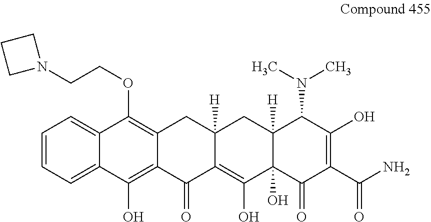

Compound 455: ¹H NMR (400 MHz, CD₃OD, hydrochloride) δ 8.34 (d, J=7.8 Hz, 1H), 7.97 (d, J=6.9 Hz, 1H), 7.74 (t, J=7.3 Hz, 1H), 7.53 (t, J=7.3 Hz, 1H), 4.46 (br s; 2H), 4.34 (br s, 2H), 4.16 (s, 1H), 4.07 (br s, 2H), 3.77 (br s, 2H), 3.40-3.36 (m, 1H), 3.08-2.98 (m, 8H), 2.72 (br s, 1H), 2.51-2.35 (m, 3H), 1.66 (br s, 1H); MS (ESI) m/z 564.48 (M+H).

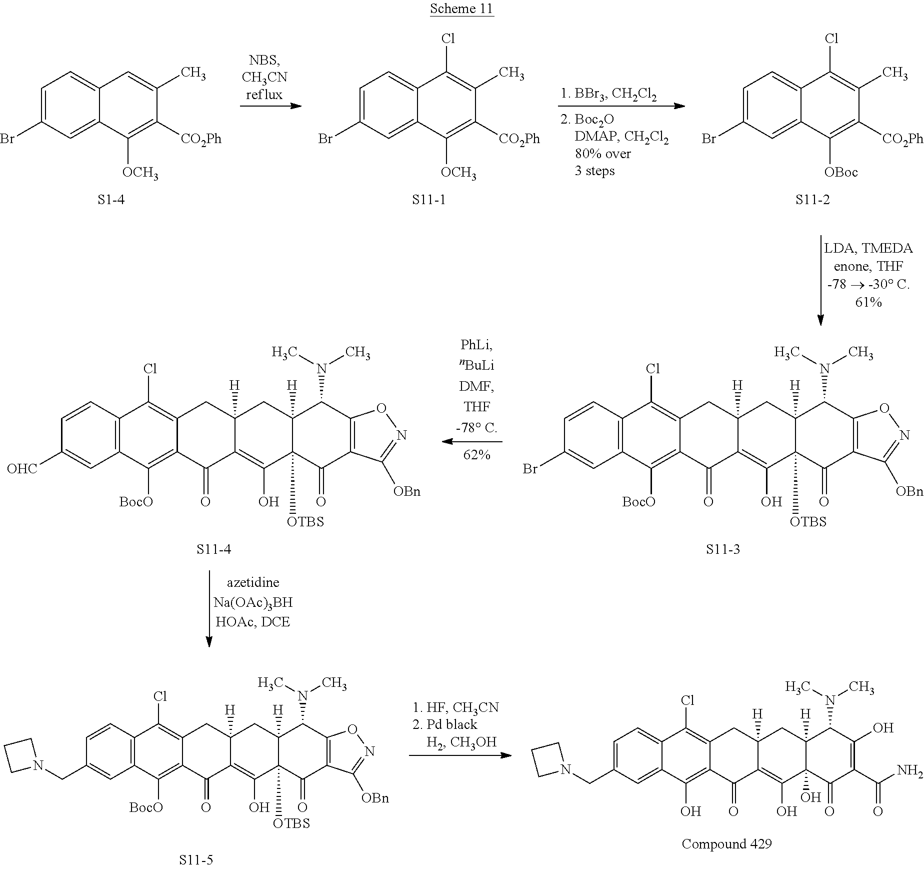

The following compounds were prepared according to Scheme 11.

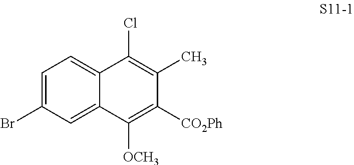

A suspension of NCS (N-chlorosuccinimide, 396 mg, 2.96 mmol, 1.1 eq) and compound S1-4 (1.00 g, 2.69 mmol, 1.0 eq) in Acetonitrile (27 mL) was heated at reflux for overnight. (both TLC and LCMS showed the reaction was complete). Then the reaction mixture was cooled to rt, and solvents were evaporated. The resulting crude product S11-1 (white solid) was dried under high vacuum and used directly for the next step.

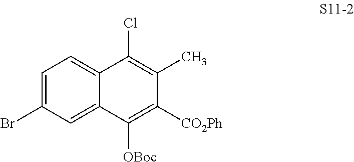

A solution of BBr₃ in CH₂Cl₂ (1.0 M, 5.38 mL, 5.38 mmol, 2.0 eq) was added slowly to a solution of the above crude compound S11-1 (2.69 mmol, 1.0 eq) in methylene chloride (30 mL) at −65° C. The resulting red solution was stirred at −65° C. for 55 min, and was poured into sat. NaHCO₃ solution (100 mL). The mixture was stirred at rt for 30 min and extracted with methylene chloride (4×60 mL). The organic extracts were combined and dried over anhydrous MgSO₄. The dried solution was filtered, and the filtrate was concentrated, providing a white solid. The crude product was used directly for the next reaction. Crude ¹H NMR (400 MHz, CDCl₃) δ 12.06 (s, 1H), 8.58 (d, J=2.4 Hz, 1H), 8.13 (d, J=9.2 Hz, 1H), 7.78 (dd, J=2.4, 9.2 Hz, 1H), 7.50-7.46 (m, 2H), 7.34 (t, J=7.3 Hz, 1H), 7.25-7.22 (m, 2H), 2.90 (s, 3H); MS (ESI) m/z 389.03, 391.03 (M−H).

Di-tert-butyl dicarbonate (616 mg, 2.82 mmol, 1.05 eq), and N,N-dimethylaminopyridine (10 mg, 0.08 mmol, 0.03 eq) were added to a solution of the above product in methylene chloride (30 mL). The resulting mixture was stirred for 40 min at rt and concentrated. The residue was purified by flash-column chromatography (1-5% ethyl acetate-hexanes) to afford the Boc protection product S11-2 (white solid, 1.06 g, 80%, three steps). ¹H NMR (400 MHz, CDCl₃) δ 8.06 (d, J=9.2 Hz, 1H), 8.02 (d, J=1.8 Hz, 1H), 7.63 (dd, J=1.8, 9.2 Hz, 1H), 7.42-7.38 (m, 2H), 7.27-7.22 (m, 3H), 2.63 (s, 3H), 1.43 (s, 9H); ¹³C NMR (100 MHz, CDCl₃) δ 164.0, 150.8, 150.3, 142.7, 132.3, 131.2, 130.7, 129.9, 129.5, 127.3, 126.5, 126.3, 125.4, 124.5, 121.9, 121.5, 84.8, 27.4, 18.3; MS (ESI) m/z 489.18, 491.10 (M−H).

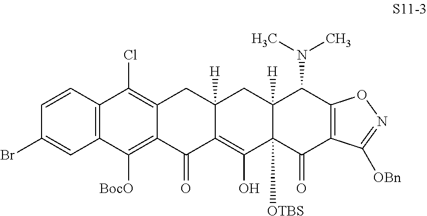

A suspension of LDA in hexanes (10 wt %, 5.4 mL, 3.6 mmol, 2.5 eq) was added slowly to a solution of phenyl ester S11-2 (1.06 g, 2.16 mmol, 1.5 eq), enone (694 mg, 1.44 mmol, 1.0 eq) and TMEDA (1.08 mL, 7.2 mmol, 5.0 eq) in tetrahydrofuran (70 mL) at −78° C. The resulting orange mixture was allowed to warm slowly to −30° C. over 2 h. The reaction mixture was then partitioned between saturated aqueous ammonium chloride solution (120 mL) and ethyl acetate (200 mL). The phases were separated and the aqueous phase was further extracted with ethyl acetate (50 mL). The organic extracts were combined, dried over anhydrous sodium sulfate, filtered, and concentrated. The residue was purified by flash column chromatography (1-5% ethyl acetate-hexanes) to afford the Michael-Dieckmann product S11-3 as a yellow solid (773 mg, 61%). ¹H NMR (400 MHz, CDCl₃) δ 15.74 (br s, 1H), 8.19 (br s, 1H), 8.04 (d, J=9.2 Hz, 1H), 7.66 (dd, J=1.8, 9.2 Hz, 1H), 7.43-7.41 (m, 2H), 7.31-7.24 (m, 3H), 5.30, 5.26 (ABq, J=12.2 Hz, 2H), 3.87 (d, J=10.4 Hz, 1H), 3.59 (dd, J=4.3, 15.9 Hz, 1H), 3.04-2.98 (m, 1H), 2.60 (t, J=15.3 Hz, 1H), 2.54-2.39 (m, 8H), 2.14 (d, J=14.0 Hz, 1H), 1.50 (s, 9H), 0.75 (s, 9H), 0.20 (s, 3H), 0.06 (s, 3H).

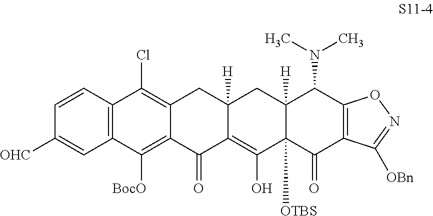

A solution of phenyllithium in di-n-butyl ether (1.8 M, 1.21 mL, 2.17 mmol, 3.0 eq) was added drop wise to a solution of bromide S11-3 (704 mg, 0.723 mmol, 1.0 eq) in tetrahydrofuran (30 mL) at −78° C., forming an orange solution.

After 5 min, a solution of n-butyllithium in hexanes (2.5 M, 434 μL, 1.08 mmol, 1.5 eq) was added drop wise at −78° C. followed 3 min later by the addition of N,N-dimethylformamide (386 μL, 5.06 mmol, 7.0 eq). The resulting dark red reaction mixture was stirred at −78° C. for 75 min. Saturated aqueous ammonium chloride solution (10 mL) was added drop wise at −78° C. The reaction mixture was allowed to warm up to 23° C., diluted with saturated aqueous ammonium chloride solution (˜60 mL), and extracted with ethyl acetate (100 mL, then 20 mL). The organic extracts were combined and the combined solution was dried over anhydrous sodium sulfate, filtered, and concentrated. The residue was purified by flash column chromatography (5-10% ethyl acetate-hexanes) to yield the formylated product 511-4 as a yellow solid (371 mg, 62%). ¹H NMR (400 MHz, CDCl₃) δ 15.79 (br s, 1H), 10.18 (d, J=4.2 Hz, 1H), 8.62 (br s, 1H), 8.38 (br s, 1H), 8.14 (br s, 1H), 7.51-7.37 (m, 5H), 5.37 (br s, 2H), 3.97 (br s, 1H), 3.73 (d, J=14.0 Hz, 1H), 3.14 (br s, 1H), 2.27-2.54 (m, 9H), 2.25 (d, J=9.8 Hz, 1H), 1.60 (s, 9H), 0.84 (s, 9H), 0.29 (s, 3H), 0.15 (s, 3H); MS (ESI) m/z 829.46 (M+H).

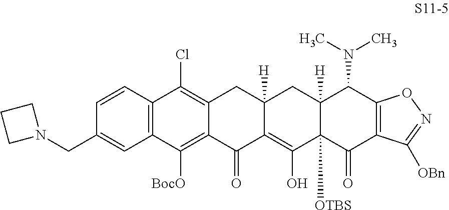

Azetidine (6.54, 0.096 mmol, 4.0 eq), acetic acid (5.5 μL, 0.096 mmol, 4.0 eq) and sodium triacetoxyborohydride (10 mg, 0.048 mmol, 2.0 eq) were added sequentially to a solution of aldehyde S11-4 (20 mg, 0.024 mmol, 1.0 eq) in 1,2-dichloroethane (1.5 mL) at 23° C. After stirring for 3.5 h, the reaction mixture was quenched by the addition of saturated aqueous sodium bicarbonate (20 mL) and extracted with methylene chloride (2×15 mL). The combined organic extracts were dried over anhydrous sodium sulfate, filtered, and concentrated to yield the crude aminated product S11-5, (MS (ESI) 870.54 (M+H)), which was used directly in the next step without further purification.

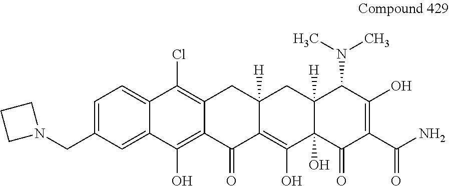

Concentrated aqueous hydrofluoric acid (48 wt %, 0.2 mL) was added to a solution of the above aminated product S11-5 in acetonitrile (0.5 mL) in a polypropylene reaction vessel at 23° C. The resulting mixture was stirred vigorously at 23° C. overnight and poured into aqueous dipotassium hydrogenphosphate (2.5 g dissolved in 20 mL water). The mixture was extracted with ethyl acetate (3×20 mL). The combined organic extracts were dried over anhydrous sodium sulfate, filtered, and concentrated, affording the crude deprotection product as a yellow solid, which was used directly in the final step without further purification.

Half of the above crude product was dissolved in methanol (1 mL) and HCl/MeOH (0.5 N, 48 μL, 0.048 mmol, 2.0 eq). Pd—C (10 wt %, 2 mg) was added in one portion into the yellow solution at 23° C. The reaction vessel was sealed and purged with hydrogen by briefly evacuating the flask followed by flushing with hydrogen gas (1 atm). The yellow mixture was stirred at 23° C. for 3.5 h. LCMS showed mostly under-reduced intermediate. The reaction mixture then was filtered through a small Celite pad, and the filtrate was concentrated. The residue was re-subjected to the reaction conditions described above, and stirred for 7 h. The reaction mixture then was filtered through a small Celite pad, and the filtrate was concentrated. The residue was purified by preparative reverse phase HPLC using a Waters Autopurification system (mass-directed fraction collection) on a Phenomenex Polymerx 10μ RP-1 100A column [10 μm, 150×21.20 mm; flow rate: 20 mL/min; Solvent A: 0.05 N aq. HCl; Solvent B: acetonitrile; injection volume 3 mL (0.05 N aq. HCl); gradient: 5→40% B over 10 min]. The peak with the desired MW, eluting at 8.75-9.29 min, was collected and freeze-dried, affording the HCl salt of the desired product Compound 429 as a yellow solid (1.62 mg, 24%, three steps). ¹H NMR (400 MHz, CD₃OD, hydrochloride) δ 8.59 (s, 1H), 8.32 (d, J=8.7 Hz, 1H), 7.85 (dd, J=1.4, 8.7 Hz, 1H), 4.60 (s, 2H), 4.27 (q, J=9.6 Hz, 2H), 4.16-4.10 (m, 3H), 3.61 (dd, J=4.1, 16.0 Hz, 1H), 3.14-2.97 (m, 8H), 2.61-2.46 (m, 3H), 2.28 (ddd, J=2.8, 5.0, 13.7 Hz, 1H), 1.74-1.64 (m, 1H); MS (ESI) m/z 568.37 (M+H).

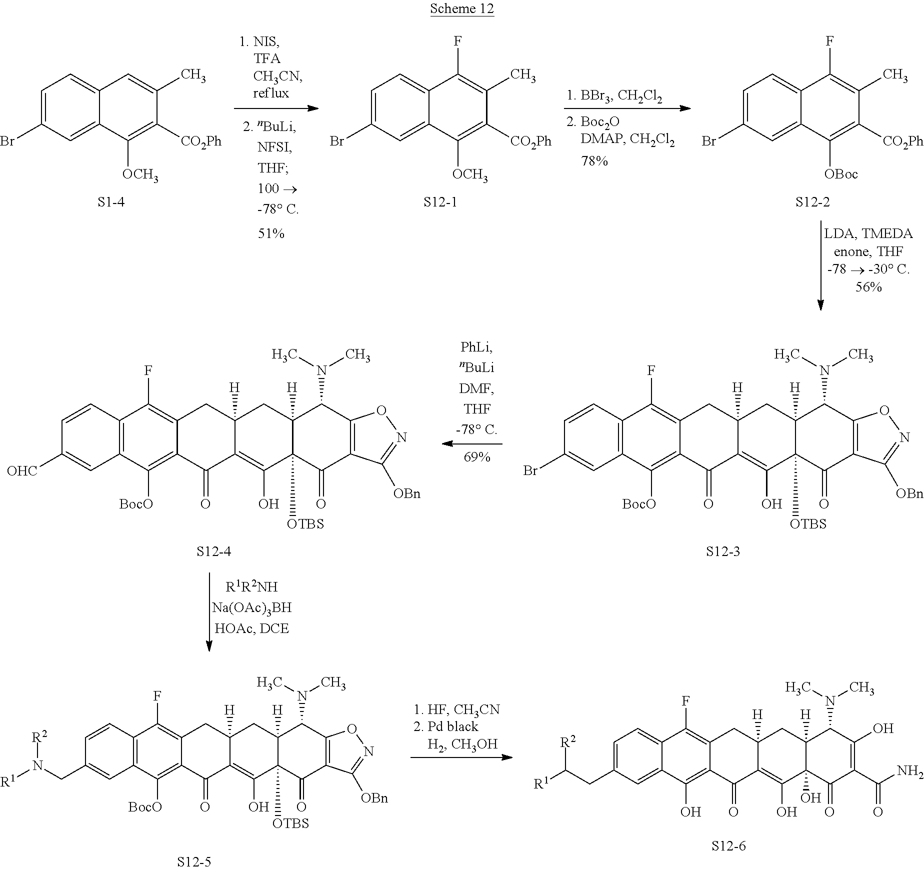

The following compounds were prepared according to Scheme 12.

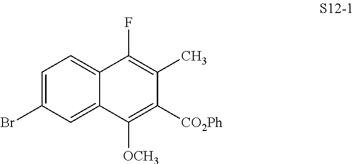

NIS (N-iodosuccinimide, 2.57 g, 11.4 mmol, 1.3 eq) and TFA (0.203 mL, 2.63 mmol, 0.3 eq) were added to a suspension of compound S1-4 (3.26 g, 8.78 mmol, 1.0 eq) in Acetonitrile (90 mL) at rt. The resulting reaction mixture was then stirred at 80° C. for about 25 h (both TLC and LCMS showed the reaction was complete). Then the reaction mixture was cooled to rt, and solvents were evaporated. The resulting off-white solid was dissolved in methylene chloride (250 mL) and the methylene chloride solution was washed with sat. NaHCO₃ (200 mL). The aqueous layer was extracted with methylene chloride (2×50 mL). The combined organic phase was dried over MgSO₄, filtered, and concentrated to afford the iodo intermediate as a pale yellow solid (4.51 g). ¹H NMR (400 MHz, CDCl₃) δ 8.24 (d, J=1.8 Hz, 1H), 8.17 (d, J=9.2 Hz, 1H), 7.66 (dd, J=1.8, 9.2 Hz, 1H), 7.48-7.44 (m, 2H), 7.32-7.27 (m, 3H), 4.08 (s, 3H), 2.76 (s, 3H).

To a solution of the above iodide product in THF (135 mL) at −100° C. was added ⁿBuLi (4.25 mL, 6.80 mmol, 1.0 eq) drop wise. After stirring at that temperature for 5 min, a solution of NFSI (N-fluorobenzenesulfonimide, 2.57 g, 8.16 mmol, 1.2 eq) in THF (17 mL) was added drop wise via a cannula. The resulting reaction mixture was warmed up slowly to −78° C. and kept at that temperature for 1 h. Phosphate buffer (pH 7, 200 mL) was added to quench the reaction. The resulting mixture was warmed up to rt and extracted with EtOAc (˜200 mL). The organic layer was separated, washed with sat. NaHCO₃ (150 mL) and brine (100 mL), dried over MgSO₄, filtered, and concentrated to afford a yellow solid, which was purified by flash-column chromatography (0-1% ethyl acetate-hexanes) to afford the fluoro product S12-1 (pale yellow solid, 1.30 g, 51%, two steps). ¹H NMR (400 MHz, CDCl₃) δ 8.25 (t, J=1.8 Hz, 1H), 7.93 (d, J=9.2 Hz, 1H), 7.66 (dd, J=1.8, 9.2 Hz, 1H), 7.48-7.44 (m, 2H), 7.32-7.27 (m, 3H), 4.06 (s, 3H), 2.48 (d, J=2.3 Hz, 3H); MS (ESI) m/z 387.09, 389.09 (M−H).

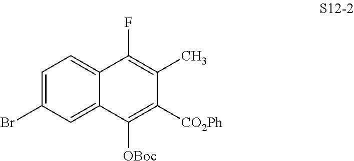

A solution of BBr₃ in CH₂Cl₂ (1.0 M, 3.67 mL, 3.67 mmol, 1.1 eq) was added slowly to a solution of compound S12-1 (1.30 g, 3.34 mmol, 1.0 eq) in methylene chloride (40 mL) at −78° C. The resulting red solution was stirred at −78° C. for 45 min, and was poured into sat. NaHCO₃ solution (250 mL). The mixture was stirred at rt for 30 min and extracted with methylene chloride (300 mL, 3×120 mL). The organic extracts were combined and dried over anhydrous MgSO₄. The dried solution was filtered, and the filtrate was concentrated, providing an off-white solid. The crude product was used directly for the next reaction.

Di-tert-butyl dicarbonate (765 mg, 3.51 mmol, 1.05 eq), and N,N-dimethylaminopyridine (10 mg, 0.08 mmol, 0.02 eq) were added to a solution of the above product in methylene chloride (40 mL). The resulting mixture was stirred for 50 min at rt and concentrated. The residue was purified by flash-column chromatography (1-3% ethyl acetate-hexanes) to afford the Boc protection product S12-2 (off-white solid, 1.23 g, 78%, two steps). ¹H NMR (400 MHz, CDCl₃) δ 8.02 (s, 1H), 7.84 (d, J=8.7 Hz, 1H), 7.60 (dd, J=1.8, 8.7 Hz, 1H), 7.41-7.38 (m, 2H), 7.26-7.24 (m, 3H), 2.48 (d, J=2.8 Hz, 3H), 1.43 (s, 9H); ¹³C NMR (100 MHz, CDCl₃) δ 163.6 (d, J=2.9 Hz, 1C), 154.0 (d, J=249.2 Hz, 1C), 151.1, 150.3, 140.4 (d, J=3.8 Hz, 1C), 131.8, 129.4, 127.3 (d, J=4.8 Hz, 1C), 126.3, 124.6 (d, J=4.8 Hz, 1C), 124.4, 123.3 (d, J=20.1 Hz, 1C), 122.3 (d, J=5.8 Hz, 1C), 122.0, 121.5, 117.8 (d, J=19.2 Hz, 1C), 84.6, 27.4, 12.0 (d, J=5.8 Hz, 1C); MS (ESI) m/z 473.16, 475.18 (M−H).

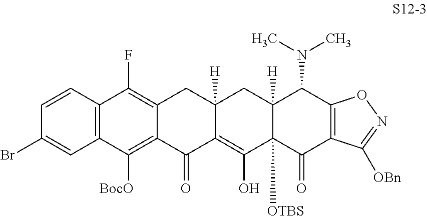

A suspension of LDA in hexanes (10 wt %, 8.07 mL, 5.40 mmol, 2.5 eq) was added slowly to a solution of phenyl ester S12-2 (1.23 g, 2.59 mmol, 1.2 eq), enone (1.04 g, 2.16 mmol, 1.0 eq) and TMEDA (1.62 mL, 10.8 mmol, 5.0 eq) in tetrahydrofuran (60 mL) at −78° C. The resulting orange mixture was allowed to warm slowly to −30° C. over 2.5 h. The reaction mixture was then partitioned between saturated aqueous ammonium chloride solution (100 mL) and ethyl acetate (100 mL). The phases were separated and the aqueous phase was further extracted with ethyl acetate (30 mL). The organic extracts were combined, dried over anhydrous sodium sulfate, filtered, and concentrated. The residue was purified by flash column chromatography (0-5% ethyl acetate-hexanes to elute the remaining ester starting material S12-2 and PhOH, then 10% ethyl acetate-hexanes to elute the desired product S12-3 and remaining enone) to afford the Michael-Dieckmann product S12-3 as a yellow solid (1.05 g, 56%) [481 mg of ester SM S12-2 and 262 mg enone SM were recovered]. ¹H NMR (400 MHz, CDCl₃) δ 15.96 (s, 1H), 8.24 (s, 1H), 7.89 (d, J=8.5 Hz, 1H), 7.70 (dd, J=1.8, 8.5 Hz, 1H), 7.51-7.50 (m, 2H), 7.39-7.31 (m, 3H), 5.39, 5.35 (ABq, J=112 Hz, 2H), 3.97 (d, J=10.4 Hz, 1H), 3.48 (dd, J=4.3, 15.3 Hz, 1H), 3.14-3.07 (m, 1H), 2.64-2.48 (m, 9H), 2.23 (d, J=14.0 Hz, 1H), 1.61 (s, 9H), 0.85 (s, 9H), 0.30 (s, 3H), 0.17 (s, 3H); MS (ESI) m/z 863.43, 865.42 (M+H).

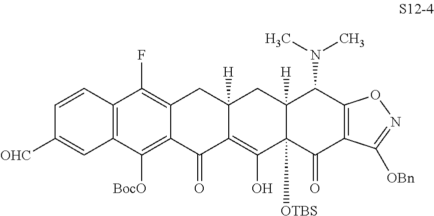

A solution of phenyllithium in di-n-butyl ether (1.03 M, 2.96 mL, 3.05 mmol, 2.5 eq) was added drop wise to a solution of bromide S12-3 (1.05 g, 1.22 mmol, 1.0 eq) in tetrahydrofuran (40 mL) at −78° C., forming a dark brownish solution. After 5 min, a solution of n-butyllithium in hexanes (1.84 M, 793 μL, 1.46 mmol, 1.2 eq) was added drop wise at −78° C. followed 2 min later by the addition of N,N-dimethylformamide (466 μL, 6.10 mmol, 5.0 eq). The resulting dark red reaction mixture was stirred at −78° C. for 90 min. Saturated aqueous ammonium chloride solution (10 mL) was added drop wise at −78° C. The reaction mixture was allowed to warm up to 23° C., diluted with saturated aqueous ammonium chloride solution (˜50 mL), and extracted with ethyl acetate (100 mL, then 30 mL). The organic extracts were combined and the combined solution was dried over anhydrous sodium sulfate, filtered, and concentrated, affording an orange oil, which was purified by flash column chromatography (5-20% ethyl acetate-hexanes) to yield the formylated product S12-4 as a yellow solid (685 mg, 69%). ¹H NMR (400 MHz, CDCl₃) δ 15.88 (s, 1H), 10.18 (s, 1H), 8.58 (s, 1H), 8.17 (d, J=8.5 Hz, 1H), 8.11 (dd, J=1.2, 8.5 Hz, 1H), 7.50-7.48 (m, 2H), 7.39-7.32 (m, 3H), 5.37, 5.34 (ABq, J=12.2 Hz, 2H), 3.94 (d, J=10.4 Hz, 1H), 3.52 (dd, J=4.3, 15.3 Hz, 1H), 3.14-3.08 (m, 1H), 2.66-2.51 (m, 9H), 2.22 (d, J=14.6 Hz, 1H), 1.59 (s, 9H), 0.82 (s, 9H), 0.26 (s, 3H), 0.13 (s, 3H); MS (ESI) m/z 813.50 (M+H).

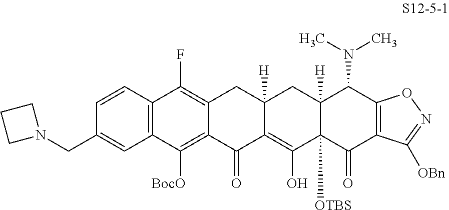

Azetidine (39 μL, 0.57 mmol, 3.0 eq), acetic acid (33 μL, 0.57 mmol, 3.0 eq) and sodium triacetoxyborohydride (61 mg, 0.29 mmol, 1.5 eq) were added sequentially to a solution of aldehyde S12-4 (156 mg, 0.19 mmol, 1.0 eq) in 1,2-dichloroethane (5 mL) at 23° C. After stirring for 1 h, the reaction mixture was quenched by the addition of saturated aqueous sodium bicarbonate (20 mL) and extracted with methylene chloride (3×20 mL). The combined organic extracts were dried over anhydrous sodium sulfate, filtered, and concentrated to yield the crude aminated product S12-5-1, which was used directly in the next step without further purification.

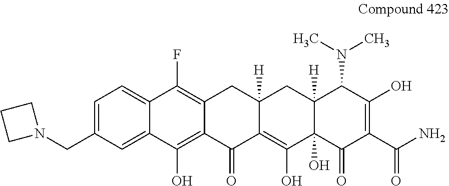

Concentrated aqueous hydrofluoric acid (48 wt %, 0.4 mL) was added to a solution of the above aminated product S12-5-1 in acetonitrile (1.0 mL) in a polypropylene reaction vessel at 23° C. The resulting mixture was stirred vigorously at 23° C. overnight and poured into aqueous dipotassium hydrogenphosphate (7 g dissolved in 40 mL water). The mixture was extracted with ethyl acetate (60 mL), and then methylene chloride (3×20 mL). The combined organic extracts were dried over anhydrous sodium sulfate, filtered, and concentrated, affording the crude TBS and Boc-deprotected product as a yellow solid, which was used directly in the final step without further purification.

Methanol (4 mL) and dioxane (2 mL) were added to the above crude product. Pd—C (10 wt %, 18 mg) was added in one portion into the yellow solution at 23° C. The reaction vessel was sealed and purged with hydrogen by briefly evacuating the flask followed by flushing with hydrogen gas (1 atm). The yellow mixture was stirred at 23° C. for 2 h. LCMS showed mostly starting material. 0.5 N HCl/MeOH (760 mL, 2.0 eq) was added. The resulting reaction mixture was stirred under hydrogen for 1 h. More Pd—C (10 wt %, 4 mg) was added. The reaction was purged with hydrogen and stirred under hydrogen (1 atm) for 25 min. LCMS analysis indicated the reaction complete. The reaction mixture then was filtered through a small Celite pad. The filtrate was concentrated, affording the crude product as a yellow solid. The residue was purified by preparative reverse phase HPLC using a Waters Autopurification system (mass-directed fraction collection) on a Phenomenex Polymerx 10μ RP-1 100A column [10 μm, 150×21.20 mm; flow rate: 20 mL/min; Solvent A: 0.05 N aq. HCl; Solvent B: acetonitrile; injection volume 4 mL (1:1 methanol: 0.05 N aq. HCl); gradient: 5→40% B over 10 min]. The peak with the desired MW, eluting at 6.72-8.28 min, was collected and freeze-dried, affording the HCl salt of the desired product Compound 423 as a yellow solid (57 mg, 47%, three steps). [The final hydrogenation goes much faster in MeOH with the bis-HCl salt of the starting material preformed]. ¹H NMR (400 MHz, CD₃OD, hydrochloride) δ 8.53 (s, 1H), 8.06 (d, J=8.2 Hz, 1H), 7.82 (dd, J=1.8, 8.7 Hz, H), 4.59 (s, 2H), 4.27 (q, J=9.6 Hz, 2H), 4.16-4.10 (m, 3H), 3.37 (dd, J=4.1, 14.6 Hz, 1H), 3.12-2.97 (m, 8H), 2.63-2.55 (m, 1H), 2.53-2.45 (m, 1H), 2.38 (t, J=14.6 Hz, 1H), 2.27 (ddd, J=2.8, 5.0, 13.7 Hz, 1H), 1.72-1.62 (m, 1H); MS (ESI) m/z 552.40 (M+H).

The following compounds were prepared similarly to Compound 423.

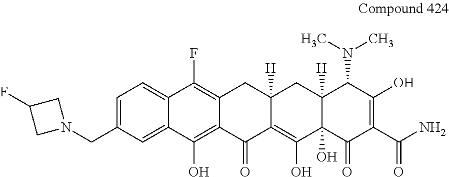

Compound 424: ¹H NMR (400 MHz, CD₃OD, hydrochloride) δ 8.57-8.54 (m, 1H), 8.07 (d, J=8.7 Hz, 1H), 7.88-7.82 (m, 1H), 5.55-5.34 (m, 1H), 4.73-4.30 (m, 6H), 4.12 (s, 1H), 3.37 (dd, J=4.1, 15.6 Hz, 1H), 3.13-2.97 (m, 8H), 2.38 (t, J=14.6 Hz, 1H), 2.28 (ddd, J=2.8, 5.0, 13.7 Hz, 1H), 1.72-1.62 (m, 1H); MS (ESI) m/z 570.36 (M+H).

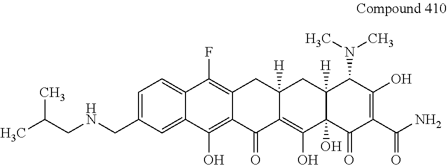

Compound 410: ¹H NMR (400 MHz, CD₃OD, hydrochloride) δ 8.60 (s, 1H), 8.08 (d, J=8.7 Hz, 1H), 7.88 (dd, J=1.8, 8.7 Hz, 1H), 4.43 (s, 2H), 4.11 (s, 1H), 3.38 (dd, J=4.1, 15.6 Hz, 1H), 3.05-2.95 (m, 10H), 2.40 (t, J=14.6 Hz, 1H), 2.27 (ddd, J=2.8, 4.6, 13.7 Hz, 1H), 2.12-2.00 (m, 1H), 1.72-1.63 (m, 1H), 1.05 (d, J=6.4 Hz, 6H); MS (ESI) m/z 568.34 (M+H).

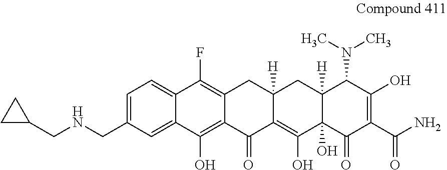

Compound 411: ¹H NMR (400 MHz, CD₃OD, hydrochloride) δ 8.59 (s, 1H), 8.08 (d, J=8.7 Hz, 1H), 7.86 (dd, J=1.8, 8.7 Hz, 1H), 4.44 (s, 2H), 4.11 (s, 1H), 3.39 (dd, J=4.1, 14.6 Hz, 1H), 3.13-2.97 (m, 10H), 2.40 (t, J=14.6 Hz, 1H), 2.27 (ddd, J=2.8, 4.6, 13.7 Hz, 1H), 2.12-2.00 (m, 1H), 1.72-1.63 (m, 1H), 1.19-1.12 (m, 1H), 0.76-0.71 (m, 2H), 0.46-0.41 (m, 2H); MS (ESI) m/z 566.38 (M+H).

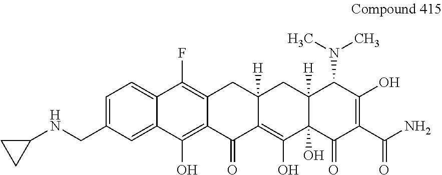

Compound 415: ¹H NMR (400 MHz, CD₃OD, hydrochloride) δ 8.59 (s, 1H), 8.08 (d, J=8.7 Hz, 1H), 7.86 (d, J=8.7 Hz, 1H), 4.53 (s, 2H), 4.10 (s, 1H), 3.38 (dd, J=4.6, 15.6 Hz, 1H), 3.12-2.97 (m, 8H), 2.88-2.83 (m, 1H), 2.40 (t, 14.2 Hz, 1H), 2.28-2.24 (m, 1H), 1.72-1.62 (m, 1H), 0.97-0.91 (m, 4H); MS (ESI) m/z 552.35 (M+H).

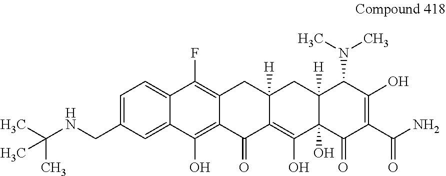

Compound 418: ¹H NMR (400 MHz, CD₃OD, hydrochloride) δ 8.60 (s, 1H), 8.08 (d, J=8.7 Hz, 1H), 7.88 (dd, J=1.8, 8.7 Hz, 1H), 4.42 (s, 2H), 4.12 (s, 1H), 3.38 (dd, J=4.1, 14.6 Hz, 1H), 3.12-2.98 (m, 8H), 2.88-2.83 (m, 1H), 2.39 (t, J=14.2 Hz, 1H), 2.28 (ddd, J=2.8, 5.0, 13.7 Hz, 1H), 1.72-1.63 (m, 1H), 1.51 (s, 9H); MS (ESI) m/z 568.41 (M+H).

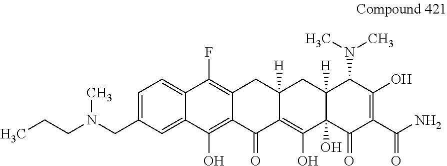

Compound 421: ¹H NMR (400 MHz, CD₃OD) δ 8.56 (s, 1H), 8.05 (d, J=8.8 Hz, 1H), 7.87 (d, J=8.4 Hz, 1H), 4.69-4.63 (m, 1H), 4.48-4.42 (m, 1H), 4.11 (s, 1H), 3.35-3.32 (m, 1H), 3.25-3.19 (m, 1H), 3.10-2.97 (m, 9H), 2.82 (d, J=3.6 Hz, 3H), 2.38-2.25 (m, 2H), 1.92-1.81 (m, 2H), 1.70-1.60 (m, 1H), 1.00 (t, J=7.2 Hz, 3H); MS (ESI) m/z 568.3 (M+H).

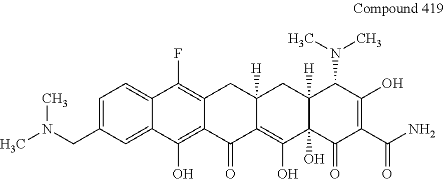

Compound 419: ¹H NMR (400 MHz, CD₃OD) δ 8.53 (s, 1H), 8.03 (d, J=8.4 Hz, 1H), 7.83 (d, J=8.4 Hz, 1H), 4.55-4.48 (m, 2H), 4.09 (s, 1H), 3.34-3.25 (m, 1H), 3.07-2.94 (m, 8H), 2.88 (s, 3H), 2.87 (s, 3H), 2.36-2.23 (m, 2H), 1.67-1.55 (m, 1H); MS (ESI) m/z 540.3 (M+H).

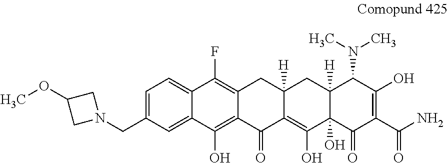

Compound 425: ¹H NMR (400 MHz, CD₃OD) δ 8.56 (s, 1H), 8.08 (d, J=7.2 Hz, 1H), 7.85 (d, J=8.4 Hz, 1H), 4.67 (d, J=11.2 Hz, 2H), 4.47-4.35 (m, 3H), 4.15-4.10 (m, 3H), 3.39 (d, J=4.0 Hz, 3H), 3.09-3.01 (m, 9H), 2.40-2.29 (m, 2H), 1.72-1.69 (m, 1H); MS (ESI) m/z 582.3 (M+H).

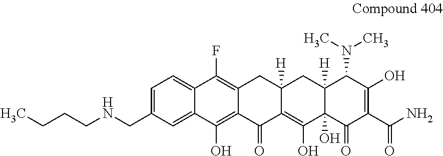

Compound 404: ¹H NMR (400 MHz, CD₃OD) δ 8.49 (d, J=1.6 Hz, 1H), 7.99 (d, J=8.4 Hz, 1H), 7.76 (dd, J=1.6, 8.4 Hz, 1H), 4.32 (s, 2H), 4.01 (s, 1H), 3.32-3.23 (m, 2H), 3.03-2.87 (m, 9H), 2.35-2.27 (m, 1H), 2.19-2.15 (m, 1H), 1.65-1.57 (m, 3H), 1.38-1.33 (m, 2H), 0.90 (t, J=7.2 Hz, 3H); MS (ESI) m/z 554.2 (M+H).

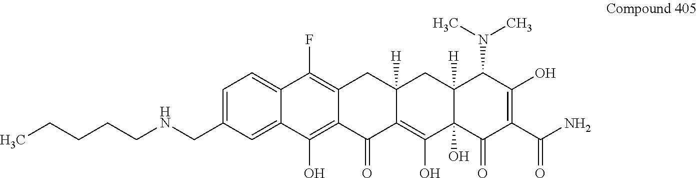

Compound 405: ¹H NMR (400 MHz, CD₃OD) δ 8.59 (s, 1H), 8.09 (d, J=8.8 Hz, 1H), 7.85 (d, J=8.4 Hz, 1H), 4.42 (s, 2H), 4.10 (s, 1H), 3.40-3.38 (m, 1H), 3.12-2.97 (m, 10H), 2.45-2.37 (m, 1H), 2.29-2.21 (m, 1H), 1.74-1.67 (m, 3H), 1.41-1.35 (m, 4H), 0.96 (t, J=6.8 Hz, 3H); MS (ESI) m/z 582.2 (M+H).

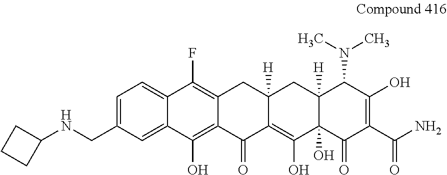

Compound 416: ¹H NMR (400 MHz, CD₃OD) δ 8.56 (s, 1H), 8.08 (d, J=8.8 Hz, 1H), 7.84 (d, J=8.4 Hz, 1H), 4.31 (s, 2H), 4.11 (s, 1H), 3.89-3.85 (m, 1H), 3.40-3.34 (m, 1H), 3.13-2.97 (m, 8H), 2.43-2.23 (m, 6H), 1.98-1.92 (m, 2H), 1.72-1.61 (m, 1H); MS (ESI) m/z 566.3 (M+H).

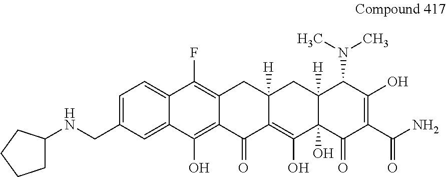

Compound 417: ¹H NMR (400 MHz, CD₃OD) δ 8.50 (s, 1H), 7.98 (d, J=8.4 Hz, 1H), 7.78 (d, J=8.4, 1H), 4.33 (s, 2H), 4.02 (s, 1H), 3.59-3.54 (m, 1H), 3.30-3.24 (m, 1H), 2.96-2.88 (m, 8H), 2.37-2.29 (m, 1H), 2.20-2.08 (m, 3H), 1.78-1.70 (m, 2H), 1.70-1.55 (m, 5H); MS (ESI) m/z 579.3 (M+H).

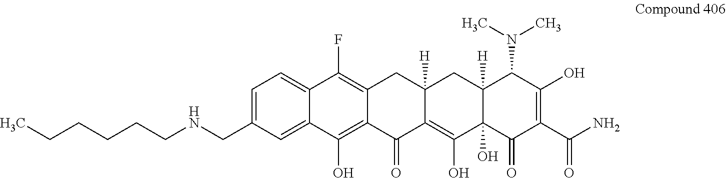

Compound 406: ¹H NMR (400 MHz, CD₃OD) δ 8.50 (s, 1H), 7.88 (d, J=8.4 Hz, 1H), 7.75 (d, J=8.8 Hz, 1H), 4.32 (s, 2H), 4.01 (s, 1H), 3.32-3.27 (m, 1H), 3.03-2.87 (m, 10H), 2.31 (m, 1H), 2.22-2.15 (m, 1H), 1.66-1.57 (m, 3H), 1.35-1.25 (m, 6H), 0.83 (t, J=7.2 Hz, 3H); MS (ESI) m/z 596.3 (M+H).

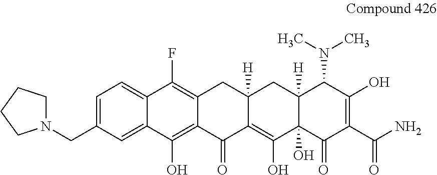

Compound 426: ¹H NMR (400 MHz, CD₃OD) δ 8.61 (s, 1H), 8.09 (d, J=8.4 Hz, 1H), 7.92 (d, J=8.8 Hz, 1H), 4.63 (s, 2H), 4.14 (s, 1H), 3.55-3.41 (m, 2H), 3.36-3.33 (m, 1H), 3.28-3.00 (m, 10H), 2.44-2.21 (m, 4H), 2.19-2.05 (m, 2H), 1.74-1.61 (m, 1H); MS (ESI) m/z 566.1 (M+H).

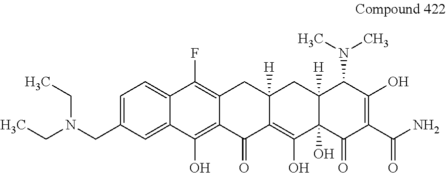

Compound 422: ¹H NMR (400 MHz, CD₃OD) δ 8.62 (s, 1H), 8.13 (d, J=8.4 Hz, 1H), 7.91 (d, J=8.8 Hz, 1H), 4.60 (s, 2H), 4.13 (s, 1H), 3.43-3.36 (m, 1H), 3.30-3.26 (m, 4H), 3.15-2.97 (m, 8H), 2.46-2.43 (m, 1H), 2.39-2.28 (m, 1H), 1.75-1.65 (m, 1H), 1.40 (t, J=7.2 Hz, 6H); MS (ESI) m/z 568.1 (M+H).

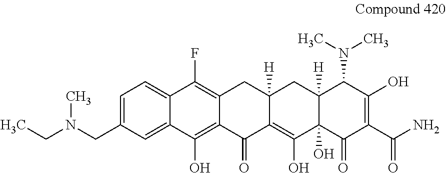

Compound 420: ¹H NMR (400 MHz, CD₃OD) δ 8.52 (s, 1H), 8.00 (d, J=8.4 Hz, 1H), 7.97 (d, J=8.8 Hz, 1H), 4.59-4.55 (m, 1H), 4.38-4.34 (m, 1H), 4.03 (s, 1H), 3.31-3.25 (m, 2H), 2.96-2.89 (m, 9H), 2.59 (s, 3H), 2.34-2.27 (m, 1H), 2.21-2.18 (m, 1H), 1.63-1.53 (m, 1H), 1.32 (t, J=7.2 Hz, 3H); MS (ESI) m/z 554.1 (M+H).

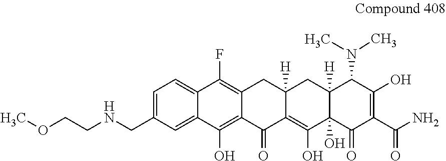

Compound 408: ¹H NMR (400 MHz, CD₃OD) δ 8.50 (s, 1H), 7.95 (d, J=8.8 Hz, 1H), 7.75 (d, J=8.4 Hz, 1H), 4.39 (s, 2H), 4.09 (s, 1H), 3.61-3.58 (m, 2H), 3.32 (s, 3H), 3.39-3.25 (m, 1H), 2.96-2.89 (m, 10H), 2.33-2.17 (m, 2H), 1.62-1.55 (m, 1H); MS (ESI) m/z 570.2 (M+H).

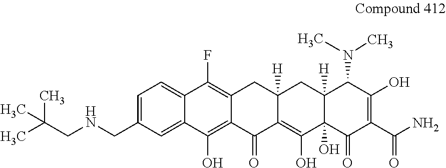

Compound 412: ¹H NMR (400 MHz, CD₃OD) δ 8.62 (s, 1H), 8.08 (d, 8.8 Hz, 1H), 7.90 (d, J=8.4 Hz, 1H), 4.47 (s, 2H), 4.11 (s, 1H), 3.40-3.35 (m, 1H), 3.08-3.01 (m, 8H), 2.98 (s, 2H), 2.43-2.35 (m, 1H), 2.31-2.25 (m, 1H), 1.73-1.63 (m, 1H), 1.04 (s, 9H); MS (ESI) m/z 582.2 (M+H).

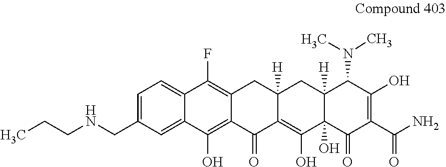

Compound 403: ¹H NMR (400 MHz, CD₃OD) δ 8.56 (s, 1H), 8.06 (d, J=8.4 Hz, 1H), 7.87 (d, J=8.4 Hz, 1H), 4.42 (s, 2H), 4.12 (s, 1H), 3.38-3.31 (m, 1H), 3.10-2.96 (m, 10H), 1.83-1.75 (m, 2H), 1.83-1.74 (m, 2H), 1.72-1.62 (m, 1H), 1.03 (t, J=7.2 Hz, 3H); MS (ESI) m/z 554.2 (M+H).

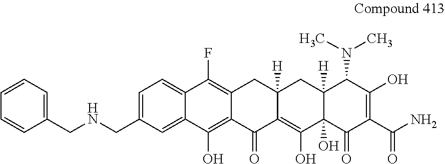

Compound 413: ¹H NMR (400 MHz, CD₃OD) δ 8.48 (s, 1H), 8.04 (d, J=8.4 Hz, 1H), 7.97 (d, J=8.4 Hz, 1H), 7.44-7.37 (m, 5H), 4.38 (s, 2H), 4.23 (s, 2H), 4.02 (s, 1H), 3.38-3.26 (m, 1H), 2.96-2.84 (m, 8H), 2.33-2.26 (m, 1H), 2.17 (m, 1H), 1.63-1.53 (m, 1H); MS (ESI) m/z 602.2 (M+H).

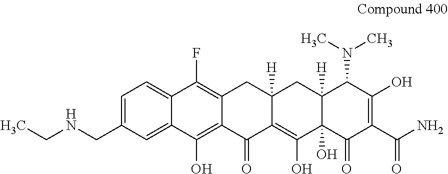

Compound 400: ¹H NMR (400 MHz, CD₃OD) δ 8.45 (s, 1H), 7.94 (d, J=8.4 Hz, 1H), 7.77 (d, J=8.4 Hz, 1H), 4.32 (s, 2H), 4.04 (s, 1H), 3.28-3.23 (m, 1H), 3.12-3.07 (m, 2H), 2.97-2.89 (m, 8H), 2.32-2.18 (m, 2H), 1.61-1.52 (m, 1H), 1.29 (t, J=7.2 Hz, 3H); MS (ESI) m/z 540.2 (M+H).

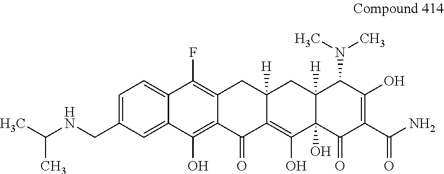

Compound 414: ¹H NMR (400 MHz, CD₃OD) δ 8.50 (s, 1H), 7.98 (d, J=8.4 Hz, 1H), 7.77 (d, J=8.4 Hz, 1H), 4.33 (s, 2H), 4.02 (s, 1H), 3.46-3.41 (m, 1H), 3.30-3.24 (m, 1H), 2.96-2.88 (m, 8H), 2.34-2.16 (m, 2H), 1.63-1.56 (m, 1H), 1.37 (d, J=7.2 Hz, 6H); MS (ESI) m/z 554.2 (M+H).

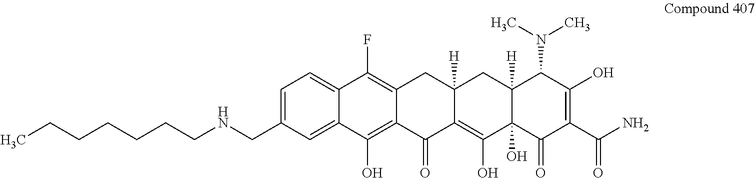

Compound 407: ¹H NMR (400 MHz, CD₃OD) δ 8.48 (s, 1H), 7.98 (d, J=8.4 Hz, 1H), 7.77 (d, J=8.8 Hz, 1H), 4.32 (s, 2H), 4.01 (s, 1H), 3.30-3.27 (m, 1H), 3.03-2.88 (m, 10H), 2.29 (m, 1H), 2.19-2.17 (m, 1H), 1.69-1.53 (m, 3H), 1.29-1.23 (m, 8H), 0.85 (t, J=7.2 Hz, 3H); MS (ESI) m/z 610.3 (M+H).

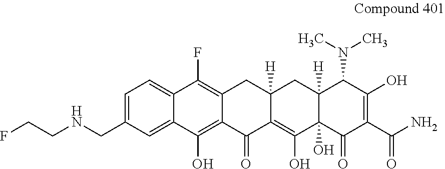

Compound 401: ¹H NMR (400 MHz, CD₃OD) δ 8.52 (s, 1H), 8.00 (d, J=8.8 Hz, 1H), 7.78 (d, J=8.4 Hz, 1H), 4.82 (t, J=4.4 Hz, 1H), 4.64 (t, J=4.4 Hz, 1H), 4.41 (s, 2H), 4.01 (s, 1H), 3.58-3.53 (m, 1H), 3.44-3.31 (m, 1H), 3.28-3.24 (m, 1H), 3.00-2.88 (m, 8H), 2.35-2.28 (m, 1H), 2.19-2.14 (m, 1H), 1.60-1.57 (m, 1H); MS (ESI) m/z 558.2 (M+H).

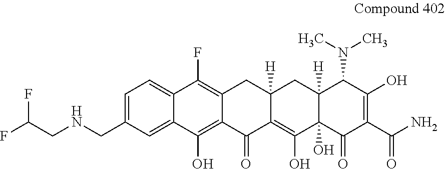

Compound 402: ¹H NMR (400 MHz, CD₃OD) δ 8.50 (s, 1H), 7.97 (d, J=8.8 Hz, 1H), 7.79 (d, J=8.4 Hz, 1H), 6.26 (tt, J=8.4, 54.0 Hz, 1H), 4.42 (s, 2H), 4.03 (s, 1H), 3.65-3.51 (m, 2H), 3.29-3.26 (m, 1H), 3.02-2.82 (m, 1.6, 8 H), 2.31-2.17 (m, 2H), 1.62-1.52 (m, 1H); MS (ESI) m/z 576.2 (M+H).

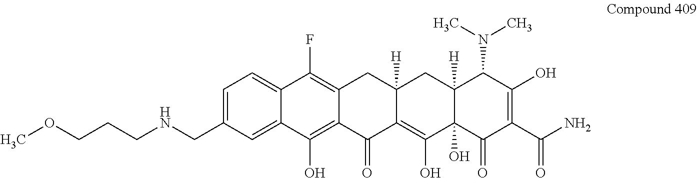

Compound 409: ¹H NMR (400 MHz, CD₃OD) δ 8.57 (s, 1H), 7.95 (d, J=8.8 Hz, 1H), 7.75 (d, J=8.4 Hz, 1H), 4.45 (s, 2H), 4.15 (s, 1H), 3.58-3.53 (m, 2H), 3.38-3.35 (m, 4H), 3.28-3.23 (m, 2H), 3.09-3.00 (m, 8H), 2.43-2.26 (m, 2H), 2.08-2.01 (m, 2H), 1.73-1.63 (m, 1H); MS (ESI) m/z 584.2 (M+H).

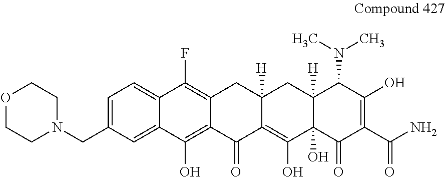

Compound 427: ¹H NMR (400 MHz, CD₃OD) δ 8.61 (s, 1H), 8.11 (d, J=8.8 Hz, 1H), 7.88 (d, J=8.4 Hz, 1H), 4.51 (s, 2H), 4.05 (s, 1H), 3.95-3.83 (m, 4H), 3.38-3.33 (m, 4H), 3.15-3.00 (m, 9H), 2.42-2.38 (m, 1H), 2.29-2.23 (m, 1H), 1.72-1.61 (m, 1H); MS (ESI) m/z 582.2 (M+H).

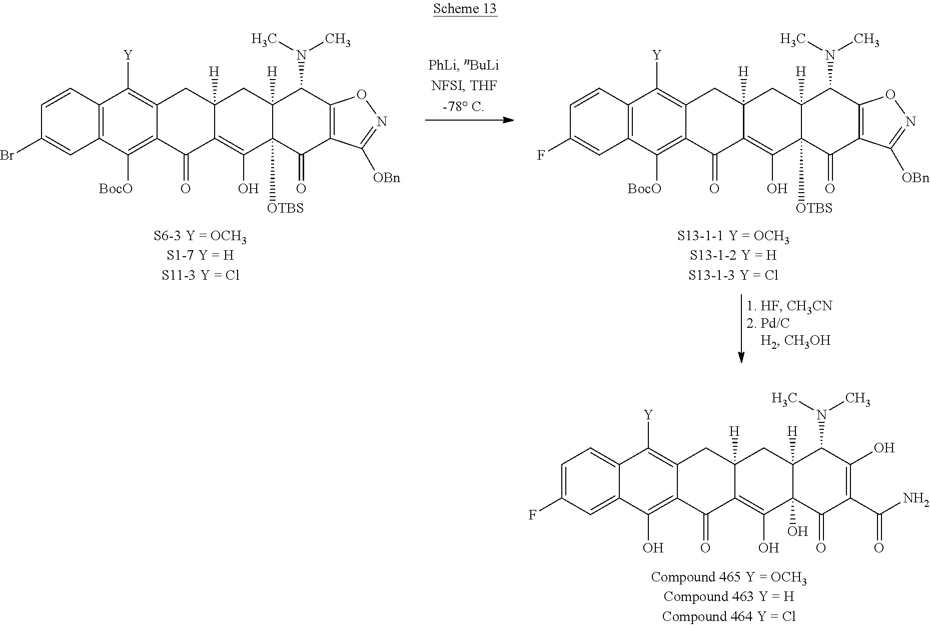

The following compounds were prepared according to Scheme 13.

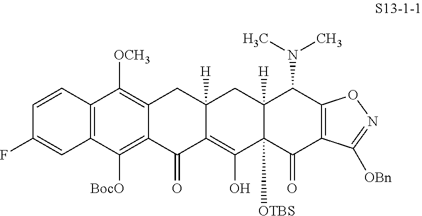

A solution of phenyllithium in di-n-butyl ether (1.8 M, 66 μL, 0.12 mmol, 2.0 eq) was added drop wise to a solution of bromide S6-3 (52 mg, 0.06 mmol, 1.0 eq) in tetrahydrofuran (2 mL) at −78° C., forming a dark red solution. After 5 min, a solution of n-butyllithium in hexanes (1.6 M, 45 μL, 0.072 mmol, 1.2 eq) was added drop wise at −78° C. followed 3 min later by the addition of N-fluorobenzenesulfonimide (66 mg, 0.21 mmol, 3.5 eq). The resulting red reaction mixture was stirred at −78° C. for 1 h. Saturated aqueous ammonium chloride solution (2 mL) was added drop wise at −78° C. The reaction mixture was allowed to warm up to 23° C., diluted with saturated aqueous ammonium chloride solution (˜20 mL), and extracted with methylene chloride (3×15 mL). The organic extracts were combined, and the combined solution was dried over anhydrous sodium sulfate, filtered, and concentrated. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; gradient: 80→100% B over 10 min, then 100% B for 5 min; mass-directed fraction collection]. Fractions with the desired MW were collected and concentrated on a RotaVap at rt to yield product S13-1-1 as a yellow solid (16.6 mg, 34%). ¹H NMR (400 MHz, CDCl₃) δ 15.95 (br s, 1H), 8.08 (dd, J=5.5, 9.2 Hz, 1H), 7.69 (br d, J=9.2 Hz, 1H), 7.50-7.48 (m, 2H), 7.41-7.32 (m, 4H), 5.37, 5.33 (ABq, J=12.2 Hz, 2H), 3.98 (d, J=10.4 Hz, 1H), 3.87 (s, 3H), 3.48 (dd, 4.9, 15.3 Hz, 1H), 3.04-2.98 (m, 1H), 2.63-2.47 (m, 9H), 2.19 (d, J=14.0 Hz, 1H), 1.59 (s, 9H), 0.81 (s, 9H), 0.26 (s, 3H), 0.12 (s, 3H); MS (ESI) m/z 815.50 (M+H).

The following compounds were prepared similarly to S13-1-1.

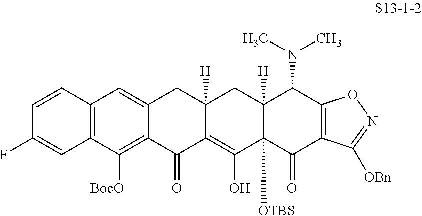

S13-1-2: ¹H NMR (400 MHz, CDCl₃) δ 16.01 (br s, 1H), 7.76 (dd, J=5.5, 9.2 Hz, 1H), 7.68 (br d, J=9.2 Hz, 1H), 7.53 (s, 1H), 7.50-7.46 (m, 2H), 7.39-7.29 (m, 4H), 5.37, 5.33 (ABq, J=12.2 Hz, 2H), 3.97 (d, J=11.0 Hz, 1H), 3.10-3.05 (m, 2H), 2.98-2.94 (m, 1H), 2.58-2.46 (m, 8H), 2.13 (d, J=14.0 Hz, 1H), 1.58 (s, 9H), 0.81 (s, 9H), 0.26 (s, 3H), 0.12 (s, 3H); ¹⁹F NMR (400 MHz, CDCl₃) δ −111.7; MS (ESI) m/z 785.47 (M+H).

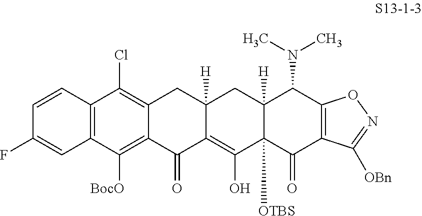

S13-1-3: ¹H NMR (400 MHz, CDCl₃) δ 15.82 (br s, 1H), 8.30 (dd, J=5.5, 9.2 Hz, 1H), 7.75-7.69 (m, 1H), 7.50-7.44 (m, 3H), 7.39-7.32 (m, 3H), 5.37, 5.33 (ABq, J=12.2 Hz, 2H), 3.94 (d, J=11.0 Hz, 1H), 3.66 (dd, J=4.3, 15.9 Hz, 1H), 3.10-3.05 (m, 1H), 2.74-2.68 (m, 1H), 2.61-2.45 (m, 8H), 2.20 (d, J=14.0 Hz, 1H), 1.57 (s, 9H), 0.82 (s, 9H), 0.26 (s, 3H), 0.12 (s, 3H); MS (ESI) m/z 819.45 (M+H).

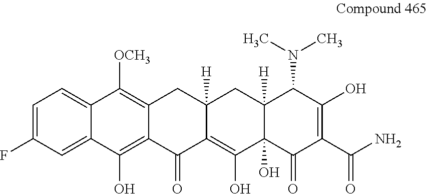

Concentrated aqueous hydrofluoric acid (48 wt %, 0.2 mL) was added to a solution of compound S13-1-1 (16.6 mg, 0.020 mmol, 1.0 eq) in acetonitrile (0.6 mL) in a polypropylene reaction vessel at 23° C. The resulting mixture was stirred vigorously at 23° C. overnight and poured into aqueous dipotassium hydrogenphosphate (2.5 g dissolved in 20 mL water). The mixture was extracted with methylene chloride (3×15 mL). The combined organic extracts were dried over anhydrous sodium sulfate, filtered, and concentrated. The residue was used directly in the final step without further purification.

Pd—C (10 wt %, 8 mg) was added in one portion into the yellow solution of the above crude product in a mixture of MeOH (1 mL) and dioxane (1 mL) at 23° C. The reaction vessel was sealed and purged with hydrogen by briefly evacuating the flask followed by flushing with hydrogen gas (1 atm). The resulting mixture was stirred at 23° C. for 40 min. LCMS analysis indicated the reaction complete. The reaction mixture was then filtered through a small Celite pad. The filtrate was concentrated. The residue was purified by preparative reverse phase HPLC on a Waters Autopurification system using a Phenomenex Polymerx 10μ RP-1 100A column [10 μm, 150×21.20 mm; flow rate, 20 mL/min; Solvent A: 0.05 N HCl/water; Solvent B: CH₃CN; injection volume: 3.0 mL (0.05 N HCl/water); gradient: 20→80% B over 10 min; mass-directed fraction collection]. Fractions with the desired MW were collected and freeze-dried to yield Compound 465 (7.8 mg, 70% for 2 steps): ¹H NMR (400 MHz, CD₃OD, hydrochloride) δ 8.04 (dd, J=5.0, 9.2 Hz, 1H), 7.92 (dd, J=2.8, 7.3 Hz, 1H), 7.49 (dt, J=2.3, 8.7 Hz, 1H), 4.11 (s, 1H), 3.78 (s, 3H), 3.35 (dd, J=4.1, 15.1 Hz, 1H), 3.06-2.95 (m, 8H), 2.32 (t, J=14.2 Hz, 1H), 2.28-2.24 (m, 1H), 1.65 (q, J=11.4 Hz, 1H); MS (ESI) m/z 513.28 (M+H).

The following compounds were prepared similarly to Compound 465.

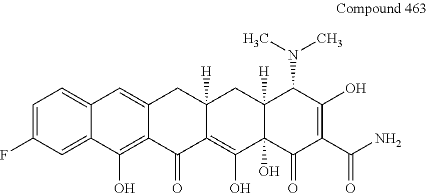

Compound 463: ¹H NMR (400 MHz, CD₃OD, hydrochloride) δ 7.92 (dd, J=2.3, 9.6 Hz, 1H), 7.79 (dd, J=5.5, 9.2 Hz, 1H), 7.44 (dt, J=2.8, 8.7 Hz, 1H), 7.17 (s, 1H), 4.09 (s, 1H), 3.09-2.96 (m, 9H), 2.65 (t, J=13.7 Hz, 1H), 2.21 (ddd, J=2.8, 4.6, 13.7 Hz, 1H), 1.68-1.58 (m, 1H); MS (ESI) m/z 483.26 (M+H).

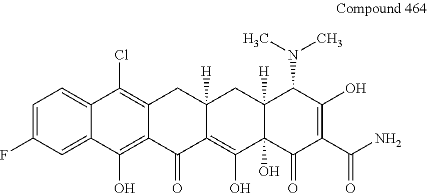

Compound 464: ¹H NMR (400 MHz, CD₃OD, hydrochloride) δ 8.28 (dd, J=5.0, 9.2 Hz, 1H), 8.05 (dd, J=1.8, 9.6 Hz, 1H), 7.62 (dt, J=2.8, 9.2 Hz, 1H), 4.11 (s, 1H), 3.58 (dd, J=4.6, 15.6 Hz, 1H), 3.05-2.97 (m, 8H), 2.78 (t, J=14.6 Hz, 1H), 2.27-2.24 (m, 1H), 1.73-1.63 (m, 1H); MS (ESI) m/z 517.23 (M+H).

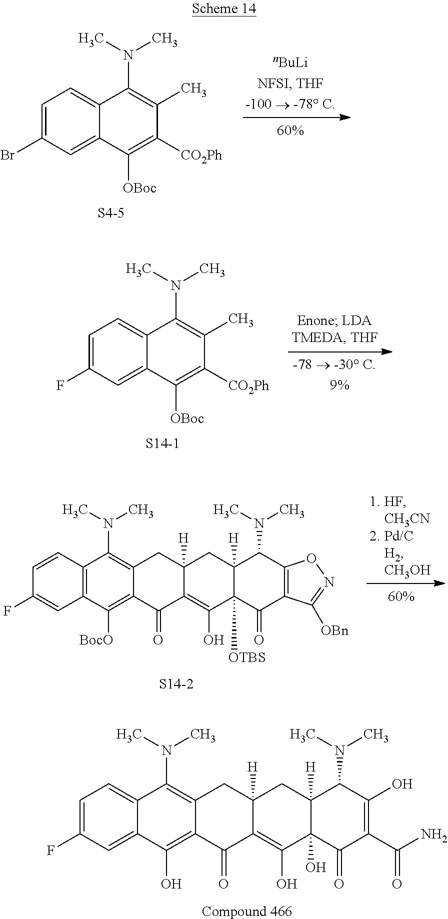

The following compounds were prepared according to Scheme 14.

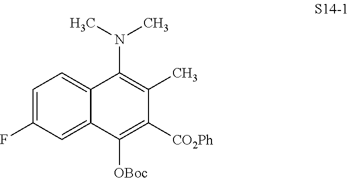

A solution of n-butyllithium in hexanes (1.6 M, 345 μL, 0.552 mmol, 1.2 eq) was added drop wise to a solution of bromide S4-5 (230 mg, 0.46 mmol, 1.0 eq) in tetrahydrofuran (10 mL) at −100° C., forming a red solution. After 5 min, a solution of N-fluorobenzenesulfonimide (290 mg, 0.92 mmol, 2.0 eq) in tetrahydrofuran (1 mL) was added drop wise via cannula. The resulting reaction mixture was allowed to warm up to −78° C. and stirred at that temperature for 1 h. Saturated aqueous ammonium chloride solution (5 mL) was added drop wise at −78° C. The reaction mixture was allowed to warm up to 23° C., diluted with saturated aqueous ammonium chloride solution (˜30 mL), and extracted with ethyl acetate (60 mL). The organic phase was separated, washed with brine (20 mL), dried over anhydrous sodium sulfate, filtered, and concentrated. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; gradient: 80→100% B over 10 min, then 100% B for 5 min; mass-directed fraction collection]. Fractions with the desired MW were collected and concentrated on a RotaVap at rt to yield product S14-1 (94.9 mg, 60% based on recovered SM S4-5 (50 mg)). ¹H NMR (400 MHz, CDCl₃) δ 8.19 (dd, J=5.5, 9.2 Hz, 1H), 7.48-7.42 (m, 3H), 7.34-7.27 (m, 4H), 2.98 (s, 6H), 2.50 (s, 3H), 1.46 (s, 9H); MS (ESI) m/z 440.34 (M+H).

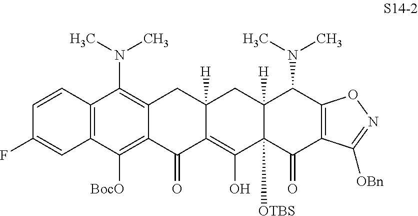

A suspension of LDA in hexanes (10 wt %, 123 μL, 0.082 mmol, 2.5 eq) was added slowly to a solution of phenyl ester S14-1 (14.6 mg, 0.033 mmol, 1.0 eq), enone (16 mg, 0.033 mmol, 1.0 eq) and TMEDA (25 μL, 0.165 mmol, 5.0 eq) in tetrahydrofuran (1.5 mL) at −78° C. The resulting brownish mixture was allowed to warm slowly to −10° C. over 1 h. The reaction mixture was then diluted with saturated aqueous ammonium chloride solution (10 mL), and extracted with methylene chloride (3×15 mL). The organic extracts were combined, dried over anhydrous sodium sulfate, filtered, and concentrated. The residue was purified by preparative reverse phase HPLC on a Waters Autopurification system using a Sunfire Prep C18 OBD column [5 μm, 19×50 mm; flow rate, 20 mL/min; Solvent A: H₂O with 0.1% HCO₂H; Solvent B: CH₃CN with 0.1% HCO₂H; gradient: 80→100% B over 10 min, then 100% B for 5 min; mass-directed fraction collection]. Fractions with the desired MW were collected and concentrated on a RotaVap at rt to yield product S14-2 (2.4 mg, 9%). ¹H NMR (400 MHz, CDCl₃) δ 15.88 (br s, 1H), 8.13 (dd, J=5.5, 9.2 Hz, 1H), 7.68 (br d, J=7.9 Hz, 1H), 7.50-7.48 (m, 2H), 7.39-7.30 (m, 4H), 5.37, 5.33 (ABq, J=12.2 Hz, 2H), 4.02 (d, J=10.4 Hz, 1H), 3.27 (dd, J=4.3, 15.3 Hz, 1H), 3.03-2.95 (m, 7H), 2.65 (t, J=15.3 Hz, 1H), 2.58-2.49 (m, 8H), 2.18 (d, J=14.0 Hz, 1H), 1.57 (s, 9H), 0.83 (s, 9H), 0.27 (s, 3H), 0.13 (s, 3H); MS (ESI) m/z 828.55 (M+H).

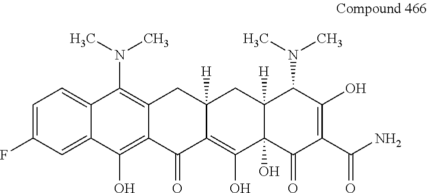

Concentrated aqueous hydrofluoric acid (48 wt %, 0.2 mL) was added to a solution of compound S14-2 (2.4 mg, 0.003 mmol, 1.0 eq) in acetonitrile (0.6 mL) in a polypropylene reaction vessel at 23° C. The resulting mixture was stirred vigorously at 23° C. overnight and poured into aqueous dipotassium hydrogenphosphate (2.5 g dissolved in 20 mL water). The mixture was extracted with methylene chloride (3×15 mL). The combined organic extracts were dried over anhydrous sodium sulfate, filtered, and concentrated. The residue was used directly in the final step without further purification.

Pd—C (10 wt %, 1.5 mg) was added in one portion into the yellow solution of the above crude product in a mixture of MeOH (0.5 mL) and dioxane (0.5 mL) at 23° C. The reaction vessel was sealed and purged with hydrogen by briefly evacuating the flask followed by flushing with hydrogen gas (1 atm). The resulting mixture was stirred at 23° C. for 2 h. LCMS analysis indicated the reaction complete. The reaction mixture was then filtered through a small Celite pad. The filtrate was concentrated. The residue was purified by preparative reverse phase HPLC on a Waters Autopurification system using a Phenomenex Polymerx 10μ RP-1 100A column [10 μm, 150×21.20 mm; flow rate, 20 mL/min; Solvent A: 0.05 N HCl/water; Solvent B: CH₃CN; injection volume: 3.0 mL (0.05 N HCl/water); gradient: 15→100% B over 10 min; mass-directed fraction collection]. Fractions with the desired MW were collected and freeze-dried to yield compound 466 (1.05 mg, 60% for 2 steps): ¹H NMR (400 MHz, CD₃OD, hydrochloride) δ 8.25 (br s, 1H), 8.08 (br s, 1H), 7.57 (br s, 1H), 4.14 (s, 1H), 3.44-3.39 (m, 1H), 3.13-2.97 (m, 8H), 2.56-2.50 (m, 1H), 2.33-2.29 (m, 1H), 1.74-1.64 (m, 1H); MS (ESI) m/z 526.30 (M+H).

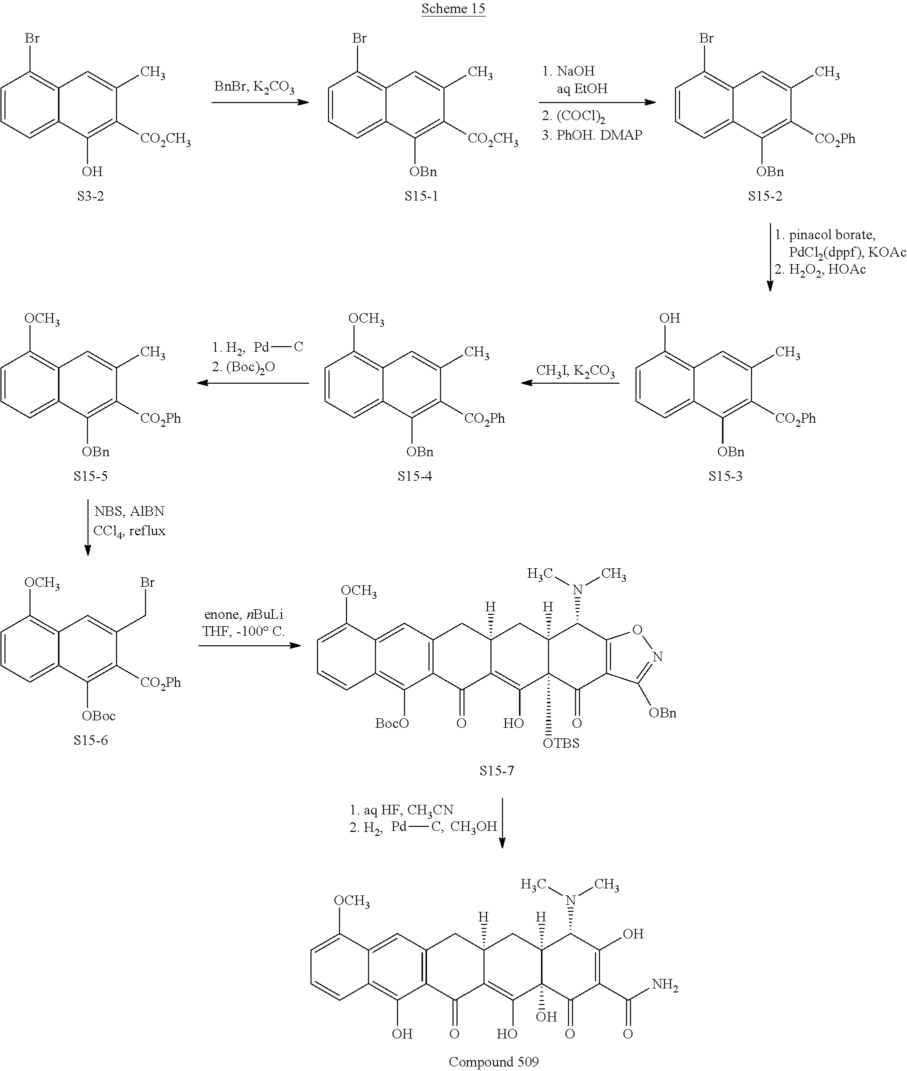

The following compounds were prepared according to Scheme 15.

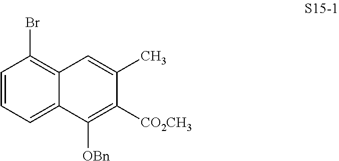

To a stirred solution of S3-2 (2.0 g, 6.8 mmol, 1.0 eq.) in acetone (25 mL) was added powdered K₂CO₃ (1.2 g, 8.7 mmol, 1.3 eq.), followed by BnBr (1.4 g, 8.2 mmol, 1.2 eq.). The resulting mixture was heated at reflux for 1.5 h, cooled to rt and filtered through a short pad of Celite. The filtrate was concentrated under reduced pressure, the residue was purified with flash chromatography (200˜300 mesh, petroleum ether/EtOAc=100:1) to give the desire product S15-1 (2.0 g, 77%) as a white solid: ¹H NMR (400 MHz, CDCl₃): δ 8.01 (d, J=8.4 Hz, 1H), 7.80 (s, 1H), 7.72 (d, J=7.6 Hz, 1H), 7.43-7.41 (m, 2H), 7.37-7.29 (m, 3H), 7.20 (t, J=8.4 Hz, 1H), 5.05 (s, 2H), 3.83 (s, 3H), 2.45 (s, 3H).

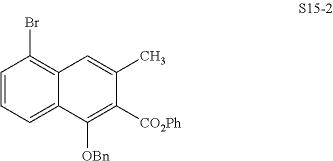

To a solution of S15-1 (2.0 g, 5.2 mmol) in absolute EtOH (10 mL) was added aqueous NaOH solution (4 N, 10 mL), the resulting mixture was heated at reflux overnight. The reaction was diluted with water (30 mL) and extracted with EtOAc (20 mL×3), the aqueous phase was acidified with dilute HCl (1 N, about 45 mL) to adjust pH˜6, and the mixture was extracted with EtOAc (40 mL×3), the combined organic layers was dried over anhydrous Na₂SO₄, filtered, and then concentrated under reduced pressure to give the desired product as a white solid, which was not purified and used for the next step directly.

To a solution of above crude product in methylene chloride (20 mL) was added oxalyl chloride (0.57 mL, 6.5 mmol, 1.3 eq.), followed by a couple of drops of DMF (caution: gas evolution). The mixture was stirred at rt for 30 min and the volatiles were evaporated under reduce pressure. The residue was further dried under high vacuum to afford the crude benzoyl chloride. The crude benzoyl chloride was re-dissolved in methylene chloride (20 mL). Pyridine (0.88 mL, 10.9 mmol, 2.1 eq.), phenol (0.54 g, 5.7 mmol, 1.1 eq.) and a catalytic amount of DMAP were added. The mixture was stirred at rt for 1 h. The solvent was evaporated. The residue was suspended in EtOAc, and the precipitate was filtered off. The organic solution was then washed with 1 N HCl (three times), H₂O, saturated aqueous NaHCO₃, and brine, dried over Na₂SO₄, filtered and then concentrated. Purification of the residue by flash chromatography (200˜300 mesh, petroleum ether/EtOAc=100:1) gave compound S15-2 (1.7 g, 73%) as an off-white solid: ¹H NMR (400 MHz, CDCl₃): δ 8.11 (d, J=8.4 Hz, 1H), 7.94 (s, 1H), 7.83 (dd, J=8.4 Hz and 1.2 Hz, 1H), 7.53-7.51 (m, 2H), 7.42-7.35 (m, 5H), 7.32-7.24 (m, 2H), 7.16 (d, J=7.6 Hz, 1H), 5.22 (s, 2H), 2.68 (s, 3H).

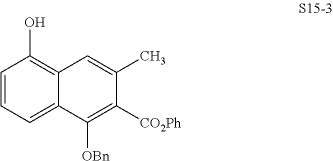

The mixture of S15-2 (2.2 g, 4.93 mmol, 1.0 eq.), bis(pinacolato)diboron (7.5 g, 29.5 mmol, 6.0 eq.), potassium acetate (3.0 g, 30.6 mmol, 6.2 eq.) and PdCl₂(dppf) (400 mg, 0.5 mmol, 0.1 eq.) were degassed for 20 min in a 250 mL flask. Then dioxane (50 ml) was added to the flask and degassed for 5 min. The reaction mixture was heated to 90° C. under N₂ atmosphere and stirred overnight at the same temperature. The reaction mixture was filtered. Then all solvent was removed under reduced pressure. The residue was purified by column on silica gel (200˜300 mesh, petroleum ether/EtOAc=100:1) to afford the borate (2.1 g, 86%) as a white solid: ¹H NMR (400 MHz, CDCl₃): δ 8.44 (s, 1H), 8.25 (d, J=8.4 Hz, 1H), 8.11 (d, J=2.4 Hz, 1H), 7.54-7.52 (m, 2H), 7.46 (dd, J=8.4 Hz and 6.8 Hz, 1H), 7.41-7.37 (m, 5H), 7.27-7.23 (m, 1H), 7.17-7.15 (m, 2H), 5.21 (s, 2H), 2.66 (s, 3H), 1.24 (s, 12H).

To a solution of the above borate (2.0 g, 4.0 mmol, 1.0 eq.) in THF (20 mL) was added carefully H₂O₂ (30%, 2.0 mL, 17.6 mmol, 4.4 eq.), followed by AcOH (1.2 mL, 20.0 mmol, 5.0 eq.), and the resulting mixture was stirred at rt overnight. TLC showed all the borate was consumed, the reaction was quenched with saturated aqueous NaHSO₃ solution (caution: till the starch iodide was negative), and extracted with EtOAc (50 mL×3). The combined organic layers was dried, filtered and then concentrated. The residue was purified with flash chromatography (200˜300 mesh, petroleum ether/EtOAc=50:1˜40:1˜10:1) to give the desired product S15-3 (1.2 g, 78%) as a white solid: ¹H NMR (400 MHz, CDCl₃): δ7.81 (s, 1H), 7.66 (d, J=8.4 Hz, 1H), 7.47 (d, J=6.8 Hz, 2H), 7.36-7.28 (m, 5H), 7.24-7.17 (m, 2H), 7.09 (d, J=7.6 Hz, 2H), 6.78 (d, J=7.2 Hz, 1H), 5.51 (br s, 1H), 5.16 (s, 2H), 2.58 (s, 3H).

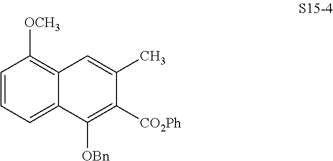

To a stirred solution of S15-3 (1.0 g, 2.6 mmol, 1.0 eq.) in acetone (15 mL) was added powdered K₂CO₃ (0.9 g, 6.5 mmol, 2.5 eq.), followed by MeI (0.74 g, 5.2 mmol, 2.0 eq.), and the resulting mixture was stirred at rt overnight. The reaction was quenched with water (50 mL), after evaporation of acetone, the residue was extracted with EtOAc (20 mL×3). The combined organic layers was dried, filtered, and then concentrated. The residue was purified with flash chromatography (200˜300 mesh, petroleum ether/EtOAc=40:1) to give the desired product S15-4 (760 mg, 74%) as a white solid: ¹H NMR (400 MHz, CDCl₃): δ7.89 (s, 1H), 7.65 (d, J=8.4 Hz, 1H), 7.48 (d, J=7.6 Hz, 2H), 7.36-7.28 (m, 6H), 7.20-7.17 (m, 1H), 7.09 (d, J=8.4 Hz, 2H), 6.82 (d, J=7.6 Hz, 1H), 5.16 (s, 2H), 3.94 (s, 3H), 2.57 (s, 3H).

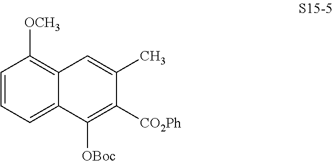

To a stirred solution of S15-4 (750 mg, 1.88 mmol, 1.0 eq.) in MeOH (10 mL) and EtOAc (10 mL) was added Pd—C (90 mg). The resulting suspension was briefly evacuated and re-filled with H₂, and the mixture was stirred under a H₂ atmosphere at rt for 2 h. After filtration of Pd—C, the filtrate was concentrated under reduced pressure. The residue was re-dissolved into methylene chloride (20 mL), and to this solution was added Boc₂O (430 mg, 1.97 mmol, 1.1 eq.) and a catalytic amount of DMAP (20 mg, 0.16 mmol, 0.09 eq.), the resulting mixture was stirred at rt for 50 min. The reaction was quenched with water (50 mL) and extracted with EtOAc (30 mL×3). The combined organic layers was dried, filtered, and then concentrated. The residue was purified with flash chromatography (200˜300 mesh, petroleum ether/EtOAc=40:1) to give the desired product S15-5 (690 mg, 90%) as a white solid: ¹H NMR (400 MHz, CDCl₃): δ8.00 (s, 1H), 7.44-7.35 (m, 4H), 7.24-7.20 (m, 3H), 6.82 (d, J=7.6 Hz, 1H), 3.94 (s, 3H), 2.60 (s, 3H), 1.40 (s, 9H).

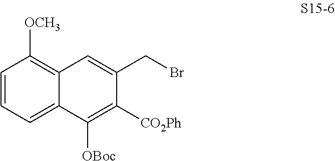

A mixture of S15-5 (204 mg, 0.50 mmol, 1.0 eq.), NBS (99 mg, 0.56 mmol, 1.1 eq.), and AIBN (20 mg, 0.12 mmol, 0.24 eq.) in CCl₄ (4 mL) was stirred at reflux for 3 h. After evaporation of the solvent, the residue was purified with flash chromatography (200˜300 mesh, petroleum ether/EtOAc=50:1) to give the desired product S15-6 (200 mg, 82%) as a white solid: ¹H NMR (400 MHz, CDCl₃): δ8.28 (s, 1H), 7.56-7.46 (m, 4H), 7.42-7.32 (m, 2H), 7.35-7.30 (m, 1H), 6.96 (d, J=6.8 Hz, 1H), 4.99 (s, 2H), 4.03 (s, 3H), 1.49 (s, 9H).

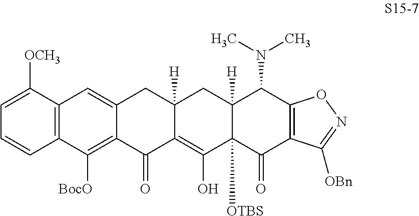

A solution of n-BuLi in hexane (2.5 M, 0.10 mL, 0.25 mmol, 4.0 eq.) was added drop wise to a solution of S15-6 (100 mg, 0.21 mmol, 3.3 eq.) and enone (30 mg, 0.062 mmol, 1.0 eq.) in dry THF (2.5 mL), which had been cooled to −100° C. using a liquid N₂/EtOH bath. After addition, the resulting mixture was allowed to warm to 0° C. gradually over 1 h. The reaction was then quenched with brine (20 mL) and extracted with EtOAc (20 mL×3). The combined organic layers was dried, filtered and then concentrated. The residue was purified with flash chromatography (200˜300 mesh, petroleum ether/EtOAc=40:1˜20:1) to give the desired product S15-7 (30 mg, 61%) as a yellow solid: ¹H NMR (400 MHz, CDCl₃): δ7.93 (s, 1H), 7.62 (d, J=8.4 Hz, 1H), 7.46-7.44 (m, 2H), 7.40-7.28 (m, 5H), 6.87 (d, J=6.8 Hz, 1H), 5.31 (s, 2H), 3.95 (s, 3H), 3.11-3.03 (m, 2H), 2.92 (d, J=10.4 Hz, 1H), 2.51-2.38 (m, 9H), 2.11-2.05 (m, 1H), 1.73-1.65 (m, 1H), 1.53 (s, 9H), 0.80 (s, 9H), 0.22 (s, 3H), 0.09 (s, 3H).

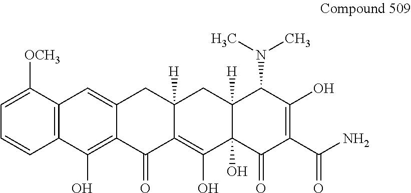

To a solution of S15-7 (30 mg, 0.0377 mmol) in CH₃CN (5.0 mL) was added an aqueous HF solution (48-50%, 2.4 mL). The resulting solution was stirred at rt for 16 hrs, the reaction mixture was poured into aqueous solution (40 mL) of K₂HPO₄ (14.4 g) and extracted three times with EtOAc (20 mL×3). The combined organic phases were washed with brine, dried, and concentrated to yield the crude intermediate.

The above crude intermediate was dissolved in THF (2.0 mL) and MeOH (2.0 mL). Pd—C (10 wt %, 5 mg) was added. The reaction flask was briefly evacuated and re-filled with H₂. The reaction mixture was stirred at rt and monitored by LCMS. After the reaction was complete, MeOH (5 mL) and 0.5 N HCl/MeOH (0.5 mL) were added. The mixture was stirred for 30 min, and filtered through a small pad of Celite. The filtrate was concentrated to give the crude product, which was purified by HPLC to give the desired product Compound 509 (8.0 mg, 16%) as a yellow solid: ¹H NMR (400 MHz, CD₃OD): δ7.87 (d, J=8.4 Hz, 1H), 7.43 (s, 1H), 7.39 (t, J=8.4 Hz, 1H), 7.07 (d, J=8.4 Hz, 1H), 4.07 (s, 1H), 3.97 (s, 2H), 3.09-2.89 (m, 9H), 2.65-2.58 (m, 1H), 2.22-2.19 (m, 1H), 1.65-1.59 (m, 1H); MS (ESI) m/z 480.2 (M+H).

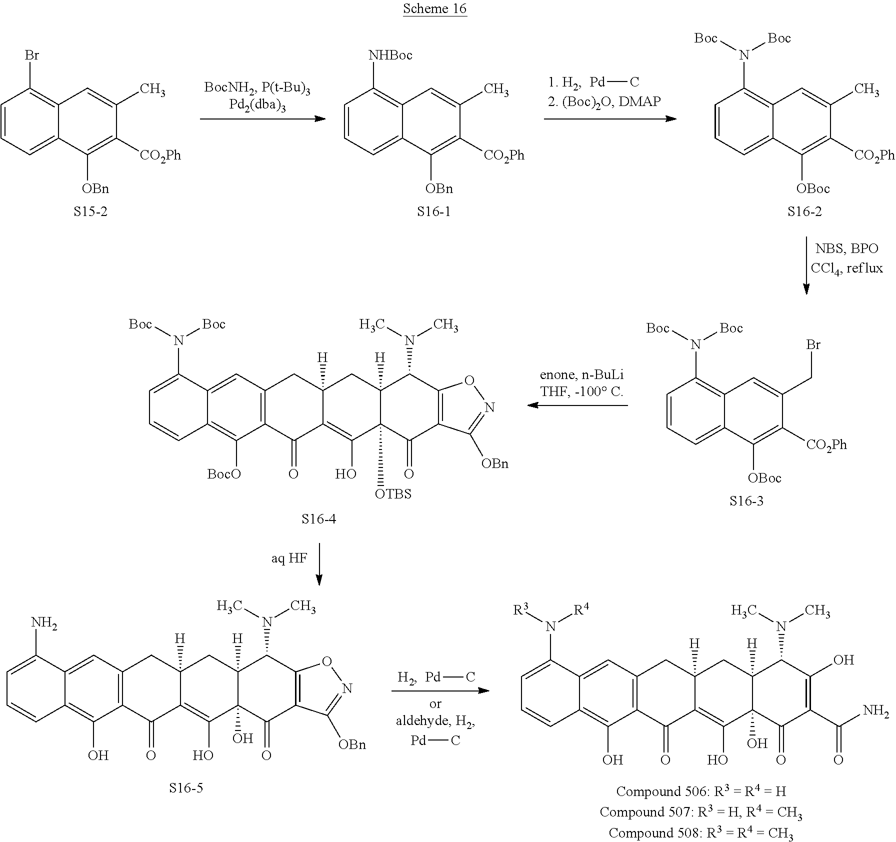

The following compounds were prepared according to Scheme 16.

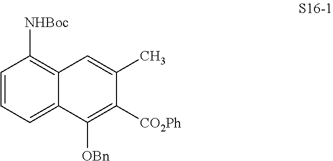

To a stirred solution of S15-2 (2.0 g, 4.48 mmol, 1.0 eq.) in anhydrous toluene (20 mL) was added BocNH₂ (786 mg, 6.7 mmol, 1.5 eq.), PhONa (779 mg, 6.7 mmol, 1.5 eq.) and Pd₂(dba)₃.CHCl₃ (125 mg, 0.11 mmol, 0.025 eq.), the mixture was degassed for 5 min and then (tBu)₃P (45.2 mg, 0.22 mmol, 0.05 eq.) was added. The resulting mixture was heated at 95° C. for 2 h. The reaction was quenched with water (50 mL) and extracted with EtOAc (50 mL×3) three times. The combined organic layers was dried, filtered, and then concentrated under reduced pressure. The residue was purified with flash chromatography (200˜300 mesh, petroleum ether/EtOAc=100:1˜50:1˜20:1) to give the desired product S16-1 (1.6 g, 74%) as a white solid: ¹H NMR (400 MHz□CDCl₃) δ 7.89 (d, J=8.4 Hz, 1H), 7.48-7.46 (m, 3H), 7.42-7.31 (m, 7H), 7.21-7.19 (m, 1H), 7.09 (d, J=8.4 Hz, 2H), 5.16 (s, 2H), 2.61 (s, 3H), 1.51 (s, 9H).

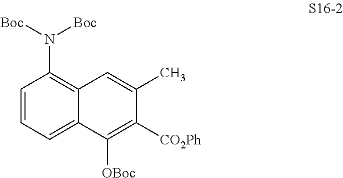

To a stirred solution of S16-1 (2.0 g, 4.13 mmol, 1.0 eq.) in MeOH (20 mL) was added Pd—C (300 mg). The resulting suspension was briefly evacuated and re-filled with H₂, and the mixture was stirred under a H₂ atmosphere at rt for 1 h. After filtration of Pd—C, the filtrate was concentrated under reduced pressure. The residue was re-dissolved into methylene chloride (20 mL), and to this solution was added Boc₂O (1.25 mg, 5.73 mmol, 1.4 eq.) and a catalytic amount of DMAP (30 mg, 0.25 mmol, 0.06 eq.), the resulting mixture was stirred at rt for 2 h. The reaction was quenched with brine (50 mL) and extracted with methylene chloride (30 mL×3). The combined organic layers was dried, filtered, and then concentrated. The residue was purified with flash chromatography (200˜300 mesh, petroleum ether/EtOAc=50:1˜15:1˜10:1) to give the desired product S16-2 (1.2 g, 49%) as a white solid: ¹H NMR (400 MHz, CDCl₃): δ 7.93 (d, J=8.4 Hz, 1H), 7.61 (s, 1H), 7.54-7.39 (m, 4H), 7.32-7.26 (m, 3H), 2.67 (s, 3H), 1.46 (s, 9H), 1.32 (s, 18H).

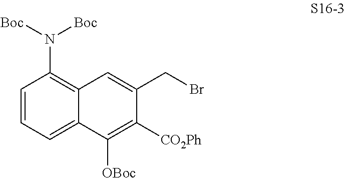

To a solution of S16-2 (1.1 g, 1.85 mmol, 1.0 eq.) in CCl₄ (20 mL) was added NBS (347 mg, 1.95 mmol, 1.05 eq.), followed by BPO (895 mg, 3.70 mmol, 2.0 eq.). The resulting mixture was refluxed for 2 h. After evaporation of the solvent under reduced pressure, the residue was purified with flash chromatography (200˜300 mesh, petroleum ether/EtOAc=50:1˜15:1) to give the desired product S16-3 (1.0 g, 81%) as a white solid: ¹H NMR (400 MHz, CDCl₃): δ7.98 (d, J=8.4 Hz, 1 H), 7.78 (s, 1H), 7.62-7.56 (m, 1H), 7.47-7.40 (m, 3H), 7.40-7.35 (m, 2H), 7.32-7.26 (m, 1H), 4.92 (s, 1H), 1.43 (s, 9H), 1.30 (s, 18H).

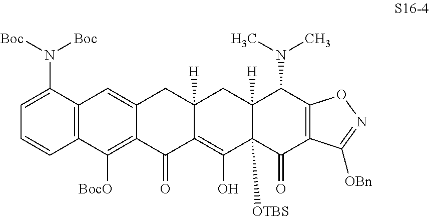

A solution of n-BuLi in hexane (2.5 M, 0.30 mL, 0.75 mmol, 3.2 eq.) was added drop wise to a solution of S16-3 (504 mg, 0.75 mmol, 3.2 eq.) and enone (113 mg, 0.23 mmol, 1.0 eq.) in dry THF (10 mL), which had been cooled to −100° C. using a liquid N₂/EtOH bath. After addition, the resulting mixture was stirred at −100° C. for 30 min and then allowed to warm to 0° C. gradually. The reaction was then quenched with brine (20 mL) and extracted with EtOAc (20 mL×3). The combined organic layers was dried, filtered and then concentrated. The residue was purified with flash chromatography (200˜300 mesh, petroleum ether/EtOAc=30:1˜20:1 10:1) to give the desired product S16-4 (123 mg, 53%) as a yellow gum: ¹H NMR (400 MHz, CDCl₃): δ8.33 (d, J=8.4 Hz, 1H), 7.77-7.71 (m, 5H), 7.66-7.48 (m, 6H), 7.35 (d, J=8.8 Hz, 2H), 5.59 (s, 2H), 4.27-4.18 (m, 1H), 3.40-3.29 (m, 2H), 3.20 (d, J=10.4 Hz, 1H), 2.90-2.68 (m, 9H), 2.28-2.18 (m, 1H), 1.56 (s, 9H), 1.50 (s, 18H), 1.08 (s, 9H), 0.50 (s, 3H), 0.37 (s, 3H).

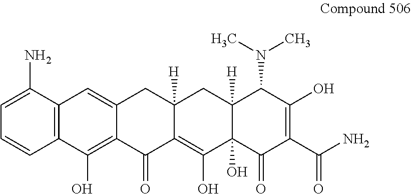

To a solution of S16-4 (29 mg, 0.0296 mmol) in THF (3.0 mL) was added an aqueous HF solution (48-50%, 1.5 mL). The resulting solution was stirred at rt for 16 hrs, the reaction mixture was poured into aqueous solution (40 mL) of K₂HPO₄ (14.4 g) and extracted three times with EtOAc. The combined organic phases were washed with brine, dried, and concentrated to yield the crude intermediate S16-5.

The above crude intermediate was dissolved in THF (2.0 mL) and MeOH (2.0 mL). Pd—C (10 wt %, 5 mg) was added. The reaction flask was briefly evacuated and re-filled with H₂. The reaction mixture was stirred at rt and monitored by LCMS. After the reaction was complete, MeOH (5 mL) and 0.5 N HCl/MeOH (0.5 mL) were added. The mixture was stirred for 30 min, and filtered through a small pad of Celite. The filtrate was concentrated to give the crude product, which was purified by HPLC to give the desired product Compound 506 (4.3 mg, 29%) as a yellow solid: ¹H NMR (400 MHz, CD₃OD): δ8.49 (d, J=8.0 Hz, 1H), 7.76 (d, J=8.0 Hz, 1H), 7.59 (t, J=8.0 Hz, 1H), 7.26 (s, 1H), 4.16 (s, 1H), 3.36-2.91 (m, 9H), 2.78-2.66 (m, 1H), 2.33-2.29 (m, 1H), 1.71-1.61 (m, 1H); MS (ESI) m/z 480.2 (M+H).

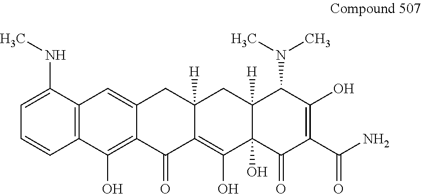

To a solution of S16-4 (70 mg, 0.0714 mmol) in THF (4.0 mL) was added an aqueous HF solution (48-50%, 2.0 mL). The resulting mixture was stirred at rt for 48 h. The mixture was poured into a solution of K₂HPO₄ (12 g) in water (30 mL), and then extracted with EtOAc. The combined organic layers was dried over anhydrous Na₂SO₄, filtered, and then concentrated to give the crude de-Boc and TBS product S16-5 (40 mg), which was not purified and used for the next step directly.

To a solution of S16-5 (20 mg, 0.0353 mmol, 1.0 eq.) and formalin (0.06 mL, 0.74 mmol, 21 eq.) in HCl/MeOH (1 N, 4.0 mL) was added Pd—C (7.0 mg). The resulting suspension was briefly evacuated and re-filled with H₂, and then the mixture was stirred at rt for 3 h. LCMS analysis indicated complete reaction. The catalyst was filtered off and concentrated under reduced pressure to yield the crude product. The crude product was purified by preparative HPLC and get the desired product Compound 507 (4.5 mg, 23%) as a yellow solid: ¹H NMR (400 MHz, CD₃OD): δ8.33 (t, J=8.4 Hz, 1H), 7.65-7.46 (m, 2H), 7.25 (s, 1H), 4.03 (s, 1H), 3.15-2.85 (m, 12H), 2.69-2.56 (m, 1H), 2.20-2.16 (m, 1H), 1.62-1.49 (m, 1H); MS (ESI) m/z 494.2 (M+H).

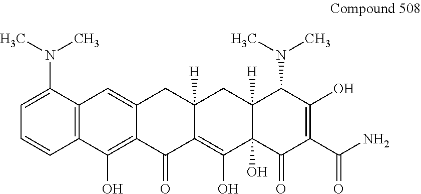

To a solution of S16-5 (30 mg, 0.053 mmol, 1.0 eq.) and formalin (0.10 mL, 1.23 mmol, 23 eq.) in HCl/MeOH (1 N, 5.0 mL) was added Pd—C (10 mg). The resulting suspension was briefly evacuated and re-filled with H₂, and then the mixture was stirred at 40˜50° C. overnight. LCMS analysis indicated complete reaction. The catalyst was filtered off and concentrated in under reduced pressure to yield the crude product. The crude product was purified by preparative HPLC and get the desired product Compound 508 (4.0 mg, 15%) as a yellow solid: ¹H NMR (400 MHz, CD₃OD): δ8.56 (d, J=8.4 Hz, 1H), 8.10 (d, J=8.4 Hz, 1H), 7.68 (t, J=8.4 Hz, 1H), 7.58 (s, 1H), 4.13 (s, 1H), 3.46 (s, 3H), 3.44 (s, 3H), 3.22-2.96 (m, 9H), 2.81-2.73 (m, 1H), 2.30-2.25 (m, 1H), 1.70-1.59 (m, 1H); MS (ESI) m/z 408.0 (M+H).

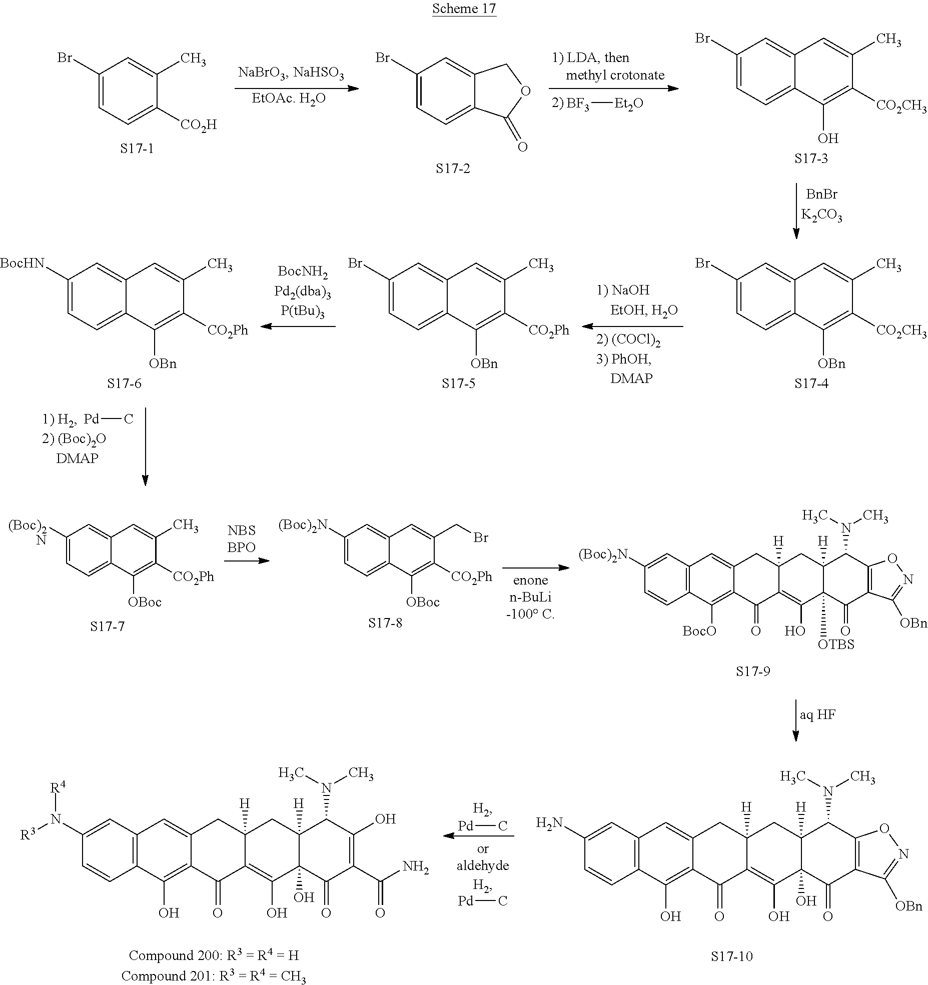

The following compounds were prepared according to Scheme 17.

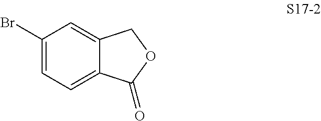

To a combined mixture of S17-1 (80 g, 0.37 mol, 1.0 eq.) in EtOAc (700 mL) and NaBrO₃ (168 g, 1.12 mol, 3.0 eq.) in water (400 mL) was added slowly a solution of NaHSO₄ (134 g, 1.12 mol, 3.0 eq.) in water (900 mL) over a period of 30 min. The resulting mixture was stirred at reflux for 24 h. After cooling down, the organic layer was separated, the aqueous phase was further extracted with EtOAc (500 mL×3). The combined organic layers was washed with saturated aqueous Na₂CO₃ solution (500 mL), dried over anhydrous Na₂SO₄, filtered, and then concentrated to dryness under reduced pressure to give the desired product S17-2 (18 g, 23%) as a white solid: ¹H NMR (400 MHz, CDCl₃): δ 7.77 (d, J=8.4 Hz, 1H), 7.67-7.65 (m, 2H), 5.29 (s, 2H).

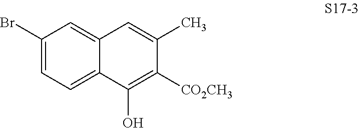

A solution of n-BuLi in hexane (2.5 M, 96 mL, 0.24 mol, 1.2 eq.) was added slowly to a stirred solution of diisopropylamine (33.8 mL, 0.24 mmol, 1.2 eq.) in anhydrous THF (800 mL), which had been cooled to −78° C. using a dry ice/acetone bath. The resulting mixture was stirred at −78° C. for 30 min, and then to this mixture was added drop wise a solution of S17-2 (42.6 g, 0.2 mol, 1.0 eq.) in anhydrous THF (200 mL). After addition, the mixture was allowed to warm to −50° C. over 3 h. Then methyl crotonate (23.4 mL, 0.22 mol, 1.1 eq.) was slowly added and the resulting mixture was gradually warmed up to rt and stirred overnight. The reaction mixture was poured into a dilute aqueous HCl solution (1 N, 150 mL) and extracted with ethyl acetate (50 mL×3), the combined organic layers was dried over anhydrous Na₂SO₄, filtered, and then concentrated.

The above crude product was re-dissolved in methylene chloride (400 mL), and BF₃.Et₂O (5.1 mL, 40 mmol, 0.2 eq.) was added drop wise. The resulting mixture was stirred at rt for 1 h. TLC showed all the starting material consumed, the reaction was quenched with water (200 mL), the organic layer was separated, and the aqueous phase was further extracted with methylene chloride (100 mL×3), the combined organic layers was dried over anhydrous Na₂SO₄, filtered, and then concentrated. The residue was purified with flash chromatography (200˜300 mesh, petroleum ether/EtOAc=50:1˜30:1) to give the desired product S17-3 (12 g, 20%) as a white solid: ¹H NMR (400 MHz, CDCl₃): δ12.67 (s, 1H), 8.14 (d, J=8.8 Hz, 1H), 7.72 (d, J=1.2 Hz, 1H), 7.43 (dd, J=8.8 Hz and 1.6 Hz, 1H), 6.92 (s, 1H), 3.93 (s, 3H), 2.56 (s, 3H).

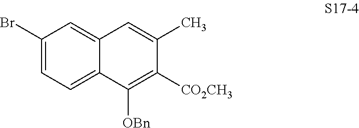

To a stirred solution of S17-3 (10.7 g, 36.3 mmol, 1.0 eq.) in acetone (250 mL) was added powdered K₂CO₃ (10.0 g, 72.5 mmol, 2.0 eq.), followed by BnBr (7.4 g, 43.5 mmol, 1.2 eq.), the resulting suspension was refluxed for 2 h. TLC showed all S17-3 was consumed, the solvent was evaporated under reduced pressure. The residue was purified using flash chromatography (200˜300 mesh, petroleum ether/EtOAc=1:0˜10:1) to give the desired product S17-4 (9.1 g, 65%) as a white solid: ¹H NMR (400 MHz, CDCl₃) δ 7.93-7.91 (m, 2H), 7.50-7.46 (m, 3H), 7.43-7.34 (m, 4H), 5.10 (s, 2H), 3.89 (s, 3H), 2.45 (s, 3H).

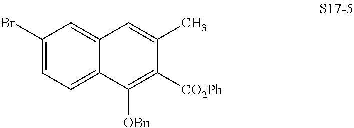

To a solution of S17-4 (12.5 g, 32.4 mmol) in absolute EtOH (50 mL) was added a dilute aqueous NaOH solution (4 N, 50 mL), the resulting mixture was heated at reflux overnight. TLC showed all S17-4 was consumed, the reaction was diluted with water (150 mL) and extracted with EtOAc (50 mL×3), the aqueous phase was acidified with dilute HCl (1 N, about 220 mL) to adjust pH˜6, and then was extracted with EtOAc (50 mL×3), the combined organic layers was dried over anhydrous Na₂SO₄, filtered, and then concentrated under reduced pressure to give the desired product as a white solid, which was not purified and used for the next step directly.

To a solution of above crude product in methylene chloride (250 mL) was added oxalyl chloride (6.13 mL, 65.0 mmol, 2.0 eq.), followed by a couple of drops of DMF (caution: gas evolution). The mixture was stirred at rt for 30 min and the volatiles were evaporated under reduce pressure. The residue was further dried under high vacuum to afford the crude benzoyl chloride. The crude benzoyl chloride was re-dissolved in methylene chloride (200 mL). Pyridine (5.14 g, 65.0 mmol, 2.0 eq.), phenol (3.66 g, 39.0 mmol, 1.2 eq.) and a catalytic amount of DMAP were added. The mixture was stirred at rt for 1 h. The solvent was evaporated. The residue was suspended in EtOAc, and the precipitate was filtered off. The organic solution was then washed with 1 N HCl (three times), H₂O, saturated aqueous NaHCO₃, and brine, dried over Na₂SO₄, filtered and then concentrated. Purification of the residue by flash chromatography (200˜300 mesh, petroleum ether/EtOAc=1:0˜10:1) gave compound S17-5 (1.7 g, 71%) as an off-white solid: ¹H NMR (400 MHz, CDCl₃): δ8.00-7.97 (m, 2H), 7.56-7.51 (m, 3H), 7.44-7.38 (m, 6H), 7.30-7.26 (m, 1H), 7.18-7.15 (m, 2H), 5.23 (s, 2H), 2.64 (s, 3H).

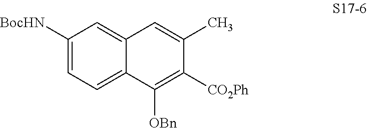

A mixture of S17-5 (3.5 g, 7.5 mmol, 1.0 eq.), BocNH₂ (1.32 g, 11.3 mmol, 1.5 eq.), Cs₂CO₃ (3.68 g, 11.3 mmol, 1.5 eq.) and Pd₂(dba)₃ (213 mg, 0.19 mmol, 0.025 eq.) was degassed for 5 min, to this was added toluene (50 mL), followed by P(tBu)₃ under N₂. The resulting mixture was stirred at 90-100° C. overnight. TLC showed most of S17-5 was consumed, after cooling down, the mixture was washed with water (100 mL) and extracted with EtOAc three times (50 mL×3). The combined organic layers was dried over anhydrous Na₂SO₄, filtered, and concentrated. The residue was purified with flash chromatography (200˜300 mesh, petroleum ether/EtOAc=30:1˜10:1) to give the desired product S17-6 (2.3 g, 64%) as a white solid: ¹H NMR (400 MHz□CDCl₃) δ 8.04 (d, J=9.2 Hz, 1H), 7.99 (s, 1H), 7.53 (dd, JD 8.0 Hz and 1.2 Hz, 2H), 7.44-7.38 (m, 6H), 7.31-7.24 (m, 2H), 7.17 (d, J=7.6 Hz, 2H), 6.67 (s, 1H), 5.23 (s, 2H), 2.61 (s, 3H), 1.56 (s, 9H).

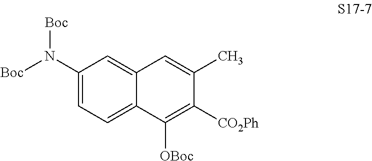

To a stirred solution of S17-6 (2.0 g, 4.13 mmol, 1.0 eq.) in MeOH (15 mL) and EtOAc (3 mL) was added Pd—C (300 mg). The resulting suspension was briefly evacuated and re-filled with H₂, and the mixture was stirred under a H₂ atmosphere at rt for 2 h. TLC showed all S17-6 was consumed, Pd—C was filtered off and the filtrate was concentrated under reduced pressure. The residue was re-dissolved into methylene chloride (20 mL), and to this solution was added Boc₂O (2.5 g, 11.57 mmol, 2.8 eq.) and a catalytic amount of DMAP (50 mg, 0.42 mmol, 0.10 eq.), the resulting mixture was stirred at rt for 1 h. The reaction was quenched with water (50 mL) and extracted with methylene chloride (20 mL×3). The combined organic layers was dried, filtered, and then concentrated. The residue was purified with flash chromatography (200˜300 mesh, petroleum ether/EtOAc=50:1˜30:1) to give the desired product S17-7 (2.2 g, 90%) as a white solid: ¹H NMR (400 MHz, CDCl₃): δ7.91 (d, J=8.8 Hz, 1H), 7.60 (s, 1H), 7.55 (s, 1H), 7.45-7.41 (m, 2H), 7.30-7.26 (m, 4H), 2.65 (s, 3H), 1.44 (s, 9H), 1.39 (s, 18H).

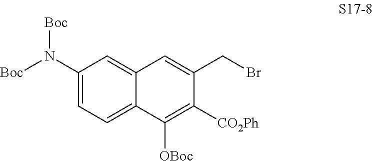

A mixture of S17-7 (2.2 g, 3.81 mmol, 1.0 eq.), NBS (691 mg, 3.88 mmol, 1.02 eq.) and BPO (1.84 g, 7.60 mmol, 2.0 eq.) in CCl₄ (20 mL) was refluxed for 5 h. The reaction was quenched with water (50 mL) and extracted with methylene chloride (20 mL×3). The combined organic layers was dried, filtered, and then concentrated. The residue was purified with flash chromatography (200˜300 mesh, petroleum ether/EtOAc=50:1˜30:1) to give the desired product S17-8 (1.9 g, 74%) as a white solid: ¹H NMR (400 MHz, CDCl₃): δ7.99 (d, J=8.8 Hz, 1H), 7.78 (s, 1H), 7.63 (d, J=2.0 Hz, 1H), 7.47-7.41 (m, 2H), 7.40-7.33 (m, 2H), 7.31-7.26 (m, 2H), 4.93 (s, 2H), 1.45 (s, 9H), 1.40 (s, 18H).

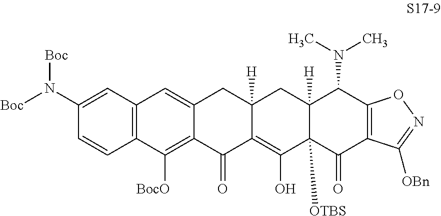

A solution of n-BuLi in hexane (2.5 M, 0.15 mL, 0.375 mmol, 3.6 eq.) was added drop wise to a solution of S17-8 (250 mg, 0.373 mmol, 3.6 eq.) and enone (50 mg, 0.104 mmol, 1.0 eq.) in dry THF (5.0 mL), which had been cooled to −100° C. using a liquid N₂/EtOH bath. After addition, the resulting mixture was allowed to warm to 0° C. gradually over 1 h. The reaction was then quenched with brine (20 mL) and extracted with EtOAc (10 mL×3). The combined organic layers was dried, filtered and then concentrated. The residue was purified with flash chromatography (200˜300 mesh, petroleum ether/EtOAc=40:1˜20:1) to give the desired product S17-9 (20 mg, 20%) as a yellow gum: ¹H NMR (400 MHz, CDCl₃): δ7.97 (d, J=9.2 Hz, 1H), 7.41 (s, 1H), 7.38-7.36 (m, 3H), 7.26-7.12 (m, 4H), 5.23 (s, 2H), 3.84 (d, J=14.4 Hz, 1H), 2.99-2.95 (m, 2H), 3.01-2.92 (m, 1H), 2.86-2.82 (m, 1H), 2.46-2.30 (m, 9H), 2.05-1.98 (m, 1H), 1.44 (s, 9H), 1.29 (s, 18H), 0.70 (s, 9H), 0.14 (s, 3H), 0.08 (s, 3H).

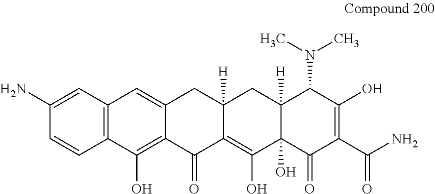

To a solution of S17-9 (18 mg, 0.0183 mmol) in CH₃CN (3.0 mL) was added an aqueous HF solution (48-50%, 1.5 mL). The resulting solution was stirred at rt for 16 hrs, the reaction mixture was poured into aqueous solution (10 mL) of K₂HPO₄ (9.0 g) and extracted with EtOAc three times (10 mL×3). The combined organic phases was dried, filtered and then concentrated to yield the crude intermediate S17-10.

The above crude intermediate was dissolved in HCl/MeOH (1 N, 3.0 mL). Pd—C (10 wt %, 4 mg) was added. The reaction flask was briefly evacuated and re-filled with H₂. The reaction mixture was stirred at rt for 1.5 h. LCMS showed the complete conversion of S17-9, the mixture was filtered through a small pad of Celite. The filtrate was concentrated to give the crude product, which was purified by HPLC to give the desired product Compound 200 (3.0 mg, 36%) as a yellow solid: ¹H NMR (400 MHz, CD₃OD): δ8.42 (d, J=8.8 Hz, 1H), 7.61 (s, 1H), 7.37 (d, J=8.8 Hz, 1H), 7.14 (s, 1H), 4.11 (s, 1H), 3.18-2.95 (m, 9H), 2.68-2.61 (m, 1H), 2.27-2.20 (m, 1H), 1.69-1.57 (m, 1H); MS (ESI) m/z 480.2 (M+H).

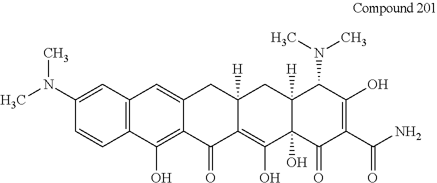

To a solution of S17-9 (45 mg, 0.0459 mmol) in THF (7.0 mL) was added an aqueous HF solution (48-50%, 3.5 mL). The resulting mixture was stirred at rt for 48 h. The mixture was poured into an aqueous solution (20 mL) of K₂HPO₄ (19 g), and then extracted with EtOAc (10 mL×3). The combined organic layers was dried over anhydrous Na₂SO₄, filtered, and then concentrated under reduced pressure to give the crude intermediate.

The above crude intermediate was dissolved in HCl/MeOH (1 N, 6.0 mL), and formalin (0.10 mL, 1.23 mmol, 35 eq.) was added, followed by Pd—C (20 mg). The resulting suspension was briefly evacuated and re-filled with H₂, and then the mixture was stirred at rt for 30 min. LCMS analysis indicated complete reaction, the catalyst was filtered off and the filtrate was concentrated in under reduced pressure to yield the crude product. The crude product was purified by HPLC to yield the desired product Compound 201 (6.0 mg, 34%) as a yellow solid: ¹H NMR (400 MHz, CD₃OD): δ8.35 (d, J=9.2 Hz, 1H), 7.40 (d, J=9.2 Hz, 1H), 7.06 (s, 1H), 4.08 (s, 1H), 3.26 (s, 6H), 3.15-2.85 (m, 9H), 2.65-2.58 (m, 1H), 2.24-2.18 (m, 1H), 1.68-1.54 (m, 1H); MS (ESI) m/z 508.1 (M+H).

### Example 1

The antibacterial activities for the compounds of the invention were studied according to the following protocols.

### Minimum Inhibitory Concentration Assay

Frozen bacterial strains were thawed and subcultured onto Mueller Hinton Broth (MHB) or other appropriate media (Streptococcus requires blood and Haemophilus requires hemin and NAD). Following incubation overnight, the strains were subcultured onto Mueller Hinton Agar and again incubated overnight. Colonies were observed for appropriate colony morphology and lack of contamination. Isolated colonies were selected to prepare a starting inoculum equivalent to a 0.5 McFarland standard. The starting inoculum was diluted 1:125 using MHB for further use. Test compounds were prepared by dilution in sterile water to a final concentration of 5.128 mg/mL. Antibiotics (stored frozen, thawed and used within 3 hours of thawing) and compounds were further diluted to the desired working concentrations.

The assays were run as follows. Fifty μL of MHB was added to wells 2-12 of a 96-well plate. One hundred μL of appropriately diluted antibiotics was added to well 1. Fifty μL of antibiotics was removed from well 1 and added to well 2 and the contents of well 2 mixed by pipetting up and down five times. Fifty μL of the mixture in well 2 was removed and added to well 3 and mixed as above. Serial dilutions were continued in the same manner through well 12. Fifty μL was removed from well 12 so that all contained 50 μL. Fifty μL of the working inoculum was then added to all test wells. A growth control well was prepared by adding 50 μL of working inoculum and 50 μL of MHB to an empty well. The plates were then incubated at 37° C. overnight, removed from the incubator and each well was read on a plate reading mirror. The lowest concentration (MIC) of test compound that inhibited the growth of the bacteria was recorded.

### Example

| | 1 | 2 | 3 | 4 | 5 | 6 | 7 | 8 | 9 | 10 | 11 | 12 |
|---|---|---|---|---|---|---|---|---|---|---|---|---|
| [Abt] | 32 | 16 | 8 | 4 | 2 | 1 | 0.5 | 0.25 | 0.125 | 0.06 | 0.03 | 0.015 |
| grow | − | − | − | − | − | + | + | + | + | + | + | + |

[abt] = antibiotic concentration in the well
Grow = bacterial growth (cloudiness)
Interpretation: MIC = 2 μg/mL

### Protocol for Determining Inoculum Concentration (Viable Count)

Ninety μl of sterile 0.9% NaCl was pipetted into wells 2-6 of a 96-well microtiter plate. Fifty 50 μl of the inoculum was pipetted into well 1. Ten μL from was removed from well 1 and added it to well 2 followed by mixing. Ten μL was removed from well two and mixed with the contents of well 3 and so on creating serial dilutions through well 6. Ten μL was removed from each well and spotted onto an appropriate agar plate. The plate was placed into a CO₂ incubator overnight. The colonies in spots that contain distinct colonies were counted. Viable count was calculated by multiplying the number of colonies by the dilution factor.

Spot from Well

| | 1 | 2 | 3 | 4 | 5 | 6 |
|---|---|---|---|---|---|---|
| Dilution | 10² | 10³ | 10⁴ | 10⁵ | 10⁶ | 10⁷ |
| Factor | | | | | | |

### Bacterial Strains

Fifteen bacterial strains, listed below, were examined in minimum inhibitory concentration (MIC) assays.

| ID | Organism | Source | Resistance | Comments | Gram Rx |
|---|---|---|---|---|---|
| SA100 | S. aureus | ATCC 13709 | MSSA | Smith strain | positive |
| | | | | (animal | |
| | | | | model) | |
| SA101 | S. aureus | ATCC 29213 | MSSA | control | positive |
| SA158 | S. aureus | MR, SK75 | tet resistant: tetK | | positive |
| | | | (efflux) | | |
| SA161 | S. aureus | Micromyx, | tet resistant: tet(M) | | positive |
| | | LLC | ribosomal protection | | |
| EF103 | E. faecalis | ATCC 29212 | tet intermediate/ | control | positive |
| | | | resistant - mechanism | | |
| | | | not specified | | |
| EF159 | E. faecalis | MR, DS160 | tet resistant: tetM | cip-R, ery-I | positive |
| | | | (rib protect) | | |
| SP106 | S. pneumoniae | ATCC 49619 | wt | control | positive |
| SP160 | S. pneumoniae | MR, 54 | tet resistant: tet M | pen-R, ery-R | positive |
| | | | (rib protect) | | |
| EC107 | E. coli | ATCC 25922 | wt | control | negative |
| EC155 | E. coli | MR, 10 | tet resistant: tetA | | negative |
| | | | (efflux) | | |
| KP109 | K. pneumoniae | ATCC 13883 | wt | | negative |
| KP153 | K. pneumoniae | MR, 1 | tet resistant: tetA | cip-R, gen-R | negative |
| | | | (efflux) | | |
| EC108 | E. cloacae | ATCC 13047 | wt | | negative |
| AB110 | A. baumanii | ATCC 19606 | wt | | negative |
| PA111 | P. aeruginosa | ATCC 27853 | wt | control | negative |

MSSA = methicillin susceptible S. aureus
wt = wild type
ATCC = American Type Culture Collection
MR = Marilyn Roberts, University of Washington
tet = tetracycline
cip = ciprofloxacin
R = resistant
gen = gentamicin
ery = erythromycin
pen = penicillin

### Results

Values of minimum inhibition concentration (MIC) for the compounds of the invention are provided in Table 7.

TABLE 7
MIC Values for Compounds of the Invention Compared to Sancycline,
Minocycline and Tigecycline.

| | | | SA161 | | | | | |
|---|---|---|---|---|---|---|---|---|
| | SA101 | SA100 | MRSA, | SA158 | EF103 | EF159 | SP106 | SP160 |
| Cmpd | 29213 | 13709 | tetM | tetK | 29212 | tetM | 49619 | tetM |
| 100 | C | B | B | B | B | C | B | B |
| 101 | B | B | B | B | B | B | B | B |
| 102 | C | B | B | B | B | B | B | B |
| 103 | B | B | B | B | B | B | B | B |
| 104 | B | B | B | B | B | B | B | B |
| 105 | B | B | B | B | B | B | B | B |
| 106 | C | B | B | B | B | B | B | B |
| 107 | C | B | B | B | B | B | C | B |
| 108 | C | C | B | B | B | B | B | B |
| 109 | C | B | B | B | B | B | C | B |
| 110 | C | C | B | B | B | B | C | C |
| 111 | C | B | B | B | B | B | B | B |
| 112 | C | B | B | B | B | B | B | B |
| 113 | C | C | B | B | B | B | B | B |
| 114 | C | B | B | B | B | B | B | B |
| 115 | B | B | B | B | B | B | B | B |
| 116 | C | C | C | C | C | C | C | C |
| 117 | A | B | B | B | B | B | A | A |
| 118 | C | B | B | B | B | B | C | B |
| 119 | B | B | B | B | B | B | A | B |
| 120 | C | B | B | B | B | B | C | B |
| 121 | B | B | B | B | B | B | A | B |
| 122 | C | B | B | B | B | B | B | B |
| 123 | B | C | B | B | B | B | B | B |
| 124 | B | B | B | B | B | B | B | B |
| 125 | B | B | B | B | B | B | A | B |
| 126 | C | B | B | B | B | B | B | B |
| 127 | C | C | B | B | B | B | C | B |
| 128 | C | B | B | B | B | B | C | B |
| 129 | C | C | B | B | B | B | C | B |
| 130 | C | C | B | B | B | B | B | B |
| 131 | B | B | B | B | B | B | B | B |
| 132 | B | B | B | B | B | B | B | B |
| 133 | C | C | B | B | B | B | C | B |
| 134 | C | C | B | B | B | B | B | B |
| 135 | B | B | B | B | B | B | B | B |
| 136 | B | B | B | B | B | B | B | B |
| 137 | B | B | B | B | B | B | B | B |
| 138 | C | C | B | B | B | B | A | B |
| 139 | C | B | B | B | B | B | B | B |
| 140 | B | B | B | B | B | B | A | B |
| 141 | C | C | B | B | B | B | B | B |
| 142 | C | B | B | B | B | B | B | B |
| 143 | B | B | B | B | B | B | B | B |
| 144 | B | B | B | B | B | B | A | B |
| 145 | B | B | B | B | B | B | B | B |
| 146 | B | B | B | B | B | B | A | B |
| 147 | C | C | B | B | B | B | C | B |
| 200 | C | C | B | B | B | B | C | B |
| 201 | C | C | B | B | B | B | C | C |
| 300 | B | B | B | B | B | B | B | B |
| 301 | B | B | B | B | B | B | B | B |
| 302 | C | C | B | B | B | B | C | B |
| 303 | B | B | B | B | B | B | C | B |
| 304 | B | B | B | B | B | B | C | B |
| 305 | B | B | B | B | B | B | C | B |
| 306 | B | B | B | B | B | B | B | B |
| 307 | C | B | B | B | B | B | B | B |
| 400 | B | B | B | B | B | B | B | B |
| 401 | B | B | B | B | B | B | A | B |
| 402 | B | B | B | B | B | B | C | B |
| 403 | C | B | B | B | B | B | A | B |
| 404 | B | B | B | B | B | B | B | B |
| 405 | B | B | B | B | B | B | B | B |
| 406 | C | C | B | B | B | B | C | B |
| 407 | C | C | B | B | B | B | C | B |
| 408 | B | B | B | B | B | B | A | B |
| 409 | B | B | B | B | B | B | A | B |
| 410 | B | B | B | B | B | B | A | B |
| 411 | B | B | B | B | B | B | A | B |
| 412 | C | B | B | B | B | B | B | B |
| 413 | C | C | B | B | B | B | C | B |
| 414 | B | B | B | B | B | B | A | B |
| 415 | B | B | B | B | B | B | B | B |
| 416 | B | B | B | B | B | B | B | B |
| 417 | B | B | B | B | B | B | A | B |
| 418 | B | B | B | A | B | B | A | B |
| 419 | B | B | B | B | B | B | B | B |
| 420 | B | B | B | B | B | B | B | B |
| 421 | B | B | B | B | B | B | B | B |
| 422 | B | B | B | B | B | B | B | B |
| 423 | B | B | B | B | B | B | B | B |
| 424 | B | B | B | B | B | B | B | B |
| 425 | B | B | B | B | B | B | B | B |
| 426 | B | B | B | B | B | B | B | B |
| 427 | C | B | B | B | B | B | C | B |
| 428 | C | C | C | C | B | C | C | B |
| 429 | C | B | B | B | B | B | B | B |
| 430 | B | B | B | B | B | B | B | B |
| 431 | B | B | NT | B | B | B | B | B |
| 432 | C | C | NT | B | B | B | C | B |
| 433 | C | C | B | B | B | B | B | B |
| 461 | C | C | C | C | C | C | C | C |
| 462 | C | C | C | C | B | C | C | B |
| 463 | B | B | B | B | B | B | C | B |
| 464 | C | C | B | B | B | B | C | C |
| 465 | B | B | B | B | B | B | C | B |
| 466 | B | B | B | B | B | B | C | B |
| 500 | C | C | C | B | B | B | C | B |
| 501 | C | C | B | B | B | B | B | B |
| 502 | C | B | C | B | B | C | B | B |
| 503 | C | C | C | B | B | C | B | B |
| 504 | C | C | B | B | B | C | B | B |
| 505 | C | C | C | B | B | C | C | B |
| 506 | C | C | B | B | B | B | C | B |
| 507 | C | C | B | B | B | B | C | C |
| 508 | C | B | B | B | B | B | C | B |
| 509 | B | B | B | B | B | B | C | B |
| 600 | C | C | B | B | B | B | C | C |
| 601 | C | B | B | B | B | B | C | C |
| 602 | C | B | B | B | B | B | C | C |
| 603 | C | C | C | C | C | B | C | C |
| 604 | C | C | B | C | B | B | C | C |
| 605 | C | C | C | C | C | C | C | C |
| 606 | C | C | C | C | C | C | C | C |
| 607 | C | C | C | C | C | C | C | C |
| 608 | C | C | C | C | C | C | C | C |
| San. | 0.5 | 1 | NT | 4 | 8 | 8 | 0.25 | 8 |
| Mino. | 0.06 | 0.06 | 8 | 0.03 | 1 | 16 | <0.015 | 2 |
| Tige. | 0.06 | 0.06 | 0.125 | 0.06 | 0.03 | 0.06 | 0.0156 | 0.0156 |

| | EC107 | EC155 | AB110 | PA111 | EC108 | KP109 | KP153 |
|---|---|---|---|---|---|---|---|
| Cmpd | 25922 | tetA | 19606 | 27853 | 13047 | 13883 | teta |
| 100 | B | B | C | C | B | B | B |
| 101 | B | B | C | C | B | B | B |
| 102 | B | B | C | C | B | B | B |
| 103 | B | B | C | C | B | B | B |
| 104 | B | B | C | C | B | B | B |
| 105 | B | B | C | C | B | B | B |
| 106 | B | C | C | C | B | B | B |
| 107 | C | C | C | C | C | C | C |
| 108 | B | B | C | C | B | B | B |
| 109 | C | C | C | C | C | C | C |
| 110 | C | C | C | C | C | C | C |
| 111 | B | B | C | C | B | B | B |
| 112 | B | B | C | C | B | B | B |
| 113 | B | B | C | C | C | C | B |
| 114 | B | B | C | C | B | B | B |
| 115 | B | B | C | C | B | B | B |
| 116 | C | C | C | C | C | C | C |
| 117 | B | B | C | B | B | B | B |
| 118 | C | C | C | C | C | C | C |
| 119 | B | B | C | B | B | B | B |
| 120 | B | B | C | C | C | C | B |
| 121 | B | B | C | C | B | B | B |
| 122 | B | B | C | C | B | B | B |
| 123 | B | B | B | C | B | B | B |
| 124 | B | B | C | C | B | B | B |
| 125 | B | B | C | C | B | B | B |
| 126 | B | B | C | C | C | B | B |
| 127 | C | C | C | C | C | C | C |
| 128 | C | C | C | C | C | C | C |
| 129 | C | C | C | C | C | C | C |
| 130 | B | B | C | C | C | C | B |
| 131 | B | B | C | C | B | B | B |
| 132 | B | B | C | C | B | B | B |
| 133 | B | B | C | C | C | B | B |
| 134 | B | B | C | C | C | C | B |
| 135 | B | B | C | C | B | B | B |
| 136 | B | B | C | C | B | B | B |
| 137 | B | B | C | C | B | B | B |
| 138 | B | B | C | C | B | B | B |
| 139 | B | B | C | C | B | B | B |
| 140 | B | B | C | C | B | B | B |
| 141 | B | B | C | C | B | C | B |
| 142 | B | B | C | C | B | B | B |
| 143 | B | B | C | B | B | B | B |
| 144 | B | B | C | C | B | B | B |
| 145 | B | B | C | C | B | B | B |
| 146 | B | B | C | C | B | B | B |
| 147 | C | C | C | C | C | C | C |
| 200 | C | C | C | C | C | C | C |
| 201 | C | C | C | C | C | C | C |
| 300 | B | B | C | C | B | B | B |
| 301 | B | B | C | C | C | C | B |
| 302 | C | C | C | C | C | C | C |
| 303 | B | B | C | C | C | B | B |
| 304 | B | B | C | C | C | B | B |
| 305 | B | B | C | C | C | B | B |
| 306 | B | B | C | C | C | C | C |
| 307 | B | B | C | C | C | B | C |
| 400 | B | B | C | B | B | B | B |
| 401 | B | B | B | C | B | B | B |
| 402 | B | C | C | C | C | C | C |
| 403 | B | B | C | B | B | B | B |
| 404 | B | B | B | C | B | B | B |
| 405 | B | B | B | C | B | B | B |
| 406 | B | B | C | C | B | B | B |
| 407 | B | B | C | C | C | B | B |
| 408 | B | B | C | C | B | B | B |
| 409 | B | B | C | C | B | B | B |
| 410 | B | B | B | C | B | B | B |
| 411 | B | B | B | B | B | B | B |
| 412 | B | B | C | C | B | B | B |
| 413 | B | B | C | C | C | B | B |
| 414 | B | B | C | B | B | B | B |
| 415 | B | B | B | C | B | B | B |
| 416 | B | B | B | C | B | B | B |
| 417 | B | B | C | B | B | B | B |
| 418 | B | B | B | B | B | B | B |
| 419 | B | B | C | C | B | B | B |
| 420 | B | B | C | C | B | B | B |
| 421 | B | B | C | C | B | B | B |
| 422 | B | B | C | C | B | B | B |
| 423 | B | B | C | C | B | B | B |
| 424 | B | B | C | C | B | B | B |
| 425 | B | B | C | C | B | B | B |
| 426 | B | B | B | C | B | B | B |
| 427 | C | B | C | C | C | C | B |
| 428 | C | C | C | C | C | C | C |
| 429 | B | B | C | C | B | B | B |
| 430 | B | B | C | C | B | B | B |
| 431 | C | C | C | C | C | C | C |
| 432 | C | C | C | C | C | C | C |
| 433 | B | C | C | C | C | C | C |
| 461 | C | C | C | C | C | C | C |
| 462 | C | C | C | C | C | C | C |
| 463 | C | C | C | C | C | C | C |
| 464 | C | C | C | C | C | C | C |
| 465 | C | B | C | C | C | C | C |
| 466 | C | C | C | C | C | C | C |
| 500 | C | C | C | C | C | C | C |
| 501 | B | C | C | C | C | C | C |
| 502 | B | C | C | C | C | C | C |
| 503 | B | C | C | C | C | C | C |
| 504 | B | C | C | C | C | C | C |
| 505 | C | C | C | C | C | C | C |
| 506 | C | C | C | C | C | C | C |
| 507 | C | C | C | C | C | C | C |
| 508 | C | C | C | C | C | C | C |
| 509 | C | C | C | C | C | C | C |
| 600 | C | C | C | C | C | C | C |
| 601 | C | C | C | C | C | C | C |
| 602 | C | C | C | C | C | C | C |
| 603 | C | C | C | C | C | C | C |
| 604 | C | C | C | C | C | C | C |
| 605 | C | C | C | C | C | C | C |
| 606 | C | C | C | C | C | C | C |
| 607 | C | C | C | C | C | C | C |
| 608 | C | C | C | C | C | C | C |
| San. | 8 | 32 | 0.25 | >32 | 8 | 8 | 32 |
| Mino. | 0.5 | 8 | 0.06 | 16 | 2 | 1 | 8 |
| Tige. | 0.03 | 0.5 | 0.25 | 8 | 0.25 | 0.125 | 1 |

A = lower than or equal to lowest MIC among three control compounds;
B = greater than lowest MIC among three control compounds, but lower than highest MIC among three control compounds;
C = greater than MIC of all three control compounds.

While this invention has been particularly shown and described with references to example embodiments thereof, it will be understood by those skilled in the art that various changes in form and details may be made therein without departing from the scope of the invention encompassed by the appended claims.

1. A compound represented by the following structural formula: 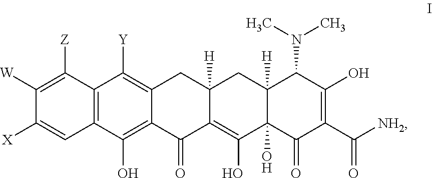
or a pharmaceutically acceptable salt thereof, wherein:
X is selected from hydrogen, fluoro, —CH₂OH, —CH₂—N(R¹)(R²), phenyl and pyridyl;
R¹ is selected from hydrogen, (C₁-C₈)alkyl, (C₀-C₆)alkylene-(C₁-C₆)alkoxy, (C₀-C₆)alkylene-(C₃-C₇)cycloalkyl, —C(O)—(C₁-C₆)alkyl, —C(O)—(C₁-C₆)alkoxy, phenyl, benzyl, pyridyl, —C(O)—N(R²)(R²), and —S(O)ₘ—(C₁-C₆)alkyl, wherein m is 1 or 2;
each R² is independently selected from hydrogen, and (C₁-C₄)alkyl; or
R¹ and R² are taken together with the nitrogen atom to which they are bound to form a (4-7 membered) heterocyclic ring optionally containing one additional heteroatom selected from S, O or N,
each (C₁-C₈)alkyl, (C₀-C₆)alkylene, (C₁-C₆)alkoxy and (C₃-C₇)cycloalkyl in the group represented by R¹ and R² and the ring formed by R¹ and R² are optionally and independently substituted with up to three substituents independently selected from halo, methyl, —CF₃, —OCH₃, —N(CH₃)₂, —NH—C(O)CH₃, —C(O)NH₂, —C(O)OCH₃, —CN or —OH;
Y is selected from hydrogen, fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy, —N(R³)(R⁴) and —O—C(O)—N(R³)(R⁴);
each of R³ and R⁴ is independently selected from hydrogen, —(C₁-C₆)alkyl, —(C₃-C₇)cycloalkyl; or
R³ and R⁴ are taken together with the nitrogen atom to which they are bound to form a (4-7 membered) heterocyclic ring optionally containing one additional heteroatom selected from S, O or N;
each (C₁-C₆)alkyl or (C₃-C₇)cycloalkyl the group represented by R³ and R⁴ and the (4-7 membered) heterocyclic ring formed by R³ and R⁴ are each optionally and independently substituted with halo or —OH;
W is selected from hydrogen, (C₁-C₆)alkoxy, and —N(R³)(R⁴); and
Z is selected from hydrogen, (C₁-C₆)alkoxy, and —N(R³)(R⁴);
wherein each phenyl, benzyl and pyridyl in the group represented by X and R¹ is optionally substituted with halo, unsubstituted C₁-C₆ alkyl, halo(C₁-C₆)alkyl, unsubstituted C₁-C₆ alkoxy, halo(C₁-C₆)alkoxy, cyano, amino, —NH—C(O)—(C₁-C₆)alkyl or nitro.
wherein:
when W, Y and Z are hydrogen, X is not hydrogen, —CH₂NHC(CH₃)₃, —CH₂NH-cyclopropyl, CH₂-morpholin-4-yl, CH₂-4-methylpiperazin-1-yl, CH₂-4-acetylpiperazin-1-yl, CH₂N(CH₃)C(O)CH₃, CH₂-1H-imidazol-1-yl; and
when W and Z are hydrogen and Y is —N(CH₃)₂, X is not —CH₂NHC(CH₃)₃, —CH₂—NH-(1-methylcyclopropyl), —CH₂—NH-(1-methylcyclopentyl), —CH₂—NH-cyclopropyl, —CH₂—NH-cyclopentyl, —CH₂—NH-cyclohexyl, —CH₂NHCH₂C(CH₃)₃, —CH₂-azetidin-1-yl.

2. The compound of claim 1, wherein one of W or Z is not hydrogen.

3. The compound of claim 1, wherein X is —CH₂—NH(R¹)(R²).

4. The compound of claim 3, wherein Y is −N(R³)(R⁴).

5. The compound of claim 3, wherein Y is hydrogen or —N(CH₃)₂.

6. The compound of claim 1 wherein the compound is represented by the following structural formula: 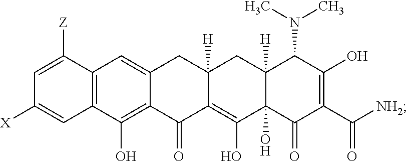
or a pharmaceutically acceptable salt thereof.

7. The compound of claim 1 wherein the compound is represented by the following structural formula: 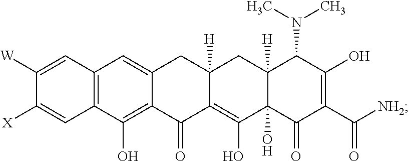
or a pharmaceutically acceptable salt thereof.

8. The compound of claim 6, wherein X is hydrogen.

9. The compound of claim 1, wherein Y is selected from fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy and —O—C(O)—N(R³)(R⁴).

10. The compound of claim 1, wherein the compound is represented by the following structural formula: 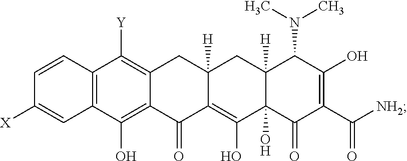
or a pharmaceutically acceptable salt thereof, wherein Y is selected from fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy and —O—C(O)—N(R³)(R⁴).

11. The compound of claim 10, wherein X is —CH₂N(R¹)R²).

12. The compound of claim 11, wherein Y is selected from (C₁-C₆)alkoxy, —O—(C₃-C₇)cycloalkyl, —O—(C₁-C₄)alkylene-N(R³)(R⁴), —O—(C₁-C₆)alkylene-(C₁-C₆)alkoxy and —O—C(O)—N(R³)(R⁴).

13. The compound of claim 11, wherein Y is selected from fluoro, chloro and —NO₂.

14. The compound of claim 1, wherein:
X is selected from hydrogen, fluoro, —CH₂OH, phenyl and pyridyl,
each phenyl and pyridyl in the group represented by X is optionally substituted with halo, unsubstituted C₁-C₆ alkyl, halo(C₁-C₆)alkyl, unsubstituted C₁-C₆ alkoxy, halo(C₁-C₆)alkoxy, cyano, amino, —NH—C(O)—(C₁-C₆)alkyl or nitro; and
Y is selected from hydrogen, fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy, —N(R³)(R⁴) and —O—C(O)—N(R³)(R⁴).

15. The compound of claim 1, wherein the compound is represented by the following structural formula: 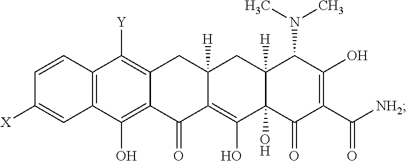
or a pharmaceutically acceptable salt thereof, wherein:
X is selected from hydrogen, fluoro, —CH₂OH, phenyl and pyridyl,
each phenyl and pyridyl in the group represented by X is optionally substituted with halo, unsubstituted C₁-C₆ alkyl, halo(C₁-C₆)alkyl, unsubstituted C₁-C₆ alkoxy, halo(C₁-C₆)alkoxy, cyano, amino, —NH—C(O)—(C₁-C₆)alkyl or nitro; and
Y is selected from hydrogen, fluoro, chloro, —NO₂, —OH, (C₁-C₆)alkoxy, (C₃-C₇)cycloalkoxy, —O—(C₁-C₆)alkylene-N(R³)(R⁴), —O—(C₁-C₆)alkylene-(C₁-C₆)alkoxy, —N(R³)(R⁴) and —O—C(O)—N(R³)(R⁴).

16. The compound of claim 2 wherein:
X is selected from phenyl and pyridyl, wherein phenyl and pyridyl in the group represented by X are optionally substituted with up to two substituents independently selected from halo, methyl, —CF₃, —OCH₃, —N(CH₃)₂, or —NH—C(O)CH₃; and
Y is hydrogen.

17. The compound of claim 1, wherein X is fluoro.

18. The compound of claim 2, wherein:
X is —CH₂—N(R¹)(R²); and
R¹ is selected from (C₀-C₆)alkylene-(C₁-C₆)alkoxy, —(C₁-C₆)alkylene-(C₃-C₇)cycloalkyl, —C(O)—(C₁-C₆)alkoxy, phenyl, benzyl, pyridyl, —C(O)—N(R²)(R²), and —S(O)ₘ—(C₁-C₆)alkyl, wherein m is 1 or 2,
wherein each phenyl, benzyl and pyridyl in the group represented R¹ is optionally substituted with halo, unsubstituted C₁-C₆ alkyl, halo(C₁-C₆)alkyl, unsubstituted C₁-C₆ alkoxy, halo(C₁-C₆)alkoxy, cyano, amino, —NH—C(O)—(C₁-C₆)alkyl or nitro; and
wherein each (C₁-C₈)alkyl, (C₀-C₆)alkylene, (C₁-C₆)alkoxy and (C₃-C₇)cycloalkyl in the group represented by R¹ is optionally substituted with halo, methyl, —CF₃, —OCH₃, —N(CH₃)₂, —NH—C(O)CH₃, —C(O)NH₂, —C(O)OCH₃, —CN or —OH.

19. The compound of claim 1, wherein the compound is represented by the following structural formula: 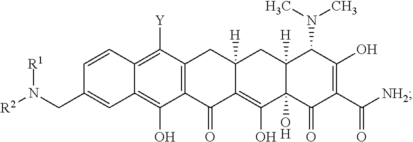
or a pharmaceutically acceptable salt thereof, wherein:
R¹ is selected from (C₀-C₆)alkylene-(C₁-C₆)alkoxy, —(C₁-C₆)alkylene-(C₃-C₇)cycloalkyl, —C(O)—(C₁-C₆)alkoxy, phenyl, benzyl, pyridyl, —C(O)—N(R²)(R²), and —S(O)ₘ—(C₁-C₆)alkyl, wherein m is 1 or 2,
wherein each phenyl, benzyl and pyridyl in the group represented R¹ is optionally substituted with halo, unsubstituted C₁-C₆ alkyl, halo(C₁-C₆)alkyl, unsubstituted C₁-C₆alkoxy, halo(C₁-C₆)alkoxy, cyano, amino, —NH—C(O)—(C₁-C₆)alkyl or nitro; and
wherein each (C₁-C₈)alkyl, (C₀-C₆)alkylene, (C₁-C₆)alkoxy and (C₃-C₇)cycloalkyl in the group represented by R¹ is optionally substituted halo, methyl, —CF₃, —OCH₃, —N(CH₃)₂, —NH—C(O)CH₃, —C(O)NH₂, —C(O)OCH₃, —CN or —OH.

20. A compound selected from any one of Compounds 117, 119, 121, 125, 138, 140, 144, 146, 401, 403, 408, 409, 410, 411, 414, 417, and 418 or a pharmaceutically acceptable salt thereof.

21. A compound selected from any one of Compounds 117, 119, 121, 125, 138, 140, 144, 401, 403, 408, 409, 410, 411, 414, 417, and 418 or a pharmaceutically acceptable salt thereof.

22. A pharmaceutical composition comprising a pharmaceutically acceptable carrier or diluent and a compound of claim 1.

23. A method for treating an infection in a subject comprising administering to the subject an effective amount of the compound of claim 1 or a pharmaceutically acceptable salt thereof.

24. The method of claim 23, wherein the infection is caused by a Gram-positive organism.

25. The method of claim 23, wherein the infection is caused by a Gram-negative organism.

26. The method of claim 23, wherein the infection is caused by an organism selected from the group consisting of rickettsiae, chlamydiae, and Mycoplasma pneumoniae.

27. The method of claim 23, wherein the infection is caused by an organism resistant to tetracycline.

28. The method of claim 23, wherein the infection is caused by an organism resistant to methicillin.

29. The method of claim 23, wherein the infection is caused by an organism resistant to vancomycin.
